(19) United States
(12) Patent Application Publication
(10) Pub. No.: US 2024/0366799 A1
(43) Pub. Date: Nov. 7, 2024

(54) CAGING-GROUP-FREE PHOTOACTIVATABLE FLUORESCENT DYES AND THEIR USE

(71) Applicant: Max-Planck-Gesellschaft zur Foerderung der Wissenschaften e. V., Muenchen (DE)

(72) Inventors: Richard LINCOLN, Heidelberg (DE); Alexey N. BUTKEVICH, Heidelberg (DE); Mariano L. BOSSI, Heidelberg (DE)

(21) Appl. No.: 18/579,446

(22) PCT Filed: Jul. 15, 2021
(86) PCT No.: PCT/EP2021/069804

§ 371 Date: Jan. 15, 2024

(51) Int. Cl. A61K 49/00; G01N 33/58

(52) U.S. Cl. CPC A61K 49/0041; A61K 49/0021; A61K 49/0026; A61K 49/0052; A61K 49/0056; A61K 49/0058; G01N 33/582

(57) ABSTRACT

The invention relates to novel caging-group-free photactivatable fluorescent dyes having the structural formula (I) as well as to the corresponding photoactivated fluorescent dyes having the structural formula (II). The invention further relates to the use of the photoactivatable compounds as such or after photoactivation, in particular as fluorescent tags, analytical reagents and labels in optical microscopy, imaging techniques, protein tracking, nucleic acid labeling, glycan analysis, capillary electrophoresis, flow cytometry or as a component of biosensors, or as analytical tools or reporters in microfluidic devices or nanofluidic circuitry.

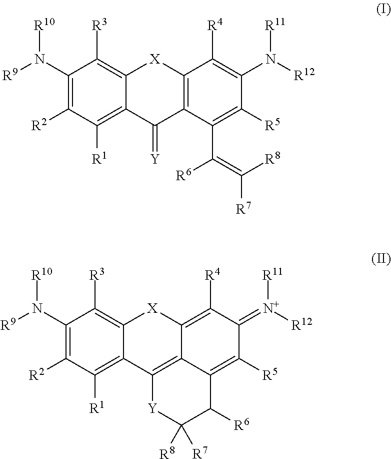

### BACKGROUND OF THE INVENTION

Fluorescence nanoscopy or super resolution microscopy techniques have extended optical imaging to reach the single nanometer-resolution range, and have enabled minimally invasive visualization of the internal nanoscale structures and dynamics of biological samples with molecular specificity. These techniques rely heavily on the fluorescent dyes employed, the chemically specific labels derived therefrom, and most critically on the intrinsic control between fluorescent and non-fluorescent states of the dye molecule. Photoactivatable or caged dyes have emerged as suitable labels for some of these nanoscopic techniques, by which the spatiotemporal control of the transition of a non-fluorescent molecule to a fluorophore is enabled through the controlled delivery of light, typically in the ultraviolet or visible range.

In single molecule localization microscopy (SMLM) methods [H. Li and J. C. Vaughan, Chem. Rev. 2018, 118, 9412-9454], such as photoactivated localization microscopy (PALM) and stochastic optical reconstruction microscopy (STORM) [S. T. Hess et al. Biophys. J. 2006, 91 (11) 4258-4272; E. Betzig et al., Science, 2016, 313, 1642-1645], photoactivatable dyes are particularly relied upon to achieve a high density of labelling required to visualize intricate biological structures while eliminating the need of reducing additives required to convert fluorophores to non-fluorescent states. In DNA-PAINT, photoactivatable fluorophores further allow application of imager DNA strands in high concentrations while simultaneously enabling precise label localization by minimizing fluorescence background [S. Jang et al., Angew. Chem. Int. Ed. 2020, 59 (29), 11758-11762].

In coordinate-targeted nanoscopy, such as stimulated emission depletion (STED) microscopy [S. W. Hell and J. Wichmann, Opt. Lett. 1994, 19, 780-782], as well as in conventional (confocal, widefield) fluorescence microscopy, photoactivatable dyes are utilized to obtain multicolor images or to add additional features to the acquisition. In a typical commercial STED microscope setup, there typically exists a limit on the number of simultaneous imaging color channels attainable with a single depletion laser. Two channel imaging is reliably demonstrated by careful selection of fluorophores, and with additional signal processing based on differences in the fluorescence lifetimes of the labels [J. Buckers et al., Optics Express 2011, 19 (4), 3130-3143] or spectral differences of the emitters [F. R. Winter et al., Sci. Rep. 2017, 7, 46492], the number of channels can be increased to three or four for selected samples. Photoactivatable dyes provide a unique opportunity for multicolour STED imaging utilizing two labels with overlapping absorption and emission spectra. In such an experiment, the photoactivatable dye is maintained in a non-fluorescent form while the other fluorophore is imaged normally, and then subsequently photobleached. The photoactivatable dye is next activated and may be imaged using the same excitation and detection channel as the first.

Photoactivatable dyes are further valuable tools in material science [Wöll and Flors, Small Methods, 2017, 1, 1700191], and are used to evaluate molecular diffusion dynamics in cellular systems via fluorescence redistribution after photoactivation (FRAPa) [D. Mazza et al., Biophys. J. 2008, 95, 3457-3469] or inverse fluorescence recovery after photobleaching (IFRAP) [S. Hauke et al. Chem. Sci. 2017, 8, 559-566]) and in single molecule tracking experiments [N. Banaz et al. J. Phys. D: Appl. Phys. 2019, 52, 064002].

Diverse strategies have been proposed for the photoactivatable dyes used for optical nanoscopy applications. One strategy relies on the incorporation of one or more photocleavable protecting groups (cages, or caging groups) which, when present, alter the chemical structure of the fluorophore and maintain the fluorophore in a dark state (caged dye). Upon photoactivation, release of the caging group yields the fluorophore. This strategy has been used extensively for triarylmethane fluorophores by installation of photocleavable carbamate protecting groups onto the amino groups of the unsubstituted or N,N′-disubstituted rhodamines (N,N,N′,N′-tetrasubstituted rhodamine dyes, such as TMR or SiR, cannot be caged using this approach) [J. B. Grimm et al., Org. Lett. 2011, 13 (24), 6354-6357; L. M. Wysocki et al., Angew. Chem. Int. Ed. 2011, 50, 11206-11209; J. B. Grimm et al., ACS Chem. Biol. 2013, 8, 1303-1310; J. B. Grimm et al., Angew. Chem. Int. Ed. 2016, 55, 1723-1727]. The most commonly used photolabile N-protecting groups are of 4,5-dialkoxy-2-nitrobenzyloxycarbonyl type, in particular nitroveratryloxycarbonyl (NVOC), and are cleaved readily upon irradiation with 405 nm light. NVOC cages have also been applied to caging the 9-position of oxazine dyes [S. Miller, WO2009/036351]. Similarly, fluorescein dyes have been protected as O,O′-bis(2-nitrobenzyl) ethers [S. Hauke et al., Chem. Sci., 2017, 8, 559-566]. Photocleavable carbamate-protected rhodamines have been further employed as fluorogenic enzyme substrates [T. G. Henares et al., Analyst 2013, 138, 3139-3141], chemosensors [S. Ye et al., Angew. Chem. Int. Ed. 2018, 57, 10173-10177], photoactivatable fluorescent tracers [K. R. Gee et al., Bioorg. Med. Chem. Lett. 2001, 2181-2183] or as fluorescent indicators of the covalently attached targeted payload release [A. T. Veetil et al., Nature Nanotech. 2017, 12, 1183-1189]. Similarly, photocleavable nitrobenzyl ethers have previously been used in the preparation of caged coumarin dyes [Li et al. U.S. Pat. No. 7,304,168B2] and their conjugates [Li et al. U.S. Pat. No. 8,153,103B2].

A major drawback of benzyl ether and carbamate cages is the significant molecular mass and hydrophobicity of these fragments. When added to the fluorescent label, they potentially lead to poor membrane permeability, loss of selectivity or affinity with self-labeling tag proteins, or technical limitations related to aggregation and precipitation of labeled antibodies. Some improvement of the aqueous solubility, photolysis rate and quantum yields of the 2-nitrobenzyl carbamate-protected rhodamine dyes has been achieved with the introduction of the carboxylate group in α-position of the 2-nitrobenzyl group [R. P. Haugland and K. R. Gee, U.S. Pat. No. 5,635,608]. A further drawback arises from the uncaging process that results in the release of potentially toxic and reactive byproducts, such as 2-nitrosobenzaldehydes, which can negatively affect live cell imaging.

Alternative small molecular weight cages, which photolyse to give presumably inert byproducts, have been developed. Transformation of the lactone ring of the rhodamine fluorophores into the corresponding cyclic α-diazoketones or 1,2,3-thiadiazoles or N-nitroso (thio) amides represents one such caging strategy [V. N. Belov et al., Angew. Chem. Int. Ed. 2010, 49, 3520-3523; S. W. Hell et al., WO2011/029459A1]. The most widely accepted α-diazoketone-caged dyes (named rhodamines NN) undergo photoinduced Wolff rearrangement upon irradiation with 360-420 nm light, converting into the fluorescent 21-carboxymethyl- or 21-methylrosamine products, with nitrogen gas as the byproduct. This strategy has been extended to a variety of substituted diazoketone-caged rhodamines [V. N. Belov et al., Chem. Eur. J. 2014, 20, 13162-13173], carbo- and Si-rhodamines [J. M Grimm et al., Nat. Methods 2016, 13 (12), 985-988; L. D. Lavis et al., WO2017/201531A1]. The significant drawback of the diazoketone-caged dyes is the varying efficiency of the photoactivation depending on the substitution pattern of the caged triarylmethane fluorophore due to the concomitant formation of non-fluorescent byproducts. This sensitivity has further been exploited toward super-resolved mapping of enzymatic activity [E. A. Halabi et al., J. Am. Chem. Soc. 2017, 139 (37), 13200-13207]. While the diazoketone labels have been successfully employed in SMLM techniques, including live-cell imaging, their application in STED nanoscopy has been limited by two-photon uncaging with the high intensity 775 nm depletion laser beam [V. N. Belov et al., Chem. Eur. J. 2014, 20, 13162-13173]. Fluorophores comprising one or more thiocarbonyl groups have also been reported as photoactivatable dyes for PALM microscopy through light-induced desulfurization to give the corresponding fluorescent oxygen analogue [J. Tang et al., J. Am. Chem. Soc., 2019, 141 (37) 14699-14706; Xiao et al. US2020/0271587].

Photoactivatable push-pull fluorophores based on the photoconversion of benzyl azides to anilines with visible light [S. J. Lord et al., J. Am. Chem. Soc. 2008, 130 (29), 9204-9205] have also been applied as labels for SMLM-based super resolution imaging [H. D. Lee et al., J. Am. Chem. Soc. 2010, 132 (43), 15099-15101]. A drawback of these caged dyes is that, despite the fluorescent aniline being the major photoproduct formed, the long-lived nitrene photolysis intermediate can undergo undesired reactions with nearby proteins, or alternatively, undergo rearrangements or oxidation to give non-fluorescent compounds [S. J. Lord et al., J. Phys. Chem B 2010, 114 (45), 14157-14167].

Photoswitchable fluorophores provide a caging-group free alternative to photoactivatable dyes in optical nanoscopy methods, where photoactivation of the dye results in a short-lived fluorescent form, which undergoes thermal- or light-driven isomerization to the initial dark state. Photochromic rhodamine amides with light-induced activation [K.-H. Knauer and R. Gleiter, Angew. Chem., 1977, 89 (2), 116-117] can be photoswitched from a nonfluorescent (closed) form to a fluorescent (open) form by absorption of one or two photons [J. Folling et al., ChemPhysChem 2008, 9 (2), 321-326], allowing for optical sectioning of thick samples with PALM with independently running acquisition (PALMIRA) nanoscopy. Oxazine auxochromes [E. Deniz et al. J. Phys. Chem. Lett. 2010, 1 (24), 3506-3509] have also been used for super-resolution imaging by SMLM methods [E. Deniz et al., J. Phys. Chem. C 2012, 116 (10), 6058-6068]. Both rhodamine-amides and oxazine auxochromes undergo thermal reversion to their initial dark form, a favourable property in SMLM nanoscopy since multiple localizations of the same fluorophore can be beneficial. Similarly, photoregulated fluxional fluorophores derived from the rhodamine B scaffold and bearing an acylhydrazone photoswitchable unit have been used in SMLM studies [E. A. Halabi et al. Nat. Comm. 2019, 10, 1232]. Photoactivation of the closed (E)-isomer of the hydrazone (dark) to the (Z)-isomer initiates the spontaneous thermal switching between open (fluorescent) and closed (dark)(Z)-isomers, resulting in the possibility of multiple fluorescent readouts from the same fluorophore.

Photoswitchable diarylethene [K. Yagi et al., J. Org. Chem., 2001, 66 (16), 5419-5423] dyes which switch with ultraviolet light from a non-fluorescent (open) form to a fluorescent (closed) form have also been applied in REversible Saturable Optical Fluorescence Transitions (RESOLFT) [B. Roubinet et. Angew. Chem. Int. Ed. 2016, 55 (49), 15429-15433] and STORM nanoscopy of biological samples [B. Roubinet et al. J. Am. Chem. Soc., 2017, 139 (19), 6611-6620]. However, the excitation with visible light necessary for visualization additionally drives these dyes to their open form and limits their application to SMLM-based nanoscopy. A further limitation of many diarylethene fluorophores remains their low solubility and photostability in aqueous solution. Significant improvements have been made through the incorporation of branched solubilizing groups [B. Roubinet et al., J. Am. Chem. Soc. 2017, 139 (19), 6611-6620; K. Uno et al. Proc. Nat. Acad. Sci. 2021, 118 (14), e2100165118], however, the anionic nature of these dyes hampers membrane permeability and thus precludes their application in live-cell imaging.

Cage-free photoactivatable compact fluorophores that undergo a single, irreversible conversion from the dark to the fluorescent form are therefore highly desirable. One such example was demonstrated in the photoactivation of silicon rhodamine analogues in which the fluorophore was masked in the form of an exocyclic double bond at the 9-position of the xanthene scaffold [M. S. Frei et al., Nat. Comm. 2019, 10, 4680; K. Johnsson et al. WO2019/122269A1]. Upon ultraviolet irradiation in aqueous solution, protonation yielded the fluorescent xanthene core. The resulting 9-alkyl-Si-pyronins were however susceptible to nucleophilic addition of water, resulting in an environment-dependent rapid equilibrium with a non-fluorescent product and limiting their applicability.

In view of this prior art, the main object underlying the present invention is the provision of new cage-free photoactivatable fluorescent dyes and labels for optical nanoscopy, including SMLM and STED techniques, which overcome or alleviate the above outlined drawbacks of the dyes and labels of the prior art. Specifically, the dyes and labels must be suitable for fixed- and live-cell imaging, demonstrate a highly efficient and rapid one- or multiphoton photoactivation, undergo unbiased photoactivation throughout the biologically-relevant pH range, and yield fluorophores with high brightness and photostability and low reactivity to intracellular nucleophiles.

This object has been achieved according to the present invention by providing the photoactivatable dyes of claim 1, the fluorescent dyes of claim 2 and the use thereof in various applications of claims 17-24. Other aspects and more specific embodiments of the invention are the subject of further claims.

### Definitions

The term “moiety” herein refers generally to a portion of a molecule, which may be a functional group, a set of functional groups, and/or a specific group of atoms within a molecule, that is responsible for a characteristic chemical, biological, and/or physical property of the molecule. The term “binding moiety”, as used herein, refers to any molecule or part of a molecule that can specifically bind to a target molecule. “Specific binding” means that a binding moiety (e.g. a molecule or part of a molecule) binds stronger to a target (another small molecule, a macromolecule such as a protein or nucleic acid, an oligomeric protein, a protein aggregate such as amyloid fibrils, a receptor etc.) for which it is specific compared to the binding to another target. A binding moiety binds stronger to a first target compared to a second target if it binds to the first target with a dissociation constant (k_{D}) which is lower than the dissociation constant for the second target. Preferably the dissociation constant (k_{D}) for the target to which the binding moiety binds specifically is more than 10-fold, preferably more than 20-fold, more preferably more than 50-fold, even more preferably more than 100-fold, 200-fold, 500-fold or 1000-fold lower than the dissociation constant (k_{D}) for the target to which the binding moiety does not bind specifically.

The term “C₁-C₄ alkyl” in the context of the present specification signifies a saturated linear or branched hydrocarbon having 1, 2, 3 or 4 carbon atoms, wherein in certain embodiments one carbon-carbon bond may be unsaturated and one CH₂ moiety may be exchanged for oxygen (ether bridge) or nitrogen (NH, or NR with R being methyl, ethyl, or propyl; amino bridge). Non-limiting examples for a C₁-C₄ alkyl are methyl, ethyl, propyl, prop-2-enyl (allyl), n-butyl, 2-methylpropyl, tert-butyl, but-3-enyl, prop-2-ynyl and but-3-ynyl. In certain embodiments, a C₁-C₄ alkyl is a methyl, ethyl, propyl or butyl moiety.

The term “C₃-C₈ cycloalkyl” in the context of the present specification signifies a saturated cyclic hydrocarbon having 3, 4, 5, 6, 7 or 8 carbon atoms in the cycle, wherein in certain embodiments one carbon-carbon bond may be unsaturated and one CH₂ moiety may be exchanged for oxygen (ether bridge) or nitrogen (NH, or NR with R being methyl, ethyl, or propyl; amino bridge). Non-limiting examples for a C₃-C₈ cycloalkyl are cyclopropyl, cyclopropylmethyl, cyclobutyl, cyclopentyl, cyclopent-1-en-1-yl, cyclopent-2-en-1-yl, cyclopent-3-en-1-yl, cyclopent-2-en-1-yl and cyclooctyl. In the context of the present specification, the term “cycloalkyl” also includes bicyclic and tricyclic cycloalkyls and cycloalkenyls, such as bicyclo[2.2.1]heptan-2-yl, bicyclo[2.2.1]hept-2-en-2-yl, tricyclo[4.1.0.0^{2,4}]heptan-5-yl and bicyclo[1.1.1]pentan-1-yl. In certain embodiments, a C₃-C₈ alkyl is a cyclopropyl, cyclopropylmethyl, cyclobutyl, cyclopentyl or cyclohexyl moiety.

A C₁-C₆ alkyl in the context of the present specification signifies a saturated linear or branched hydrocarbon having 1, 2, 3, 4, 5 or 6 carbon atoms, wherein one carbon-carbon bond may be unsaturated and one CH₂ moiety may be exchanged for oxygen (ether bridge) or nitrogen (NH, or NR with R being methyl, ethyl, or propyl; amino bridge). Non-limiting examples for a C₁-C₆ alkyl include the examples given for C₁-C₄ alkyl above, and additionally 3-methylbut-2-enyl, 2-methylbut-3-enyl, 3-methylbut-3-enyl, n-pentyl, 2-methylbutyl, 3-methylbutyl, 1,1-dimethylpropyl, 1,2-dimethylpropyl, 1,2-dimethylpropyl, pent-4-ynyl, 3-methyl-2-pentyl, and 4-methyl-2-pentyl. In certain embodiments, a C₅ alkyl is a pentyl or cyclopentyl moiety and a C₆ alkyl is a hexyl or cyclohexyl moiety.

The term “unsubstituted Cₙ alkyl” when used herein in the narrowest sense relates to the moiety —CₙH₂ₙ— if used as a bridge between moieties of the molecule, or —CₙH₂ₙ₊₁ if used in the context of a terminal moiety. It may still contain fewer H atoms if a cyclical structure or one or more (non-aromatic) double bonds are present.

The term “Cₙ alkylene” in the context of the present specification signifies a saturated linear or branched hydrocarbon comprising one or more double bonds. An unsubstituted alkylene consists of C and H only. A substituted alkylene may comprise one or several substituents as defined herein for substituted alkyl.

The term “Cₙ alkylyne” in the context of the present specification signifies a saturated linear or branched hydrocarbon comprising one or more triple bonds and may also comprise one or more double bonds in addition to the triple bond(s). An unsubstituted alkylyne consists of C and H only. A substituted alkylyne may comprise one or several substituents as defined herein for substituted alkyl.

The terms “unsubstituted Cₙ alkyl” and “substituted Cₙ alkyl” include a linear alkyl comprising or being linked to a cyclic structure, for example a cyclopropane, cyclobutane, cyclopentane or cyclohexane moiety, unsubstituted or substituted depending on the annotation or the context of mention, having linear alkyl substitutions. The total number of carbon and (where appropriate) N, O or other heteroatoms in the linear chain or cyclical structure adds up to n.

The term “substituted alkyl” in its broadest sense refers to an alkyl as defined above in the broadest sense that is covalently linked to an atom that is not carbon or hydrogen, particularly to an atom selected from N, O, F, B, Si, P, S, Se, Cl, Br and I, which itself may be (if applicable) linked to one or several other atoms of this group, or to hydrogen, or to an unsaturated or saturated hydrocarbon (alkyl or aryl in their broadest sense). In a narrower sense, substituted alkyl refers to an alkyl as defined above in the broadest sense that is substituted in one or several carbon atoms by groups selected from amine NH₂, alkylamine NHR, imide NH, alkylimide NR, amino (carboxyalkyl) NHCOR or NRCOR, hydroxyl OH, oxyalkyl OR, oxy (carboxyalkyl) OCOR, carbonyl O and its ketal or acetal (OR) 2, nitrile CN, isonitrile NC, cyanate CNO, isocyanate NCO, thiocyanate CNS, isothiocyanate NCS, fluoride F, chloride Cl, bromide Br, iodide I, phosphonate PO₃H₂, PO₃R², phosphate OPO₃H₂ and OPO₃R², sulfhydryl SH, sulfalkyl SR, sulfoxide SOR, sulfonyl SO₂R, sulfonylamide SO₂NHR, sulfonate SO₃H and sulfonate ester SO₃R, wherein the R substituent as used in the current paragraph, different from other uses assigned to R in the body of the specification, is itself an unsubstituted or substituted C₁ to C₁₂ alkyl in its broadest sense, and in a narrower sense, R is methyl, ethyl or propyl unless otherwise specified.

It is understood that mention of moieties SO₃H or COOH or other acidic groups imply presence of the deprotonated form in the alternative, assuming appropriate conditions that allow dissociation. It is also understood that mention of amino (NH₂, NHR, NR²) or imino (—CH═NR, —CR═NH, —CR═NR) moieties or other basic groups imply presence of the protonated form in the alternative, assuming appropriate conditions that allow protonation.

The term “amino substituted alkyl” or “hydroxyl substituted alkyl” refers to an alkyl according to the above definition that is modified by one or several amine or hydroxyl groups NH₂, NHR, NR² or OH, wherein the R substituent as used in the current paragraph, different from other uses assigned to R in the body of the specification, is itself an unsubstituted or substituted C₁ to C₁₂ alkyl in its broadest sense, and in a narrower sense, R is methyl, ethyl or propyl unless otherwise specified. An alkyl having more than one carbon may comprise more than one amine or hydroxyl.

Unless otherwise specified, the term “substituted alkyl” refers to alkyl in which each C is only substituted by at most one amine or hydroxyl group, in addition to bonds to the alkyl chain, terminal methyl, or hydrogen.

The term “carboxyl substituted alkyl” refers to an alkyl according to the above definition that is modified by one or several carboxyl groups COOH, or derivatives thereof, particularly carboxamides CONH₂, CONHR and CONR², or carboxylic esters COOR, with R having the meaning as laid out in the preceding paragraph and different from other meanings assigned to R in the body of this specification.

Non-limiting examples of “amino-substituted alkyl” include —CH₂NH₂, —CH₂NHCH₃, —CH₂NHCH₂CH₃, —CH₂CH₂NH₂, —CH₂CH₂NHCH₃, —CH₂CH₂NHCH₂CH₃, —(CH₂)₃NH₂, —(CH₂)₃NHCH₃, —(CH₂)₃NHCH₂CH₃, —CH₂CH(NH₂)CH₃, —(CH₂)₃CH₂NHCH₃, —CH₂CH(NHCH₃)CH₃, —CH₂CH(NHCH₂CH₃)CH₃, —(CH₂)₃CH₂NH₂, —(CH₂)₃CH₂NHCH₂CH₃, —CH(CH₂NH₂)CH₂CH₃, —CH(CH₂NHCH₃)CH₂CH₃, —CH(CH₂NHCH₂CH₃)CH₂CH₃, —CH₂CH(CH₂NHCH₂CH₃)CH₃, —CH(NHCH₂CH₃)(CH₂)₂NHCH₂CH₃, —CH₂CH(NHCH₂CH₃)CH₂NHCH₂CH₃, —CH₂CH(NH CH₂CH₃)(CH₂)₂NHCH₂CH₃, —CH₂CH(CH₂NH₂)CH₃, —CH(NH₂)(CH₂)₂NH₂, —CH₂CH(NH₂)CH₂NH₂, —CH₂CH(NH₂)(CH₂)₂NH₂, —CH₂CH(CH₂NH₂) 2, —CH₂CH(CH₂NHCH₃)CH₃, —CH(NHCH₃)(CH₂)₂NHCH₃, —CH₂CH(NHCH₃)CH₂NHCH₃, —CH₂CH(NHCH₃)(CH₂)₂NHCH₃, —CH₂CH(CH₂NHCH₃)₂ and —CH₂CH(CH₂NHCH₂CH₃)₂ for terminal moieties and —CH₂CHNH₂—, —CH₂CH(NHCH₃)—, —CH₂CH(NHCH₂CH₃)— for an amino substituted alkyl moiety bridging two other moieties.

Non-limiting examples of “hydroxy-substituted alkyl” include —CH₂OH, —(CH₂)₂OH, —(CH₂)₃OH, —CH₂CH(OH)CH₃, —(CH₂)₄OH, —CH(CH₂OH)CH₂CH₃, —CH₂CH(CH₂OH)CH₃, —CH(OH)(CH₂)₂OH, —CH₂CH(OH)CH₂OH, —CH₂CH(OH)(CH₂)₂OH and —CH₂CH(CH₂OH)₂ for terminal moieties and —CH(OH)—, —CH₂CH(OH)—, —CH₂CH(OH)CH₂—, —(CH₂)₂CH(OH)CH₂—, —CH(CH₂OH)CH₂CH₂—, —CH₂CH(CH₂OH)CH₂—, —CH(OH)CH₂CH(OH)—, —CH₂CH(OH)CH(OH)—, —CH₂CH(OH)(CH₂)₂CH(OH)- and —CH₂CH(CH₂OH)CH(OH)— for a hydroxyl substituted alkyl moiety bridging two other moieties. The term “halogen” refers to one or several atoms selected (independently) from F, Cl, Br, I. The term “halogen-substituted alkyl” refers to an alkyl according to the above definition that is modified by one or several halogen atoms selected (independently) from F, C₁, Br, I.

The term “fluoro substituted alkyl” refers to an alkyl according to the above definition that is modified by one or several fluoride groups F. Non-limiting examples of fluoro-substituted alkyl include —CH₂F, —CHF₂, —CF₃, —(CH₂)₂F, —(CHF)₂H, —(CHF)₂F, —C₂F₅, —(CH₂)₃F, —(CHF)₃H, —(CHF)₃F, —C₃F₇, —(CH₂)₄F, —(CHF)₄H, —(CHF)₄F and —C₄F₉.

The term “alkoxy” in the context of the present invention signifies an alkyl or cycloalkyl group, as defined above, connected to the rest of the molecule via an oxygen atom, such as, for example, methoxy, ethoxy, 1-propoxy, 2-propoxy, tert-butoxy, cyclopropyloxy, allyloxy, and the higher homologs and isomers such as, for example, cyclohexyloxy.

The term “C₁-C₈ alkoxycarbonyl” in the context of the present invention signifies a C₁-C₈ alkoxy group, as defined above, connected to the rest of the molecule via a carbonyl group. Some non-limiting examples of such alkoxycarbonyl are, for example, carbomethoxy-C(═O)OCH₃, carboethoxy-C(═O)OCH₂CH₃ and tert-butyloxycarbonyl (Boc)-C(═O)OC(CH₃) 3 groups.

The term “aryl” in the context of the present invention signifies a cyclic aromatic C₅-C₁₀ hydrocarbon that may comprise a heteroatom (e.g. N, O, S). Examples of aryl include, without being restricted to, phenyl and naphthyl, and any heteroaryl. A “heteroaryl” is an aryl that comprises one or several nitrogen, oxygen and/or sulphur atoms. Examples for heteroaryl include, without being restricted to, pyrrole, thiophene, furan, imidazole, pyrazole, thiazole, oxazole, pyridine, pyrimidine, thiazine, quinoline, benzofuran and indole. An aryl or a heteroaryl in the context of the invention additionally may be substituted by one or more alkyl groups. The term “alkylaryl” in the context of the present invention a substituted alkyl in the broadest sense as defined above, substituted in one or several carbon atoms with an aryl or heteroaryl as defined above. Some non-limiting examples of alkylaryl include benzyl, 2-phenylethyl, 2-(2-furyl) ethyl and 3-(1-indolyl) propyl.

A “substituted aryl” or “substituted heteroaryl” or “substituted alkylaryl” may comprise one or several substituents as defined herein for substituted alkyl.

The term “acyl” in the context of the present invention signifies an alkyl, cycloalkyl, aryl or alkylaryl group, as defined above, connected to the rest of the molecule via a carbonyl group-C(═O)—, such as, for example, acetyl, propionyl, benzoyl, 2-furoyl, 4-methoxybenzoyl, cinnamyl, Boc-Gly-.

The term “alkylsulfonyl” in the context of the present invention signifies an alkyl, cycloalkyl, aryl or alkylaryl group, as defined above, connected to the rest of the molecule via a sulfonyl group-SO₂—, such as, for example, mesyl, tosyl, trifluoromethanesulfonyl, vinylsulfonyl, dansyl, 4-nitrobenzenesulfonyl.

“Capable of forming a hybrid” in the context of the present invention relates to sequences that under the conditions existing within the cytosol of a mammalian cell, are able to bind selectively to their target sequence. Such hybridizing sequences may be contiguously reverse-complimentary to the target sequence, or may comprise gaps, mismatches or additional non-matching nucleotides. The minimal length for a sequence to be capable of forming a hybrid depends on its composition, with C or G nucleotides contributing more to the energy of binding than A or T/U nucleotides, and the backbone chemistry.

“Nucleotides” in the context of the present invention are nucleic acid or nucleic acid analogue building blocks, oligomers of which are capable of forming selective hybrids with RNA oligomers on the basis of base pairing. The term nucleotides in this context includes the classic ribonucleotide building blocks adenosine, guanosine, uridine (and ribosylthymin), cytidine, the classic deoxyribonucleotides deoxyadenosine, deoxyguanosine, thymidine, deoxyuridine and deoxycytidine. It further includes analogues of nucleic acids such as phosphorothioates, peptide nucleic acids (PNA; N-(2-aminoethyl)-glycine units linked by peptide linkage, with the nucleobase attached to the alpha-carbon of the glycine) or locked nucleic acids (LNA; RNA building blocks methylene-bridged between 2′-oxygen and 4′-carbon). The hybridizing sequence may be composed of any of the above nucleotides, or mixtures thereof.

The term “active ester” as used herein, refers to any ester-containing compound capable of reacting with functional groups, such as an amine or sulfhydryl groups, in particular with amine and sulfhydryl groups in a biomolecule. Some non-limiting examples of active esters are N-hydroxysuccinimidyl ester, N-hydroxysulfosuccinimidyl ester, N-hydroxyphthalimidyl ester, tetrafluorophenyl ester, and pentafluorophenyl ester. In the context of the present specification, the term “active ester” is also extended to include acyl fluorides and acyl azides.

“Structurally identical” substituents are understood as having the same number of atoms from which they are composed and the same connectivity between those atoms; for example, the substituents R⁹=isopropyl and R¹²=isopropyl are structurally identical, and the substituents —NR⁹R¹⁰=dimethylamino and —NR¹¹R¹²=dimethylamino are structurally identical.

It is understood that any position wherein H (hydrogen atom) is present it can be substituted with D (deuterium atom), in particular if such substitution improves the properties (e.g. increases photostability and fluorescence quantum yield) of the fluorescent dyes of the present invention. Where ionizable moieties are disclosed, it is understood that any salt, particularly any pharmaceutically acceptable salt of such molecule is encompassed by the invention. The salt comprises the ionized molecule of the invention and an oppositely charged counterion. Non-limiting examples of anionic salt forms include acetate, benzoate, besylate, bitatrate, bromide, carbonate, chloride, citrate, edetate, edisylate, embonate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate, phosphate, diphosphate, salicylate, disalicylate, stearate, succinate, sulfate, tartrate, tosylate, triethiodide and valerate. Non-limiting examples of cationic salt forms include aluminium, benzathine, calcium, ethylene diamine, lysine, magnesium, meglumine, potassium, procaine, sodium, tromethamine and zinc.

The following abbreviations are used throughout the following text and claims: BSA, bovine serum albumin; Da, Dalton (unified atomic mass unit); DIPEA, N,N-diisopropylethylamine; DMF, N,N-dimethylformamide; DMSO, dimethyl sulfoxide; LED, light emitting diode; NHS, N-hydroxysuccinimide; PBS, phosphate buffered saline; PVA, polyvinyl alcohol; TFA, trifluoroacetic acid; THF, tetrahydrofuran.

### DESCRIPTION OF THE INVENTION

The present invention provides novel compounds which are photoactivatable bridged benzophenone derivatives of type I bearing an alkenyl subsituent (—CR⁶═CR⁷R⁸) ortho to an exocylic double bond (C═Y) so that, upon activation with UV, visible or infrared light, they cyclize to generate the fluorescent xanthylium-type dyes II:

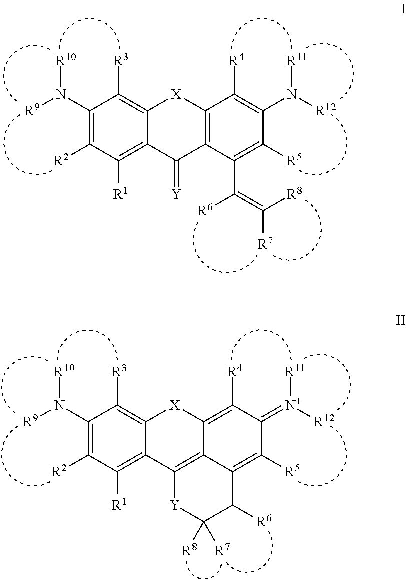

The present invention relates to novel compounds, in particular photoactivatable fluorescent dyes, which have the general structural formula I below:

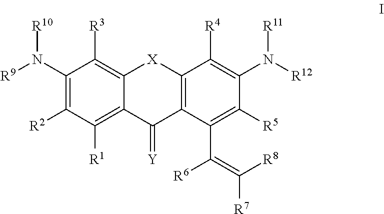

wherein:
R¹, R², R³, R⁴, R⁵, R⁶, R⁷ and R⁸, independently of each other are selected from H, halogen, SO₃H, CO₂H, CN, NO₂, CO₂R, SO₂R (with R being selected from C₁ to C₄ unsubstituted alkyl) and an unsubstituted or substituted (particularly unsubstituted or halogen-, amino-, hydroxyl-, SO₃H- and/or carboxyl substituted) moiety selected from C₁-C₂₀ alkyl, C₃-C₈ cycloalkyl, C₁-C₂₀ alkoxy, C₂-C₂₀ alkylene, C₂-C₂₀ alkylyne, C₇-C₂₀ alkylaryl, phenyl and 5- or 6-membered ring heteroaryl, or a combination thereof;
and where the substituents R⁶ and R⁷, taken together with the atoms to which they are bound, may form a 5-8 membered ring structure; and/or where the substituents R⁷ and R⁸, taken together with the atoms to which they are bound, may form a 5-8 membered ring structure; R⁹, R¹⁰, R¹¹, R¹² are:
a. independently selected from H, unsubstituted and substituted C₁-C₈ alkyl, C₃-C₈ cycloalkyl, C₁-C₈ acyl, C₁-C₈ alkoxycarbonyl, and C₇-C₁₂ alkylaryl, and unsubstituted phenyl or phenyl substituted by unsubstituted alkyl, halogen, alkoxy, NO₂, CO₂H, CO₂R and/or CONR²— with each R in CO₂R or CONR² being selected independently from C₁ to C₄ unsubstituted alkyl-; or
b. R⁹ together with R¹⁰ and a nitrogen atom to which they are bound, and/or R¹¹ together with R¹² and a nitrogen atom to which they are bound form a 3-7 membered ring structure; or
c. R⁹ and/or R¹¹ are independently selected from H and unsubstituted and substituted C₁-C₈ alkyl, C₃-C₈ cycloalkyl, and C₇-C₁₂ alkylaryl; and R¹⁰ together with R² or R³ and the atoms to which they are bound form a 5-7 membered ring structure, and/or R¹² together with R⁴ or R⁵ and the atoms to which they are bound form a 5-7 membered ring structure;
d. R⁹ together with R² and the atoms to which they are bound form a 5-7 membered ring structure, and/or R¹⁰ together with R³ and the atoms to which they are bound form a 5-7 membered ring structure, and/or R¹¹ together with R⁴ and the atoms to which they are bound form a 5-7 membered ring structure, and/or R¹² together with R⁵ and the atoms to which they are bound form a 5-7 membered ring structure;
   X is independently selected from:
a. O or S atom or SO₂ group;
b. NR¹³ or P(═O)R¹³ group, where R¹³ is selected from H, unsubstituted and substituted C₁-C₁₂ alkyl, C₃-C₈ cycloalkyl, C₁-C₂₀ alkoxy, C₁-C₂₀ alkoxycarbonyl, C₂-C₂₀ acyl, C₂-C₂₀ alkylsulfonyl, C₂-C₂₀ alkylene, C₂-C₂₀ alkylyne and C₇-C₂₀ alkylaryl, phenyl and 5- or 6-membered ring heteroaryl, or a combination thereof;
c. SiR¹⁴R¹⁵ or GeR¹⁴R¹⁵ group, where R¹⁴ and R¹⁵ are each independently selected from unsubstituted and substituted C₁-C₁₂ alkyl, C₃-C₈ cycloalkyl, C₁-C₂₀ alkoxy, C₂-C₂₀ alkylene, C₂-C₂₀ alkylyne and C₇-C₂₀ alkylaryl, phenyl and 5- or 6-membered ring heteroaryl, or a combination thereof,
   or where both substituents R¹⁴ and R¹⁵, taken together with the Si or Ge to which they are attached, form a 4-7 membered ring structure;
d. CR¹⁶R¹⁷ group, where R¹⁶ and R¹⁷ are each independently selected from H, F, CF₃, CN, COR¹⁸, CO₂R¹⁸, SO₂R¹⁸, CONR¹⁸R¹⁹ (where R¹⁸ and R¹⁹ in COR¹⁸, CO₂R¹⁸, SO₂R¹⁸, and CONR¹⁸R¹⁹ are each independently selected from unsubstituted and substituted C₁-C₁₂ alkyl, C₃-C₈ cycloalkyl, C₁-C₂₀ alkoxy, C₂-C₂₀ alkylene, C₂-C₂₀ alkylyne and C₇-C₂₀ alkylaryl, phenyl and 5- or 6-membered ring heteroaryl, or a combination thereof), unsubstituted and substituted C₁-C₁₂ alkyl, C₃-C₈ cycloalkyl, C₁-C₂₀ alkoxy, C₂-C₂₀ alkylene, C₂-C₂₀ alkylyne and C₇-C₂₀ alkylaryl, phenyl and 5- or 6-membered ring heteroaryl, or a combination thereof,
   or where both substituents R¹⁶ and R¹⁷, taken together with the C atom to which they are attached, form a 4-7 membered ring structure;
      Y is independently selected from:
a. O or S atom;
b. NR²⁰ group, where R²⁰ is selected from H, unsubstituted and substituted C₁-C₁₂ alkyl, C₃-C₈ cycloalkyl, C₁-C₂₀ alkoxy, C₂-C₂₀ acyl, C₂-C₂₀ alkylsulfonyl, C₂-C₂₀ alkylene, C₂-C₂₀ alkylyne and C₇-C₂₀ alkylaryl, phenyl and 5- or 6-membered ring heteroaryl, or a combination thereof;
c. CR²¹R²² group, where R²¹ and R²² are each independently selected from H, F, CF₃, CN, COR²³, CO₂R²³, SO₂R²³, CONR²³R²⁴ (where R²³ and R²⁴ in COR²³, CO₂R²³, SO₂R²³, CONR²³R²⁴ are each independently selected from unsubstituted and substituted C₁-C₁₂ alkyl, C₃-C₈ cycloalkyl, C₁-C₂₀ alkoxy, C₂-C₂₀ alkylene, C₂-C₂₀ alkylyne and C₇-C₂₀ alkylaryl, phenyl and 5- or 6-membered ring heteroaryl, or a combination thereof), unsubstituted and substituted C₁-C₁₂ alkyl, C₃-C₈ cycloalkyl, C₁-C₂₀ alkoxy, C₂-C₂₀ alkylene, C₂-C₂₀ alkylyne and C₇-C₂₀ alkylaryl, unsubstituted and substituted phenyl, unsubstituted and substituted 5- or 6-membered ring heteroaryl, or a combination thereof,
   or where both substituents R²¹ and R²², taken together, form a 4-7 membered ring structure.

Representative examples of compounds of the general structural formula I above are substituted derivatives of 3,6-diamino-1-vinyl-9H-xanthen-9-one (X═O, Y═O), 3,6-diamino-1-vinyl-9H-thioxanthen-9-one (X═S, Y═O), 3,6-diamino-1-vinyl-9H-thioxanthen-9-one 10,10-dioxide (X═SO₂, Y═O), 3,6-diamino-1-vinylacridin-9 (10H)-one (X═NR¹³, Y═O), 3,7-diamino-1-vinyl-10H-acridophosphin-10-one 5-oxide (X═P(═O)R¹³, Y═O), 3,7-diamino-1-vinyldibenzo[b,e]silin-10 (5H)-one (X═SiR¹⁴R¹⁵, Y═O), 3,7-diamino-1-vinyldibenzo[b,e]germin-10 (5H)-one (X═GeR¹⁴R¹⁵, Y═O), 3,6-diamino-1-vinylanthracen-9 (10H)-one (X═CR¹⁶R¹⁷, Y═O), substituted derivatives of the corresponding thioketones (X═O, S, SO₂, NR¹³, P(═O)R¹³, SIR¹⁴R¹⁵, GeR¹⁴R¹⁵, CR¹⁶R¹⁷; Y═S), substituted derivatives of the corresponding imines (X═O, S, SO₂, NR¹³, P(═O)R¹³, SiR¹⁴R¹⁵, GeR¹⁴R¹⁵, CR¹⁶R¹⁷; Y═NR²⁰), and substituted derivatives of the corresponding methylidene derivatives (X═O, S, SO₂, NR¹³, P(═O)R¹³, SiR¹⁴R¹⁵, GeR¹⁴R¹⁵, CR¹⁶R¹⁷; Y═CR²¹R²²).

Further novel compounds of the invention, in particular fluorescent dyes, which are obtainable by irradiation with light (UV, visible or infrared) through a one-photon absorption process or a multiphoton absorption process of any of the compounds of general formula I described above have the general structural formula II below:

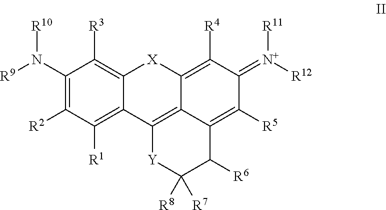

where R¹, R², R³, R⁴, R⁵, R⁶, R⁷, R⁸, R⁹, R¹⁰, R¹¹, R¹², X and Y are defined as above.

Representative examples of compounds of the general structural formula II above are substituted 5,6-dihydropyrano-(Y═O), 5,6-dihydrothiopyrano-(Y═S), 2,3-dihydro-1H-pyrido-(Y═NR²⁰) and 2,3-dihydro-1H-benzo-(Y═CR²¹R²²) fused pyronine (X═O), thiopyronine (X═S, SO₂), 3,6-diaminoacridin-10-ium (X═NR¹³), P-pyronine (X═P(═O)R¹³), Si-pyronine (X═SiR¹⁴R¹⁵), Ge-pyronine (X═GeR¹⁴R¹⁵) and carbopyronine (X═CR¹⁶R¹⁷) fluorophores.

In one specific embodiment, the compound of general structural formula I or II is covalently linked (particularly through any one of substituents R¹, R², R³, R⁴, R⁵, R⁶, R⁷, R⁸, R⁹, R¹⁰, R¹¹ and R¹² or through any of the groups X and Y) to a binding moiety M according to the general definition above.

In a more specific embodiment, the binding moiety M is selectively attachable by covalent bond to a protein or nucleic acid, in particular under conditions prevailing in cell culture or inside of a living cell (e.g. pH ranging from 4.5 to 8.0 across different organelles, glutathione (GSH) concentration ranging between 0.5 and 15 mM, temperature between 30° C. and 38° C. for mammalian cells), particularly a moiety able to form an ester bond, an ether bond, an amide or thioamide bond, a sulfide or disulfide bond, a carbon-carbon bond, a carbon-nitrogen bond such as a Schiff base, or a moiety able to react in a click-chemistry reaction with a corresponding reactive or functional group. In a more specific embodiment, said binding moiety M is selected from —COCH═CH₂, —SO₂CH═CH₂,—COCH₂l, —COC═CH, —N═C═S, —CO—NHS or another active ester, biotin, an azide or a tetrazine moiety, a diazoalkane or diazoketone moiety, a diazirine moiety, an alkyne, a strained alkyne such as a bicyclo[6.1.0]nonyne moiety or cyclooctyne moiety, a strained alkene such as trans-cyclooctene moiety or norbornene moiety or a maleimide.

In another specific embodiment, the binding moiety M is a substrate of a haloalkane halotransferase, particularly when M is a 1-chlorohexyl moiety as exemplarily shown below:

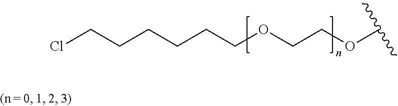

In another specific embodiment, the binding moiety M is a substrate of O⁶-alkylguanine-DNA-alkyltransferase, particularly a (substituted) O⁶-benzylguanine, O²-benzylcytosine or 4-benzyloxy-6-chloropyrimidine-2-amine moiety as exemplarily shown below:

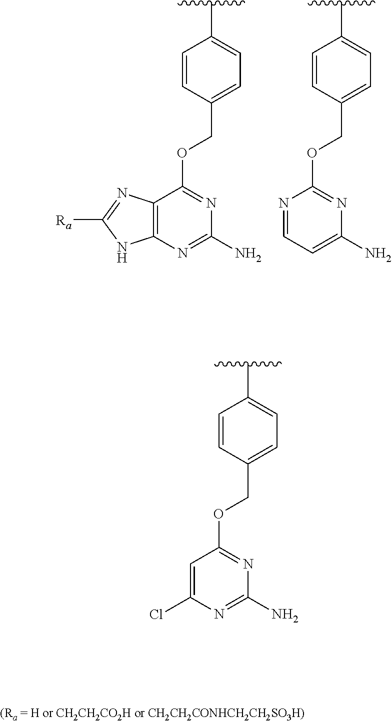

In another specific embodiment, the binding moiety M is a substrate of dihydrofolate reductase, particularly a 4-demethyltrimethoprim moiety as exemplarily shown below:

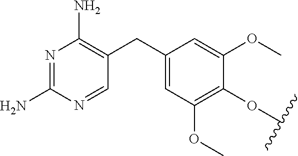 [SMILES: *Oc1c(OC)cc(Cc2cnc(N)nc2N)cc1OC]

In another specific embodiment, the binding moiety M is a moiety capable of selectively interacting non-covalently with a biomolecule (particularly a protein or nucleic acid) wherein said moiety and said biomolecule form a complex having a dissociation constant k_{D} of 10⁻⁶ mol/L or less. In a more specific embodiment, the said binding moiety M is selected from de-N-Boc-docetaxel, de-N-Boc-cabazitaxel, de-N-Boc-larotaxel or another taxol derivative, a phalloidin derivative, a jasplakinolide derivative, a bis-benzimide DNA stain, pepstatin A or triphenylphosphonium, as exemplarily shown below:

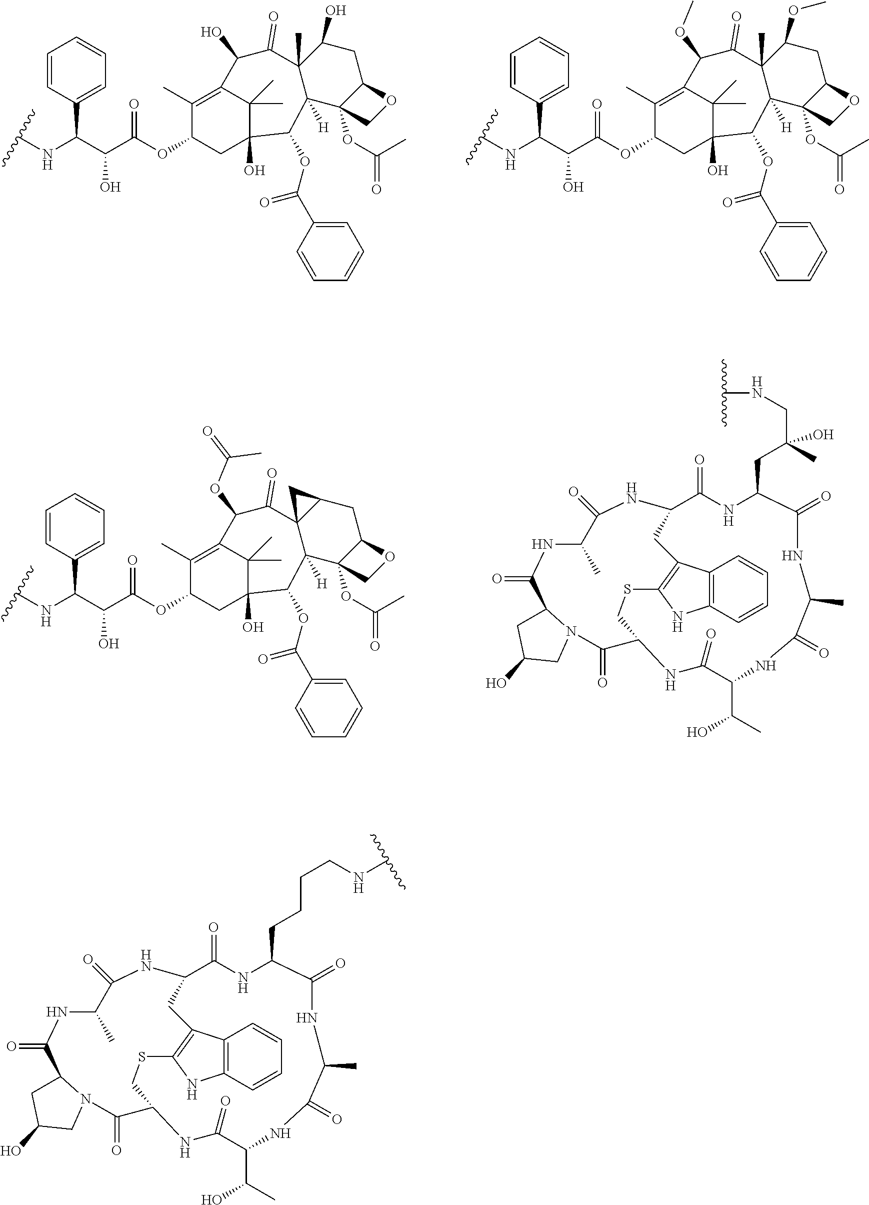

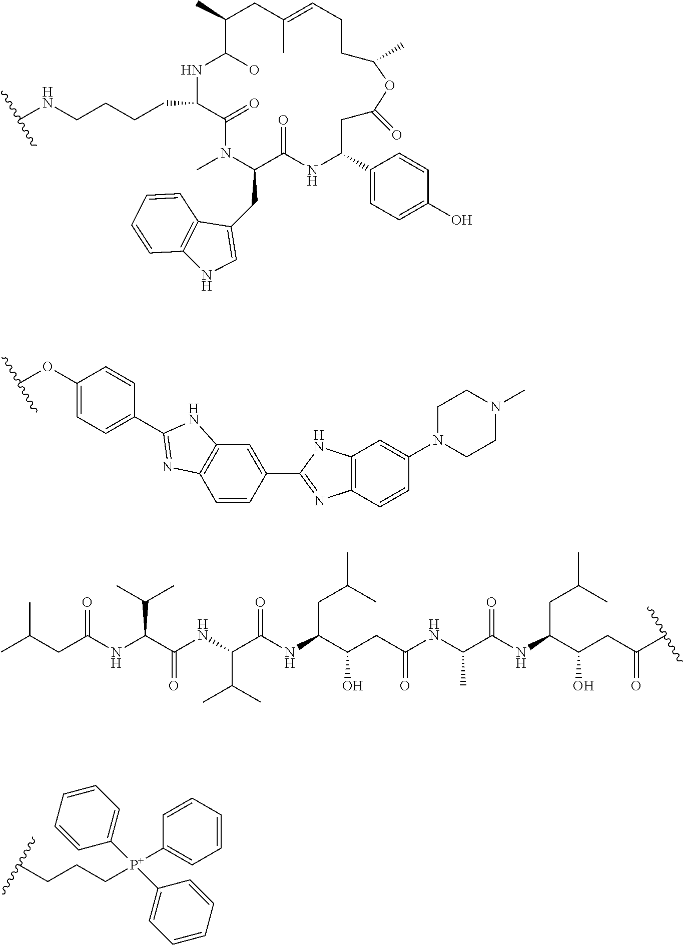

In another specific embodiment, the binding moiety M is an oligonucleotide having a sequence length between 10 and 40 nucleotides.

In another specific embodiment, the binding moiety M is a lipid, particularly a sphingosine derivative such as a ceramide, or a phospholipid such as dioleoylphosphatidylethanolamine (DOPE) or dipalmitoylphosphatidylethanolamine (DPPE), or a fatty acid.

In one specific embodiment, the compound of general structural formula I, in particular photoactivatable fluorescent dye, has one of the structural formulas I-1-I-32:

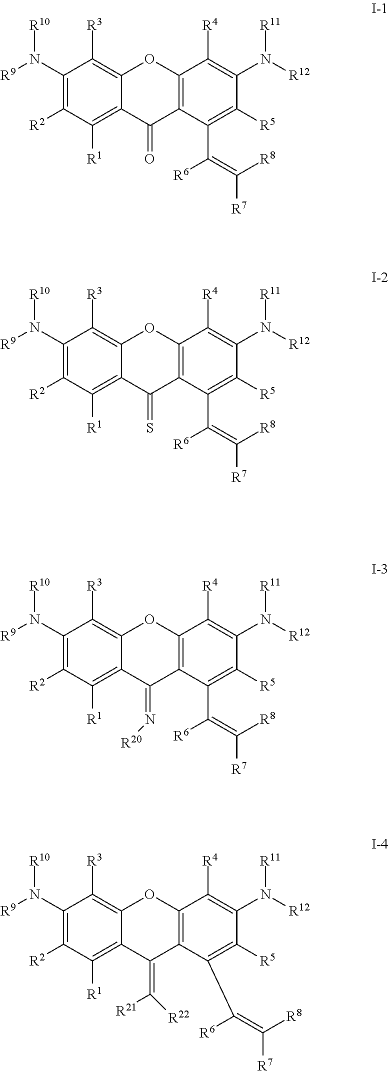

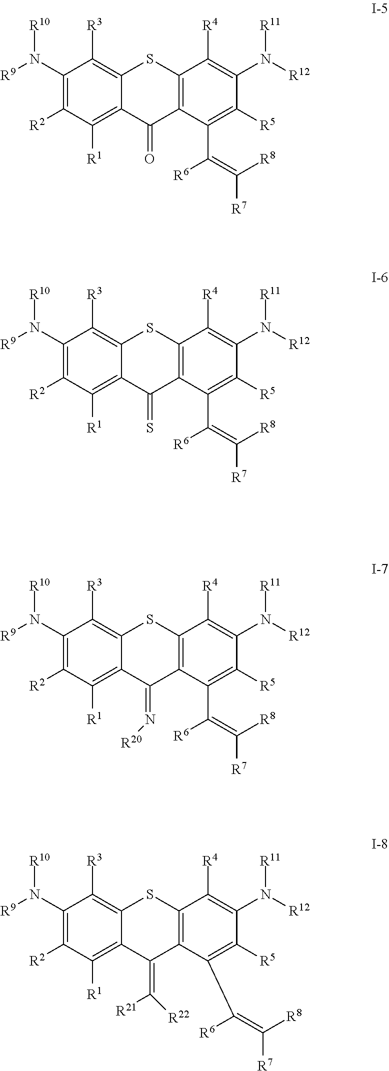

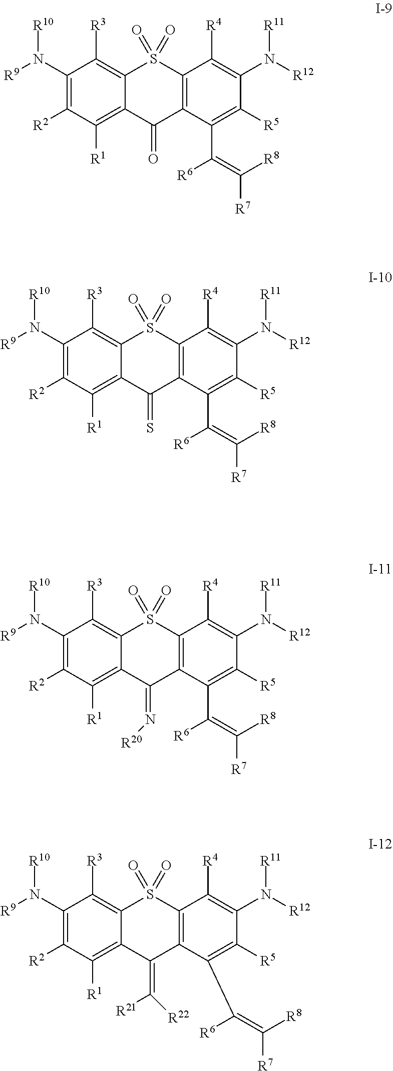

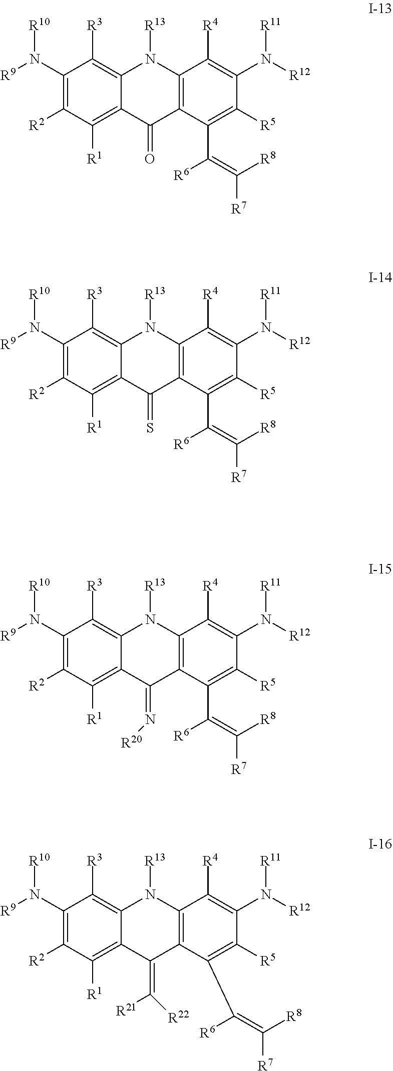

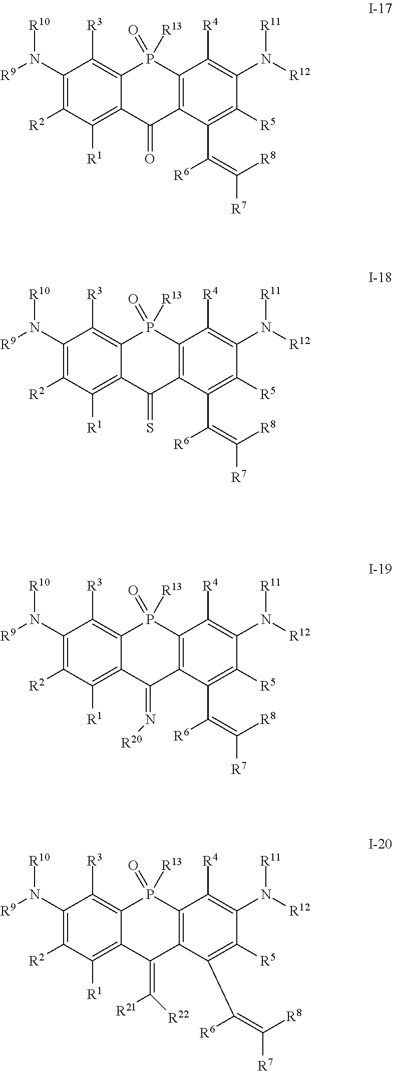

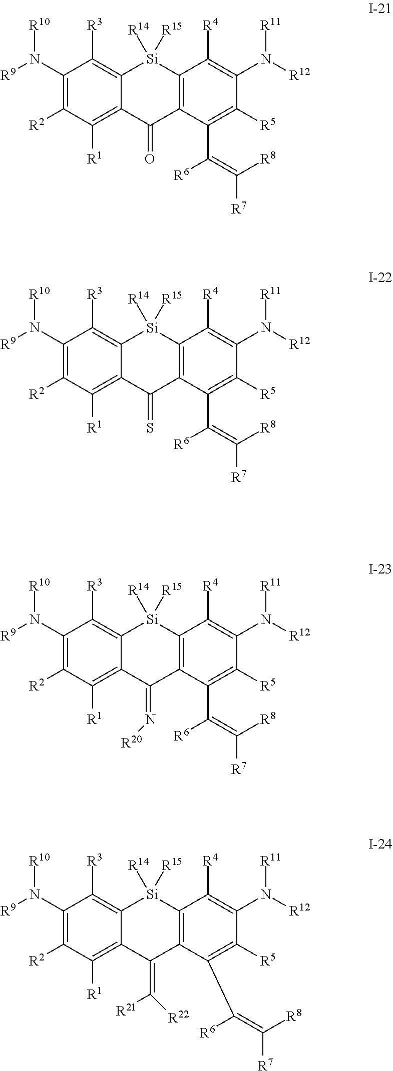

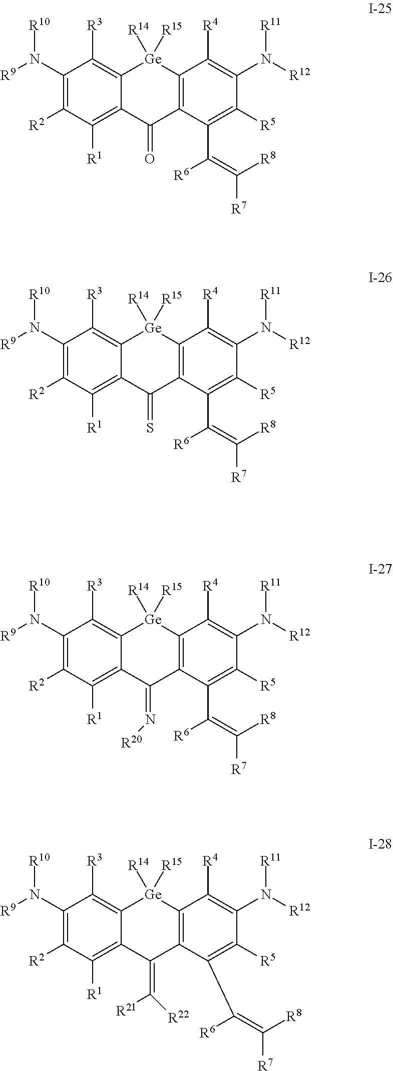

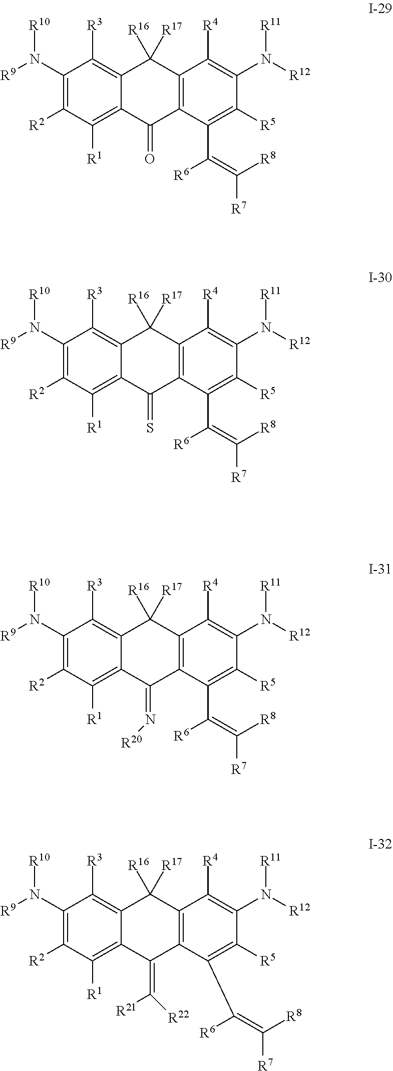

where R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² are defined as above, and wherein any one of substituents R¹, R², R³, R⁴, R⁵, R⁶, R⁷, R⁸, R⁹, R¹⁰, R¹¹, R¹² or one of the substituents R¹³, R¹⁴, R¹⁵, R¹⁶, R¹⁷, R¹⁸, R¹⁹, R²⁰, R²¹, R²² (if present) independently of any other is H or a moiety having a molecular weight between 15 and 1500 Da. In a more specific embodiment:
a) the substituents R⁹, R¹⁰, R¹¹, R¹² are selected from H and methyl, or any of the substituents-NR⁹R¹⁰ and —NR¹¹R¹² represents an azetidine ring, and
b) one of substituents R¹, R², R³, R⁴, R⁵, R⁶, R⁷, R⁸ or one of the R¹³, R¹⁴, R¹⁵, R¹⁶, R¹⁷, R¹⁸, R¹⁹, R²⁰, R²¹, R²² (if present) is H or a moiety having a molecular weight between 15 and 1500 Da, and
c) the other substituents R¹, R², R³, R⁴, R⁵ are selected from H and F, and
d) the other substituents R⁶, R⁷, R⁸ are selected from H and methyl, and
e) the other substituents R¹³, R¹⁴, R¹⁵ (if present) are selected from methyl, ethyl, isopropyl or phenyl,
f) the other substituents R¹⁶, R¹⁷ (if present) are methyl,
g) the other substituents R²⁰, R²¹, R²² (if present) are selected from H and methyl.

In another specific embodiment, the compound of general structural formula II, in particular a fluorescent dye, in particular when produced by irradiation with light (UV, visible or infrared) of any of the compounds of general formula I-1-I-32, has one of the structural formulas II-1-II-32:

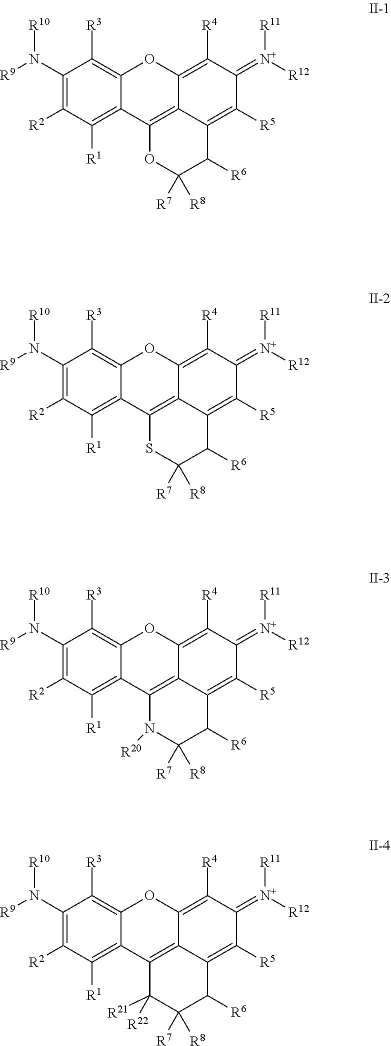

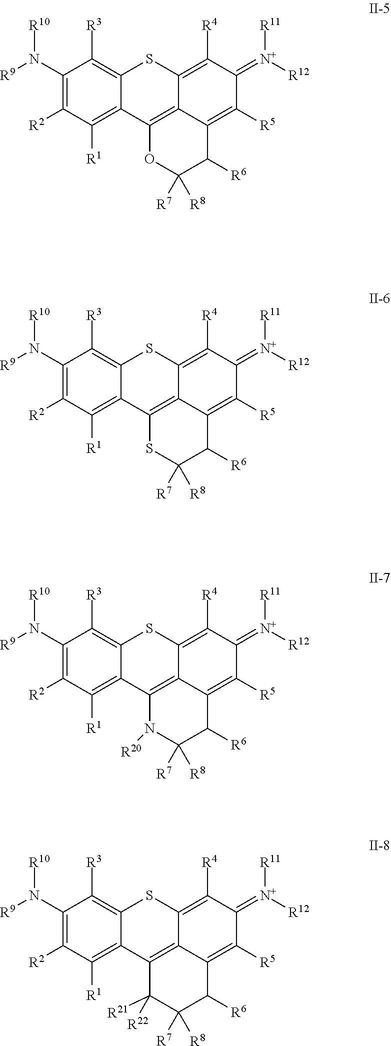

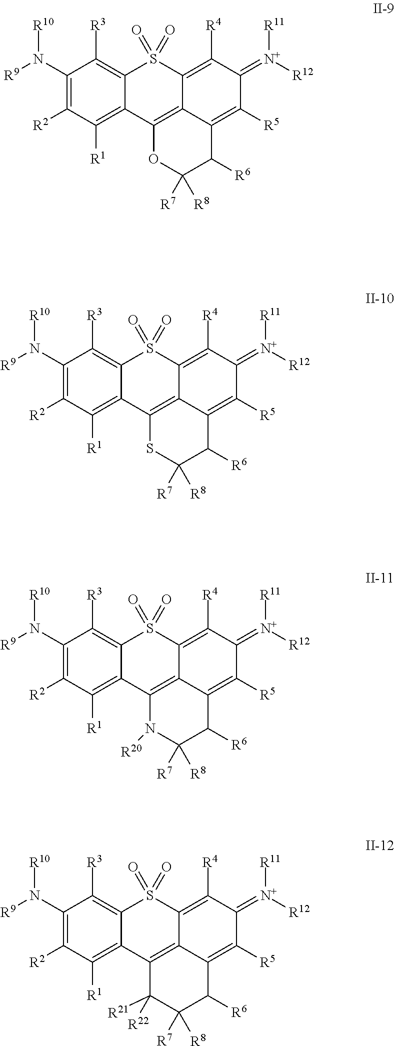

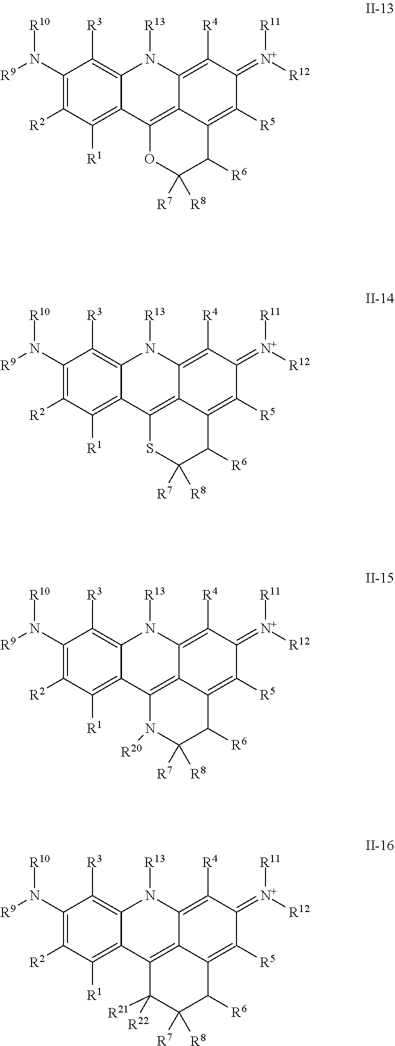

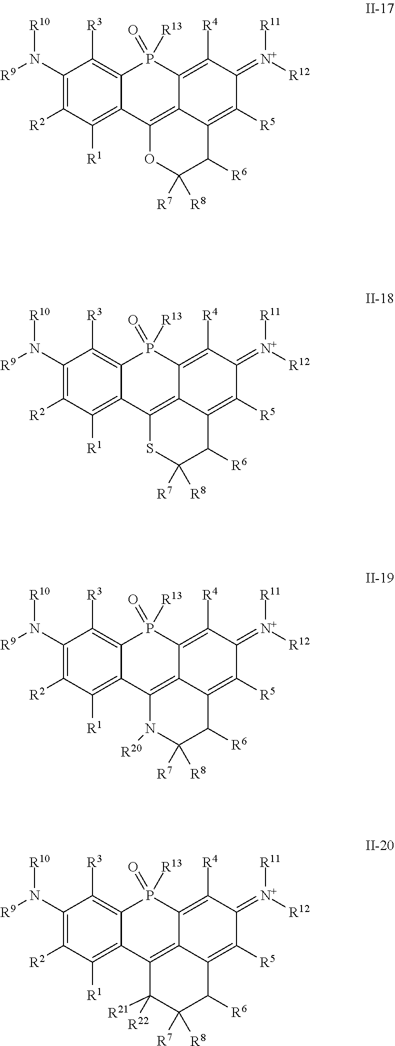

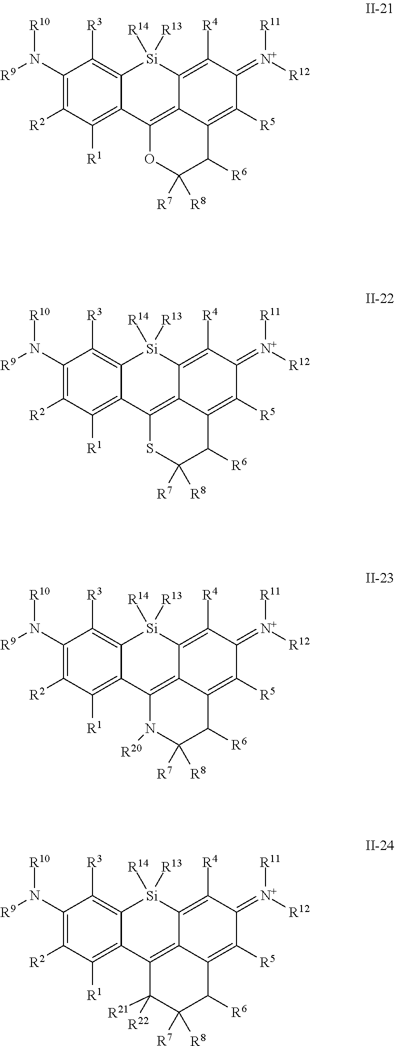

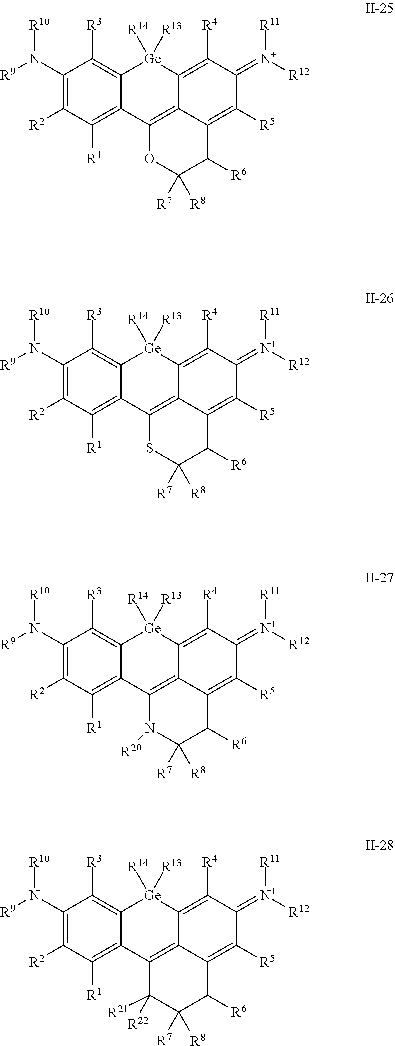

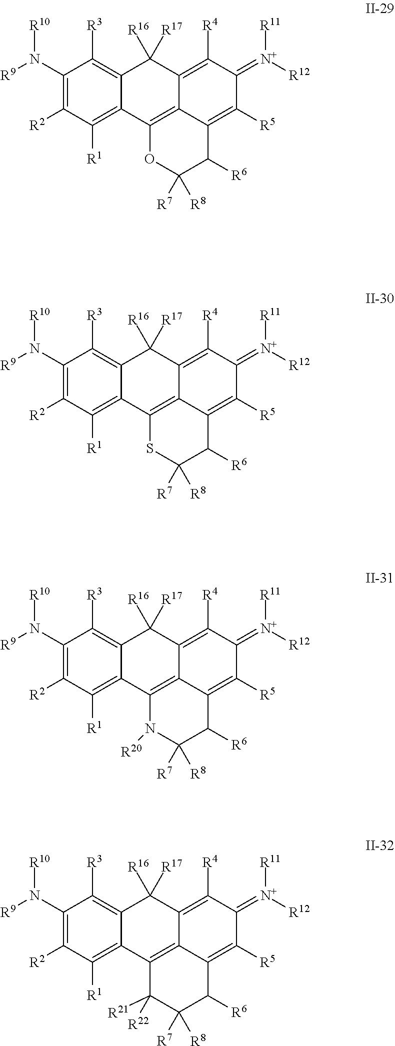

where R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² are defined as above, and wherein any one of substituents R¹, R², R³, R⁴, R⁵, R⁶, R⁷, R⁸, R⁹, R¹⁰, R¹¹, R¹² or one of the substituents R¹³, R¹⁴, R¹⁵, R¹⁶, R¹⁷, R¹⁸, R¹⁹, R²⁰, R²¹, R²² (if present) independently of any other is H or a moiety having a molecular weight between 15 and 1500 Da.

In a more specific embodiment:
a) the substituents R⁹, R¹⁰, R¹¹, R¹² are selected from H and methyl, or any of the substituents —NR⁹R¹⁰ and —NR¹¹R¹² represents an azetidine ring, and
b) one of substituents R¹, R², R³, R⁴, R⁵, R⁶, R⁷, R⁸ or one of the R¹³, R¹⁴, R¹⁵, R¹⁶, R¹⁷, R¹⁸, R¹⁹, R²⁰, R²¹, R²² (if present) is H or a moiety having a molecular weight between 15 and 1500 Da, and
c) the other substituents R¹, R², R³, R⁴, R⁵ are selected from H and F, and
d) the other substituents R⁶, R⁷, R⁸ are selected from H and methyl, and
e) the other substituents R¹³, R¹⁴, R¹⁵ (if present) are selected from methyl, ethyl, isopropyl or phenyl,
f) the other substituents R¹⁶, R¹⁷ (if present) are methyl,
g) the other substituents R²⁰, R²¹, R²² (if present) are selected from H and methyl.

In another specific embodiment, the said moiety having a molecular weight between 15 and 1500 Da is characterized by a general formula-L-M, wherein L is a linker covalently connecting the compound of structure I-1-I-32 or II-1-II-32 to the binding moiety M as defined above, and L is a covalent bond or a linker consisting of 1 to 50 atoms having an atomic weight of 12 or higher (in addition to the number of hydrogen atoms required to satisfy the valence rules). In a more specific embodiment, the said moiety having a molecular weight between 15 and 1500 Da is characterized by a general formula

-Lₘ^{A1}-L_{m′}^{J1}-Lₙ^{A2}-L_{n′}^{J2},-Lₚ^{A3}-L_{p′}^{J3},-L_{q}^{A4},-L_{q′}^{J4},-Mₛ, wherein
L^{A1}, L^{A2}, L^{A3} and L^{A4} independently of each other are selected from C₁ to C₁₂ unsubstituted or amino-, hydroxyl-, carboxyl- or fluoro substituted alkyl or cycloalkyl, (CH₂—CH₂—O), with r being an integer from 1 to 20, alkylaryl, alkylaryl-alkyl, and unsubstituted or alkyl-, halogen-, amino-, alkylamino-, imido-, nitro-, hydroxyl-, oxyalkyl-, carbonyl-, carboxyl-, sulfonyl- and/or sulfoxyl substituted aryl or heteroaryl;
L^{J1}, L^{J2}, L^{J3} and L^{J4} independently of each other are selected from —NRC(═O)—, —C(═O)N(R)—, —NRC(═O)O—, —OC(═O)N(R)—, —C(R)═N—, —N═C(R)—, —C(═O)—, —OC(═O)—, —C(═O)O—, —N(R)—, —O—, —P(═O)(OR)—, —P(═O)(OR)O—, —OP(═O)(OR)—, —OP(═O)(OR)O—, —S—, —SO—, —SO₂—, —SO₂N(R)—, —N(R) SO₂N(R)—, —N(R) SO₂— with R selected from H and unsubstituted or amino-, hydroxyl-, carboxyl-, sulfonate- or fluoro-substituted C₁ to C₆ alkyl, particularly when R is selected from H and methyl;
m, m′, n, n′, p, p′, q, q′ and s independently from each other are selected from 0 and 1, and the binding moiety M is defined as above.

In a more specific embodiment, the compound of the present invention, in particular a photoactivatable fluorescent dye or fluorescent dye, has the general structure I-1-I-32 or II-1-II-32, and the said moiety is represented by one of the following structures:

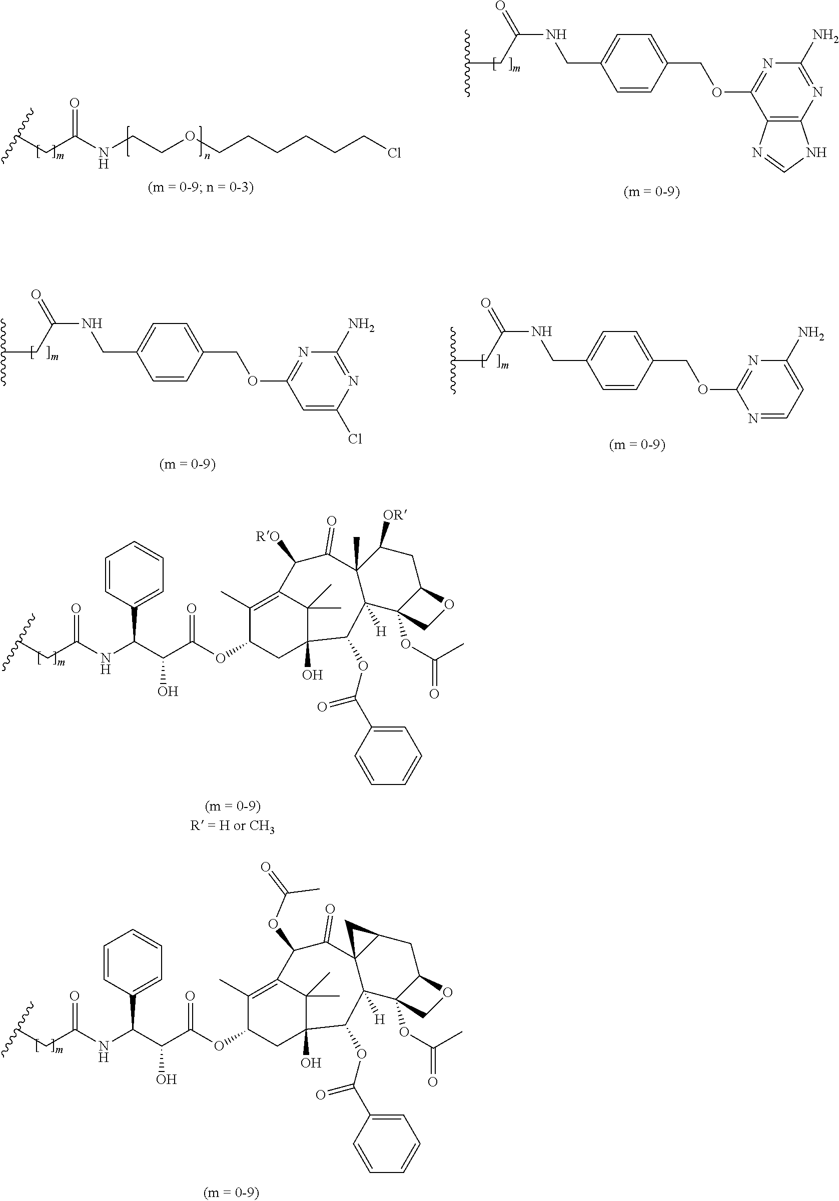

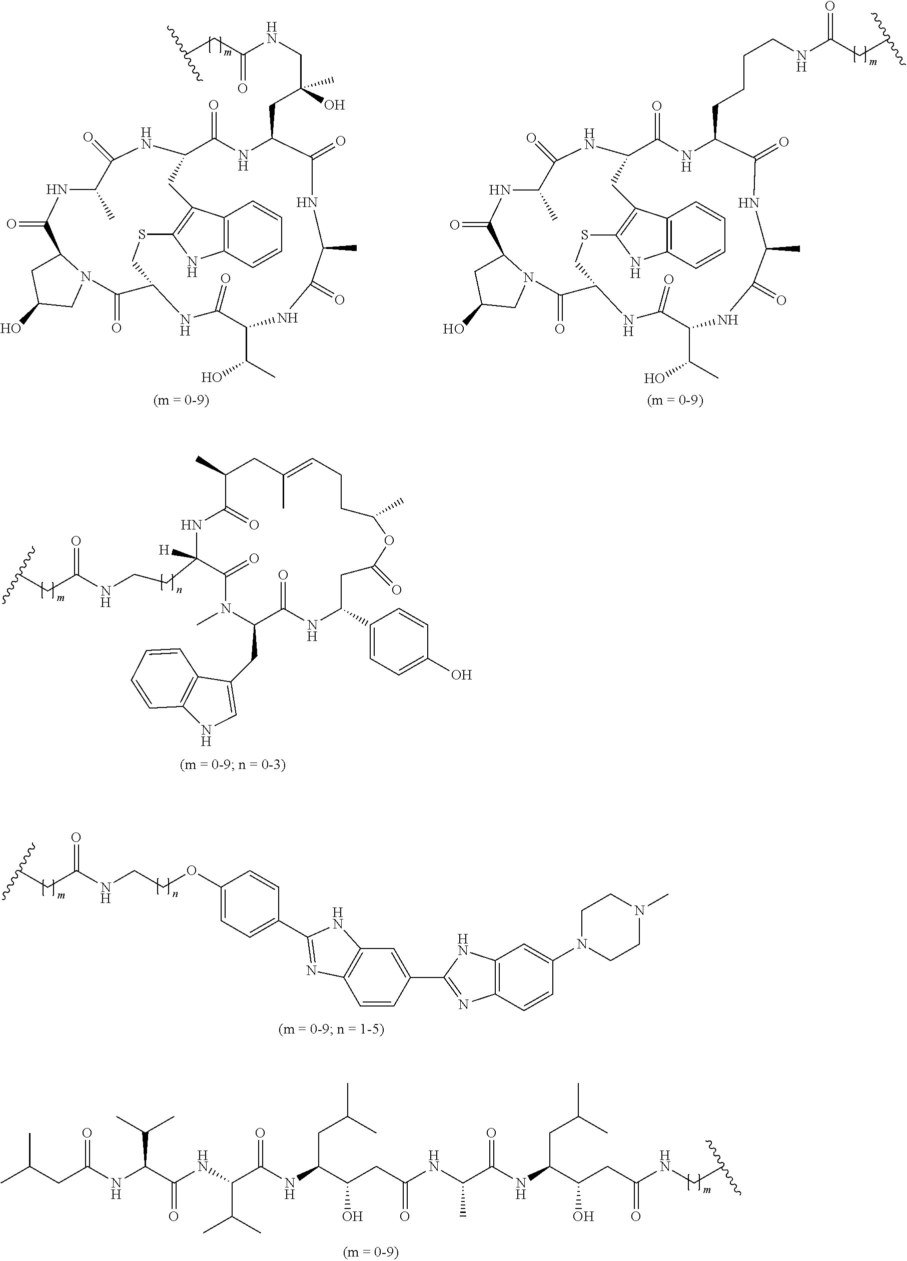

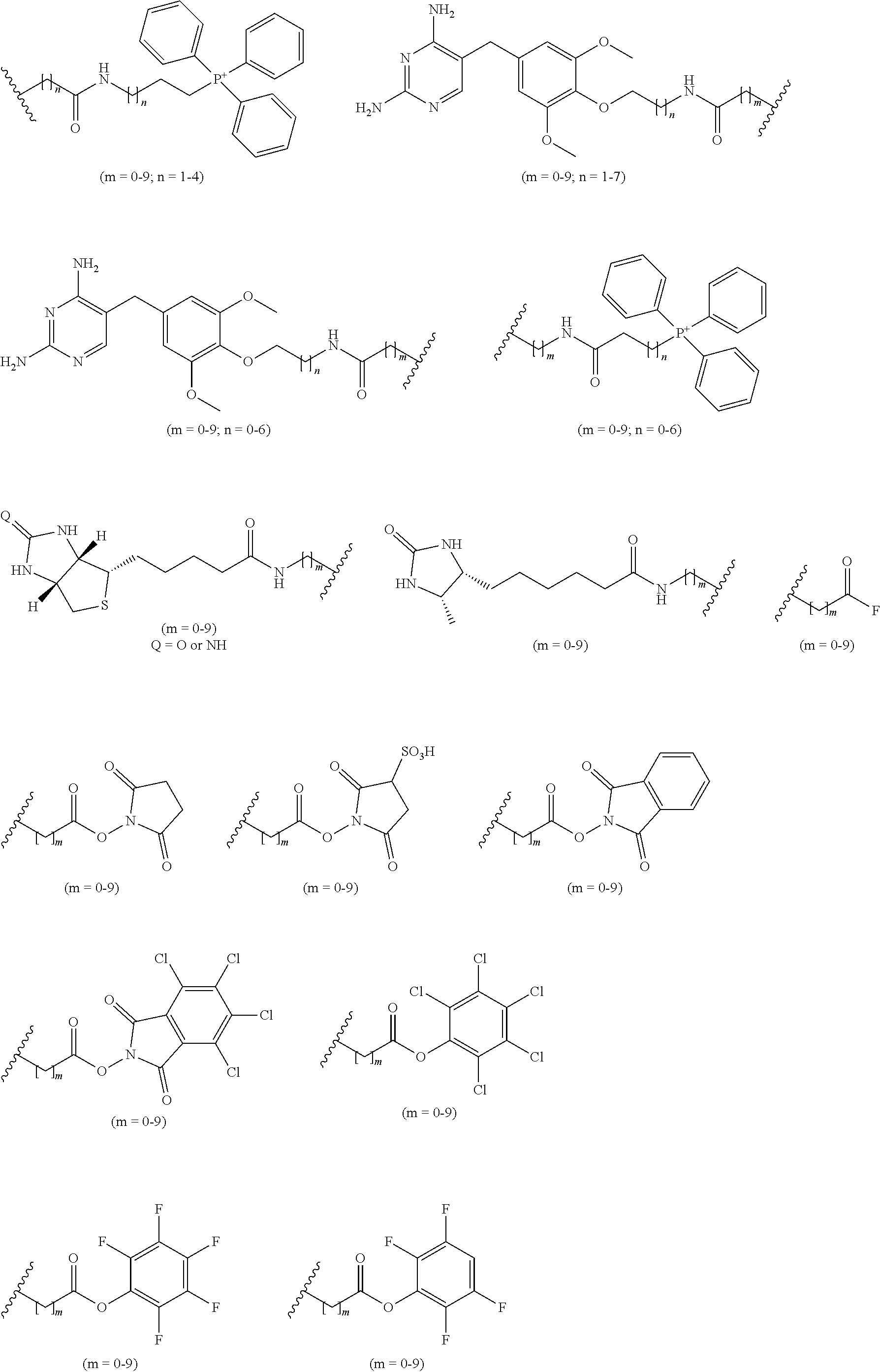

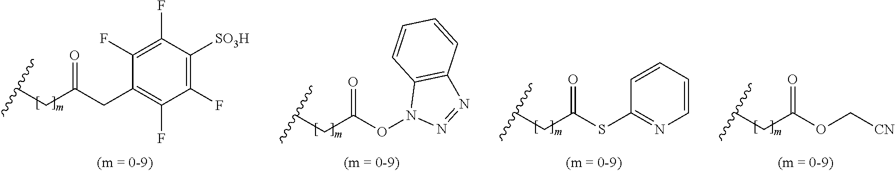

In another specific embodiment, the compound of the present invention, in particular a photoactivatable fluorescent dye or fluorescent dye, has the general structure I-1-I-32 or II-1-II-32, and:
a. R⁹ and R¹⁰, and/or R¹¹ and R¹², are independently selected from H, unsubstituted and amino-, hydroxy-, carboxy-, sulfonate- and/or fluoro-substituted C₁-C₆ alkyl, C₁-C₄ acyl, C₁-C₄ alkoxycarbonyl (including tert-butyloxycarbonyl or Boc group) and C₃-C₆ cycloalkyl,
particularly when R⁹ and R¹⁰, and/or R¹¹ and R¹², are independently selected from H, methyl, ethyl, n-propyl, isopropyl, n-butyl, isobutyl, tert-butyl, allyl and CH₂CF₃; or
b. R⁹ together with R¹⁰, and/or R⁹ together with R¹⁰, are independently forming an unsubstituted or alkyl-, amino-, hydroxy-, carboxy-, sulfonate- and/or fluoro-substituted C₃-C₆ alkyl, particularly-(CH₂)₃—, —(CH₂)₄—, —(CH₂)₅—, —(CH₂)₂O(CH₂)₂—, —(CH₂)₂SO₂ (CH₂)₂— or —(CH₂)₂NR²³ (CH₂)₂— with R²³ being selected from H and unsubstituted C₁ to C₄ alkyl (particularly methyl); or
c. R⁹ and/or R¹¹ are independently selected from H, unsubstituted and alkyl-substituted (particularly methyl-substituted), amino-, hydroxy-, carboxy-, sulfonate- and/or fluoro-substituted C₁-C₆ alkyl, C₁-C₄ acyl, C₁-C₄ alkoxycarbonyl (including tert-butyloxycarbonyl or Boc group) and C₃-C₆ cycloalkyl, and R¹⁰ together with R² or R³, and/or R¹² together with R⁴ or R⁵, is an alkyl or heteroalkyl bridge selected from —(CH₂)₂—, —(CH₂)₃—, —CH₂CH═CH— or —(CH₂)₄- or —CH₂—O—, —CH₂—NR—, —CH₂—S—, —CH₂—SO₂—, —(CH₂)₂O—, —(CH₂)₂NR—, —(CH₂)₂S—, —(CH₂)₂SO₂—, —CH₂—O—CH₂—, —CH₂NR—, —CH₂S—CH₂—, —CH₂—SO₂—CH₂-(with R selected from H and unsubstituted or amino-, hydroxyl-, carboxyl, sulfonate or fluoro substituted C₁ to C₆ alkyl, particularly when R is selected from H and methyl) and a mono- or dimethyl substituted derivative of any one of the foregoing alkyl or heteroalkyl bridge moieties; or
d. R¹⁰ and/or R¹¹ are independently selected from H, unsubstituted and alkyl-substituted (particularly methyl-substituted), amino-, hydroxy-, carboxy-, sulfonate- and/or fluoro-substituted C₁-C₆ alkyl, C₁-C₄ acyl, C₁-C₄ alkoxycarbonyl (including tert-butyloxycarbonyl or Boc group) and C₃-C₆ cycloalkyl, and R⁹ together with R², and/or R¹² together with R⁵, form a fused annular structure according to any one of the following substructures:

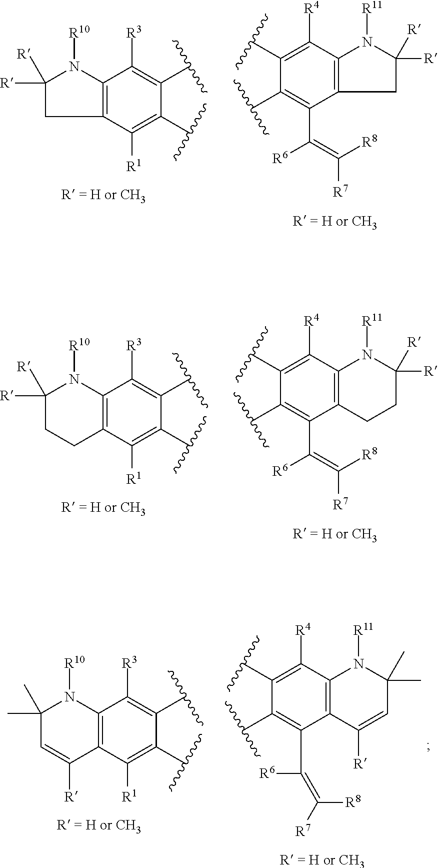

or
e. R⁹ and/or R¹² are independently selected from H, unsubstituted and alkyl-substituted (particularly methyl-substituted), amino-, hydroxy-, carboxy-, sulfonate- and/or fluoro-substituted C₁-C₆ alkyl, C₁-C₄ acyl, C₁-C₄ alkoxycarbonyl (including tert-butyloxycarbonyl or Boc group) and C₃—C; cycloalkyl, and R¹⁰ together with R³, and/or R¹¹ together with R⁴, form a fused annular structure according to any one of the following substructures:

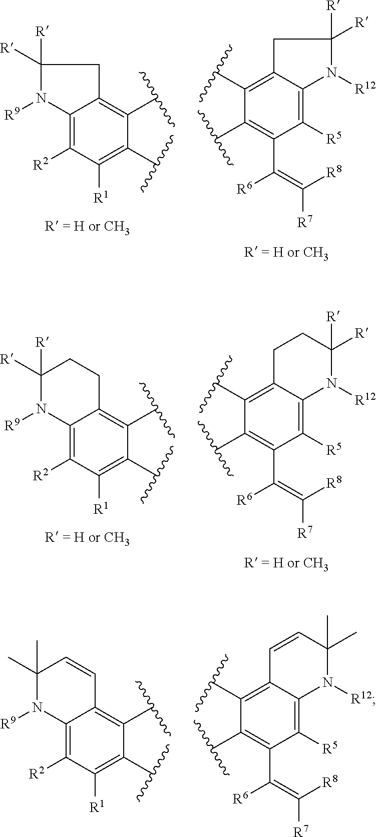

or
f. R⁹ together with R², and R¹⁰ together with R³, and/or R¹² together with R⁵, and R¹¹ together with R⁴, form a fused biannular structure according to any one of the following substructures:

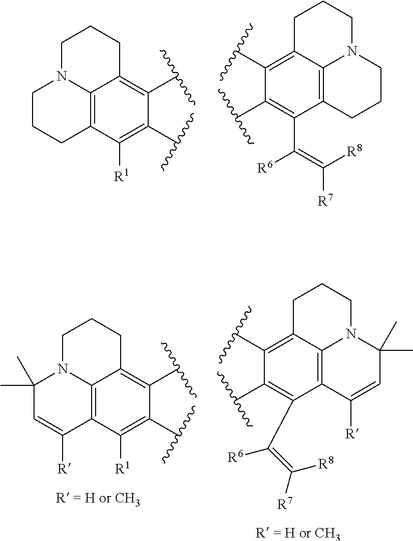

In another specific embodiment, the compound of the present invention, in particular a photoactivatable fluorescent dye or fluorescent dye, has the general structure I-1-I-32 or II-1-II-32, and R¹ is structurally identical to the substituent-CR⁶═CR⁷R⁸, in particular when the substituents R² and R⁵ are structurally identical, and/or the substituents-NR⁹R¹⁰ and —NR¹¹R¹² are structurally identical, and/or the substituents R³ and R⁴ are structurally identical.

In another more specific embodiment, the compound of the present invention, in particular a photoactivatable fluorescent dye or fluorescent dye, has the general structure I-1-I-32 or II-1-II-32, and:
R¹ is H, and/or
R², R³, R⁴ and R⁵ are independently selected from H, halogen, CN, and/or
R⁹, R¹⁰, R¹¹ and R¹² are individually unsubstituted or amino-, hydroxyl- or halogen-substituted C₁ to C₄ alkyl, or C₃ to C₆ cycloalkyl, or R⁹ together with R¹⁰ together with the N atom to which they are bound, and R¹¹ together with R¹² together with the N atom to which they are bound form an unsubstituted or methyl-, hydroxy-, methoxy-, or halogen-substituted aziridine, azetidine, pyrrolidine, piperidine, piperazine, morpholine, thiomorpholine-S,S-dioxide, and/or
R¹³, R¹⁴, R¹⁵ (if present) are selected from methyl, ethyl, isopropyl or phenyl,
R¹⁶, R¹⁷ (if present) are methyl,
one of the substituents R⁶, R⁷, R⁸ and R²⁰, R²¹, R²² (if present) is selected from a) unsubstituted or amino-, hydroxyl-, carboxyl- and/or halogen-substituted C₂ to C₁₂ alkyl or C₃ to C₇ cycloalkyl; or b)-Lₘ^{A1}-Lₘ′^{J1},—Lₙ^{A2},—Lₙ′^{J2},—Lₚ^{A3}-Lₚ′^{J3}-L_{q}^{A4},—L_{q′}^{J4}, -Mₛ, wherein L^{A1}, L^{A2}, L^{A3}, L^{A4}, L^{A1}, L^{J2}, L^{J3}, L^{J4} m, m′, n, n′, p, p′, q, q′, s and M have the definitions recited above, and
the other substituents R⁶, R⁷, R⁸ and R²⁰, R²¹, R²² (if present) are selected from H or methyl.

In another more specific embodiment, the compound of the present invention, in particular a photoactivatable fluorescent dye or fluorescent dye, has the general structure I-1-I-32 or II-1-II-32, and the substituents-NR⁹R¹⁰ and/or —NR¹¹R¹² are represented by one of the following structures:

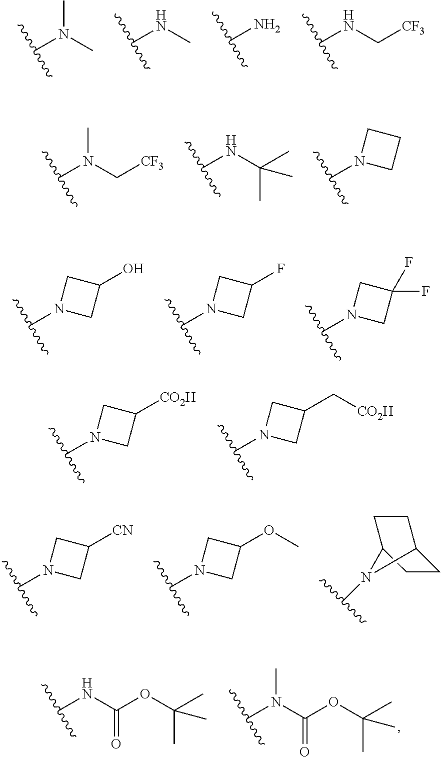

particularly when the substituents —NR⁹R¹⁰ and —NR¹¹R¹² are structurally identical.

In another more specific embodiment, the compound of the present invention, in particular a photoactivatable fluorescent dye or fluorescent dye, has the general structure I-1-I-32 or II-1-II-32, and the fragment-CR⁶═CR⁷R⁸ is represented by one of the following structures:

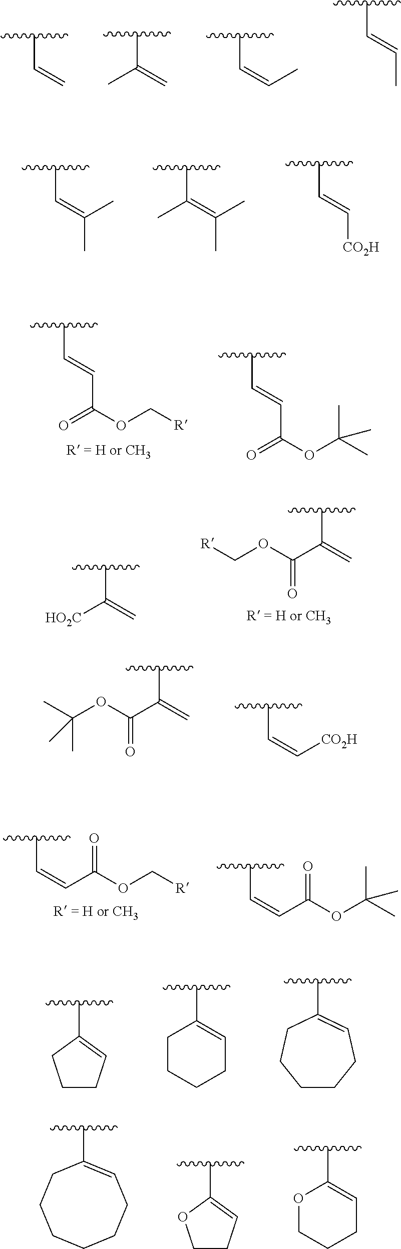

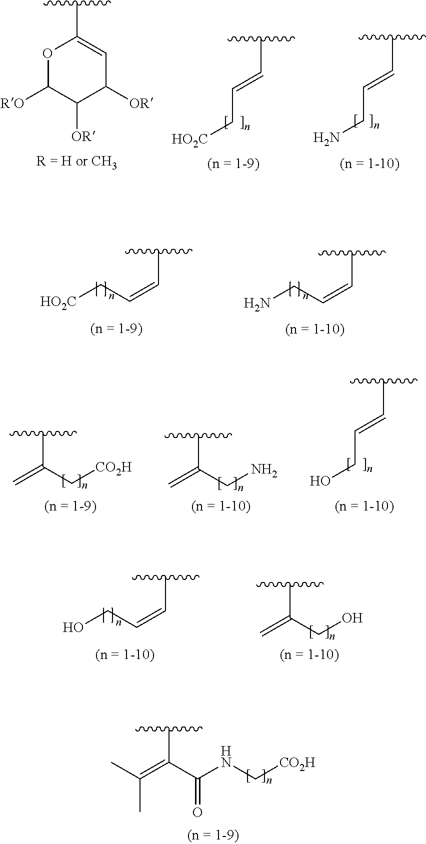

In another more specific embodiment, the compound of the present invention, in particular a photoactivatable fluorescent dye or fluorescent dye, has the general structure I-1-I-32 or II-1-II-32, and the substituent ═Y is represented by one of the following structures:

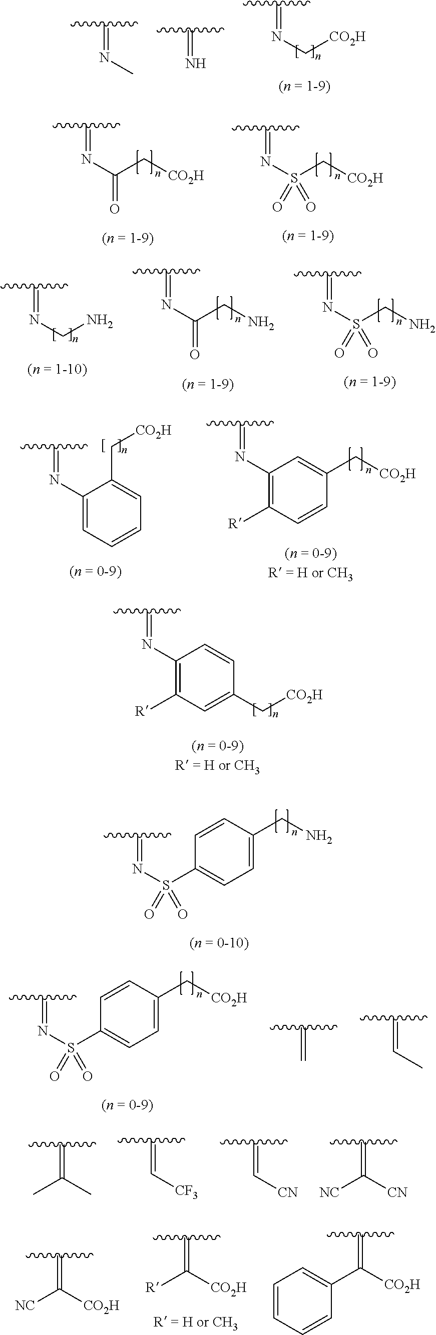

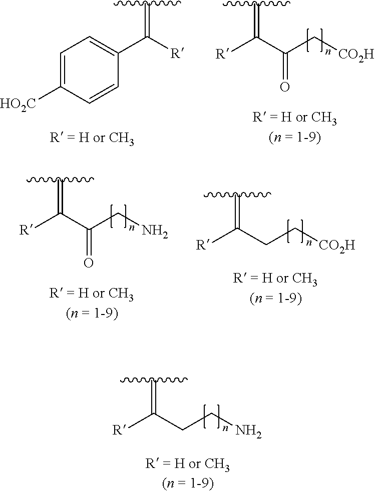

In another more specific embodiment, the compound of the present invention, in particular a photoactivatable fluorescent dye or fluorescent dye, has the general structure I-1-I-32 or II-1-II-32, and the group —X— is represented by one of the following structures:

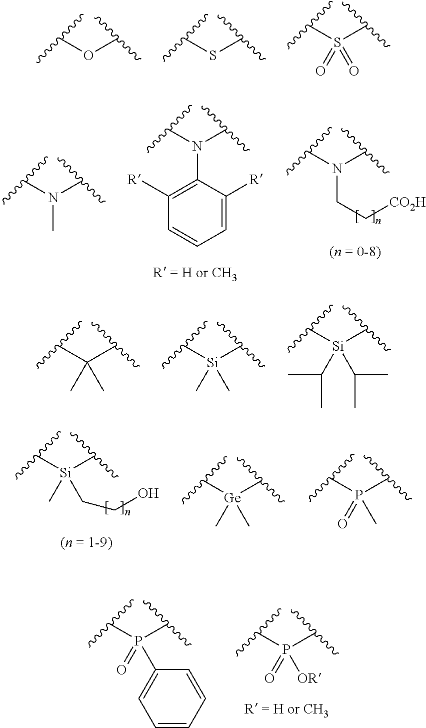

In another more specific embodiment, the compound of the present invention, in particular a photoactivatable fluorescent dye or fluorescent dye, having the general structure I-1-I-32 or II-1-II-32, is represented by one of the following structures:

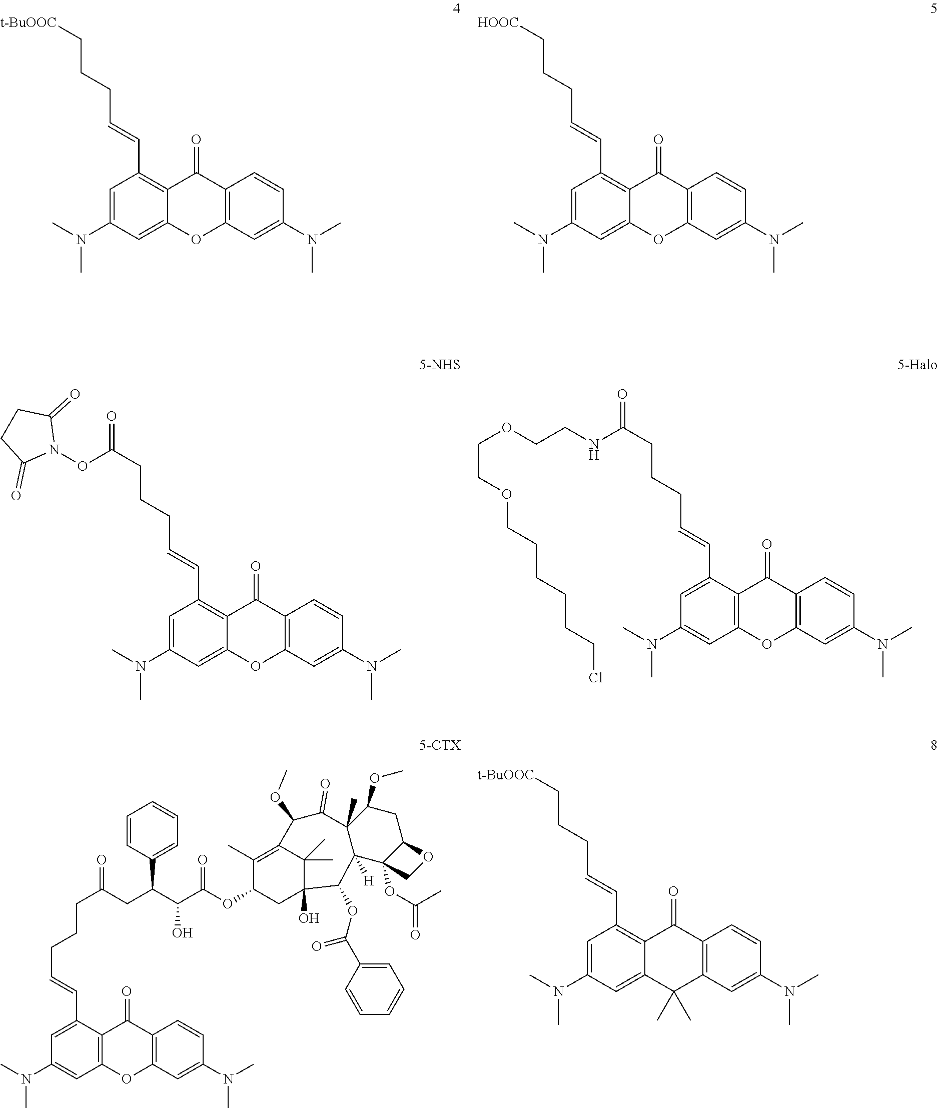

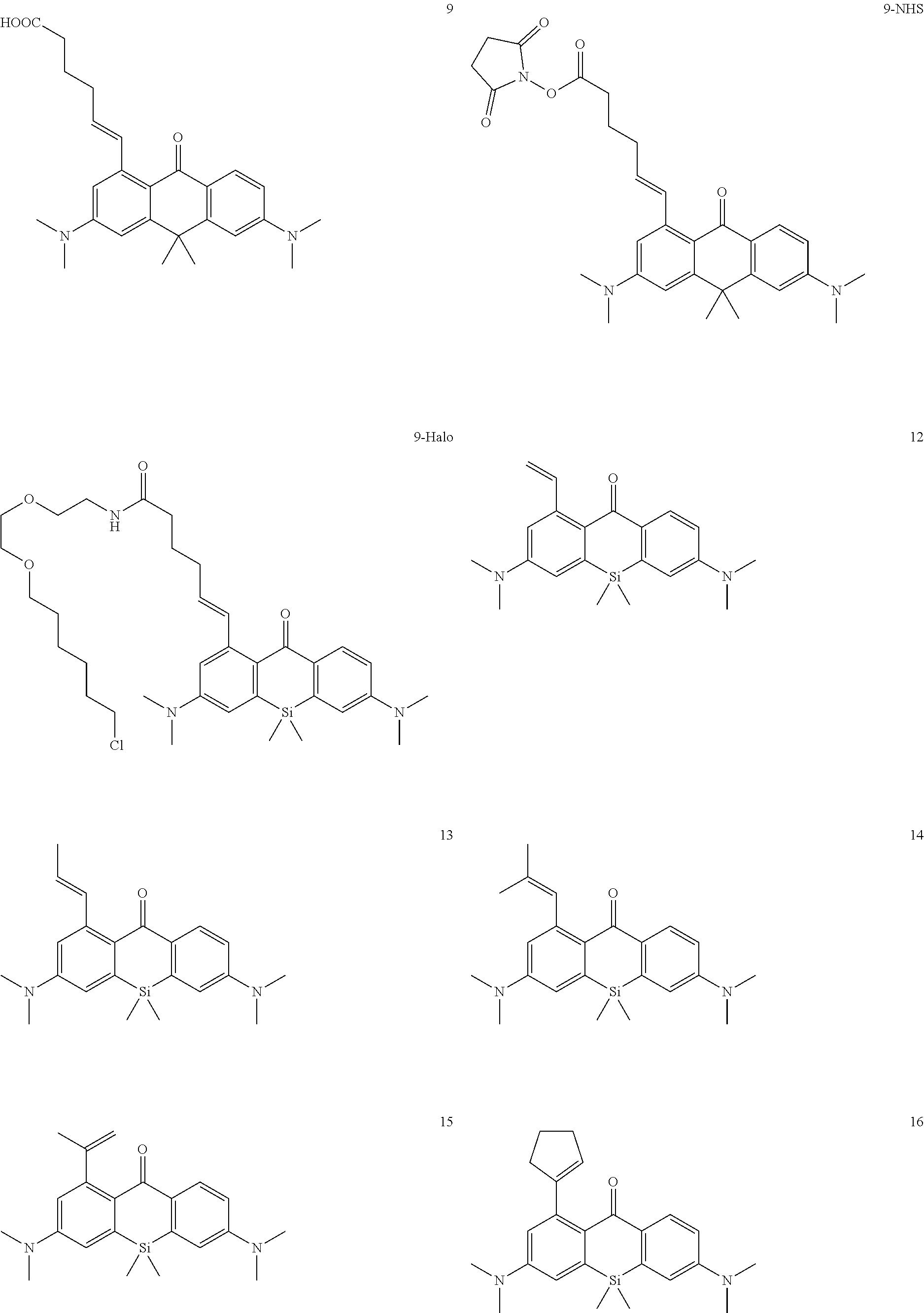

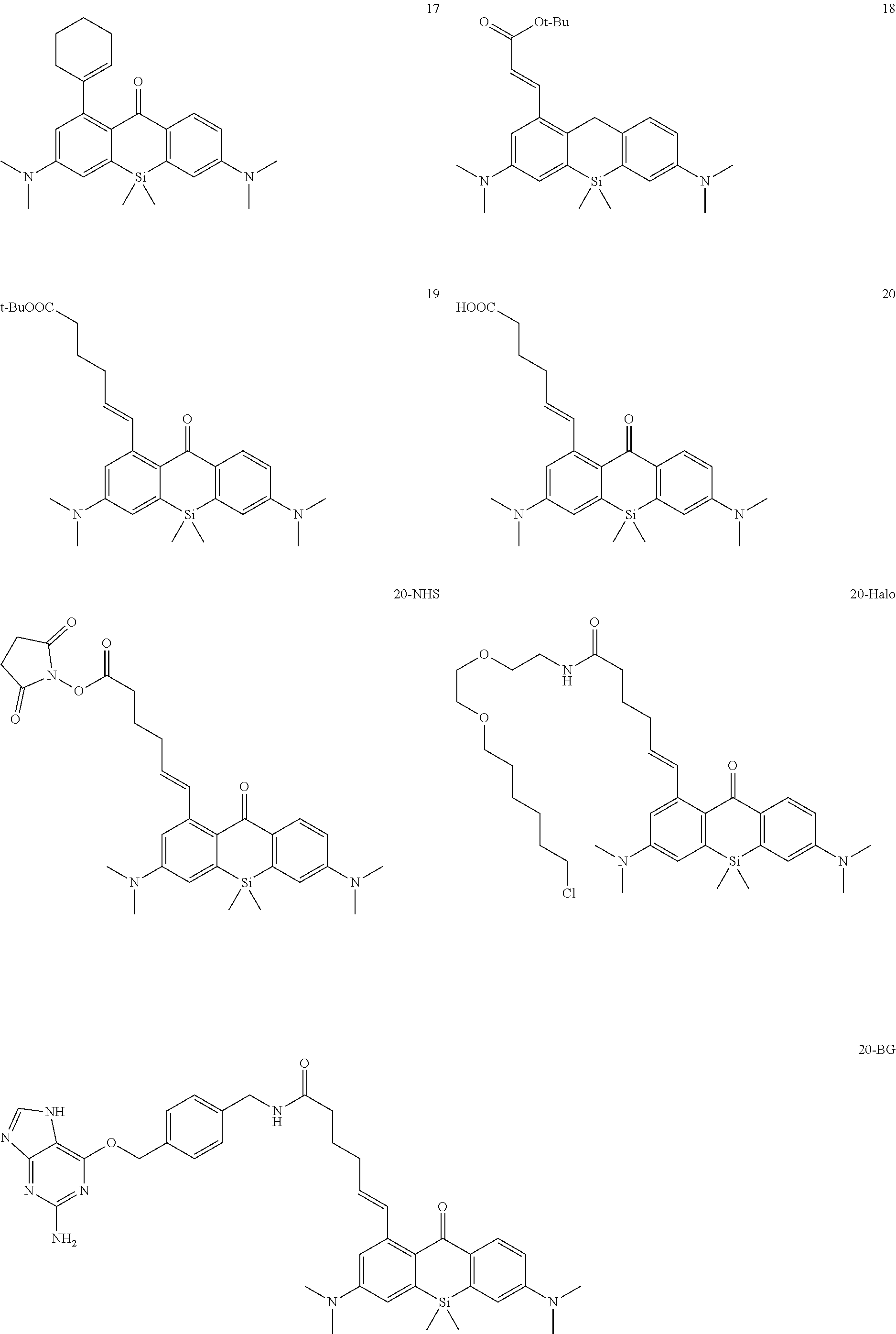

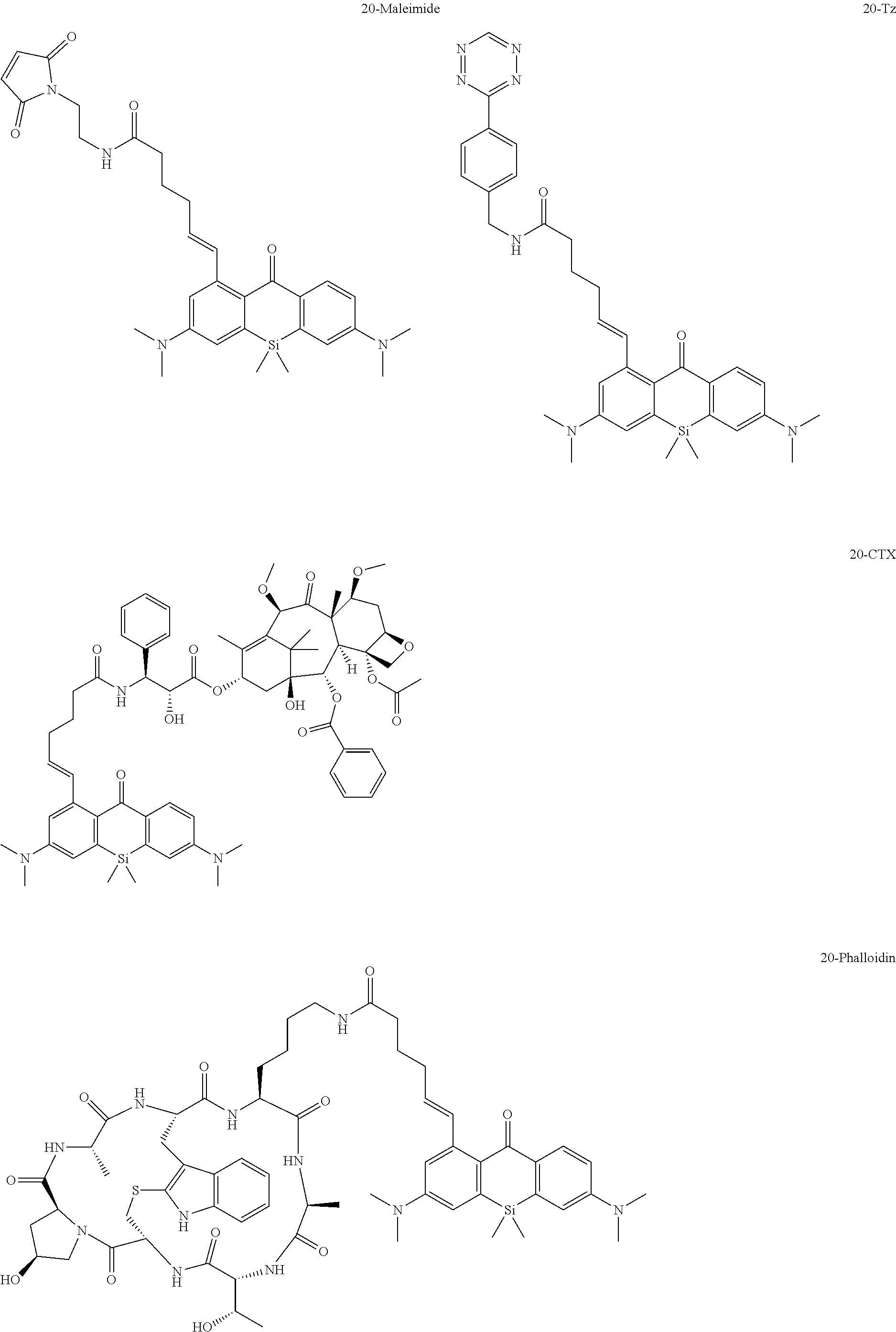

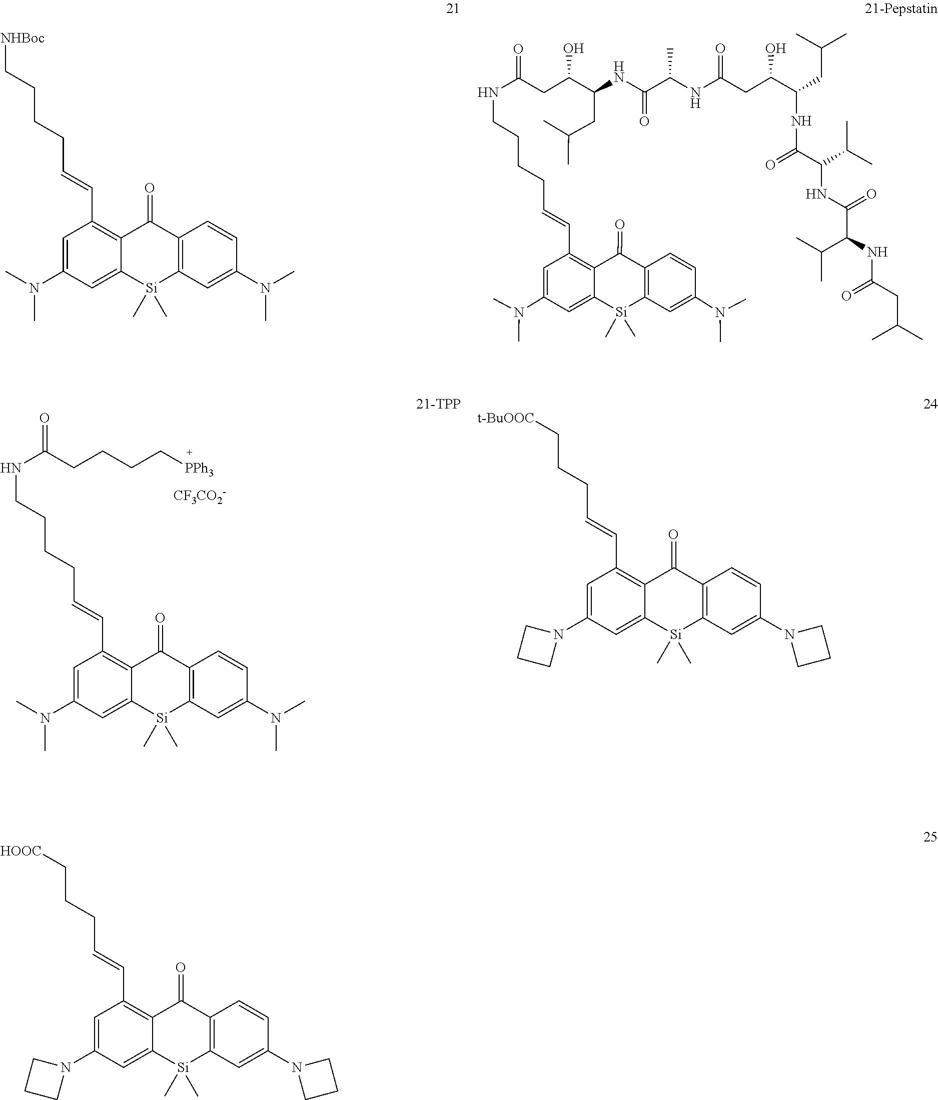

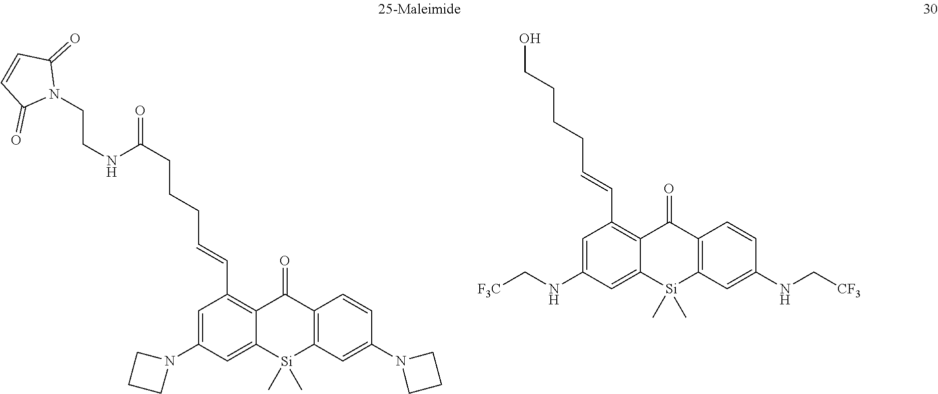 [SMILES: C[Si]1(C)c2cc(N3CCC3)ccc2C(=O)c2c(/C=C/CCCC(=O)NCCN3C(=O)C=CC3=O)cc(N3CCC3)cc21 ; C[Si]1(C)c2cc(NCC(F)(F)F)ccc2C(=O)c2c(/C=C/CCCCO)cc(NCC(F)(F)F)cc21]

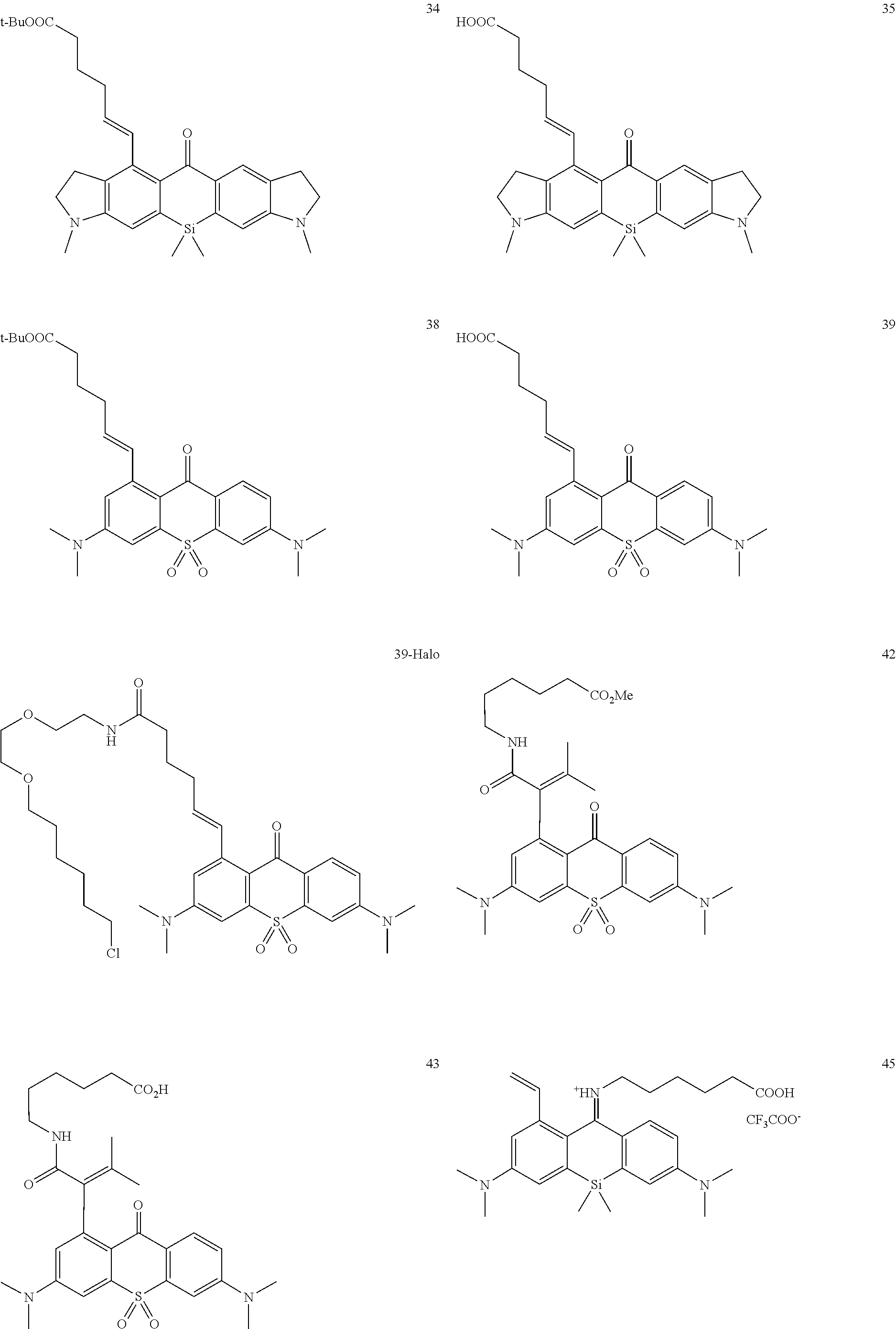

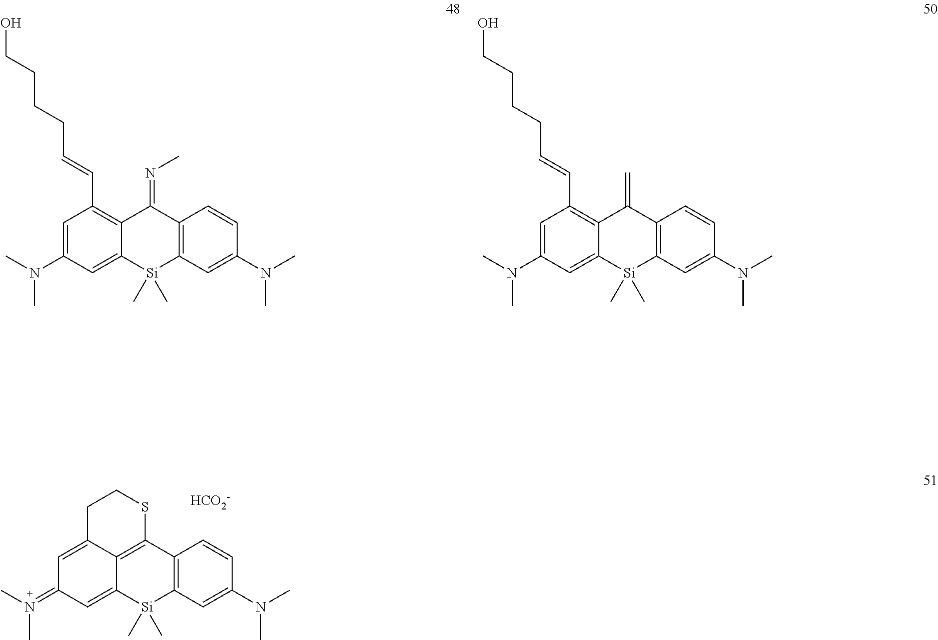

### General Characteristics of the Novel Compounds (Photoactivatable Fluorescent Dyes) of the Invention

The photoactivatable fluorescent dyes of the present invention are intended to be used in particular as photoactivatable fluorescent labels in super-resolution fluorescence microscopy methods in the context of fixed or living cells and extracellular matrix. General descriptions of various super-resolution imaging methods are presented in [Godin et al. Biophys J. 2014, 107, 1777-1784] and [Sahl, S. J.; Hell, S. W. High-Resolution 3D Light Microscopy with STED and RESOLFT. In: High Resolution Imaging in Microscopy and Ophthalmology: New Frontiers in Biomedical Optics; Bille, J. F., Ed.; Springer International Publishing, 2019; pp 3-32; DOI: 10.1007/978-3-030-16638-0_1], and representative applications of super-resolution microscopy in cell biology are presented in [Sahl et al. Nat. Rev. Mol. Cell Biol. 2017, 18, 685-701].

The requirements imposed by these methods and met by the photoactivatable fluorescent dyes of the present invention are as follows:
1) Intact cell membrane permeability for both the inactivated non-fluorescent form (corresponding to the general structure I) and the activated (fluorescent) form (corresponding to the general structure II) of the fluorescent label;
2) Reliable photoactivation kinetics of the compounds (photoactivatable fluorescent dyes) of general structure I, with relatively high photoactivation quantum yield which can be further tuned by variation of the substituents R¹, R², R³, R⁴, R⁵, X, Y and in particular R⁶, R⁷, R⁸, R⁹, R¹⁰, R¹¹ and R¹²;
3) Good solubility in aqueous media and low propensity of the labels and labeled antibodies, derived from the compounds of the general structures I and II, to aggregation and off-targeting in live and fixed cells or organisms;
4) Photostability of the photoactivated fluorescent dyes of the general structure II against ultraviolet (UV, >300 nm), visible and infrared (IR) light, which allows to achieve a high photon budget (emitted photon count per molecule before photobleaching), sufficient for any fluorescence microscopy technique, including superresolution techniques;
5) The mechanism of photoactivation of the dyes of the general structure I ensures a single well defined photochemical activation pathway and thus minimizes the possibility of formation of dark non-fluorescent side products (as in the case of diazoketone-caged rhodamines and rhodamine analogues) and the release of reactive or toxic byproducts arising from the cage groups (as in the case of various 2-nitrobenzyl- or 2-nitrobenzyloxycarbonyl-caged fluorescent dyes);
6) Compact structure and low molecular weight (MW<500 Da) for many typical compounds of the general structure I (see Examples below) and low number of functional groups, thus generally conforming to Lipinski's rule of five (≤5 hydrogen bond donors, ≤10 hydrogen bond acceptors, n-octanol/water partition coefficient log P≤5.6 [A. K. Ghose et al. J. Comb. Chem. 1999, 1 (1), 55-68]).
7) Low chemical reactivity of the photoactivated (fluorescent) dyes, corresponding to the general structure II, ensuring the stability of the labels inside the living cells in the natural presence of oxidizing (molecular oxygen) and nucleophilic species (functional proteins and reduced glutathione), and in diverse imaging media compositions for fixed-cell imaging;
8) High chemical stability across a broad pH range of compounds of the general structures I and II; in addition, a reliable photoactivation mechanism operating across the biologically relevant pH range and in diverse solvents and media, such as in aqueous solvents, alcohols, and polymer films.

### Applications

As follows from the above-mentioned characteristics, the compounds (photoactivatable dyes or photoactivated dyes) of the present invention are suitable for various applications, in particular in the field of optical microscopy and bioimaging techniques.

The most basic aspect of the present invention relates to the use of a novel compound as defined above or of a conjugate or derivative comprising the same as photoactivatable fluorescent dyes.

In a more specific embodiment, these compounds, derivatives or conjugates may be used for staining a biological sample, in particular whole organisms, mammalian and non-mammalian cells including insect, plant, fungi, bacteria cells and viral particles.

In a more specific embodiment, these compounds, derivatives or conjugates may be used for tracking and monitoring dynamic processes in a sample or in an object or tracking and monitoring the behavior of single molecules within a sample or an object.

In another specific embodiment, these compounds, derivatives or conjugates may be used as components in inorganic, bio-inorganic, organic or macromolecular composites as materials for optical memories, data storage, photo-lithography, photo-activatable paints and inks.

In another specific embodiment, these compounds, derivatives or conjugates may be used as fluorescent tags, analytical reagents and labels in optical microscopy, imaging techniques, protein tracking, nucleic acid labeling, glycan analysis, flow cytometry or as a component of biosensors, or as analytical tools or reporters in microfluidic devices or nanofluidic circuitry.

In another more specific embodiment, these compounds, derivatives or conjugates as such or after photoactivation may be used as energy donors or acceptors (reporters) in applications based on fluorescence energy transfer (FRET) process or as energy acceptors (reporters) in applications based on bioluminescence resonance energy transfer (BRET) process.

In another specific embodiment, the optical microscopy and imaging methods may comprise stimulated emission depletion microscopy [STED] or any of its improved versions with reduced phototoxicity (e.g, FastRESCue STED), when additional color multiplexing is achieved by combining the compounds, derivatives or conjugates of the present invention together with any other STED-compatible fluorescent dyes in a single sample under study.

In another specific embodiment, the optical microscopy and imaging methods may comprise single molecule switching techniques (SMS: diffraction unlimited optical resolution achieved by recording the fluorescence signals of single molecules, reversibly or irreversibly switched between emitting and non-emitting states, such as single molecule localization microscopy [SMLM], photoactivation localization microscopy [PALM, PALMIRA, fPALM], stochastic optical reconstruction microscopy [STORM], minimal photon fluxes [MINFLUX] or their parallelized implementations, fluorescence correlation spectroscopy [FCS], fluorescence recovery after photobleaching [FRAP], and fluorescence lifetime imaging [FLIM].

In another specific embodiment, additional color multiplexing may be achieved by these compounds, derivatives or conjugates as such or after photoactivation together with any other fluorescent dyes in a single sample or object under study.

In another specific embodiment, the activation of spatiotemporal subpopulations of photoactivatable dyes of the present invention allows imaging with the photoactivated fluorophore molecules while protecting the remaining photoactivatable dyes from photobleaching.

The presently-disclosed subject matter further includes a method of using the compounds described herein. In some embodiments, the method comprises utilizing the photoactivated fluorescent labels of the present invention as a reporter for enzyme activity, as a fluorescent tag, as a photosensitizer, as a pH indicator, as a redox indicator, as an intracellular environment polarity indicator, as an optical sensor of transmembrane potential, as a sensor for a target substance (an analyte), as an agent for imaging experiments, and/or as an imaging agent for super-resolution microscopy.

The presently-disclosed method for detecting a target substance can further comprise a detecting step that includes detecting an emission light from the compound, the emission light indicating the presence of the target substance, or a ratiometric detection step which comprises detecting an emission light before and after photoactivating the dyes of the present invention within the sample.

In some embodiments the method for using the compounds comprises photoactivating a compound of the present invention by exposing the sample to a UV or blue light. As described herein, the photoactivating light source can produce an excitation wavelength from ultraviolet light to blue light in the visible range. In specific embodiments the excitation wavelength can be in a range of 200 nm to about 500 nm, or preferably in a range of about 350 nm to about 450 nm.

In some embodiments the method for using the compounds comprises photoactivating a compound of the present invention by exposing the sample to an orange, red or infrared (IR) light making use of multiphoton excitation conditions. As described herein, the photoactivating light source can be an orange, red or IR laser of sufficiently high power. In specific embodiments the excitation wavelength can be in a range of 500 nm to about 1500 nm, or preferably in a range of about 700 nm to about 1100 nm.

In some embodiments the method for using the compounds further comprises exposing the photoactivated compound to an excitation light. As described herein, the excitation light can include any wavelength matching the absorption of the compound, from ultraviolet light to near infrared light, by either a one-photon or multi-photon process. In specific embodiments the absorption wavelength can be in a range of 200 nm to about 1200 nm, or preferably in a range of about 400 nm to about 820 nm.

In some embodiments the detecting step is performed by use of fluorescence spectroscopy or by the naked eye. In some embodiments the detecting step is performed with a microscope. In some embodiments the detecting step is performed with a fluorimeter or a microplate reader, or within a flow cell. In some embodiments the presence of a target substance can indicate the occurrence or absence of a particular biological function, as will be appreciated by those skilled in the art. In some embodiments the method is performed in a live cell, a tissue and/or a subject.

Some embodiments of detection methods comprise contacting the sample with two or more embodiments of compounds that are selective for different target substances. Methods for detecting two or more target substances with two or more of the presently-disclosed compounds are referred to herein as “multiplex” detection methods.

In some of the present multiplex methods, two or more distinct target substances and/or two or more regions of one target substance are detected using two or more probes, wherein each of the probes is labeled with a different embodiment of the present compounds. The presently-disclosed compounds can be used in multiplex detection methods for a variety of target substances, whereby the first compound can be selective for a first target substance, is excited with a first absorption wavelength and can be emitting a first emission light, and the second compound can be selective for a second target substance, is excited with a second absorption wavelength and can be emitting a second emission light, while both compounds are sharing the same photoactivation conditions (multiplexing by excitation or emission wavelengths). In other embodiments of the multiplex methods, the photoactivatable compounds of the present studies are employed together where the first compound is photoactivated with an activation wavelength and a minimum intensity of light that does not photoactivate the second compound. The second compound is photoactivated with either a second activation wavelength of light suitable for photoactivation (multiplexing by photoactivation wavelength) or a greater light intensity with the first activation wavelength (multiplexing by photoactivation kinetics). In other embodiments of the multiplex methods, the photoactivatable compounds of the present studies are employed together with the common fluorophores that do not require a photoactivating step to be used in fluorescence detection methods (multiplexing by photoactivation). In some embodiments the emission wavelengths of the first and second compounds are different from one another, and in other embodiments the excitation (absorption) wavelengths of the first and second compounds are different from one another (multiplexing by Stokes shift), providing an efficient means for detecting a plurality of different target substances in one setting.

As a non-limiting illustrative example, imaging in samples can be performed in cells transiently or endogenously expressing protein fusions with SNAP- or Halo-Tag proteins labelled with the photoactivatable dyes of the present invention (e.g., 20-BG or 20-Halo) and commonly used STED-compatible “always-on” fluorescent dyes (i.e. non photoactivatable or non-caged compounds) for a different target structure (such as Abberior Live 560-Tubulin probe for beta-tubulin) and emitting in the same spectral detection channel. The commonly used “always-on” dyes can then be imaged in confocal microscopy using their respective excitation lasers (e.g., 561 nm) and detection channels (e.g., 571-630 nm). These fluorescent dyes can then be photobleached using high power of 561 nm light without photoactivating the dyes of corresponding to general structure I of the present invention. The photoactivatable dyes can then be activated by illumination with an activation laser (e.g. a 405 nm) and imaged using the same excitation lasers and detection channels as before for the standard always-on fluorophores. This imaging routine allows to duplex every available color channel on a commercial confocal or STED system (e.g. STED 595/775 quad scanning microscope, Abberior Instruments, Göttingen, Germany), but can be adapted to suit another confocal or STED instrument from a different supplier or a custom-built STED nanoscopy setup (see FIG. 5).

As another non-limiting illustrative example, imaging in samples can be performed with one or more than one pair of primary and mutually orthogonal secondary antibodies, labelled with photoactivatable dyes of the present invention (e.g., 5-NHS and 20-NHS) each with different rates of photoactivation. The compound with the greater rate of photoactivation (in this case, 20) can be selectively photoactivated by low intensity of illumination with e.g. a 355 nm or a 405 nm activation laser and imaged using their respective excitation lasers (e.g., 561 nm) and detection channels. The second compound (in this case, 5) can be next photoactivated by a high intensity illumination with e.g. a 355 nm or a 405 nm activation laser and imaged using the respective excitation and detection channels. This imaging routine allows multiple photoactivatable dyes of the present invention to be used in combination on a commercial confocal or STED system (e.g. STED 595/775 quad scanning microscope, Abberior Instruments, Göttingen, Germany), but can be adapted to suit another confocal or STED instrument from a different supplier or a custom-built STED nanoscopy setup (See FIG. 7).

### General Approach for Synthesizing the Novel Compounds, in Particular Photoactivatable Fluorescent Dyes, of the Invention

For the preparation of the novel compounds, the present inventors have identified the following synthetic sequences leading to the example compounds Ia of the present invention and starting (as in following illustrative and non-limiting examples) from the advanced intermediates of type A1-A7, described previously in the literature (A1: [P. Horváth et al. J. Org. Chem. 2015, 80 (3), 1299-1311]; A2: [A. N. Butkevich et al. Angew. Chem. Int. Ed., 2016, 55 (10), 3290-3294]; A3: [A. N. Butkevich et al. J. Am. Chem. Soc. 2017, 139, 12378-12381]; A4: [S. Shen et al. RSC Adv., 2017, 7 (18), 10922-10927]; A5: [S. Jia et al. ACS Chem. Biol. 2018, 13 (7), 1844-1852]; A6: [G. Lukinavičius et al. J. Am. Chem. Soc. 2016, 138 (30), 9365-9368]; A7: [J. Liu et al. ACS App. Mat. Interfaces 2016, 8 (35), 22953-22962]).

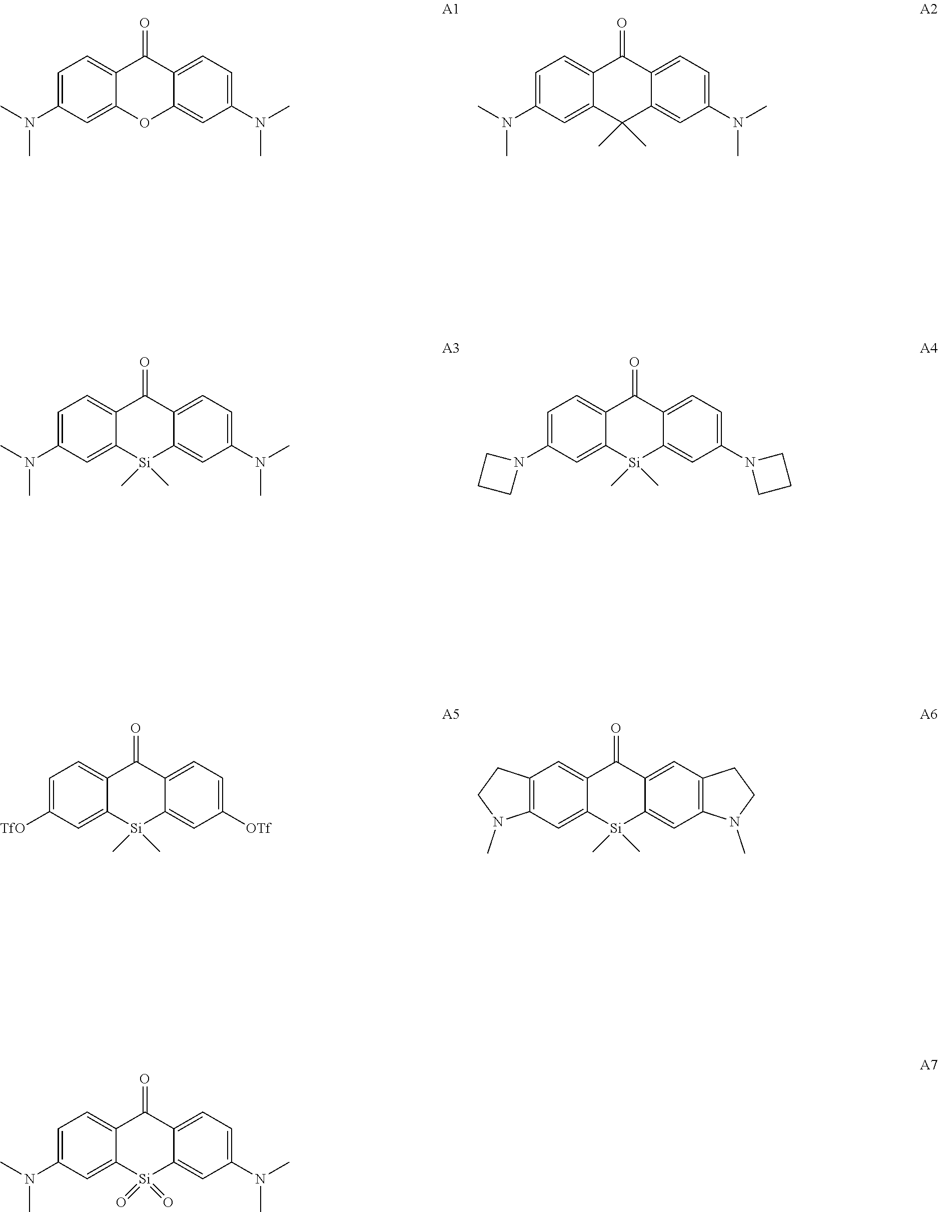

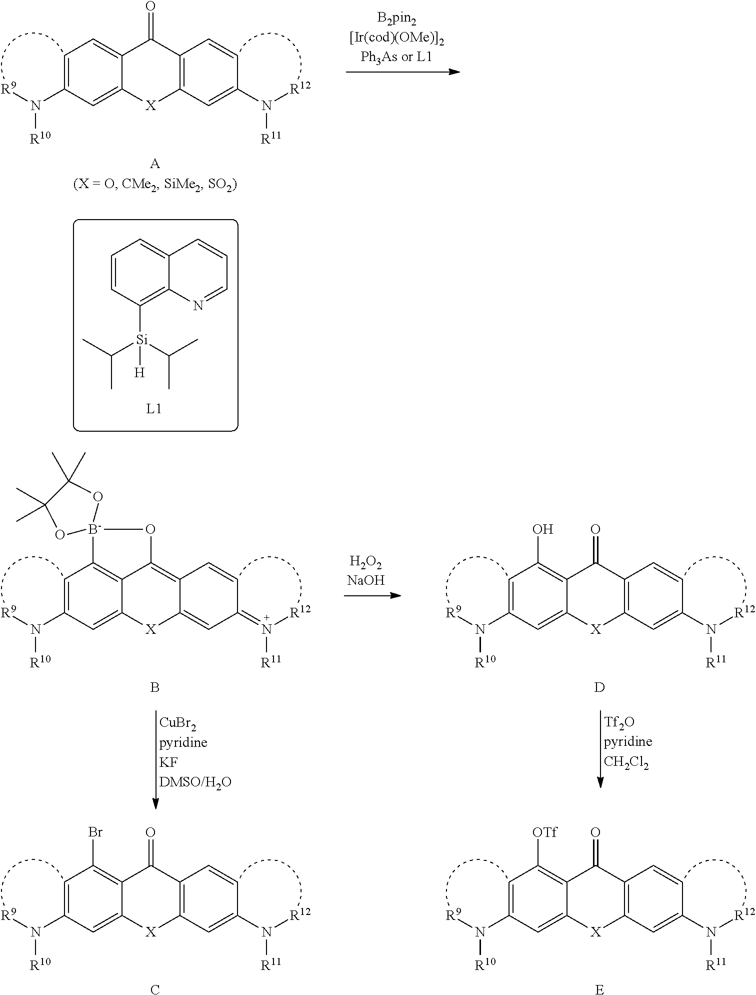

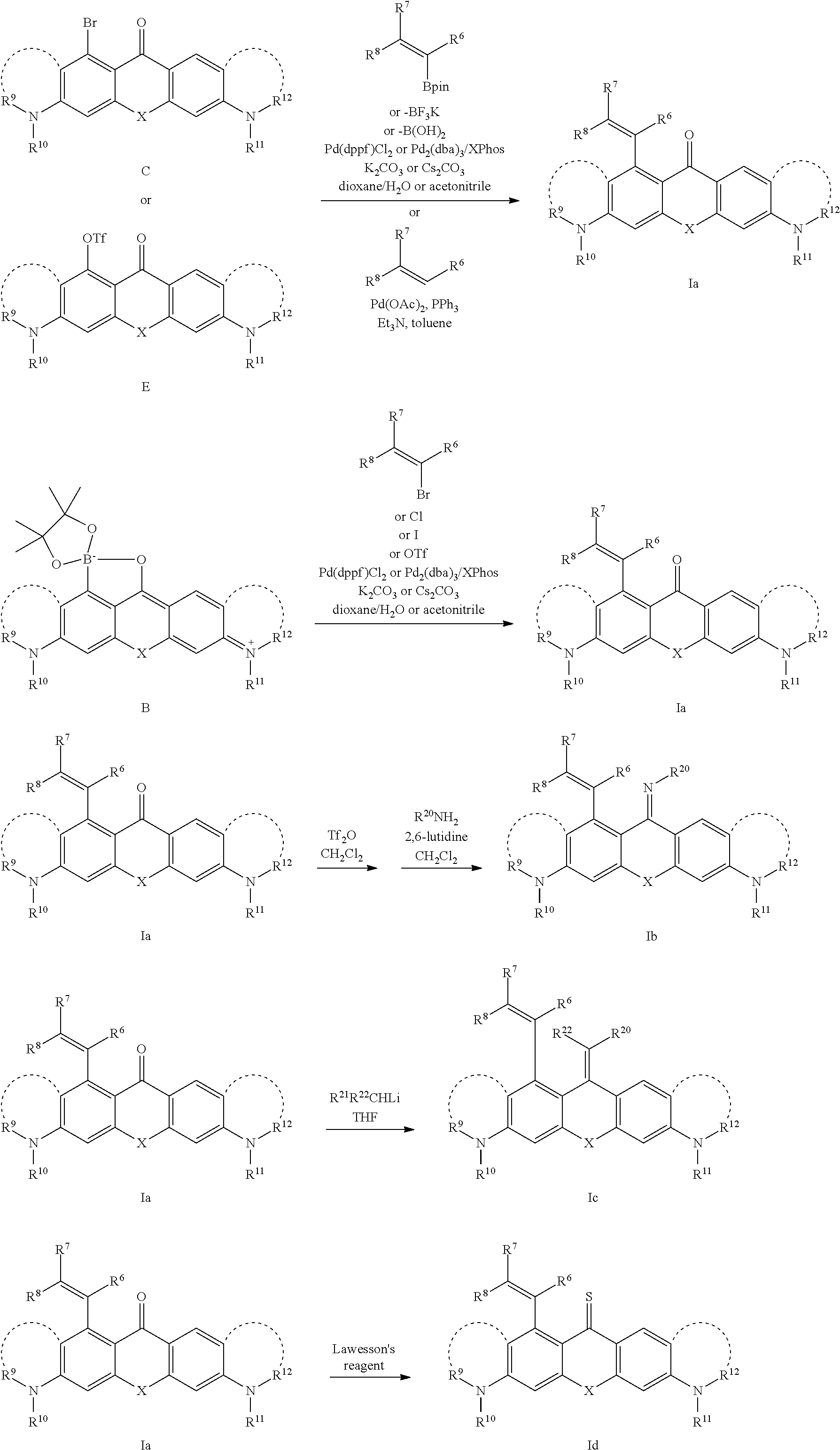

However, any suitable synthetic method can be used to synthesize compounds according to the present invention, with variations including the choice of reagents and catalysts, reaction conditions and the order of synthetic steps.

### BRIEF DESCRIPTION OF THE FIGURES

FIG. 1 shows absorption (A) and emission (B) changes during photo-induced activation of compound 13 with violet light (405 nm) in an aqueous buffered solution at pH 7 (phosphate buffer, 100 mM); HPLC 2D-maps of absorption spectra vs. retention time for samples of the solution before (C) and after (D) the photo-induced activation; and chromatograms (E) of these samples at the wavelengths corresponding to the respective absorption maxima.

FIG. 2 shows photo-fatigue resistance of compound 20 and established commercial fluorophores to excitation light (530 nm) in an aqueous buffered solution at pH 7 (phosphate buffer, 100 mM).

FIG. 3 shows live-cell dual-color/channel confocal imaging of vimentin filaments labelled with compound 20-Halo (A & C) and tubulin labelled with 6-SiR-CTX (B & D) in U2OS cells. Images were recorded before (A-B) and after photo-activation (C-D) with 405 nm light.

FIG. 4 shows live-cell confocal (A) and STED (B) image of vimentin filaments labelled with compound 20-Halo in U2OS cells. The compound was pre-activated by irradiation with a 405 nm laser. (C) The same samples was imaged in a confocal microscope, before pre-activation (top half of the image), and a 2-photon activation laser was switched on approximately in the middle of the scanning (bottom half of the image, as indicated by the arrows).

FIG. 5 shows live-cell single-color/channel confocal imaging U2OS cells stably expressing Vimentin-HaloTag fusion construct labelled with compound 20-Halo and Abberior Live 560-Tubulin. Sequential imaging was performed before photo-activation (A), after photo-bleaching of Abberior Live 560-Tubulin (B), and after photo-activation of compound 20-Halo (C). Absorption and emission spectra of Abberior Live 560-Tubulin (marked as AL-560) and 20-Halo (after photoactivation) is shown (D).

FIG. 6 shows Single Molecule Localization Microscopy superresolution images of nuclear pore complexes (A-C) and microtubules (D) in COS7 cells. Samples were fixed and immunolabelled with a primary antibody against NUP-98 from rabbit and an anti-rabbit secondary antibody labelled with 5-NHS (A-C) or a primary antibody against alpha-tubulin from mouse and an anti-mouse secondary labelled with 9-NHS (D).

FIG. 7 shows confocal imaging of a fixed Cos7 cells immunolabeled with anti-alpha-tubulin primary antibody from mouse and anti-clathrin primary antibody from rabbit, and anti-mouse secondary antibody labelled with 20-NHS and anti-rabbit secondary antibody labelled with 5—NHS. Sequential imaging was performed before photoactivation (A, B), after low photoactivation dose selective for 20 (C, D), and after high photoactivation dose sufficient to convert compound 5 (E, F).

FIG. 8 shows fluorescence patterning in a polymer film (PVA) doped with compound 5. Selected areas were irradiated with light of 405 nm. Confocal images of the same area were recorded in a before (A) and after photo-patterning (B), and in a wide-field fluorescence microscope (C), only of the patterned structure.

The present invention is further illustrated by the following specific but non-limiting examples.

### Example 1

### Synthesis of the Starting Materials, Photoactivatable Compounds and Photoactivatable Labels

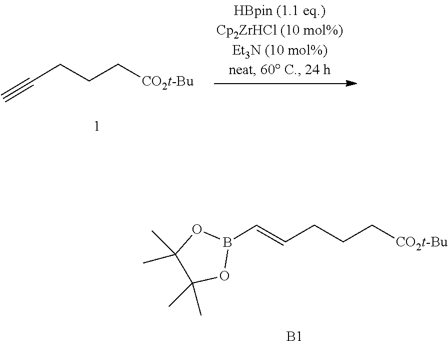 [SMILES: C#CCCCC(=O)OC(C)(C)C ; CC(C)(C)OC(=O)CCC/C=C/B1OC(C)(C)C(C)(C)O1 ; "B1"]

Compound B1 was prepared according to the method reported in S. Bera, X. Hu. Angew. Chem. Int. Ed. 2019, 58 (39), 13854-13859. In a dried 10 mL crimp-top tube (a 2-5 mL Biotage microwave vial was used), compound 1 (1 g, 6 mmol; prepared according to the literature procedure: E. Bomal et al. Chem. Eur. J. 2020, 26 (41), 8907-8915) and Schwartz's reagent (zirconocene chloride hydride; 155 mg, 0.6 mmol, 10 mol %) were placed, and the contents of the vial were degassed on a Schlenk line. Pinacolborane (HBpin; 950 μL, 6.55 mmol, ˜1.1 equiv) was then injected followed by triethylamine (84 μL, 0.6 mmol, 10 mol %), the vial was placed in a 60° C. oil bath and the reaction mixture was stirred for 24 h. The mixture was then cooled down to rt, diluted with diethyl ether and filtered through a 2 cm plug of silica, washing with diethyl ether (50 mL). The filtrate was evaporated and the product was isolated by flash chromatography on Biotage Isolera system (40 g RediSep Rf cartridge, gradient 0% to 20% ethyl acetate/hexane) to give colorless oil, yield 1.28 g (72%).

¹H NMR (400 MHZ, CDCl₃): δ 6.60 (dt, J=18.0, 6.4 Hz, 1H), 5.45 (dt, J=18.0, 1.6 Hz, 1H), 2.26-2.14 (m, 4H), 1.72 (p, J=7.6 Hz, 2H), 1.44 (s, 9H), 1.27 (s, 12H).

¹³C NMR (101 MHz, CDCl₃): δ 173.0, 153.4, 119.6 (br.), 83.2, 80.2, 35.2, 35.1, 28.2, 24.9, 23.7. HRMS (ESI) m/z: [M+H]⁺ Calcd for C₁₆H₂₉BO₄ 297.2235; Found 297.2231.

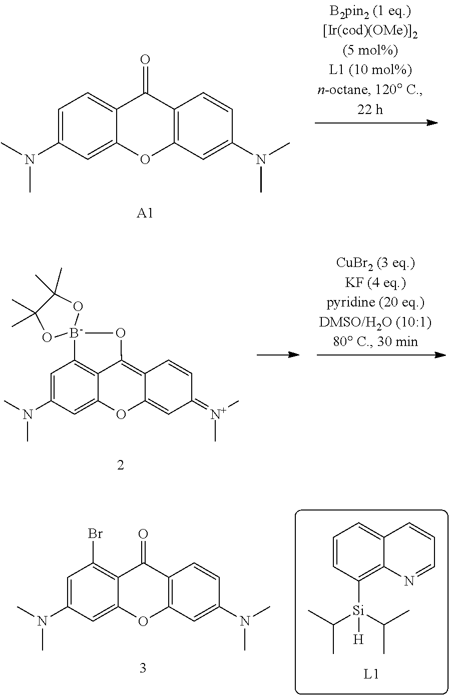

Compound 3. In a 25 mL round-bottom flask, the mixture of compound A1 (382 mg, 1.35 mmol; known compound: P. Horvath et al. J. Org. Chem. 2015, 80 (3), 1299-1311), bis(pinacolato) diboron (344 mg, 1.35 mmol, 1 equiv), [Ir (cod)(OMe)]₂ (45 mg, 0.068 mmol, 5 mol %) and ligand L1 (8-(diisopropylsilyl) quinoline; known compound: B. Ghaffari et al. J. Am. Chem. Soc. 2014, 136, 14345-14348)(33 mg, 0.135 mmol, 10 mol %) in dry n-octane (13 mL) was degassed on a Schlenk line and stirred at 120° C. for 22 h. On cooling, the reaction mixture was diluted with dichloromethane and filtered through a plug of Celite, washing with dichloromethane. The filtrate was evaporated to dryness in a 50 mL round-bottom flask and the obtained crude 2 was used directly in the next step.

To the residue of crude compound 2, potassium fluoride (313 mg, 5.40 mmol, 4 equiv), copper (II) bromide (903 mg, 4.05 mmol, 3 equiv) were added, followed by DMSO (12 mL), water (1.2 mL) and pyridine (2.2 mL, 27 mmol, 20 equiv). The reaction mixture was stirred at 80° C. for 30 min. Upon cooling to rt, the reaction mixture was diluted with ethyl acetate and poured into water (100 mL). The product was extracted with ethyl acetate (3×50 mL), the combined extracts were washed with brine (50 mL) and dried over Na₂SO₄. The product was isolated by flash chromatography on Biotage Isolera system (40 g RediSep Rf cartridge, gradient 0% to 20% ethyl acetate/dichloromethane) to give 327 mg (67%) of 3 as yellow solid.

¹H NMR (400 MHZ, CDCl₃): δ 8.08 (d, J=9.0 Hz, 1H), 6.86 (d, J=2.6 Hz, 1H), 6.64 (dd, J=9.0, 2.5 Hz, 1H), 6.40 (d, J=2.6 Hz, 1H), 6.35 (d, J=2.5 Hz, 1H), 3.06 (s, 6H), 3.03 (s, 6H).

¹³C NMR (101 MHz, CDCl₃): δ 174.1, 159.0, 157.0, 154.4, 152.9, 128.1, 122.4, 115.6, 112.2, 109.5, 109.2, 97.7, 96.5, 40.3, 40.1.

HRMS (ESI) m/z: [M+H]⁺ Calcd for C₁₇H₁₇BrN₂O₂ 361.0546; Found 361.0546.

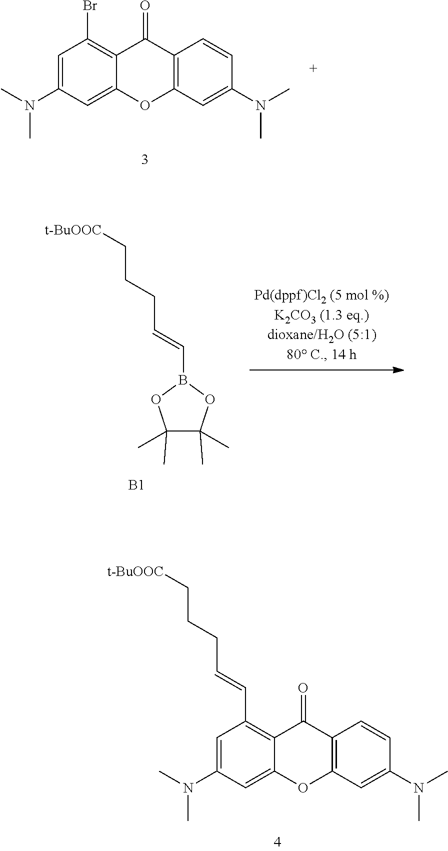

Compound 4. In a 10 ml tube, compound 3 (36 mg, 0.10 mmol), compound B1 (44 mg, 0.15 mmol), K₂CO₃ (18 mg, 0.13 mmol) and Pd(dppf) Cl₂·CH₂Cl₂ (4.1 mg, 5 μmol, 5 mol %) were loaded. Dioxane (1.0 mL) and water (0.2 mL) were added. The mixture was sparged with argon for 30 min, and the tube sealed and stirred at 80° C. for 14 h. Upon cooling, the reaction mixture was diluted with ethyl acetate (10 mL) and washed with sat. aq. NH₄Cl (10 mL), and brine (10 ml). The organics were dried over Na₂SO₄, filtered, evaporated. The product was isolated by flash chromatography on Biotage Isolera system (12 g Interchim SiHP 30 μm cartridge, gradient 0% to 50% ethyl acetate/hexane) and freeze-dried from dioxane to yield 40 mg (89%) of 4 as a pale yellow solid.

¹H NMR (400 MHZ, CDCl₃): δ 8.08 (d, J=9.0 Hz, 1H), 7.91 (d, J=15.7 Hz, 1H), 6.66 (dd, J=9.0, 2.4 Hz, 1H), 6.63 (dd, J=2.6, 0.6 Hz, 1H), 6.42 (dd, J=5.2, 2.5 Hz, 2H), 6.01 (dt, J=15.5, 6.9 Hz, 1H), 3.09 (s, 6H), 3.08 (s, 6H), 2.39-2.28 (m, 4H), 1.90-1.78 (m, 2H), 1.45 (s, 9H).

¹³C NMR (101 MHz, CDCl₃): δ 176.6, 173.3, 159.2, 157.2, 154.1, 153.0, 142.3, 132.4, 131.3, 127.7, 113.0, 109.0, 107.7, 97.0, 96.5, 80.0, 40.2, 40.1, 35.3, 32.5, 28.2, 25.0.

HRMS (ESI) m/z: [M+H]⁺ Calcd for C₂₇H₃₄N₂O₄: 451.2591, found: 451.2590.

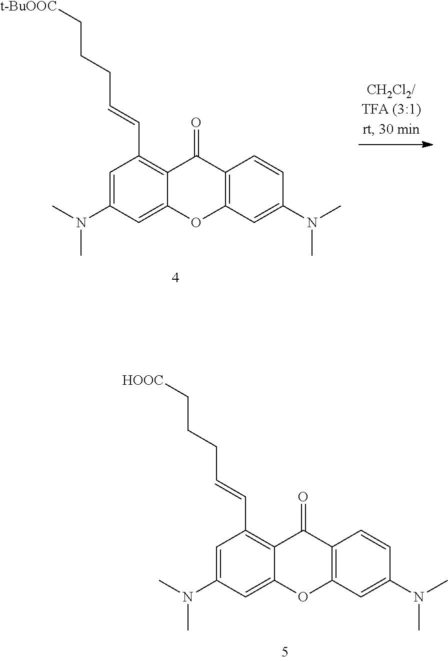

Compound 5. To a solution of compound 4 (34 mg; 0.075 mmol) in CH₂Cl₂ (600 μL) trifluoroacetic acid (200 μL) was added dropwise. The resulting reaction mixture was stirred for 30 minutes at rt, protected from light. The volatiles were removed in vacuo by coevaporation with toluene (3×10 ml). The product was freeze-dried from dioxane to give 30 mg (˜100%, remainder dioxane) of 5 as a pale yellow solid.

¹H NMR (400 MHZ, DMSO-d₆): δ 12.02 (s, 1H), 7.85 (d, J=3.7 Hz, 1H), 7.82 (d, J=3.0 Hz, 1H), 6.75 (dd, J=9.0, 2.4 Hz, 1H), 6.68 (d, J=2.5 Hz, 1H), 6.49-6.45 (m, 2H), 6.10 (dt, J=15.7, 6.7 Hz, 1H), 3.06 (s, 6H), 3.05 (s, 6H), 2.32 (t, J=7.4 Hz, 2H), 2.23 (q, J=6.8 Hz, 2H), 1.73 (p, J=7.4 Hz, 2H).

¹³C NMR (101 MHz, DMSO-d₆): δ 174.9, 174.5, 158.6, 156.5, 154.1, 152.8, 141.0, 131.4, 131.1, 127.0, 111.8, 109.2, 107.9, 107.0, 96.6, 96.0, 39.6, 33.2, 32.0, 24.1.

HRMS (ESI) m/z: [M+H]⁺ Calcd for C₂₃H₂₆N₂O₄: 395.1965, found: 395.1961.

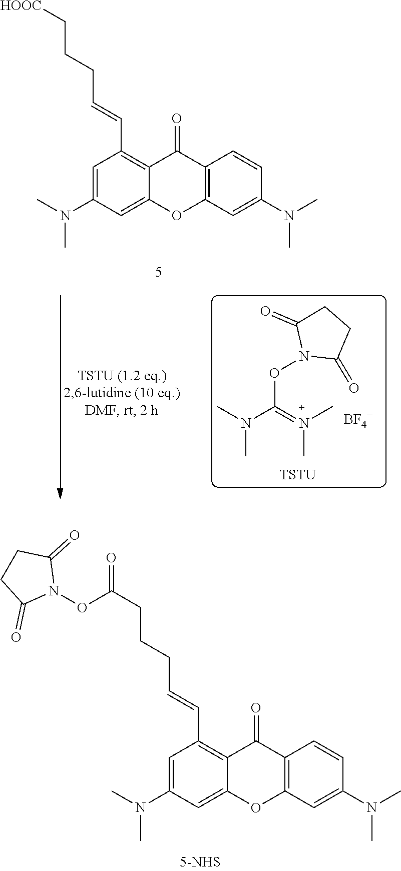

Compound 5-NHS. In an amber vial, compound 5 (23 mg, 0.058 mmol) and TSTU (N,N,N′, N′-tetramethyl-O—(N-succinimidyl) uronium tetrafluoroborate; 35 mg, 0.12 mmol) were dissolved in DMF (500 μL) and 2,6-lutidine (62 mg, 0.58 mmol) was added and stirred for 2 h at rt. The volatiles were removed in vacuo. The product was isolated by flash chromatography on a Biotage Isolera system (12 g Interchim SiHP 30 μm cartridge, gradient 20% to 100% ethyl acetate/hexane) and freeze-dried from 1,4-dioxane to give 5-NHS as an off-white solid. Yield 24 mg (81%, remainder dioxane).

¹H NMR (400 MHZ, CDCl₃): δ 8.07 (d, J=9.0 Hz, 1H), 7.94 (d, J=15.7 Hz, 1H), 6.67 (dd, J=9.0, 2.5 Hz, 1H), 6.62 (dd, J=2.6, 0.6 Hz, 1H), 6.44 (d, J=2.6 Hz, 1H), 6.42 (d, J=2.4 Hz, 1H), 5.99 (dt, J=15.6, 6.8 Hz, 1H), 3.10 (s, 6H), 3.08 (s, 6H), 2.83 (s, 4H), 2.74 (dd, J=8.1, 7.2 Hz, 2H), 2.48-2.39 (m, 2H), 2.02 (p, J=7.6 Hz, 2H).

¹³C NMR (101 MHZ, CDCl₃): δ 176.6, 169.1, 168.7, 159.2, 157.2, 154.2, 153.0, 142.0, 133.3, 129.9, 127.7, 112.9, 109.0, 107.8, 97.1, 96.5, 67.1, 40.2, 40.1, 32.0, 30.6, 25.6, 24.4.

HRMS (ESI) m/z: [M+H]⁺ Calcd for C₂₇H₂₉N₃O₆: 492.2129, found: 492.2130.

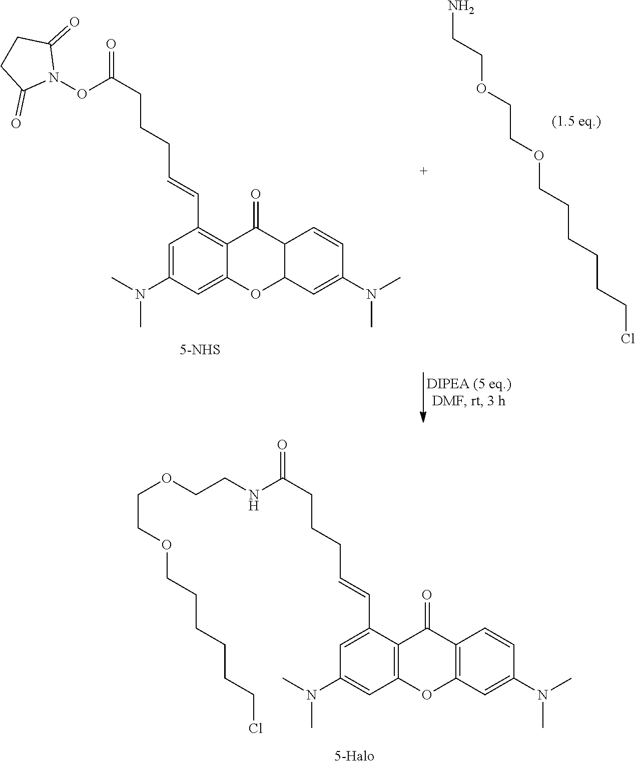 [SMILES: CN(C)C1=CC2Oc3cc(N(C)C)cc(/C=C/CCCC(=O)ON4C(=O)CCC4=O)c3C(=O)C2C=C1 ; NCCOCCOCCCCCCCl ; CN(C)c1ccc2c(=O)c3c(/C=C/CCCC(=O)NCCOCCOCCCCCCCl)cc(N(C)C)cc3oc2c1]

Compound 5-Halo. In an amber vial, compound 5-NHS (9.9 mg, 0.02 mmol) and HaloTag (02) Amine (6.7 mg, 0.03 mmol) were dissolved in DMF (100 μL). DIPEA (N,N-diisopropylethylamine; 12.9 mg, 0.1 mmol) was added and stirred for 3 hours at rt. The volatiles were removed in vacuo. The product was isolated by flash chromatography on a Biotage Isolera system (12 g Interchim SiHP 30 μm cartridge, gradient 0% to 100% A/B, A: 100% ethyl acetate, B: 2% methanol-98% ethyl acetate) and freeze-dried from dioxane to yield 9.7 mg (81%) of 5-Halo as an off-white solid.

HRMS (ESI) m/z: [M+H]⁺ Calcd for C₃₃H₄₆ClN₃O₅: 600.3199, found: 600.3202.

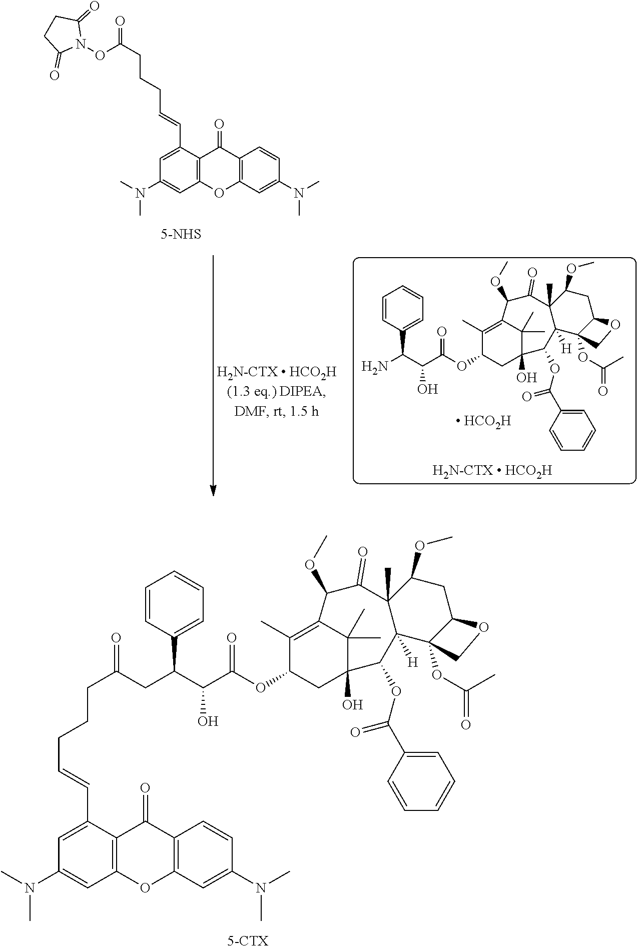 [SMILES: CN(C)c1ccc2c(=O)c3c(/C=C/CCCC(=O)ON4C(=O)CCC4=O)cc(N(C)C)cc3oc2c1 ; CO[C@H]1C(=O)[C@]2(C)[C@@H](OC)C[C@H]3OC[C@@]3(OC(C)=O)[C@H]2[C@H](OC(=O)c2ccccc2)[C@]2(O)C[C@H](OC(=O)[C@H](O)[C@@H](N)c3ccccc3)C(C)=C1C2(C)C ; "· HCO2H" ; CO[C@H]1C(=O)[C@]2(C)[C@@H](OC)C[C@H]3OC[C@@]3(OC(C)=O)[C@H]2[C@H](OC(=O)c2ccccc2)[C@]2(O)C[C@H](OC(=O)[C@H](O)[C@@H](CC(=O)CCC/C=C/c3cc(N(C)C)cc4oc5cc(N(C)C)ccc5c(=O)c34)c3ccccc3)C(C)=C1C2(C)C]

Compound 5-CTX. In an amber vial, compound 5-NHS (3.0 mg, 6.1 μmol) and H₂N-CTX·HCO₂H [A. N. Butkevich et al. ACS Chem. Biol. 2018, 13 (2), 475-480](6.3 mg, 8.0 μmol, 1.3 equiv) were dissolved in DMF (100 μL). DIPEA (30 μL) was added, and the reaction mixture was at rt for 1.5 h. The solvents were removed in vacuo, and the product the product was isolated by preparative HPLC (Interchim Uptisphere Strategy PhC4 250×21.2 mm 5 μm, solvent flow rate 18 mL/min, gradient 40% to 85% A: B, A-acetonitrile+0.1% (v/v) formic acid, B-water+0.1% (v/v) formic acid) and freeze-dried from dioxane to give 3.0 mg (44%) of 5-CTX as yellowish solid.

HRMS (ESI) m/z: [M+H]⁺ Calcd for C₆₃H₇₃N₃O₁₅: 1112.5114, found: 1112.5113.

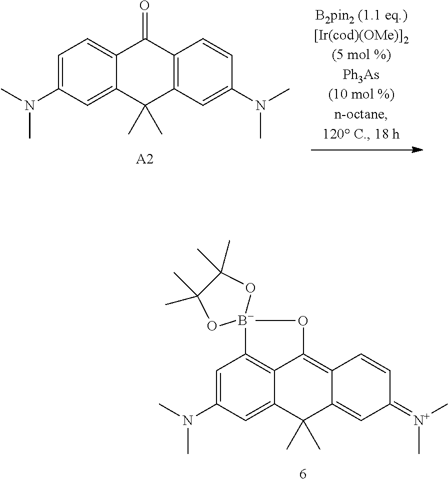

Compound 6. In a 10 ml tube, the mixture of compound A2 (92 mg, 0.30 mmol; known compound: [A. N. Butkevich et al. Angew. Chem. Int. Ed., 2016, 55 (10), 3290-3294]), bis(pinacolato) diboron (85 mg, 0.33 mmol, 1.1 equiv), [Ir (cod)(OMe)]₂ (12 mg, 0.018 mmol, 5 mol %), triphenylarsine (10 mg, 0.033 mmol, 10 mol %) in dry n-octane (3 mL) was degassed on a Schlenk line and stirred at 120° C. for 18 h. On cooling, the reaction mixture was diluted with dichloromethane and filtered through a plug of Celite, washing with dichloromethane. The filtrate was evaporated to dryness and the product was isolated by flash chromatography on a Biotage Isolera system (12 g Interchim SiHP 30 μm cartridge, gradient 0% to 10% ethyl acetate/dichloromethane) and freeze-dried from dioxane to 59 mg (45%) of 6 as an orange solid.

¹H NMR (400 MHZ, CDCl₃): δ 8.20 (d, J=8.9 Hz, 1H), 6.85 (d, J=2.2 Hz, 1H), 6.76 (d, J=2.5 Hz, 1H), 6.70 (dd, J=8.9, 2.5 Hz, 1H), 6.61 (d, J=2.2 Hz, 1H), 3.13 (s, 6H), 3.12 (s, 6H), 1.62 (s, 6H), 1.47 (s, 12H).

¹³C NMR (101 MHZ, CDCl₃): δ 182.6, 155.2, 154.6, 153.9, 150.6, 130.2, 123.3, 116.0, 113.3, 110.4, 108.3, 107.3, 81.8, 40.5, 40.2, 38.9, 33.2, 25.3.

HRMS (ESI) m/z: [M]⁺ Calcd for C₂₀H₂₄BN₂O₂: 335.1929, found: 335.1928-corresponds to the pinacol ester hydrolysis product (6a).

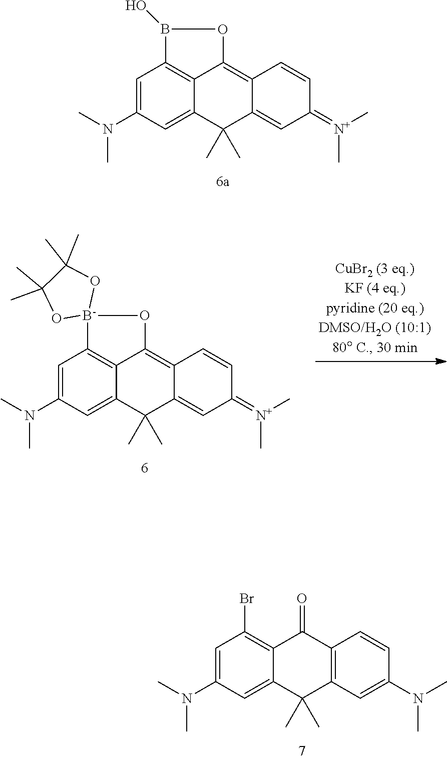

Compound 7. In a 10 mL round-bottom flask, compound 6 (59 mg, 0.14 mmol), potassium fluoride (33 mg, 0.56 mmol, 4 equiv), copper (II) bromide (94 mg, 0.42 mmol, 3 equiv) were placed, followed by addition of DMSO (1.2 mL), water (120 μL) and pyridine (220 μL, 2.7 mmol, 20 equiv). The reaction mixture was stirred at 80° C. for 30 min. Upon cooling to rt, the mixture was diluted with ethyl acetate and poured into water (10 mL). The product was extracted with ethyl acetate (3×10 mL), the combined extracts were washed with brine (50 mL) and dried over Na₂SO₄. The product was isolated by flash chromatography on Biotage Isolera system (25 g Interchim SiHP 30 μm cartridge, gradient 0% to 10% ethyl acetate/dichloromethane and freeze-dried from 1,4-dioxane to give 39 mg (72%) of 7 as yellow solid.

¹H NMR (400 MHZ, CDCl₃): δ 8.22 (d, J=8.8 Hz, 1H), 7.00 (d, J=2.6 Hz, 1H), 6.79 (d, J=2.6 Hz, 1H), 6.75 (dd, J=8.9, 2.5 Hz, 1H), 6.70 (d, J=2.5 Hz, 1H), 3.09 (s, 6H), 3.08 (s, 6H), 1.70 (s, 6H).

¹³C NMR (101 MHz, CDCl₃): δ 180.4, 154.3, 153.0, 151.8, 150.3, 129.6, 124.0, 120.9, 118.0, 117.5, 111.0, 108.2, 106.8, 40.2, 40.0, 38.9, 34.1.

HRMS (ESI) m/z: [M+H]⁺ Calcd for C₂₀H₂₃BrN₂O: 387.1067, found: 387.1066.

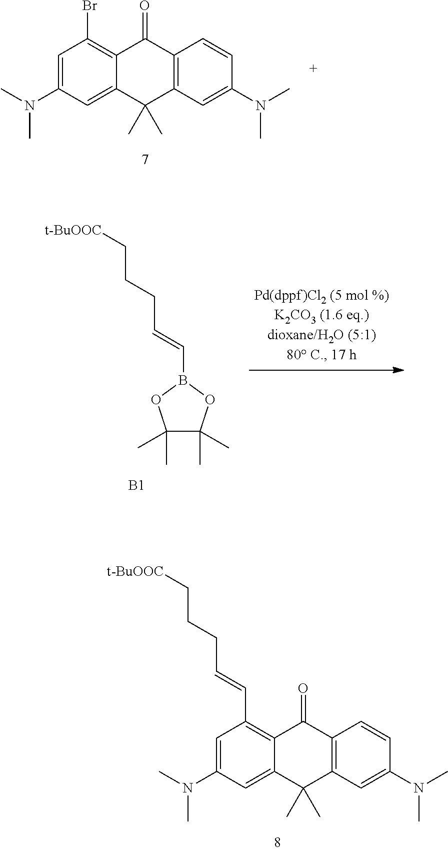

Compound 8. In a 10 ml tube, compound 7 (33 mg, 0.085 mmol), compound B1 (37 mg, 0.13 mmol, 1.5 equiv), K₂CO₃ (20 mg, 0.14 mmol, 1.6 equiv) and Pd(dppf) Cl₂·CH₂Cl₂ (3.4 mg, 4.2 μmol, 5 mol %) were loaded. Dioxane (1.0 mL) and water (0.2 mL) were added. The mixture was sparged with argon for 30 min, the tube was sealed and the reaction mixture stirred at 80° C. for 17 h. Upon cooling, the reaction mixture was diluted with ethyl acetate (10 mL) and washed with sat. aq. NH₄Cl (10 mL) and brine (10 ml). The organics were dried over Na₂SO₄, filtered, evaporated. The product was isolated by flash chromatography on a Biotage Isolera system (12 g Interchim SiHP 30 μm cartridge, gradient 0% to 50% ethyl acetate/hexane) and freeze-dried from dioxane to yield 23 mg (58%) of 8 as a yellow solid.

¹H NMR (400 MHz, CDCl₃): δ 8.19 (d, J=8.5 Hz, 1H), 7.64 (d, J=15.6 Hz, 1H), 6.79-6.71 (m, 3H), 6.67 (dd, J=2.7, 0.6 Hz, 1H), 5.90 (dt, J=15.4, 6.9 Hz, 1H), 3.11 (s, 6H), 3.09 (s, 6H), 2.38-2.30 (m, 4H), 1.85 (p, J=7.6 Hz, 2H), 1.71 (s, 6H), 1.45 (s, 9H).

¹³C NMR (101 MHZ, CDCl₃): δ 182.9, 173.4, 153.5, 152.8, 151.9, 151.1, 143.1, 134.8, 129.3, 129.1, 121.6, 117.8, 111.2, 110.9, 107.9, 107.2, 79.9, 40.2, 40.1, 38.8, 35.3, 34.2, 32.5, 28.2, 25.1.

HRMS (ESI) m/z: [M+H]⁺ Calcd for C₃₀H₄₀N₂O₃: 477.3112, found: 477.3112.

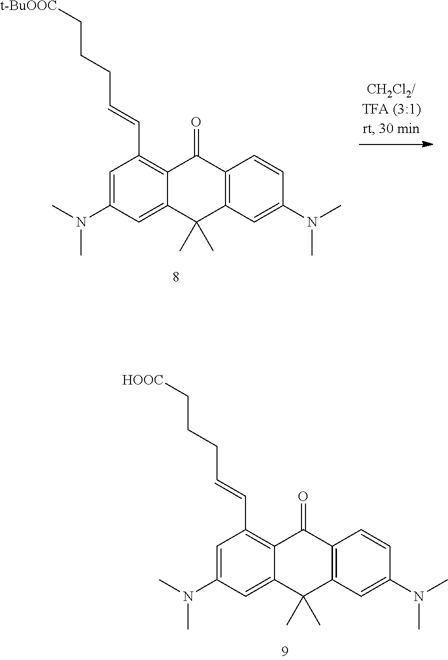

Compound 9. To a solution of compound 8 (17.5 mg, 0.075 mmol) in CH₂Cl₂ (300 μL), trifluoroacetic acid (100 μL) was added dropwise. The resulting reaction mixture was stirred for 60 minutes at rt, protected from light. The volatiles were removed in vacuo by coevaporation with toluene (3×10 ml). The product was isolated by flash chromatography on a Biotage Isolera system (12 g Interchim SiHP 30 μm cartridge, gradient 20% to 100% ethyl acetate/hexane) and freeze-dried from dioxane to yield 16.5 mg (˜100%, remainder dioxane) of 9 as a yellow solid.

¹H NMR (400 MHZ, CDCl₃): δ 8.19 (d, J=8.5 Hz, 1H), 7.64 (d, J=15.6 Hz, 1H), 6.79-6.71 (m, 3H), 6.67 (dd, J=2.7, 0.6 Hz, 1H), 5.90 (dt, J=15.4, 6.9 Hz, 1H), 3.11 (s, 6H), 3.09 (s, 6H), 2.38-2.30 (m, 4H), 1.85 (p, J=7.6 Hz, 2H), 1.71 (s, 6H), 1.45 (s, 9H).

¹³C NMR (101 MHz, CDCl₃): δ 153.5, 152.8, 151.9, 151.1, 143.1, 134.8, 129.3, 129.1, 111.2, 110.9, 107.9, 107.2, 40.2, 40.1, 38.8, 35.3, 34.2, 32.5, 28.2, 25.1.

HRMS (ESI) m/z: [M+H]⁺ Calcd for C₂₆H₃₂N₂O₃: 421.2486, found: 421.2486.

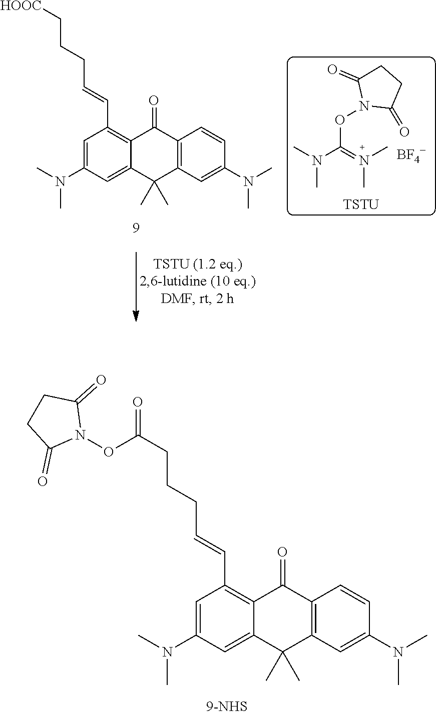

Compound 9-NHS. In an amber vial, compound 9 (8.6 mg, 0.020 mmol) and TSTU (12 mg, 0.04 mmol) were dissolved in DMF (200 μL). 2,6-Lutidine (21 mg, 0.20 mmol) was added and the reaction mixture was stirred for 3 h at rt. The volatiles were removed in vacuo. The product was isolated by flash chromatography on a Biotage Isolera system (12 g Interchim SiHP 30 μm cartridge, gradient 20% to 80% ethyl acetate/hexane) and freeze-dried from dioxane to yield 9.2 mg (89%) of 9-NHS as a pale yellow solid.

HRMS (ESI) m/z: [M+H]⁺ Calcd for C₃₀H₃₅N₃O₅: 518.2649, found: 518.2647.

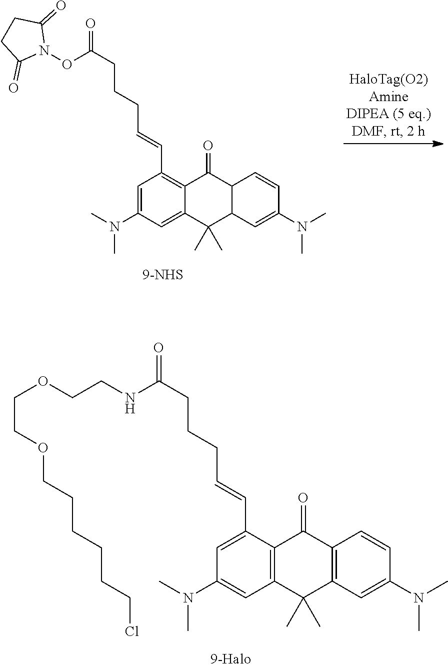 [SMILES: CN(C)C1=CC2C(C=C1)C(=O)c1c(/C=C/CCCC(=O)ON3C(=O)CCC3=O)cc(N(C)C)cc1C2(C)C ; CN(C)c1ccc2c(c1)C(C)(C)c1cc(N(C)C)cc(/C=C/CCCC(=O)NCCOCCOCCCCCCCl)c1C2=O]

Compound 9-Halo. To a solution of compound 9-NHS (7.2 mg, 0.014 mmol) in DMF (100 μL) was added HaloTag (02) Amine (4.7 mg, 0.021 mmol) and DIPEA (9.0 mg, 0.070 mmol), and the mixture stirred for 2 h at rt. The volatiles were removed in vacuo. The product was isolated by flash chromatography on a Biotage Isolera system (12 g Interchim SiHP 30 μm cartridge, 100% ethyl acetate) and freeze-dried from dioxane to yield 5.2 mg (59%) of 9-Halo as an orange oil.

HRMS (ESI) m/z: [M+H]⁺ Calcd for C₃₆H₅₂ClN₃O₄: 626.3719, found: 626.3714.

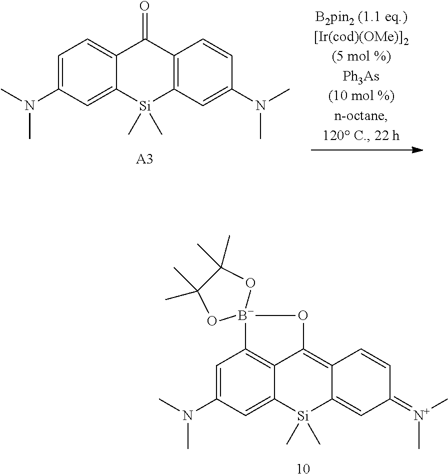

Compound 10. In a 25 mL round-bottom flask, the mixture of compound A3 (324 mg, 1 mmol; known compound: [A. N. Butkevich et al. J. Am. Chem. Soc. 2017, 139, 12378-12381]), bis(pinacolato) diboron (280 mg, 1.1 mmol, 1.1 equiv), [Ir (cod)(OMe)]₂ (33 mg, 0.05 mmol, 5 mol %) and triphenylarsine (31 mg, 0.01 mmol, 10 mol %) in dry n-octane (10 mL) was degassed on a Schlenk line and stirred at 120° C. for 22 h. On cooling, the reaction mixture was evaporated on Celite and the product was isolated by flash chromatography on Biotage Isolera system (10 g Biotage Sfär Duo cartridge, gradient 0% to 100% A: B, A=20% ethyl acetate in dichloromethane, B=dichloromethane) and freeze-dried from dioxane to give 322 mg (72%) of 10 as an orange solid.

¹H NMR (400 MHZ, CDCl₃): δ 8.38 (d, J=9.0 Hz, 1H), 7.02 (d, J=2.5 Hz, 1H), 6.80 (d, J=2.7 Hz, 1H), 6.75 (dd, J=9.1, 2.7 Hz, 1H), 6.72 (br.s, 1H), 3.14 (s, 12H), 1.43 (s, 12H), 0.39 (s, 6H).

¹³C NMR (101 MHZ, CDCl₃): δ 187.5, 153.4, 153.3, 145.9, 139.2, 133.8, 122.4, 115.6, 115.1, 114.7, 112.3, 80.5, 40.5, 40.1, 25.7,-1.2.

HRMS (ESI) m/z: [M]⁺ Calcd for C₁₉H₂₄BN₂O₂Si 351.1698; Found 351.1693-corresponds to the pinacol ester hydrolysis product (10a):

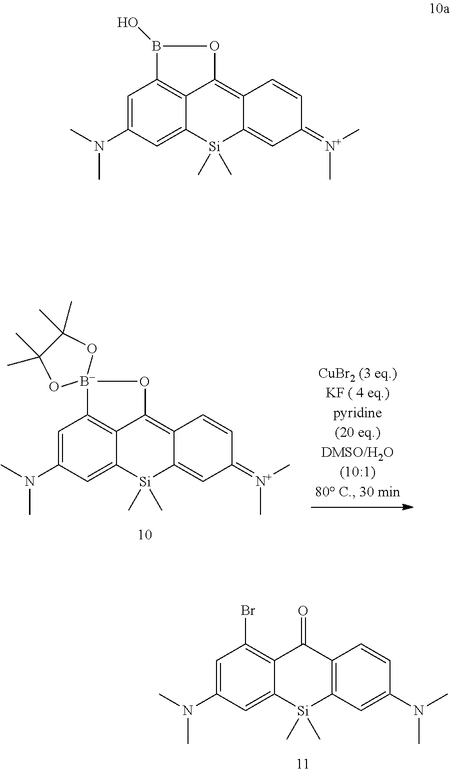

Compound 11. In a 25 mL round-bottom flask, compound 10 (259 mg, 0.58 mmol), potassium fluoride (135 mg, 2.32 mmol, 4 equiv), copper (II) bromide (388 mg, 1.74 mmol, 3 equiv) were placed, followed by addition of DMSO (5 mL), water (500 μL) and pyridine (940 μL, 11.6 mmol, 20 equiv). The reaction mixture was stirred at 80° C. for 30 min. Upon cooling to rt, the mixture was diluted with ethyl acetate and poured into water (80 mL). The product was extracted with ethyl acetate (4×30 mL), the combined extracts were washed with brine (50 mL) and dried over Na₂SO₄. The product was isolated by flash chromatography on Biotage Isolera system (25 g Interchim SiHP 30 μm cartridge, gradient 0% to 100% A/B, A: 10% ethyl acetate in dichloromethane, B: dichloromethane) and freeze-dried from dioxane to give 212 mg (91%) of 11 as yellow solid.

¹H NMR (400 MHZ, CDCl₃): δ 8.20 (d, J=8.9 Hz, 1H), 7.06 (d, J=2.7 Hz, 1H), 6.83 (dd, J=9.0, 2.8 Hz, 1H), 6.76 (br.d, J=2.8 Hz, 1H), 6.74 (d, J=2.7 Hz, 1H), 3.07 (s, 6H), 3.05 (s, 6H), 0.46 (s, 6H).

¹³C NMR (101 MHz, CDCl₃): δ 186.3, 151.2, 150.9, 142.3, 138.4, 131.5, 127.9, 125.3, 120.1, 114.1, 113.8, 113.6, 40.3, 40.0,-1.0.

HRMS (ESI) m/z: [M+H]⁺ Calcd for C₁₉H₂₃BrN₂OSi 403.0836; Found 403.0830.

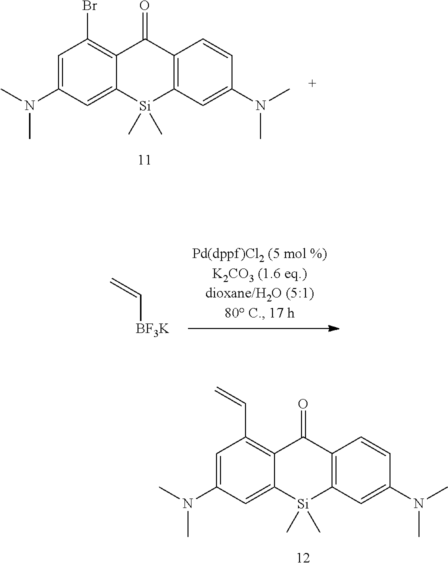

Compound 12. In a 10 ml tube, compound 11 (40 mg, 0.10 mmol), potassium vinyltrifluoroborate (15 mg, 0.11 mmol, 1.1 equiv), K₂CO₃ (22 mg, 0.16 mmol, 1.6 equiv) and Pd(dppf) Cl₂·CH₂Cl₂ (4.1 mg, 5.0 μmol, 5 mol %) were loaded. Dioxane (1.0 mL) and water (0.2 mL) were added. The mixture was sparged with argon for 30 min, and the tube sealed and stirred at 80° C. for 17 h. Upon cooling, the reaction mixture was diluted with ethyl acetate (10 mL) and washed with sat. aq. NH₄Cl (10 mL) and brine (10 mL). The organics were dried over Na₂SO₄, filtered, evaporated. The product was isolated by flash chromatography on Biotage Isolera system (12 g Interchim SiHP 30 μm cartridge, gradient 0% to 30% ethyl acetate/hexane) and freeze-dried from 1,4-dioxane to yield 31 mg (87%) of 12 as a yellow solid.

¹H NMR (400 MHZ, CDCl₃): δ 8.25 (d, J=8.9 Hz, 1H), 7.58 (dd, J=17.2, 10.7 Hz, 1H), 6.82 (dd, J=9.0, 2.8 Hz, 1H), 6.80-6.75 (m, 3H), 5.44 (dd, J=17.2, 1.8 Hz, 1H), 5.24 (dd, J=10.8, 1.8 Hz, 1H), 3.10 (s, 6H), 3.07 (s, 6H), 0.46 (s, 6H).

¹³C NMR (101 MHZ, CDCl₃): δ 187.7, 151.2, 150.8, 144.1, 141.8, 141.3, 139.0, 131.9, 131.3, 128.4, 114.5, 113.9, 113.7, 113.3, 112.5, 40.1, 40.0.

HRMS (ESI) m/z: [M+H]⁺ Calcd for C₂₁H₂₇N₂OSi: 351.1887, found: 351.1888.

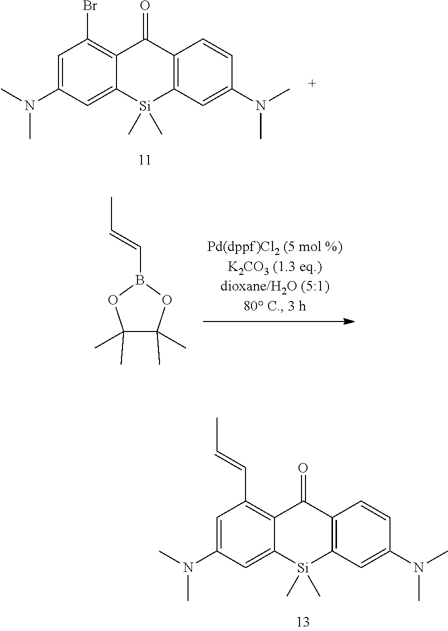

Compound 13. In a 10 ml tube, compound 11 (40 mg, 0.10 mmol), trans-1-propenylboronic acid pinacol ester (18 mg, 0.11 mmol, 1.1 equiv), K₂CO₃ (18 mg, 0.13 mmol, 1.3 equiv) and Pd(dppf) Cl₂·CH₂Cl₂ (4.1 mg, 5.0 μmol, 5 mol %) were loaded. Dioxane (1.0 mL) and water (0.2 mL) were added. The mixture was sparged with argon for 30 min, the tube was sealed and the mixture was stirred at 80° C. for 3 h. Upon cooling, the reaction mixture was diluted with ethyl acetate (10 mL) and washed with sat. aq. NH₄Cl (10 mL) and brine (10 mL). The organics were dried over Na₂SO₄, filtered, evaporated. The product was isolated by flash chromatography on Biotage Isolera system (12 g Interchim SiHP 30 μm cartridge, gradient 0% to 30% ethyl acetate/hexane) and freeze-dried from dioxane to yield 16 mg (43%) of 13 as a yellow solid.

¹H NMR (400 MHZ, CDCl₃): δ 8.25 (d, J=8.9 Hz, 1H), 7.33-7.26 (m, 1H), 6.82 (dd, J=9.0, 2.8 Hz, 1H), 6.75 (dt, J=5.4, 2.8 Hz, 3H), 5.93 (dq, J=15.4, 6.6 Hz, 1H), 3.09 (s, 6H), 3.07 (s, 6H), 1.95 (dd, J=6.6, 1.7 Hz, 3H), 0.45 (s, 6H).

¹³C NMR (101 MHz, CDCl₃): δ 187.9, 151.1, 150.7, 143.9, 141.2, 138.9, 135.3, 132.2, 131.3, 124.6, 114.1, 113.9, 113.7, 113.3, 40.1, 40.0, 18.7,-1.0.

HRMS (ESI) m/z: [M+H]⁺ Calcd for C₂₂H₂₈N₂OSi: 365.2044, found: 365.2044.

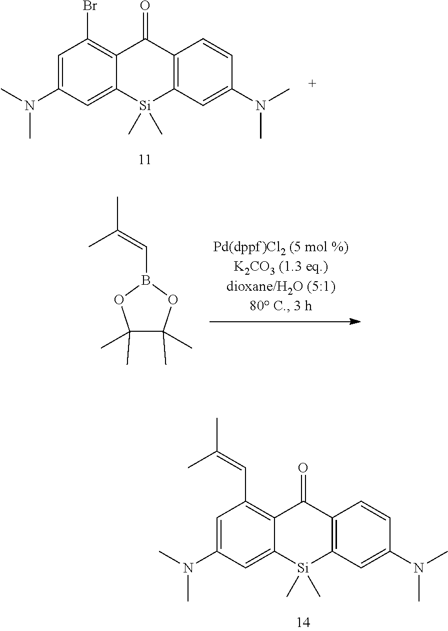

Compound 14. In a 10 ml tube, compound 11 (40 mg, 0.10 mmol), 2-methyl-1-propenylboronic acid pinacol ester (20 mg, 0.11 mmol, 1.1 equiv), K₂CO₃ (18 mg, 0.13 mmol, 1.3 equiv) and Pd(dppf) Cl₂·CH₂Cl₂ (4.1 mg, 5.0 μmol, 5 mol %) were loaded. Dioxane (1.0 mL) and water (0.2 mL) were added. The mixture was sparged with argon for 30 min, the tube was sealed and the mixture was stirred at 80° C. for 3 h. Upon cooling, the reaction mixture was diluted with ethyl acetate (10 mL) and washed with sat. aq. NH₄Cl (10 mL) and brine (10 mL). The organics were dried over Na₂SO₄, filtered, evaporated. The product was isolated by flash chromatography on Biotage Isolera system (12 g Interchim SiHP 30 μm cartridge, gradient 0% to 30% ethyl acetate/hexane) and freeze-dried from dioxane to yield 28 mg (74%) of 14 as a yellow solid.

¹H NMR (400 MHZ, CDCl₃): δ 8.26 (d, J=9.0 Hz, 1H), 6.89-6.85 (m, 1H), 6.81 (dd, J=8.9, 2.8 Hz, 1H), 6.75 (dd, J=10.3, 2.8 Hz, 2H), 6.60 (dd, J=2.9, 0.8 Hz, 1H), 3.07 (d, J=0.9 Hz, 12H), 1.97 (d, J=1.4 Hz, 3H), 1.74 (d, J=1.5 Hz, 3H), 0.46 (s, 6H).

¹³C NMR (101 MHz, CDCl₃): δ 187.3, 151.0, 150.1, 143.4, 141.3, 139.0, 132.0, 131.4, 129.7, 129.6, 128.7, 117.0, 113.7, 113.5, 113.2, 67.1, 40.1, 40.0, 26.3, 19.5,-0.9.

HRMS (ESI) m/z: [M+H]⁺ Calcd for C₂₃H₃₁N₂OSi: 379.2200, found: 379.2200.

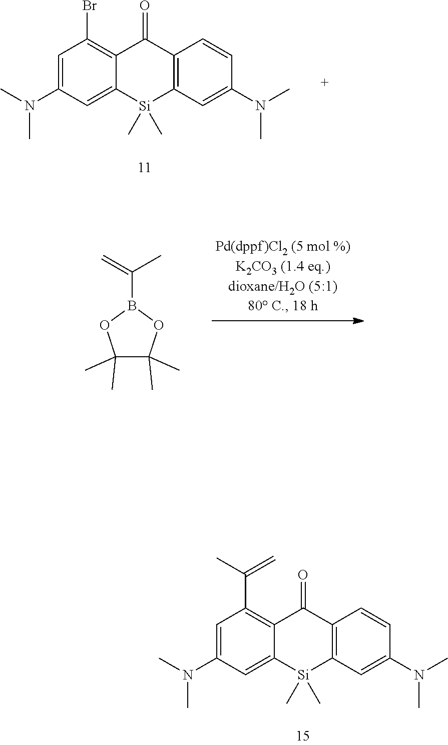

Compound 15. In a 10 ml tube, compound 12 (40 mg, 0.10 mmol), isopropenylboronic acid pinacol ester (18 mg, 0.11 mmol, 1.1 equiv), K₂CO₃ (20 mg, 0.14 mmol, 1.4 equiv) and Pd(dppf) Cl₂·CH₂Cl₂ (4.1 mg, 5.0 μmol, 5 mol %) were loaded. Dioxane (1.0 mL) and water (0.2 mL) were added. The mixture was sparged with argon for 30 min, and the tube was sealed and the mixture was stirred at 80° C. for 18 h. Upon cooling, the reaction mixture was diluted with ethyl acetate (10 mL) and washed with sat. aq. NH₄Cl (10 mL) and brine (10 mL). The organics were dried over Na₂SO₄, filtered, evaporated. The product was isolated by flash chromatography on Biotage Isolera system (12 g Interchim SiHP 30 μm cartridge, gradient 0% to 30% ethyl acetate/hexane) and freeze-dried from dioxane to yield 29 mg (81%) of 15 as a light orange solid.

¹H NMR (400 MHZ, CDCl₃): δ 8.26 (d, J=8.9 Hz, 1H), 6.82 (dd, J=8.9, 2.8 Hz, 1H), 6.76 (dd, J=8.3, 2.8 Hz, 2H), 6.57 (d, J=2.8 Hz, 1H), 5.01 (dq, J=2.9, 1.4 Hz, 1H), 4.80 (dd, J=2.2, 0.9 Hz, 1H), 3.08 (s, 6H), 3.07 (s, 6H), 2.12 (dd, J=1.4, 0.8 Hz, 3H), 0.47 (s, 6H).

¹³C NMR (101 MHZ, CDCl₃): δ 186.6, 151.9, 151.2, 150.6, 149.3, 141.4, 139.0, 131.8, 131.3, 128.0, 115.5, 113.8, 113.8, 113.3, 109.4, 40.1, 40.0, 24.5,-0.9.

HRMS (ESI) m/z: [M+H]⁺ Calcd for C₂₂H₂₈N₂OSi: 365.2044, found: 365.2045.

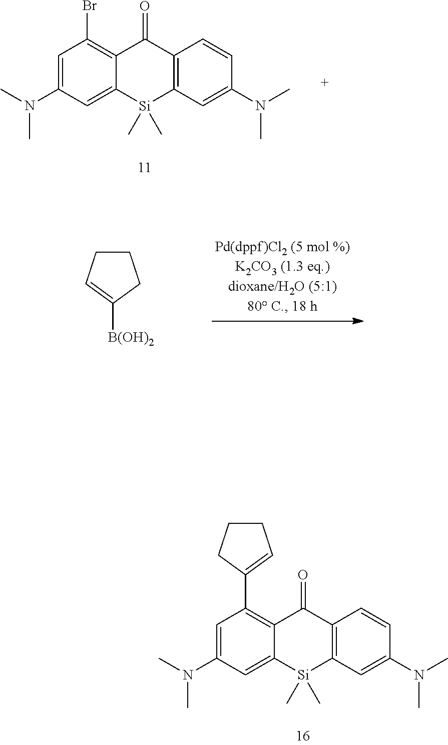

Compound 16. In a 10 ml tube, compound 11 (40 mg, 0.10 mmol), 1-cyclopentenylboronic acid (21 mg, 0.11 mmol, 1.1 equiv), K₂CO₃ (18 mg, 0.13 mmol, 1.3 equiv) and Pd(dppf) Cl₂·CH₂Cl₂ (4.1 mg, 5.0 μmol, 5 mol %) were loaded. Dioxane (1.0 mL) and water (0.2 mL) were added. The mixture was sparged with argon for 30 min, and the tube was sealed and the mixture was stirred at 80° C. for 18 h. Upon cooling, the reaction mixture was diluted with ethyl acetate (10 mL) and washed with sat. aq. NH₄Cl (10 mL) and brine (10 mL). The organics were dried over Na₂SO₄, filtered, evaporated. The product was isolated by flash chromatography on Biotage Isolera system (12 g Interchim SiHP 30 μm cartridge, gradient 0% to 30% ethyl acetate/hexane) and freeze-dried from dioxane to yield 23 mg (59%) of 16 as yellow solid.

¹H NMR (400 MHZ, CDCl₃): δ 8.20 (d, J=8.8 Hz, 1H), 6.81 (dd, J=8.9, 2.8 Hz, 1H), 6.76 (dd, J=13.2, 2.8 Hz, 2H), 6.60 (d, J=2.8 Hz, 1H), 5.58 (p, J=2.0 Hz, 1H), 3.07 (s, 6H), 3.07 (s, 6H), 2.60-2.50 (m, 4H), 2.08 (tt, J=8.0, 6.7 Hz, 2H), 0.46 (s, 6H).

¹³C NMR (101 MHZ, CDCl₃): δ 187.1, 151.1, 150.5, 149.8, 144.2, 141.0, 138.9, 132.2, 131.1, 128.9, 123.7, 115.8, 113.8, 113.8, 113.2, 40.1, 40.0, 36.5, 33.2, 24.6,-1.0.

HRMS (ESI) m/z: [M+H]⁺ Calcd for C₂₄H₃₀N₂OSi: 391.2227, found: 392.2225.

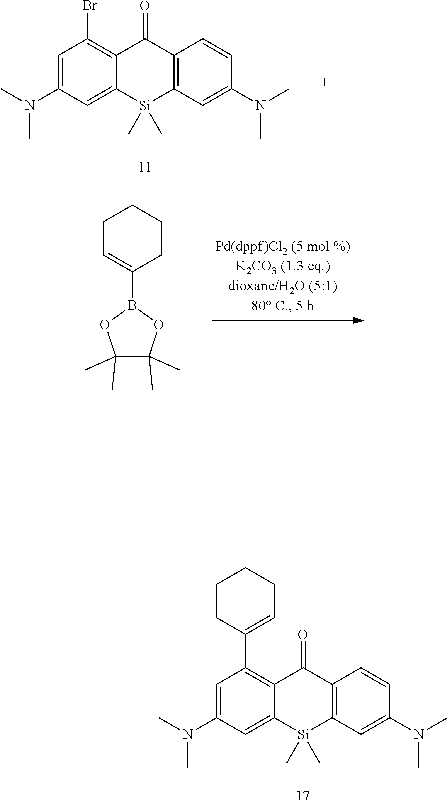

Compound 17. In a 10 ml tube, compound 11 (40 mg, 0.10 mmol), 1-cyclohexen-1-ylboronic acid pinacol ester (23 mg, 0.11 mmol, 1.1 equiv), K₂CO₃ (18 mg, 0.13 mmol, 1.3 equiv) and Pd(dppf) Cl₂·CH₂Cl₂ (4.1 mg, 5.0 μmol, 5 mol %) were loaded. Dioxane (1.0 mL) and water (0.2 mL) were added. The mixture was sparged with argon for 30 min, and the tube was sealed and the mixture was stirred at 80° C. for 18 h. The reaction mixture was diluted with ethyl acetate (10 mL) and washed with sat. aq. NH₄Cl (10 mL) and brine (10 mL). The organics were dried over Na₂SO₄, filtered, evaporated. The product was isolated by flash chromatography on Biotage Isolera system (12 g Interchim SiHP 30 μm cartridge, gradient 0% to 40% ethyl acetate/hexane) and freeze-dried from dioxane to yield 17 mg (42%) of 17 as a light yellow solid.

¹H NMR (400 MHZ, CDCl₃): δ 8.21 (d, J=8.9 Hz, 1H), 6.81 (dd, J=8.9, 2.8 Hz, 1H), 6.77 (d, J=2.7 Hz, 1H), 6.73 (d, J=2.9 Hz, 1H), 6.53 (d, J=2.9 Hz, 1H), 5.50 (tt, J=3.7, 1.5 Hz, 1H), 3.08 (s, 6H), 3.07 (s, 6H), 2.26-2.13 (m, 4H), 1.88-1.79 (m, 2H), 1.78-1.68 (m, 2H), 0.46 (s, 6H).

¹³C NMR (101 MHz, CDCl₃): δ 187.0, 151.0, 150.6, 149.8, 144.5, 141.1, 138.9, 132.3, 131.2, 120.3, 116.0, 113.8, 113.5, 113.2, 40.1, 40.0, 30.5, 25.6, 23.5, 22.4,-0.9.

HRMS (ESI) m/z: [M+H]⁺ Calcd for C₂₅H₃₂N₂OSi: 405.2357, found: 405.2356.

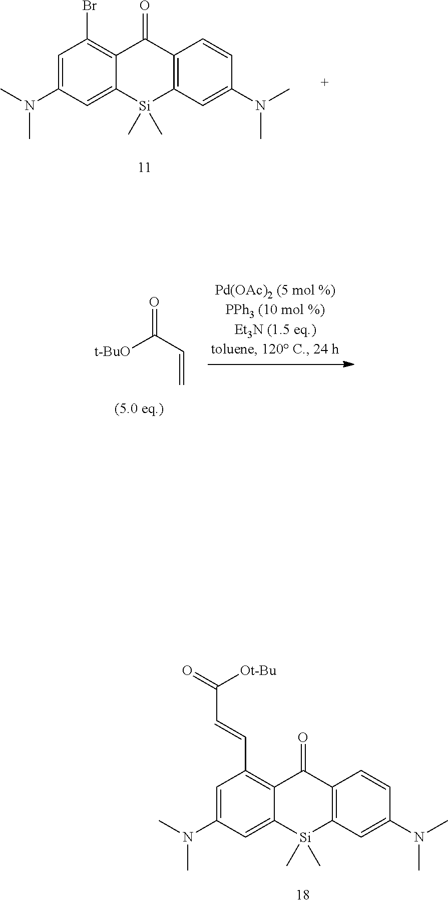

Compound 18. In a 10 ml tube, compound 11 (80 mg, 0.20 mmol), Pd(OAc)₂ (2.2 mg, 0.01 mmol, 5 mol %), triphenylphosphine (5.3 mg, 0.020 mol, 10 mol %), and toluene (2 mL) were degassed on a Schlenk line. tert-Butyl acrylate (128 mg, 1.0 mmol, 5 equiv) and triethylamine (30 mg, 0.30 mmol, 1.5 equiv) were added, the tube was sealed and the mixture was stirred at 120° C. for 24 h. The reaction mixture was diluted with ethyl acetate and poured into sat. aq. NH₄Cl (20 mL), and extracted with ethyl acetate (3×10 ml). The combined organics were washed with brine (50 mL), dried over Na₂SO₄, filtered, and evaporated. The product was isolated by flash chromatography on Biotage Isolera system (12 g Interchim SiHP 30 μm cartridge, gradient 0% to 50% ethyl acetate/hexane) and freeze-dried from dioxane to yield 41 mg (45%) of 18 as a yellow solid.

¹H NMR (400 MHZ, CDCl₃): δ 8.40 (d, J=15.6 Hz, 1H), 8.29 (d, J=9.0 Hz, 1H), 6.86-6.82 (m, 1H), 6.81 (d, J=2.8 Hz, 1H), 6.76 (d, J=2.7 Hz, 1H), 6.72 (d, J=2.7 Hz, 1H), 6.02 (d, J=15.5 Hz, 1H), 3.10 (s, 6H), 3.08 (s, 6H), 1.56 (s, 9H), 0.47 (s, 6H).

¹³C NMR (101 MHZ, CDCl₃): δ 186.8, 166.8, 151.3, 150.6, 149.7, 141.6, 141.2, 139.0, 131.6, 131.1, 119.1, 115.4, 114.1, 113.7, 113.3, 80.0, 40.1, 40.0, 28.3,-1.0.

HRMS (ESI) m/z: [M+H]⁺ Calcd for C₂₆H₃₄N₂O₃Si: 451.2411, found: 451.2411.

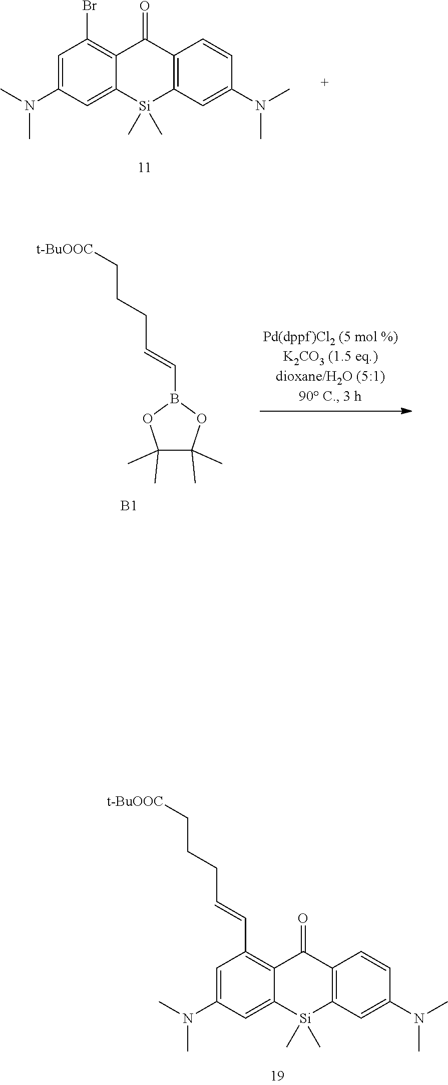

Compound 19. In a 100 mL round bottom flask, compound 11 (200 mg, 0.50 mmol), compound B1 (220 mg, 0.75 mmol, 1.5 equiv), K₂CO₃ (104 mg, 0.75 mmol, 1.5 equiv) and Pd(dppf) Cl₂·CH₂Cl₂ (20 mg, 25 μmol, 5 mol %) were loaded. Dioxane (10 mL) and water (2 mL) were added. The mixture was sparged with argon for 30 min and then stirred at 90° C. for 3 h. Upon cooling, the reaction mixture was diluted with ethyl acetate (50 mL) and washed with sat. aq. NH₄Cl (50 mL) and brine (50 mL). The organics were dried over Na₂SO₄, filtered, evaporated. The product was isolated by flash chromatography on Biotage Isolera system (25 g Interchim SiHP 30 μm cartridge, gradient 0% to 40% ethyl acetate/hexane) and freeze-dried from dioxane to yield 195 mg (79%) of 19 as a yellow solid.

¹H NMR (400 MHz, CDCl₃): δ 8.23 (d, J=8.9 Hz, 1H), 7.31-7.26 (m, 1H), 6.81 (dd, J=9.0, 2.8 Hz, 1H), 6.77-6.72 (m, 3H), 5.89 (dt, J=15.4, 6.9 Hz, 1H), 3.09 (s, 6H), 3.07 (s, 6H), 2.37-2.28 (m, 4H), 1.83 (p, J=7.6 Hz, 2H), 1.45 (s, 9H), 0.45 (s, 6H).

¹³C NMR (101 MHz, CDCl₃): δ 173.4, 151.1, 150.7, 143.5, 141.1, 138.9, 134.9, 132.3, 131.2, 128.8, 114.2, 113.74, 113.68, 113.2, 80.0, 40.1, 40.0, 35.2, 32.5, 28.2, 25.1,-1.0.

HRMS (ESI) m/z: [M+H]⁺ Calcd for C₂₉H₄₂N₂O₃Si: 495.3037, found: 495.3032.

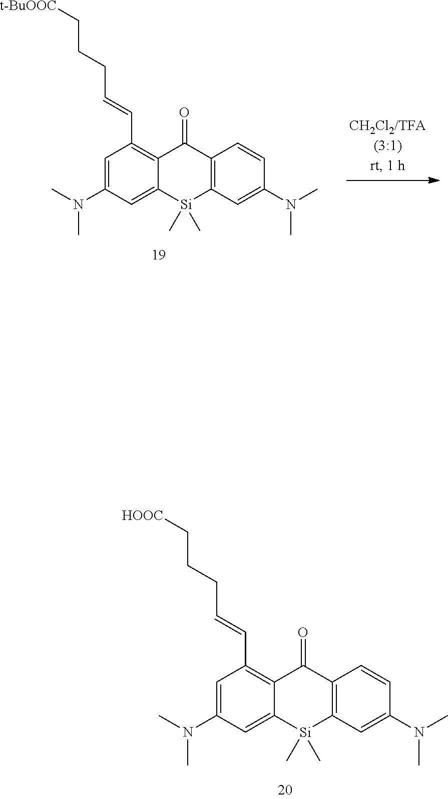

Compound 20. To a solution of compound 19 (195 mg, 0.40 mmol) in CH₂Cl₂ (3.0 ml), trifluoroacetic acid (1.0 mL) was added dropwise. The resulting reaction mixture was stirred for 60 minutes at rt, protected from light. The volatiles were removed in vacuo by coevaporation with toluene (3×10 ml). The product was isolated by flash chromatography on a Biotage Isolera system (25 g Interchim SiHP 30 μm cartridge, gradient 10% to 50% ethyl acetate/hexane) and freeze-dried from 1,4-dioxane to yield 172 mg (99%) of 20 as an orange solid.

¹H NMR (400 MHZ, DMSO-d₆): δ 12.03 (s, 1H), 7.98 (d, J=8.9 Hz, 1H), 7.16 (dt, J=15.5, 1.5 Hz, 1H), 6.89-6.80 (m, 3H), 6.73 (d, J=2.8 Hz, 1H), 5.88 (dt, J=15.5, 6.8 Hz, 1H), 3.06 (s, 6H), 3.03 (s, 6H), 2.33 (t, J=7.4 Hz, 2H), 2.21 (q, J=6.8 Hz, 2H), 1.71 (p, J=7.4 Hz, 2H), 0.43 (s, 6H).

¹³C NMR (101 MHZ, DMSO-d₆): δ 186.5, 174.5, 150.9, 150.4, 142.4, 140.7, 138.3, 134.0, 131.0, 130.4, 128.4, 126.9, 114.3, 113.8, 113.0, 112.7, 39.5, 39.4, 33.1, 31.8, 24.2,-1.2.

HRMS (ESI) m/z: [M+H]⁺ Calcd for C₂₅H₃₂N₂O₃Si: 437.2255, found: 437.2257.

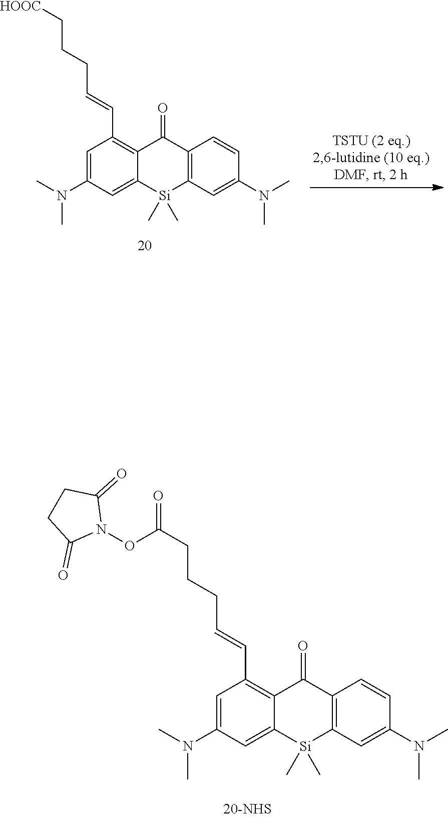

Compound 20-NHS. In an amber vial, compound 20 (8.0 mg, 0.018 mmol) and TSTU (11 mg, 0.036 mmol, 2 equiv) were dissolved in DMF (200 μL). 2,6-Lutidine (19 mg, 0.18 mmol) was added, and the reaction mixture was stirred for 3 h at rt. The volatiles were removed in vacuo. The product was isolated by flash chromatography on a Biotage Isolera system (12 g Interchim SiHP 30 μm cartridge, gradient 20% to 80% ethyl acetate/hexane) and freeze-dried from dioxane to yield 7.5 mg (78%) of 20-NHS as a beige solid.

¹H NMR (400 MHZ, DMSO-d₆): δ 7.99 (d, J=8.9 Hz, 1H), 7.21-7.12 (m, 1H), 6.88-6.80 (m, 3H), 6.73 (d, J=2.8 Hz, 1H), 5.88 (dt, J=15.5, 6.8 Hz, 1H), 3.06 (s, 6H), 3.03 (s, 6H), 2.85-2.78 (m, 6H), 2.30 (q, J=7.2, 6.5 Hz, 2H), 1.85 (p, J=7.4 Hz, 2H), 0.43 (s, 6H).

¹³C NMR (101 MHZ, DMSO-d₆) δ 170.2, 169.0, 150.9, 150.5, 142.3, 140.7, 138.4, 134.7, 130.9, 130.5, 127.5, 126.9, 114.4, 113.8, 113.0, 112.8, 31.2, 30.3, 29.6, 25.4, 24.1,-1.2.

HRMS (ESI) m/z: [M+H]⁺ Calcd for C₂₉H₃₅N₃O₅Si: 534.2419, found: 534.2415.

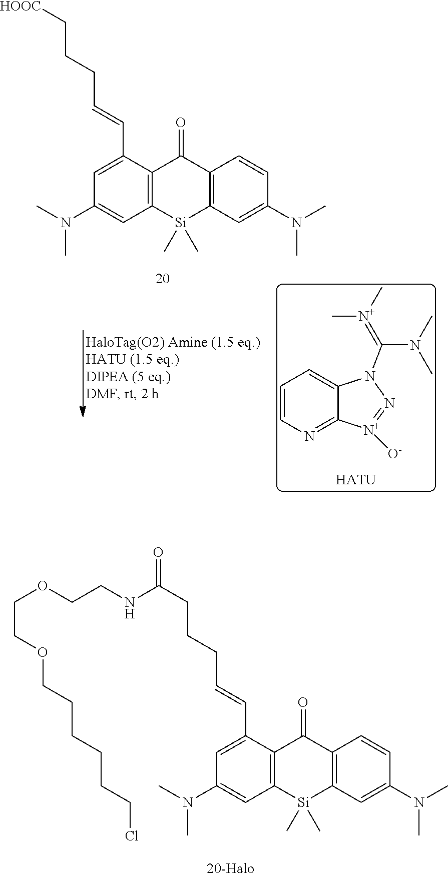

Compound 20-Halo. In an amber vial, compound 20 (22 mg, 0.050 mmol), HaloTag (02) Amine (17 mg, 0.075 mmol, 1.5 equiv), and HATU (1-[bis(dimethylamino) methylene]-1H-1,2,3-triazolo[4,5-b]pyridinium 3-oxid hexafluorophosphate; 29 mg, 0.075 mmol, 1.5 equiv) were dissolved in DMF (200 μL). DIPEA (32 mg, 0.25 mmol, 5 equiv) was added and the mixture stirred for 2 h at rt. The volatiles were removed in vacuo. The product was isolated by flash chromatography on a Biotage Isolera system (12 g Interchim SiHP 30 μm cartridge, 100% ethyl acetate) and freeze-dried from dioxane to yield 20-Halo as a yellow solid. Yield 33 mg (84%, remainder dioxane).

¹H NMR (400 MHZ, DMSO-d₆): δ 7.99 (d, J=8.9 Hz, 1H), 7.92 (t, J=5.6 Hz, 1H), 7.16-7.09 (m, 1H), 6.89-6.79 (m, 3H), 6.71 (d, J=2.8 Hz, 1H), 5.84 (dt, J=15.5, 6.9 Hz, 1H), 3.57 (t, J=6.6 Hz, 2H), 3.48-3.44 (m, 2H), 3.44-3.39 (m, 4H), 3.31-3.27 (m, 4H), 3.24 (q, J=5.8 Hz, 2H), 3.06 (s, 6H), 3.03 (s, 6H), 2.22-2.13 (m, 4H), 1.75-1.60 (m, 4H), 1.46-1.37 (m, 2H), 1.37-1.10 (m, 6H), 0.43 (s, 6H).

¹³C NMR (101 MHZ, DMSO-d₆): δ 186.7, 172.2, 151.1, 150.6, 142.7, 140.9, 138.5, 134.4, 131.0, 130.6, 128.5, 126.9, 114.4, 113.9, 113.1, 112.9, 70.2, 69.6, 69.4, 69.2, 45.3, 38.6, 34.7, 32.0, 31.9, 29.0, 26.1, 25.1, 24.9, −1.1.

HRMS (ESI) m/z: [M+H]⁺ Calcd for C₃₅H₅₂ClN₃O₄Si: 642.3488, found: 642.3487.

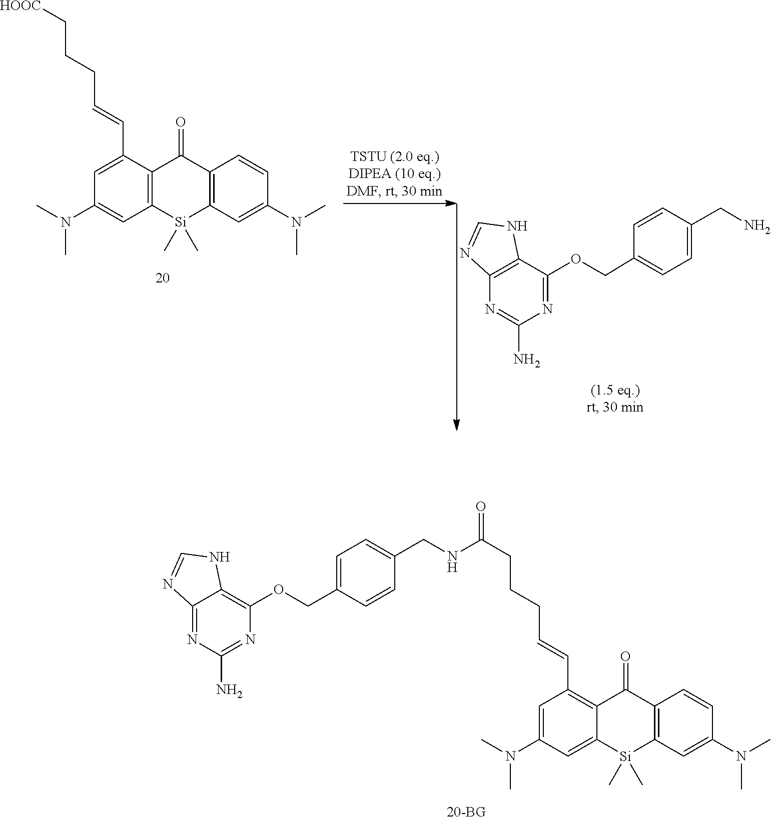

Compound 20-BG. In an amber vial, compound 20 (10.4 mg, 0.024 mmol) and TSTU (14 mg, 0.048 mmol, 2 equiv) were dissolved in DMF (240 μL). DIPEA (31 mg, 0.24 mmol) was added, and the mixture was stirred for 30 min at rt. 6-((4-(Aminomethyl) benzyl) oxy)-7H-purin-2-amine (20.0 mg, 0.036 mmol, 1.5 equiv) was then added, and the reaction mixture was stirred at rt for 30 min. The volatiles were removed in vacuo. The product was isolated by flash chromatography on a Biotage Isolera system (12 g Interchim SiHP 30 μm cartridge, gradient 0% to 10% methanol/dichloromethane) and freeze-dried from dioxane to yield 15.1 mg (92%) of 20-BG as a yellow solid.

HRMS (ESI) m/z: [M+H]⁺ Calcd for C₃₈H₄₄N₈O₃Si: 689.3378, found: 689.3378.

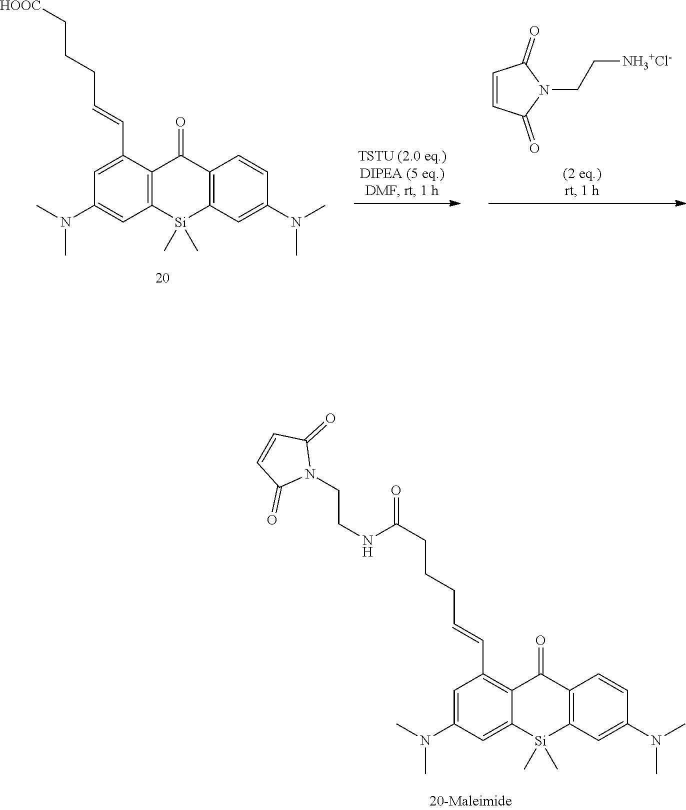

Compound 21-Maleimide. In an amber vial, compound 20 (22 mg, 0.050 mmol) and TSTU (30 mg, 0.10 mmol) were dissolved in DMF (500 μL) and DIPEA (64 mg, 0.25 mmol) was added and stirred for 60 min. at rt. 1-(1-Aminoethyl) maleimide hydrochloride (18 mg, 0.10 mmol) was added and the reaction was stirred at rt for 60 min. The volatiles were removed in vacuo. The product was isolated by flash chromatography on a Biotage Isolera system (12 g Interchim SiHP 30 μm cartridge, gradient 50% to 100% ethyl acetate/hexane) and freeze-dried from dioxane to yield 18.8 mg (67%) of 20-Maleimide as a yellow solid.

¹H NMR (400 MHZ, CDCl₃): δ 8.24 (t, J=5.9 Hz, 1H), 8.04 (d, J=9.0 Hz, 1H), 7.01 (d, J=15.5 Hz, 1H), 6.85 (dd, J=9.0, 2.8 Hz, 1H), 6.76 (d, J=2.7 Hz, 1H), 6.75 (d, J=2.8 Hz, 1H), 6.71 (d, J=2.8 Hz, 1H), 6.50 (s, 2H), 5.67 (dt, J=15.4, 7.2 Hz, 1H), 3.78 (dd, J=6.6, 4.6 Hz, 2H), 3.68-3.58 (m, 2H), 3.10 (s, 12H), 2.41-2.33 (m, 2H), 2.31-2.21 (m, 2H), 1.93-1.82 (m, 2H), 0.46 (s, 6H).

¹³C NMR (101 MHz, CDCl₃): δ 188.1, 175.3, 171.0, 151.4, 151.1, 144.2, 141.8, 139.5, 136.8, 134.1, 131.5, 131.4, 128.7, 127.7, 114.3, 114.1, 113.9, 113.5, 40.2, 40.1, 38.5, 38.1, 34.3, 31.2, 24.8,-0.8.

HRMS (ESI) m/z: [M+H]⁺ Calcd for C₃₁H₃₈N₄O₄Si: 559.2735, found: 559.2735.

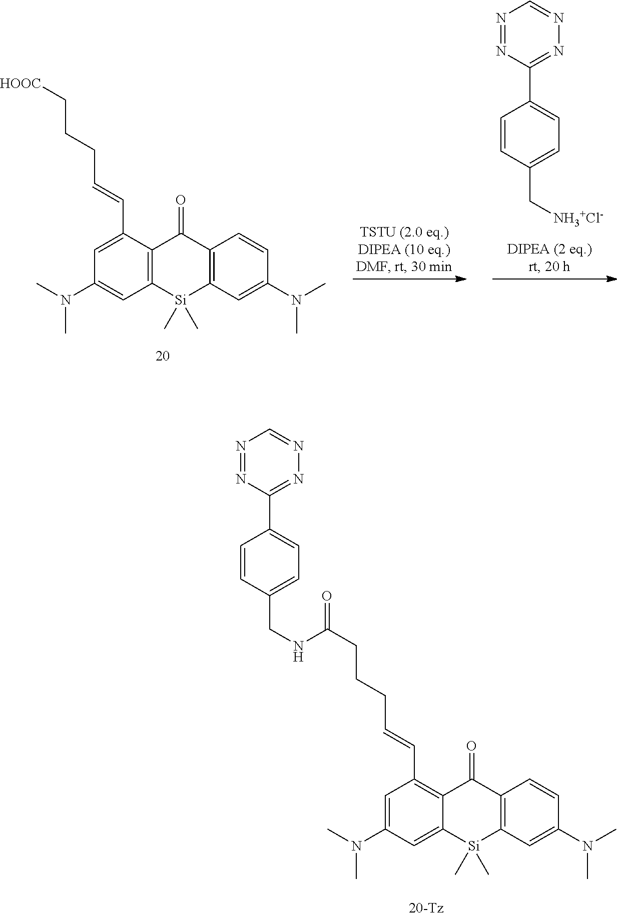

Compound 20-Tz. In an amber vial, compound 20 (12.0 mg, 0.027 mmol) and TSTU (16 mg, 0.053 mmol, 2 equiv) were dissolved in DMF (250 μL). DIPEA (33 mg, 0.25 mmol) was added, and the mixture was stirred for 30 min at rt. 4-(1,2,4,5-Tetrazin-3-yl) benzylamine hydrochloride (Sigma-Aldrich, Cat. Nr. 761591; 9.0 mg, 0.040 mmol, 1.5 equiv) and additional DIPEA (8.2 mg, 0.06 mmol, 2.2 equiv) were added, and the reaction was stirred at rt overnight. The volatiles were removed in vacuo. The product was isolated by flash chromatography on a Biotage Isolera system (12 g Interchim SiHP 30 μm cartridge, gradient 50% to 100% ethyl acetate/hexane) and freeze-dried from dioxane to yield 7.0 mg (43%) of 20-Tz as an orange solid.

¹H NMR (400 MHZ, DMSO-d₆): δ 10.57 (s, 1H), 8.52 (t, J=6.0 Hz, 1H), 8.45 (d, J=8.4 Hz, 2H), 7.93 (d, J=8.9 Hz, 1H), 7.56 (d, J=8.4 Hz, 2H), 7.15 (d, J=15.6 Hz, 1H), 6.84 (t, J=3.0 Hz, 2H), 6.79 (dd, J=9.0, 2.8 Hz, 1H), 6.73 (d, J=2.8 Hz, 1H), 5.88 (dt, J=15.5, 6.9 Hz, 1H), 4.45 (d, J=5.8 Hz, 2H), 3.30 (s, 6H), 3.05 (s, 6H), 3.02 (s, 7H), 2.32 (t, J=7.2 Hz, 2H), 2.22 (q, J=6.8 Hz, 2H), 1.78 (p, J=7.3 Hz, 2H), 0.42 (s, 6H).

¹³C NMR (101 MHz, DMSO-d₆): δ 186.7, 172.4, 165.4, 158.1, 151.0, 150.6, 145.1, 142.6, 140.9, 138.5, 134.3, 131.0, 130.6, 130.3, 128.6, 128.1, 127.8, 126.9, 114.5, 113.9, 113.0, 112.9, 41.9, 39.3, 39.4, 34.7, 32.0, 25.1,-1.1.

HRMS (ESI) m/z: [M+H]⁺ Calcd for C₃₄H₃₉N₇O₂Si: 606.3007, found: 606.3007.

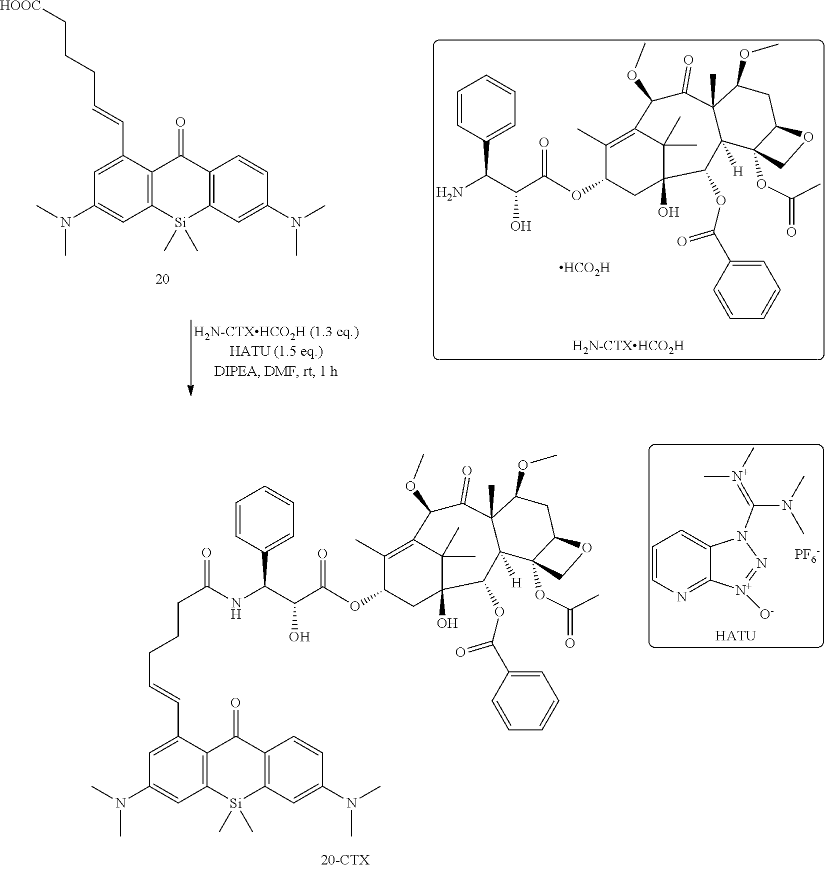

Compound 20-CTX. In an amber vial, a solution of HATU (13 mg, 0.035 mmol, 1.5 equiv) in DMF (50 μL) was added to the mixture of compound 20 (10 mg, 0.023 mmol), H₂N-CTX·HCO₂H [A. N. Butkevich et al. ACS Chem. Biol. 2018, 13 (2), 475-480](23 mg, 0.03 mmol, 1.3 equiv) in DIPEA (100 μL) and DMF (100 μL). The reaction mixture was stirred at rt for 1 h. The solvents were removed in vacuo, and the product the product was isolated by preparative HPLC (Interchim Uptisphere Strategy PhC4 250×21.2 mm 5 μm, solvent flow rate 18 mL/min, gradient 50% to 90% A: B, A-acetonitrile+0.1% (v/v) formic acid, B-water+0.1% (v/v) formic acid) and freeze-dried from dioxane to give 17 mg (64%) of yellow solid.

¹H NMR (400 MHZ, acetone-d₆): δ 8.27 (d, J=9.0 Hz, 1H), 8.19 (d, J=9.0 Hz, 1H), 8.14-8.08 (m, 2H), 7.68-7.62 (m, 1H), 7.62-7.52 (m, 4H), 7.43-7.34 (m, 3H), 7.32-7.26 (m, 1H), 6.96 (t, J=3.1 Hz, 2H), 6.81 (dd, J=9.1, 2.8 Hz, 1H), 6.77 (d, J=2.8 Hz, 1H), 6.23 (td, J=9.2, 1.8 Hz, 1H), 5.78-5.62 (m, 3H), 4.99 (d, J=7.2 Hz, 1H), 4.95 (dd, J=9.7, 2.0 Hz, 1H), 4.84 (s, 1H), 4.82-4.77 (m, 1H), 4.18-4.11 (m, 2H), 3.93 (dd, J=10.7, 6.5 Hz, 1H), 3.86 (d, J=7.0 Hz, 1H), 3.77 (s, 1H), 3.39 (s, 3H), 3.29 (s, 3H), 3.12 (s, 6H), 3.10 (s, 6H), 2.82 (s, 3H), 2.71 (ddd, J=14.1, 9.7, 6.5 Hz, 1H), 2.57-2.39 (m, 2H), 2.37 (s, 3H), 2.32-2.12 (m, 3H), 1.98 (d, J=1.5 Hz, 3H), 1.92-1.70 (m, 2H), 1.67 (s, 3H), 1.66-1.57 (m, 1H), 1.41 (s, 1H), 1.21 (s, 3H), 1.18 (s, 3H), 0.47 (s, 6H).

¹³C NMR (101 MHz, acetone-d₆): δ 205.5, 188.4, 174.0, 173.9, 173.5, 171.1, 166.6, 152.4, 152.0, 145.1, 142.5, 140.8, 140.0, 139.6, 137.6, 136.6, 134.0, 132.5, 132.4, 131.3, 130.9, 129.4, 129.3, 128.9, 128.4, 128.3, 128.2, 115.4, 114.9, 114.6, 114.0, 84.8, 83.6, 82.0, 81.7, 78.7, 78.6, 76.7, 75.7, 74.9, 74.8, 72.3, 67.6, 57.6, 57.4, 57.2, 56.4, 48.1, 44.30, 44.28, 40.1, 40.0, 36.8, 34.9, 32.9, 32.1, 27.4, 25.9, 23.0, 21.9, 14.8, 10.8, −0.86, −0.88.

HRMS (ESI) m/z: [M+H]⁺ Calcd for C₆₅H₇₉N₃O₁₄Si 1154.5404; Found 1154.5407.

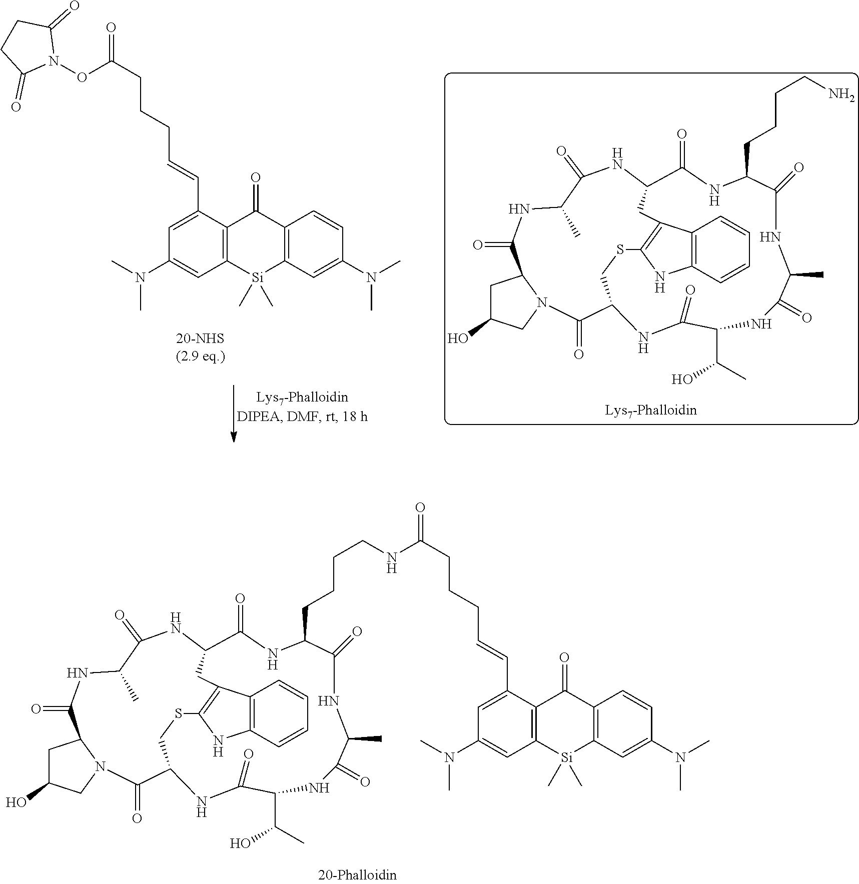 [SMILES: CN(C)c1ccc2c(c1)[Si](C)(C)c1cc(N(C)C)cc(/C=C/CCCC(=O)ON3C(=O)CCC3=O)c1C2=O ; C[C@@H]1NC(=O)[C@H](CCCCN)NC(=O)[C@@H]2Cc3c([nH]c4ccccc34)SC[C@H](NC(=O)[C@@H]([C@H](C)O)NC1=O)C(=O)N1C[C@@H](O)C[C@H]1C(=O)N[C@@H](C)C(=O)N2 ; C[C@@H]1NC(=O)[C@H](CCCCNC(=O)CCC/C=C/c2cc(N(C)C)cc3c2C(=O)c2ccc(N(C)C)cc2[Si]3(C)C)NC(=O)[C@@H]2Cc3c([nH]c4ccccc34)SC[C@H](NC(=O)[C@@H]([C@H](C)O)NC1=O)C(=O)N1C[C@@H](O)C[C@H]1C(=O)N[C@@H](C)C(=O)N2]

Compound 20-Phalloidin. In an amber vial, compound 20-NHS (2.0 mg, 3.8 μmol, 2.9 equiv) and Lys⁷-Phalloidin (trifluoroacetate salt, Bachem Cat. Nr. H-7636; 1 mg, 1.3 μmol) were dissolved in DMF (200 μL). DIPEA (90 μL) was added, and the reaction mixture was at rt for 18 h. The solvents were removed in vacuo, and the product the product was isolated by preparative HPLC (Interchim Uptisphere Strategy PhC4 250×21.2 mm 5 μm, solvent flow rate 18 mL/min, gradient 40% to 85% A: B, A-acetonitrile+0.1% (v/v) formic acid, B-water+0.1% (v/v) formic acid) and freeze-dried from dioxane to give 1.5 mg (97%) of 20-Phalloidin as light pink solid.

HRMS (ESI) m/z: [M+2H]²⁺ Calcd for C₆₀H₇₉N₁₁O₁₁SSi: 595.7798, found: 595.7786.

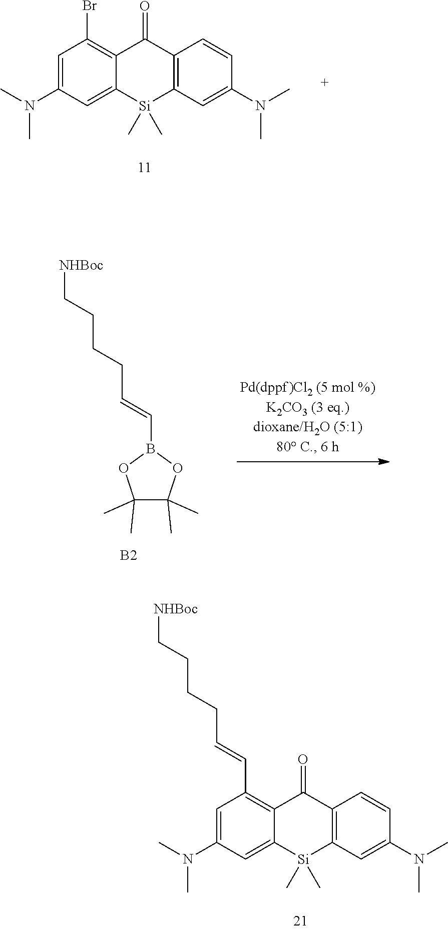

Compound 21. In a 10 mL round-bottom flask, compound 11 (80 mg, 0.20 mmol), compound B2 [N. L. Reed et al. Org. Lett. 2018, 20, 7345-7350](88 mg, 0.26 mmol, 1.3 equiv), Pd(dppf) Cl₂·CH₂Cl₂ (8.2 mg, 0.01 mmol, 5 mol %) and K₂CO₃ (82 mg, 0.6 mmol, 3 equiv) were placed, dioxane (1.7 mL) and water (0.3 mL) were added, the mixture was degassed and stirred at 80° C. for 6 h. On cooling, the reaction mixture was diluted with ethyl acetate and poured into brine (50 mL), extracted with ethyl acetate (3×25 mL), and the combined extracts were dried over Na₂SO₄. The product was isolated by flash chromatography on Biotage Isolera system (12 g Interchim SiHP 30 μm cartridge, gradient 20% to 80% ethyl acetate/hexane) and freeze-dried from dioxane to give 101 mg (97%) of 21 as yellow solid.

¹H NMR (400 MHZ, CDCl₃): δ 8.23 (d, J=8.9 Hz, 1H), 7.25 (d, J=15.5 Hz, 1H), 6.81 (dd, J=9.0, 2.8 Hz, 1H), 6.78-6.73 (m, 3H), 5.88 (dt, J=15.5, 6.8 Hz, 1H), 4.84 (br.s, 1H), 3.19 (q, J=6.5 Hz, 2H), 3.09 (s, 6H), 3.07 (s, 6H), 2.36-2.27 (m, 2H), 1.68-1.53 (m, 4H), 1.44 (s, 9H), 0.45 (s, 6H).

¹³C NMR (101 MHZ, CDCl₃): δ 188.1, 156.3, 151.2, 150.8, 143.8, 141.3, 139.1, 134.8, 132.3, 131.4, 129.3, 128.4, 114.3, 113.9, 113.8, 113.4, 40.6, 40.2, 40.1, 32.6, 29.4, 28.6, 26.7,-0.8.

HRMS (ESI) m/z: [M+H]⁺ Calcd for C₃₀H₄₃N₃O₃Si 522.3146; Found 522.3143.

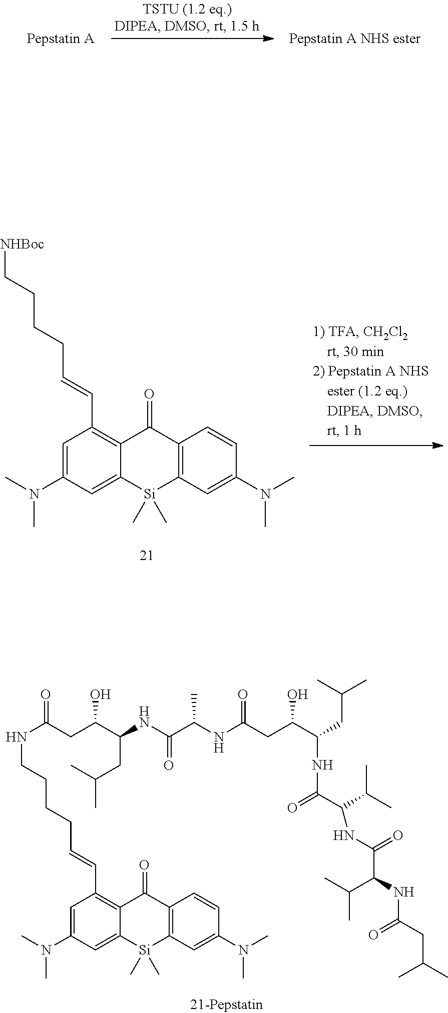

Compound 21-Pepstatin. To a solution of Pepstatin A (19 mg, 27.6 μmol, 1.2 equiv) in the mixture of DIPEA (40 μL) and DMSO (800 μL), a solution of TSTU (N,N,N′,N′-tetramethyl-O—(N-succinimidyl) uronium tetrafluoroborate; 10 mg, 33 μmol, 1.2 equiv relative to Pepstatin A) in DMSO (100 μL) was added, and the reaction mixture was stirred at rt for 1.5 h to give the solution of Pepstatin A NHS ester in DMSO.

Separately, in a 10 mL round-bottom flask, a solution of compound 22 (12 mg, 23 μmol) in dichloromethane (3 mL) and trifluoroacetic acid (300 μL) was stirred at rt for 30 min. The mixture was then diluted with toluene (2 mL), evaporated to dryness and chased twice with 3 mL toluene. Afterwards, the solution of Pepstatin A NHS ester in DMSO was added to the dry residue followed by additional DIPEA (50 μL). The reaction mixture was stirred at rt for 1 h, excess DIPEA was evaporated and the product was isolated from the crude mixture by preparative HPLC (Interchim Uptisphere Strategy PhC4 250×21.2 mm 5 μm, solvent flow rate 18 mL/min, gradient 40% to 90% A: B, A-acetonitrile+0.1% (v/v) formic acid, B-water+0.1% (v/v) formic acid) and freeze-dried from dioxane-water to give 13 mg (52%) of light yellow solid.

¹H NMR (400 MHZ, DMSO-d₆): δ 8.00-7.89 (m, 2H), 7.85-7.71 (m, 2H), 7.47 (d, J=8.8 Hz, 1H), 7.33 (d, J=9.1 Hz, 1H), 7.17 (dt, J=15.5, 1.4 Hz, 1H), 6.88-6.81 (m, 3H), 6.72 (d, J=2.7 Hz, 1H), 5.88 (dt, J=15.5, 6.8 Hz, 1H), 4.87 (d, J=5.1 Hz, 1H), 4.83 (d, J=4.9 Hz, 1H), 4.25 (p, J=7.1 Hz, 1H), 4.18 (dd, J=8.8, 7.3 Hz, 1H), 4.13 (dd, J=8.9, 7.2 Hz, 1H), 3.88-3.73 (m, 4H), 3.15-3.04 (m, 1H), 3.05 (s, 6H), 3.03 (s, 6H), 2.19 (q, J=6.8 Hz, 2H), 2.15-2.00 (m, 5H), 2.01-1.87 (m, 3H), 1.60-1.43 (m, 6H), 1.41-1.30 (m, 1H), 1.28-1.17 (m, 5H), 0.89-0.75 (m, 28H), 0.43 (s, 6H).

¹³C NMR (101 MHz, DMSO-d₆): δ 186.7, 172.2, 171.6, 171.1, 170.8, 170.70, 170.65, 162.3, 151.0, 150.5, 142.5, 140.8, 138.4, 133.6, 131.1, 130.6, 129.2, 127.0, 114.4, 113.9, 113.1, 112.7, 69.2, 69.0, 58.0, 57.8, 50.7, 50.5, 48.3, 44.4, 38.6, 38.4, 35.8, 34.4, 32.3, 30.8, 30.4, 30.3, 30.1, 28.8, 26.4, 25.7, 24.2, 23.5, 23.3, 22.3, 21.9, 21.6, 21.1, 19.30, 19.25, 18.4, 18.3, 18.2,-1.1.

HRMS (ESI) m/z: [M+2H]²⁺ Calcd for C₅₉H₉₆N₈O₉Si 545.3608; Found 545.3599.

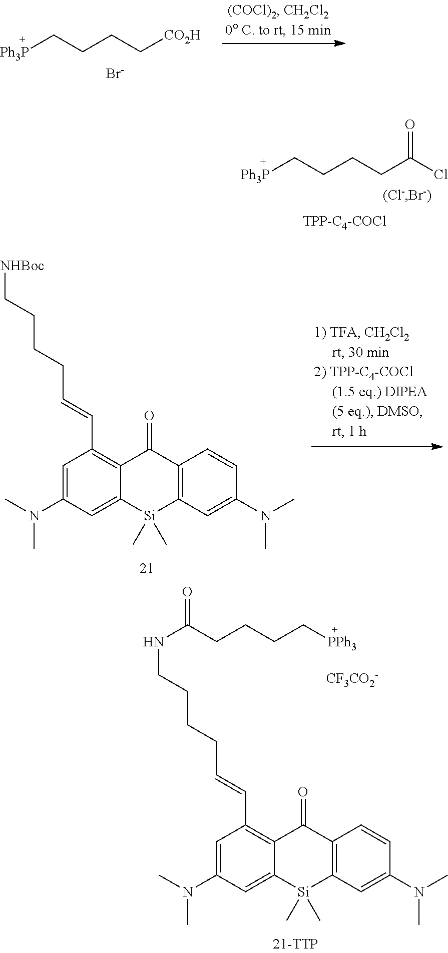

Compound 21-TPP. A suspension of 4-(carboxybutyl) triphenylphosphonium bromide (44 mg, 0.1 mmol) in dry dichloromethane (1 mL) was cooled in ice-water bath, and oxalyl chloride (85 μL, 1.0 mmol, 10 equiv) was added. The mixture was allowed to warm up to rt and stirred for 15 min. The resulting clear light-yellow solution was evaporated to dryness and chased with dry dichloromethane (1 mL). The residue was dissolved in dry dichloromethane (1 mL) to give ˜0.1 M solution of TPP-C₄—COCl.

Separately, in a 10 mL round-bottom flask, a solution of compound 21 (12 mg, 23 μmol) in dichloromethane (3 mL) and trifluoroacetic acid (300 μL) was stirred at rt for 30 min. The mixture was then diluted with toluene (2 mL), evaporated to dryness and chased twice with 3 mL toluene.

Afterwards, the residue was dissolved in dry dichloromethane (1 mL), and DIPEA (30 μL) and the solution of TPP-C₄—COCl (350 μL of ˜0.1 M in dichloromethane, ˜35 μmol) were added. The reaction mixture was stirred at rt for 1 h, the solvents were evaporated and the product was isolated from the residue by preparative HPLC (12 g Interchim Uptisphere Strategy PhC4 250×21.2 mm 5 μm, solvent flow rate 18 mL/min, gradient 40% to 90% A: B, A-acetonitrile+0.1% (v/v) TFA, B-water+0.1% (v/v) TFA) and freeze-dried from dioxane to give 20 mg (98%) of light pink viscous oil (trifluoroacetate salt).

¹H NMR (400 MHZ, DMSO-d₆): δ 7.97 (d, J=8.9 Hz, 1H), 7.92-7.85 (m, 3H), 7.82-7.71 (m, 12H), 7.16 (dt, J=15.5, 1.5 Hz, 1H), 6.87 (d, J=2.7 Hz, 1H), 6.85 (d, J=2.8 Hz, 1H), 6.82 (dd, J=9.0, 2.7 Hz, 1H), 6.71 (d, J=2.8 Hz, 1H), 5.87 (dt, J=15.5, 6.8 Hz, 1H), 3.62-3.51 (m, 2H), 3.05 (s, 6H), 3.03 (s, 6H), 2.21-2.07 (m, 4H), 1.71 (p, J=7.3 Hz, 2H), 1.58-1.46 (m, 2H), 1.45-1.39 (m, 4H), 0.43 (s, 6H).

¹⁹F NMR (376 MHZ, DMSO-d₆): δ-74.5.

³¹P NMR (162 MHZ, DMSO-d₆): δ 23.9.

¹³C NMR (101 MHz, DMSO-d₆): δ 186.7, 171.3, 158.1 (q, ²J_{C-F}=35.1 Hz), 151.0, 150.5, 142.5, 140.8, 138.5, 134.92, 134.89, 133.64, 133.59, 133.5, 131.1, 130.5, 130.3, 130.2, 128.1 (q, ¹J_{C-F}=213.7 Hz), 118.9, 118.1, 117.5, 114.6, 114.5, 114.0, 113.15, 112.7, 38.3, 34.4, 32.2, 30.4, 28.8, 26.31, 26.25, 26.1, 21.4, 21.3, 20.2, 19.7,-1.1 (C—P multiplets were not interpreted).

HRMS (ESI) m/z: [M+H]²⁺ Calcd for C₄₈H₅₈N₃O₂PSi 383.7012; Found 383.7008.

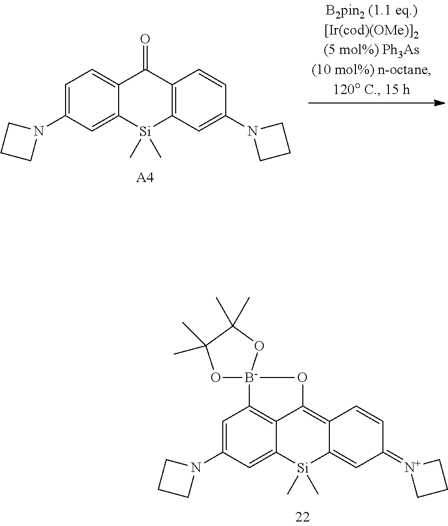

Compound 22. In a 10 mL tube, compound A4 [S. Shen et al., RSC Adv., 2017, 7 (18) 10922-10927](86 mg, 0.25 mmol), bis(pinacolato) diboron (70 mg, 0.28 mmol, 1.1 equiv), (1,5-cyclooctadiene)(methoxy) iridium (I) dimer (8.2 mg, 0.012 mmol, 5 mol %), triphenylarsine (7.7 mg, 0.025 mmol, 10 mol %) in dry n-octane (2.5 mL) was degassed on a Schlenk line and stirred at 120° C. for 15 h. The reaction mixture was diluted with dichloromethane and evaporated onto Celite. The product was isolated by flash chromatography on a Biotage Isolera system (25 g Interchim SiHP 30 μm cartridge, gradient 0% to 20% ethyl acetate/dichloromethane) and freeze-dried from 1,4-dioxane to yield 70 mg (80%) of 22 as a yellow solid.

¹H NMR (400 MHZ, CDCl₃): δ 8.35 (d, J=8.8 Hz, 1H), 6.66 (d, J=2.2 Hz, 1H), 6.45 (d, J=2.4 Hz, 1H), 6.41 (dd, J=8.7, 2.5 Hz, 1H), 6.33 (d, J=2.2 Hz, 1H), 4.18-4.03 (m, 8H), 2.53-2.36 (m, 4H), 1.41 (s, 12H), 0.35 (s, 6H).

¹³C NMR (101 MHz, CDCl₃): δ 187.4, 153.7, 153.4, 145.7, 139.1, 133.7, 131.5, 122.6, 114.0, 113.2, 113.0, 110.7, 80.3, 51.3, 51.2, 25.6, 16.44, 16.36,-1.4.

HRMS (ESI) m/z: [M]⁺ Calcd for C₂₁H₂₄BN₂O₂Si: 375.1699, found: 375.1701-corresponds to the pinacol ester hydrolysis product (22a):

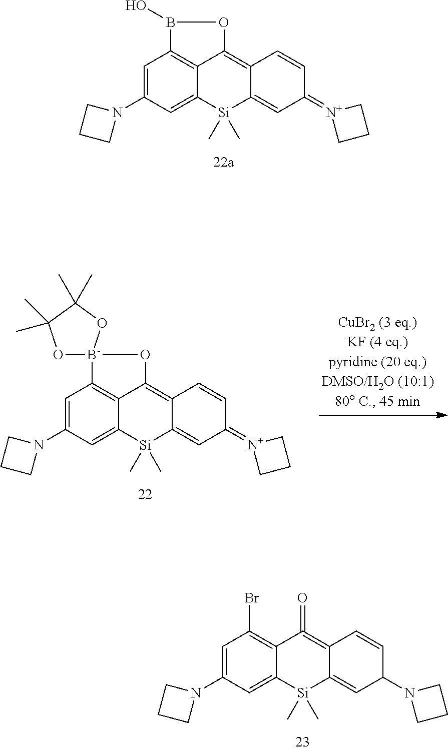

Compound 23. In a 25 ml flask, compound 22 (64 mg, 0.13 mmol), copper (II) bromide (90 mg, 0.40 mmol, 3 equiv) and potassium fluoride (31 mg, 0.54 mmol, 4 equiv) were loaded. DMSO (5 mL), water (500 μL) and pyridine (210 mg, 2.7 mmol, 20 equiv) were added, and the reaction mixture was stirred at 80° C. for 45 minutes. The reaction mixture was then quenched with water (20 mL) and extracted with ethyl acetate (3×20 mL). The combined organics were washed with brine (75 mL), dried over Na₂SO₄, filtered, and evaporated. The product was isolated by flash chromatography on Biotage Isolera system (12 g Interchim SiHP 30 μm cartridge, gradient 0% to 10% ethyl acetate/dichloromethane) and freeze-dried from dioxane to yield 46 mg (83%) of 23 as a yellow solid.

¹H NMR (400 MHZ, CDCl₃): δ 8.15 (d, J=8.6 Hz, 1H), 6.75 (d, J=2.4 Hz, 1H), 6.51 (dd, J=8.7, 2.5 Hz, 1H), 6.46-6.40 (m, 2H), 4.01 (td, J=7.3, 2.0 Hz, 8H), 2.50-2.35 (m, 4H), 0.43 (s, 6H).

¹³C NMR (101 MHZ, CDCl₃): δ 186.6, 152.4, 151.8, 142.2, 138.0, 132.8, 131.1, 128.6, 124.8, 118.8, 112.8, 112.4, 112.3, 51.8, 51.7, 16.7, 16.6,-1.3.

HRMS (ESI) m/z: [M+H]⁺ Calcd for C₂₁H₂₃BrN₂OSi: 427.0836, found: 427.0833.

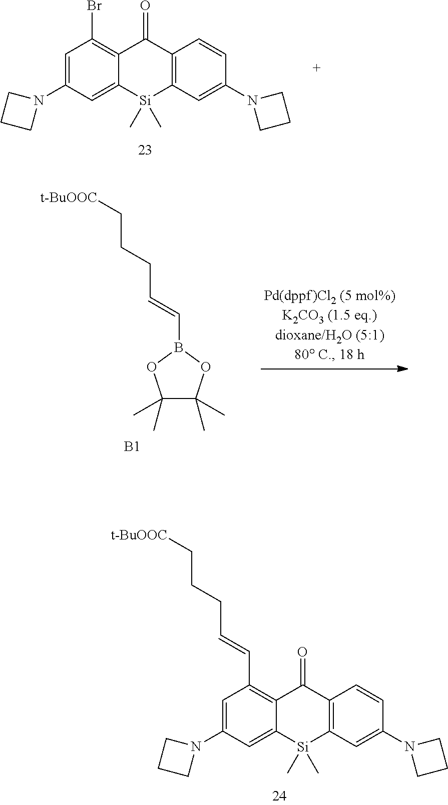

Compound 24. In a 10 ml tube, compound 23 (43 mg, 0.10 mmol), compound B1 (44 mg, 0.15 mmol), K₂CO₃ (21 mg, 0.15 mmol) and Pd(dppf) Cl₂·CH₂Cl₂ (4.1 mg, 5.0 μmol, 5 mol %) were loaded. Dioxane (1.0 mL) and water (0.2 mL) were added. The mixture was sparged with argon for 30 min, and the tube was sealed and the mixture was stirred at 80° C. for 18 h. The reaction mixture was quenched with sat. aq. NH₄Cl (10 mL), and extracted with ethyl acetate (3×10 mL). The combined organics were washed with brine (50 mL), dried over Na₂SO₄, filtered, and evaporated. The product was isolated by flash chromatography on Biotage Isolera system (12 g Interchim SiHP 30 μm cartridge, gradient 0% to 30% ethyl acetate/hexane) and freeze-dried from dioxane to yield 37 mg (72%) of 24 as yellow solid.

¹H NMR (400 MHZ, CDCl₃): δ 8.17 (d, J=8.7 Hz, 1H), 7.21 (dt, J=15.6, 1.5 Hz, 1H), 6.50 (dd, J=8.7, 2.6 Hz, 1H), 6.48-6.41 (m, 3H), 5.88 (dt, J=15.4, 6.9 Hz, 1H), 4.06-3.96 (m, 8H), 2.49-2.36 (m, 4H), 2.36-2.25 (m, 4H), 1.82 (p, J=7.6 Hz, 2H), 1.45 (s, 9H), 0.42 (s, 6H).

¹³C NMR (101 MHz, CDCl₃): δ 188.3, 173.3, 152.3, 151.9, 143.2, 140.9, 138.7, 134.3, 133.2, 131.0, 129.1, 129.0, 113.1, 112.6, 112.4, 112.2, 79.9, 51.82, 51.81, 35.2, 32.5, 28.2, 25.1, 16.7,-1.2.

HRMS (ESI) m/z: [M+H]⁺ Calcd for C₃₁H₄₀N₂O₃Si: 517.2881, found: 517.2879.

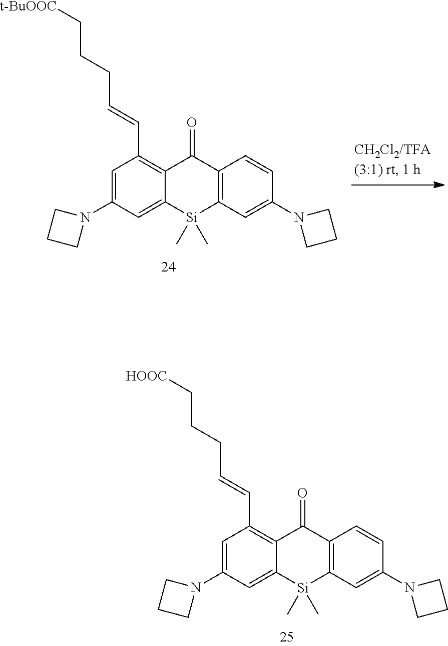

Compound 25. To a solution of compound 24 (32 mg; 0.062 mmol) in CH₂Cl₂ (600 μL) trifluoroacetic acid (200 μL) was added dropwise. The resulting reaction mixture was stirred for 60 minutes at rt, protected from light. The volatiles were removed in vacuo by coevaporation with toluene (3×10 ml). The product was isolated by flash chromatography on Biotage Isolera system (12 g Interchim SiHP 30 μm cartridge, gradient 20% to 80% ethyl acetate/hexane) and freeze-dried from 1,4-dioxane to yield 19 mg (67%) of 25 as yellow solid.

¹H NMR (400 MHZ, DMSO-d₆): δ 12.01 (s, 1H), 7.95 (d, J=8.7 Hz, 1H), 7.09 (d, J=15.5 Hz, 1H), 6.55 (d, J=2.5 Hz, 1H), 6.53 (d, J=2.5 Hz, 1H), 6.50 (dd, J=8.7, 2.5 Hz, 1H), 6.42 (d, J=2.5 Hz, 1H), 5.85 (dt, J=15.5, 6.8 Hz, 1H), 4.02-3.90 (m, 8H), 2.41-2.27 (m, 6H), 2.19 (q, J=6.7 Hz, 2H), 1.70 (p, J=7.4 Hz, 2H), 0.40 (s, 6H).

¹³C NMR (101 MHz, DMSO-d₆): δ 186.9, 174.5, 152.2, 151.8, 142.3, 140.5, 138.2, 133.6, 132.0, 130.4, 128.7, 127.8, 113.3, 112.8, 112.0, 111.5, 51.5, 51.4, 33.1, 31.9, 24.3, 16.2,-1.3.

HRMS (ESI) m/z: [M+H]⁺ Calcd for C₂₇H₃₂N₂O₃Si: 461.2255, found: 461.2253.

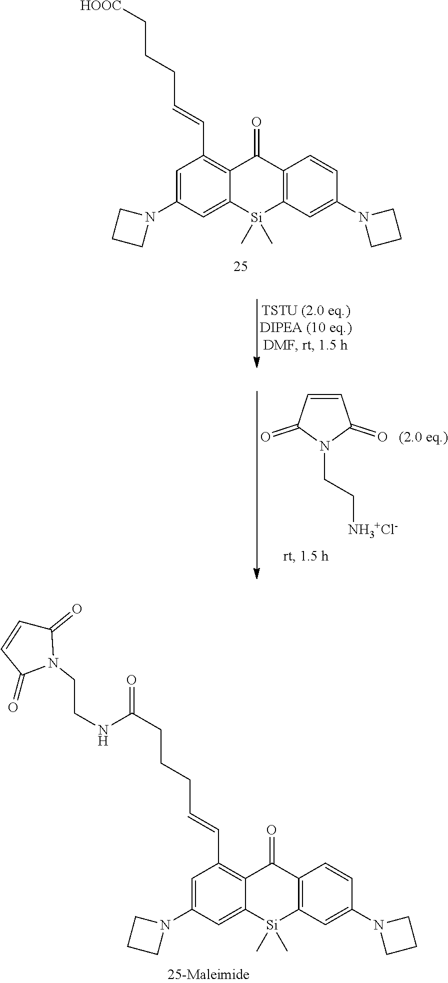

Compound 27-Maleimide. In an amber vial, compound 27 (8.4 mg, 0.018 mmol) and TSTU (12 mg, 0.036 mmol) were dissolved in DMF (180 μL) and DIPEA (23 mg, 0.18 mmol) was added and stirred for 1.5 hours at rt, then 1-(2-aminoethyl) maleimide hydrochloride was added and the reaction mixture was stirred for 1.5 hours at rt. The volatiles were removed in vacuo. The product was isolated by flash chromatography on a Biotage Isolera system (12 g Interchim SiHP 30 μm cartridge, gradient 50% to 100% ethyl acetate/hexane) and freeze-dried from 1,4-dioxane to yield 6.7 mg (89%) of 27-Maleimide as a yellow solid.

¹H NMR (400 MHZ, CDCl₃): δ 8.01 (d, J=8.7 Hz, 2H), 6.97 (d, J=15.4 Hz, 1H), 6.54 (dd, J=8.7, 2.5 Hz, 1H), 6.50 (s, 2H), 6.46 (d, J=2.5 Hz, 1H), 6.43 (d, J=2.5 Hz, 1H), 6.40 (d, J=2.5 Hz, 1H), 5.66 (dt, J=15.4, 7.2 Hz, 1H), 4.03 (t, J=7.3 Hz, 8H), 3.80-3.75 (m, 2H), 3.62 (q, J=5.8 Hz, 2H), 2.44 (p, J=7.3 Hz, 4H), 2.37-2.30 (m, 2H), 2.25 (q, J=6.7, 6.3 Hz, 2H), 1.92-1.82 (m, 2H), 0.43 (s, 6H).

¹³C NMR (101 MHZ, CDCl₃): δ 174.4, 170.9, 152.4, 152.0, 143.8, 141.4, 139.1, 136.1, 133.9, 132.3, 131.0, 128.7, 113.0, 112.7, 112.6, 112.2, 51.8, 51.7, 38.2, 38.1, 34.3, 31.1, 24.7, 16.7,-1.1.

HRMS (ESI) m/z: [M+H]⁺ Calcd for C₃₃H₃₈N₄O₄Si: 583.2735, found: 583.2733.

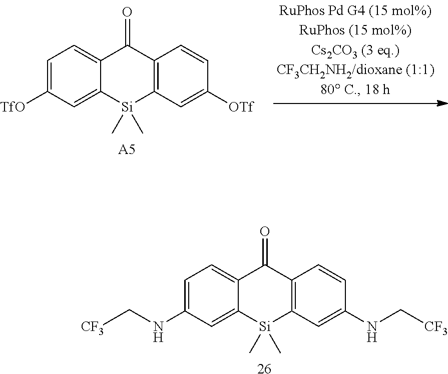

Compound 26. In a dried 10 mL crimp-top tube (a 2-5 ml Biotage microwave vial was used), compound A5 (134 mg, 0.25 mmol; prepared according to the literature procedure: S. Jia et al. ACS Chem. Biol. 2018, 13 (7), 1844-1852), RuPhos Pd G4 (32 mg, 37.5 μmol, 15 mol %), RuPhos (18 mg, 37.5 μmol, 15 mol %) and Cs₂CO₃ (245 mg, 0.75 mmol, 3 equiv) were placed, and the contents of the vial were degassed on a Schlenk line. Anhydrous dioxane (1 mL) and 2,2,2-trifluoroethylamine (1 mL) were then injected, the vial was placed in a 80° C. oil bath and the reaction mixture was stirred for 18 h. The mixture was then cooled down to rt, diluted with dichloromethane and filtered through a 1 cm plug of Celite, washing with dichloromethane and ethyl acetate (30 mL each). The filtrate was evaporated on silica and the product was isolated by flash chromatography on Biotage Isolera system (24 g Interchim SiHP 30 μm cartridge, gradient 20% to 80% ethyl acetate/hexane) and freeze-dried from dioxane to give light yellow solid containing 1 equiv of dioxane. Yield 125 mg (96%).

¹H NMR (400 MHZ, CDCl₃): δ 8.38-8.34 (m, 2H), 6.85-6.80 (m, 4H), 4.44 (t, J=6.8 Hz, 2H), 3.88 (qd, J=8.7, 6.8 Hz, 4H), 0.44 (s, 6H).

¹⁹F NMR (376 MHZ, CDCl₃): δ-72.1.

¹³C NMR (101 MHZ, CDCl₃): δ 185.2, 148.5, 141.0, 132.5, 132.2, 124.9 (q, ¹J_{C-F}=280.1 Hz), 116.0, 114.3, 45.35 (q, ²J_{C-F}=34.2 Hz), -1.3.

HRMS (ESI) m/z: [M+H]⁺ Calcd for C₁₉H₁₈F₆N₂OSi 433.1165; Found 433.1165.

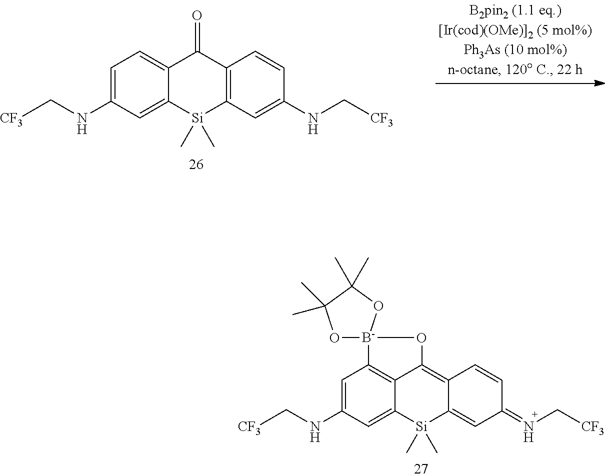

Compound 27. In a 10 mL round-bottom flask, the mixture of compound 26 (60 mg, 0.139 mmol), bis(pinacolato) diboron (29 mg, 0.153 mmol, 1.1 equiv), [Ir (cod)(OMe)]₂ (4.6 mg, 6.95 μmol, 5 mol %) and triphenylarsine (4.3 mg, 13.9 μmol, 10 mol %) in dry n-octane (2 mL) was degassed on a Schlenk line and stirred at 120° C. for 22 h. On cooling, the reaction mixture was diluted with dichloromethane, evaporated on Celite and the product was isolated by flash chromatography on Biotage Isolera system (12 g Interchim SiHP 30 μm cartridge, gradient 2% to 50% ethyl acetate: dichloromethane) and freeze-dried from dioxane to give 59 mg (76%) of 27 as bright yellow solid.

¹H NMR (400 MHZ, DMSO-ds): δ 8.09 (d, J=8.9 Hz, 1H), 7.54 (t, J=6.9 Hz, 1H), 7.20 (t, J=7.0 Hz, 1H), 7.18 (d, J=2.4 Hz, 1H), 7.04 (d, J=2.3 Hz, 1H), 6.99 (dd, J=8.9, 2.4 Hz, 1H), 6.84 (d, J=2.3 Hz, 1H), 4.27-4.08 (m, 2H), 1.25 (s, 12H), 0.42 (s, 6H).

¹⁹F NMR (376 MHZ, DMSO-d₆): δ-70.4.

¹³C NMR (101 MHz, DMSO-d₆): δ 185.8, 152.5, 151.5, 145.2, 139.5, 132.1, 131.8, 125.6 (q, ¹J_{C-F}=281.3 Hz), 125.5 (q, ¹J_{C-F}=281.0 Hz), 123.2, 116.8, 115.8, 115.4, 114.1, 79.9, 43.2 (q, ²J_{C-F}=32.7 Hz), 43.0 (d, ²J_{C-F}=32.3 Hz), 25.3,-1.7.

HRMS (ESI) m/z: [M+H]⁺ Calcd for C₂₅H₂₉BF₆N₂O₃Si 559.2022; Found 559.2023.

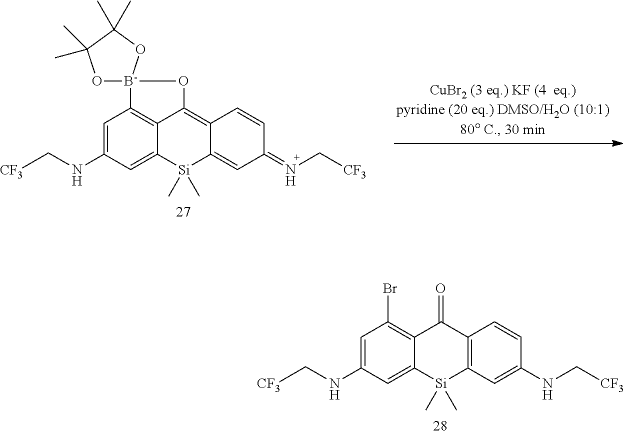

Compound 28. In a 10 mL round-bottom flask, compound 27 (53 mg, 0.095 mmol), potassium fluoride (22 mg, 0.380 mmol, 4 equiv), copper (II) bromide (64 mg, 0.285 mmol, 3 equiv) were placed, followed by addition of DMSO (1 mL), water (100 μL) and pyridine (155 μL, 1.90 mmol, 20 equiv). The reaction mixture was stirred at 80° C. for 30 min. Upon cooling to rt, the mixture was diluted with ethyl acetate and poured into water (80 mL). The product was extracted with ethyl acetate (3×25 mL), the combined extracts were washed with brine (50 mL) and dried over Na₂SO₄. The product was isolated by flash chromatography on Biotage Isolera system (12 g Interchim SiHP 30 μm cartridge, gradient 10% to 60% ethyl acetate: hexane) and freeze-dried from dioxane to give 50 mg (95%, contains 0.5 equiv. dioxane) of 28 as yellow solid.

¹H NMR (400 MHZ, CDCl₃): δ 8.13 (d, J=8.6 Hz, 1H), 7.08 (d, J=2.5 Hz, 1H), 6.81 (dd, J=8.6, 2.6 Hz, 1H), 6.79 (d, J=2.5 Hz, 1H), 6.77 (d, J=2.6 Hz, 1H), 4.40 (t, J=7.0 Hz, 1H), 4.37 (t, J=6.9 Hz, 1H), 3.92-3.79 (m, 4H), 0.44 (s, 6H).

¹⁹F NMR (376 MHZ, CDCl₃): δ-72.06,-72.13.

¹³C NMR (101 MHz, CDCl₃): δ 186.6, 148.2, 147.8, 142.7, 138.7, 134.9, 131.8, 131.1, 125.3, 124.9 (q, ¹J_{C-F}=280.2 Hz), 124.7 (q, ¹J_{C-F}=280.2 Hz), 120.9, 115.7, 115.4, 114.4, 45.4 (q, ²J_{C-F}=34.0 Hz), 45.2 (q, ²J_{C-F}=34.4 Hz), -1.4.

HRMS (ESI) m/z: [M+H]⁺ Calcd for C₁₉H₁₇BrF₆N₂OSi 513.0252; Found 513.0250.

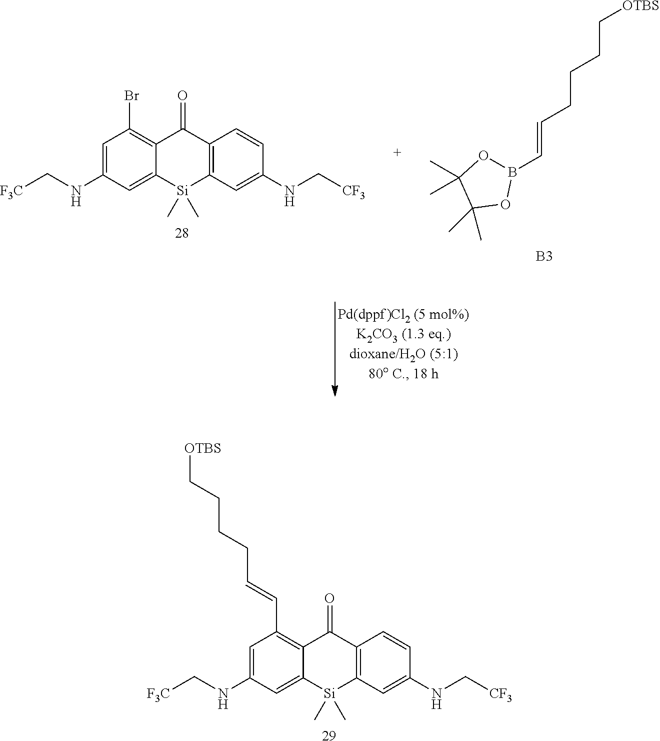

Compound 29. In a 10 mL tube, compounds 28 (46 mg, 0.090 mmol), B3 (52 mg, 0.15 mmol, 1.6 equiv; known compound: Wang et al. Tet. Lett., 2005, 46 (50), 8777-8780), K₂CO₃ (16 mg, 0.12 mmol, 3 equiv) and Pd(dppf) Cl₂·CH₂Cl₂ (3.7 mg, 4.5 μmol, 5 mol %) were loaded. Dioxane (900 μL) and water (180 μL) were added. The mixture was sparged with argon for 30 min, and the tube was sealed and the mixture was stirred at 80° C. for 18 h. The reaction mixture was quenched with sat. aq. NH₄Cl (10 mL) and extracted with ethyl acetate (3×10 mL). The combined organics were washed with brine (50 mL), dried over Na₂SO₄, filtered, and evaporated. The product was isolated by flash chromatography on Biotage Isolera system (12 g Interchim SiHP 30 μm, gradient 0% to 20% ethyl acetate/hexane) and freeze-dried from dioxane to yield 50 mg (86%) of 29 as an orange oil.

¹H NMR (400 MHZ, CDCl₃): δ 8.16 (dd, J=8.1, 1.0 Hz, 1H), 7.17 (dt, J=15.7, 1.5 Hz, 1H), 6.82-6.72 (m, 4H), 5.94 (dt, J=15.5, 6.9 Hz, 1H), 4.33-4.26 (m, 2H), 3.94-3.82 (m, 4H), 3.66 (t, J=6.4 Hz, 2H), 2.30 (qd, J=7.1, 1.5 Hz, 2H), 1.67-1.50 (m, 4H), 0.90 (s, 9H), 0.43 (s, 6H), 0.06 (s, 6H).

¹³C NMR (101 MHZ, CDCl₃): δ 188.2, 147.8, 147.3, 143.9, 141.4, 139.2, 135.3, 132.7, 131.5, 131.3, 124.9 (q, ¹J_{C-F}=280.2 Hz), 115.3, 114.4, 114.1, 63.2, 45.34 (q, ²J_{C-F}=34.0 Hz), 45.20 (q, ²J_{C-F}=33.8 Hz), 45.06, 32.9, 32.6, 26.0, 25.8, 18.4,-1.4,-5.2.

¹⁹F NMR (376 MHZ, CDCl₃): δ-72.09,-72.17.

HRMS (ESI) m/z: [M+H]⁺ Calcd for C₃₁H₄₂F₆N₂O₂Si₂: 645.2762, found: 645.2762.

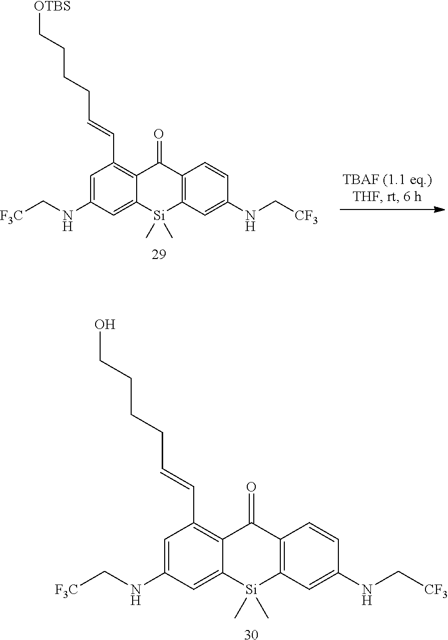

Compound 30. To a solution of compound 29 (43 mg, 0.066 mmol) in THF (660 μL), tetrabutylammonium fluoride trihydrate (TBAF; 19 mg, 0.073 mmol) was added. After stirring for 6 hr. at rt, sat. aq. NH₄Cl (10 mL) was added and the reaction mixture was extracted with ethyl acetate (3×10 mL). The combined extracts were dried over Na₂SO₄, filtered, evaporated, the product was isolated by flash chromatography on a Biotage Isolera system (12 g Interchim SiHP 30 μm cartridge, gradient 20% to 80% ethyl acetate/hexane) and freeze-dried from 1,4-dioxane to yield 28 mg (80%) of 30 as a beige solid.

¹H NMR (400 MHZ, DMSO-d₆): δ 7.91 (d, J=8.8 Hz, 1H), 7.10 (d, J=15.8 Hz, 1H), 6.97 (d, J=2.5 Hz, 1H), 6.94 (d, J=2.6 Hz, 1H), 6.91-6.83 (m, 4H), 5.88 (dt, J=15.5, 6.8 Hz, 1H), 4.37 (t, J=5.1 Hz, 1H), 4.19-4.02 (m, 4H), 3.44 (q, J=5.8 Hz, 2H), 2.23-2.14 (m, 2H), 1.56-1.45 (m, 4H), 0.39 (s, 6H).

¹³C NMR (101 MHZ, DMSO-d₆): δ 186.8, 149.4, 148.9, 142.6, 140.8, 138.5, 132.8, 130.6, 129.8, 128.5, 127.1, 124.3, 115.0, 114.7, 113.8, 113.6, 60.6, 43.3 (q, J=32.3 Hz), 43.2 (q, J=32.7 Hz), 32.4, 32.1, 25.5,-1.4.

¹⁹F NMR (376 MHZ, DMSO-d₆) δ-70.40,-70.48.

HRMS (ESI) m/z: [M+H]⁺ Calcd for C₂₅H₂₈F₆N₂O₂Si: 531.1897, found: 531.1893.

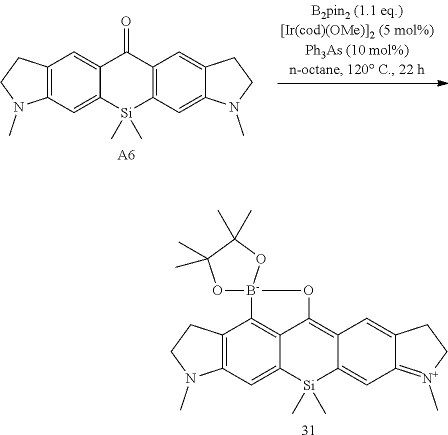

Compound 31. In a 10 mL round-bottom flask, the mixture of compound A6 [G. Lukinavičius et al. J. Am. Chem. Soc. 2016, 138 (30), 9365-9368](174 mg, 0.50 mmol), bis(pinacolato) diboron (140 mg, 0.55 mmol, 1.1 equiv), [Ir (cod)(OMe)]₂ (16.6 mg, 0.025 mmol, 5 mol %) and triphenylarsine (15.3 mg, 0.05 mmol, 10 mol %) in dry n-octane (3.5 mL) was degassed on a Schlenk line and stirred at 120° C. for 22 h. On cooling, the reaction mixture was diluted with dichloromethane, evaporated on Celite and the product was isolated by flash chromatography on Biotage Isolera system (12 g Interchim SiHP 30 μm cartridge, gradient 5% to 50% ethyl acetate/dichloromethane) and freeze-dried from dioxane to give 164 mg (69%) of 31 as orange-red solid.

¹H NMR (400 MHZ, CDCl₃): δ 8.12 (t, J=1.1 Hz, 1H), 6.49 (s, 1H), 6.39 (s, 1H), 3.58 (dd, J=9.0, 8.1 Hz, 2H), 3.53 (dd, J=9.0, 8.1 Hz, 2H), 3.20 (dd, J=9.0, 8.1 Hz, 2H), 3.06 (ddd, J=9.0, 8.1, 1.1 Hz, 2H), 2.95 (s, 6H), 2.90 (s, 6H), 1.41 (br.s, 12H), 0.36 (s, 6H).

¹³C NMR (101 MHz, CDCl₃): δ 186.3, 156.5, 156.3, 146.5, 139.6, 134.1, 133.5, 131.4, 127.4, 124.1, 109.0, 108.3, 80.3, 54.9, 54.5, 34.3, 33.9, 27.60, 27.56, 26.2 (br), 25.8 (br), -1.3.

HRMS (ESI) m/z: [M]⁺ Calcd for C₂₁H₂₄BN₂O₂Si 375.1699; Found 375.1694-corresponds to the pinacol ester hydrolysis product (31a):

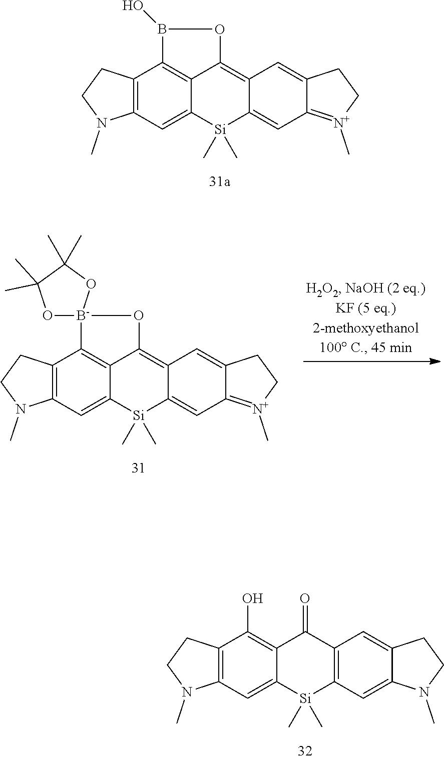 [SMILES: CN1CCc2c1cc1c3c2B(O)OC3=c2cc3c(cc2[Si]1(C)C)=[N+](C)CC3 ; CN1CCc2c1cc1c3c2[B-]2(OC3=c3cc4c(cc3[Si]1(C)C)=[N+](C)CC4)OC(C)(C)C(C)(C)O2 ; CN1CCc2cc3c(cc21)[Si](C)(C)c1cc2c(c(O)c1C3=O)CCN2C]

Compound 32. In a 100 mL round-bottom flask, hydrogen peroxide (1 ml of 30% aq. solution) was added to the mixture of compound 31 (217 mg, 0.46 mmol), potassium fluoride (133 mg, 2.30 mmol, 5 equiv) and sodium hydroxide (1 mL of 1 N aq. solution, ˜1 mmol, ˜2 equiv) 16.6 mg, 0.025 mmol, 5 mol %) in 2-methoxyethanol (22 mL). The reaction mixture was stirred at 100° C. for 30 min, cooled down to rt and the second portion of hydrogen peroxide (1 ml of 30% aq. solution) was added. After stirring for further 15 min at 100° C., the mixture was cooled down, diluted with ethyl acetate (50 mL) and poured into brine. The aqueous phase was adjusted to pH 3-4 with 1 N HCl and it was extracted with ethyl acetate (3×30 mL). The combined organic layers were washed with aq. Na₂S₂O₃, brine and dried over Na₂SO₄. The filtrate was evaporated on silica, the product was isolated by flash chromatography on Biotage Isolera system (24 g Interchim SiHP 30 μm cartridge, gradient 10% to 70% ethyl acetate/hexane) and freeze-dried from dioxane to give 36 mg (21%) of 32 as orange-yellow solid.

¹H NMR (400 MHZ, CDCl₃): δ 14.80 (s, 1H), 8.21 (t, J=1.3 Hz, 1H), 6.48 (s, 1H), 6.20 (s, 1H), 3.54 (t, J=8.6 Hz, 2H), 3.49 (t, J=8.6 Hz, 2H), 3.08-3.00 (m, 4H), 2.91 (s, 3H), 2.90 (s, 3H), 0.42 (s, 6H).

¹³C NMR (101 MHz, CDCl₃): δ 189.7, 161.7, 157.8, 155.1, 143.1, 140.8, 132.2, 130.6, 125.9, 115.20, 115.15, 108.1, 103.3, 55.4, 54.8, 34.5, 34.4, 28.1, 24.9,-0.7.

HRMS (ESI) m/z: [M+H]⁺ Calcd for C₂₁H₂₄N₂O₂Si 365.1680; Found 365.1678.

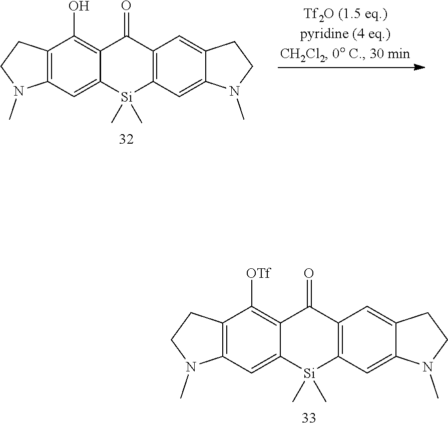

Compound 33. A solution of compound 32 (31 mg, 84.9 μmol) and pyridine (27 μL, 340 μmol, 4 equiv) in dry dichloromethane (4 mL) was cooled in ice-water bath, and trifluoromethanesulfonic anhydride (127 μL of 1 M solution in dichloromethane, ˜127 μmol, ˜1.5 equiv) was added dropwise. The solution has gradually turned green. After 30 min, the reaction mixture was quenched by addition of sat. aq. NaHCO₃ (20 mL) and extracted with dichloromethane (3×20 mL). The combined extracts were washed with brine and dried over Na₂SO₄. The product was isolated by flash chromatography on Biotage Isolera system (12 g Interchim SiHP 30 μm cartridge, gradient 10% to 80% ethyl acetate/hexane) and freeze-dried from dioxane to give 23 mg (55%) of 33 as green-yellow solid.

¹H NMR (400 MHZ, CDCl₃): δ 8.02 (t, J=1.3 Hz, 1H), 6.45 (s, 1H), 6.43 (s, 1H), 3.55 (t, J=8.4 Hz, 2H), 3.46 (t, J=8.4 Hz, 2H), 3.15 (t, J=8.4 Hz, 2H), 3.03 (td, J=8.4, 1.3 Hz, 2H), 2.91 (s, 3H), 2.88 (s, 3H), 0.45 (s, 6H).

¹³C NMR (101 MHZ, CDCl₃): δ 185.8, 156.6, 154.9, 146.5, 143.6, 137.9, 133.4, 132.7, 126.1, 125.0, 124.0, 118.8 (q, ¹J_{C-F}=320.4 Hz), 107.6, 107.5, 55.0, 54.9, 34.8, 34.4, 28.2, 26.2,-1.1.

¹⁹F NMR (376 MHz, CDCl₃): δ-74.3.

HRMS (ESI) m/z: [M+H]⁺ Calcd for C₂₂H₂₃F₃N₂O₄SSi 497.1173; Found 497.1169.

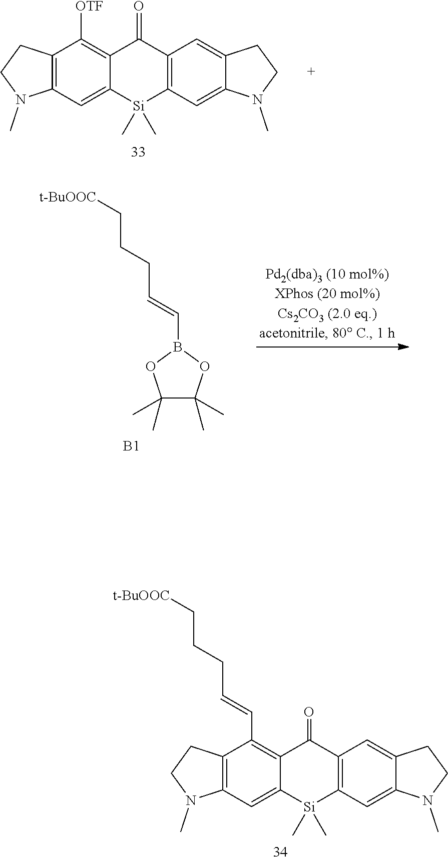

Compound 34. In a 25 mL flask, compound 33 (23 mg, 0.046 mmol), compound B1 (20 mg, 0.069 mmol, 1.5 equiv), Cs₂CO₃ (30 mg, 0.092 mmol, 2 equiv), Pd₂ (dba) 3 (4.2 mg, 4.6 μmol, 10 mol %), and XPhos (4.4 mg, 9.2 μmol, 20 mol %) were loaded. Dry acetonitrile (1.0 mL) was added, the mixture was degassed and stirred at 80° C. for 1 h. Upon cooling, the reaction mixture was quenched with sat. aq. NH₄Cl (10 mL) and extracted with ethyl acetate (3×10 mL). The combined organics were washed with brine (50 mL), dried over Na₂SO₄, filtered, and evaporated. The product was isolated by flash chromatography on Biotage Isolera system (12 g Interchim SiHP 30 μm cartridge, gradient 0% to 40% ethyl acetate/hexane) and freeze-dried from dioxane to yield 12 mg (50%) of 34 as yellow solid.

¹H NMR (400 MHZ, CDCl₃): δ 8.01 (d, J=1.2 Hz, 1H), 6.89 (dt, J=16.0, 1.4 Hz, 1H), 6.47 (d, J=5.8 Hz, 2H), 5.57 (dt, J=16.1, 6.9 Hz, 1H), 3.46-3.35 (m, 4H), 3.06 (t, J=8.2 Hz, 2H), 3.01 (t, J=8.1 Hz, 2H), 2.88 (s, 3H), 2.87 (s, 3H), 2.38-2.25 (m, 4H), 1.80 (p, J=7.5 Hz, 2H), 1.45 (s, 9H), 0.43 (s, 6H).

¹³C NMR (101 MHz, CDCl₃): δ 188.2, 173.3, 154.4, 154.3, 138.2, 134.4, 132.2, 131.8, 131.3, 129.7, 125.8, 107.5, 79.9, 55.3, 55.0, 35.1, 35.0, 34.9, 32.7, 29.3, 28.16, 28.15, 25.2,-1.1.

HRMS (ESI) m/z: [M+H]⁺ Calcd for C₃₁H₄₀N₂O₃Si: 517.2881, found: 517.2879.

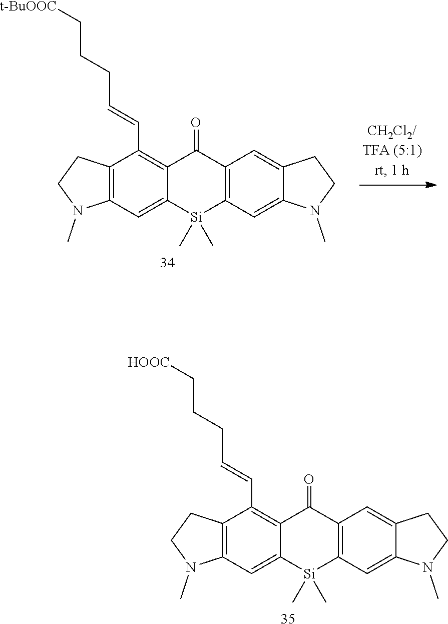

Compound 35. To a solution of compound 34 (11 mg, 0.021 mmol) in CH₂Cl₂ (1.0 mL), trifluoroacetic acid (200 μL) was added dropwise. The resulting reaction mixture was stirred for 1 h at rt, protected from light. The volatiles were removed in vacuo by coevaporation with toluene (3×10 ml). The product was isolated by flash chromatography on Biotage Isolera system (12 g Interchim SiHP 30 μm cartridge, gradient 0% to 10% methanol/dichloromethane) and freeze-dried from dioxane to yield 9.0 mg (92%) of 35 as a yellow solid.

¹H NMR (400 MHZ, DMSO-d₆): δ 12.02 (br, s, 1H), 7.76 (s, 1H), 6.76 (d, J=16.2 Hz, 1H), 6.65 (s, 1H), 6.63 (s, 1H), 5.49 (dt, J=16.1, 6.9 Hz, 1H), 3.45-3.26 (m, 4H), 3.01-2.91 (m, 4H), 2.84 (s, 3H), 2.84 (s, 3H), 2.33 (t, J=7.5 Hz, 2H), 2.18 (q, J=7.0 Hz, 2H), 1.68 (p, J=7.4 Hz, 2H), 0.39 (s, 6H).

¹³C NMR (101 MHZ, DMSO-d₆): δ 186.7, 174.6, 154.4, 154.3, 140.2, 137.6, 137.3, 133.3, 131.9, 131.5, 130.8, 129.8, 129.2, 125.0, 107.7, 54.5, 54.3, 34.6, 34.4, 33.1, 32.2, 28.8, 27.6, 24.5,-1.2.

HRMS (ESI) m/z: [M+H]⁺ Calcd for C₂₇H₃₂N₂O₃Si: 461.2255, found: 461.2252.

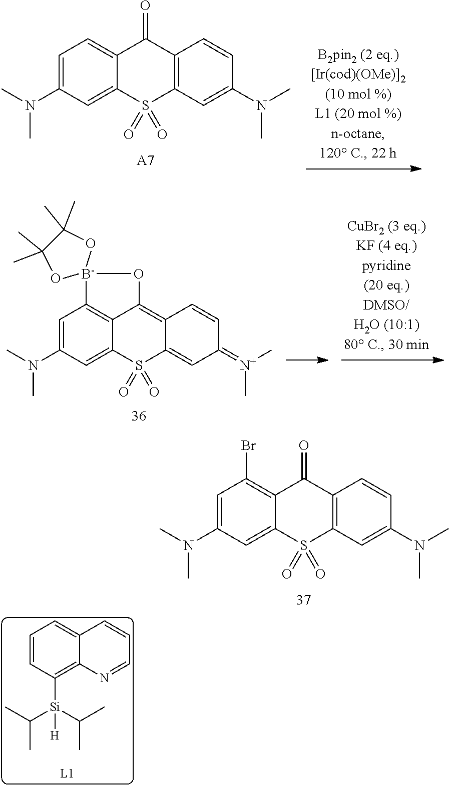

Compound 37. In a 25 mL round-bottom flask, the mixture of compound A7 (200 mg, 0.61 mmol; known compound: J. Liu et al. ACS Appl. Mater. Interfaces 2016, 8, 22953-22962), bis(pinacolato) diboron (308 mg, 1.21 mmol, 2 equiv), [Ir (cod)(OMe)]₂ (40 mg, 0.061 mmol, 10 mol %) and ligand L1 (8-(diisopropylsilyl) quinoline; known compound: B. Ghaffari et al. J. Am. Chem. Soc. 2014, 136, 14345-14348)(30 mg, 0.121 mmol, 20 mol %) in dry n-octane (8 mL) was degassed on a Schlenk line and stirred at 120° C. for 22 h. On cooling, the reaction mixture was diluted with dichloromethane and evaporated to dryness in a 50 mL round-bottom flask and the obtained crude 36 was used directly in the next step.

To the residue of crude compound 36, potassium fluoride (141 mg, 2.42 mmol, 4 equiv) and copper (II) bromide (405 mg, 1.82 mmol, 3 equiv) were added, followed by DMSO (6 mL), water (600 μL) and pyridine (980 μL, 12.1 mmol, 20 equiv). The reaction mixture was stirred at 80° C. for 30 min. Upon cooling to rt, the reaction mixture was diluted with ethyl acetate and poured into 0.5 N HCl (100 mL). The product was extracted with ethyl acetate (4×30 mL), the combined extracts were washed with water, brine (50 mL each) and dried over Na₂SO₄. The product was isolated by flash chromatography on Biotage Isolera system (25 g Interchim SiHP 30 μm cartridge, gradient 0% to 10% ethyl acetate/dichloromethane) to give 79 mg (32%) of 37 as yellow solid.

¹H NMR (400 MHZ, CDCl₃): δ 8.13 (d, J=9.0 Hz, 1H), 7.31 (d, J=2.7 Hz, 1H), 7.17 (d, J=2.6 Hz, 1H), 7.10 (d, J=2.7 Hz, 1H), 6.87 (dd, J=9.0, 2.6 Hz, 1H), 3.15 (s, 12H).

¹³C NMR (101 MHz, CDCl₃) δ 175.8, 152.8, 151.9, 144.3, 140.5, 131.5, 125.3, 121.2, 120.9, 117.1, 115.1, 105.4, 103.7, 40.34, 40.26.

HRMS (ESI) m/z: [M+H]⁺ Calcd for C₁₇H₁₇BrN₂O₃S 409.0216; Found 409.0217.

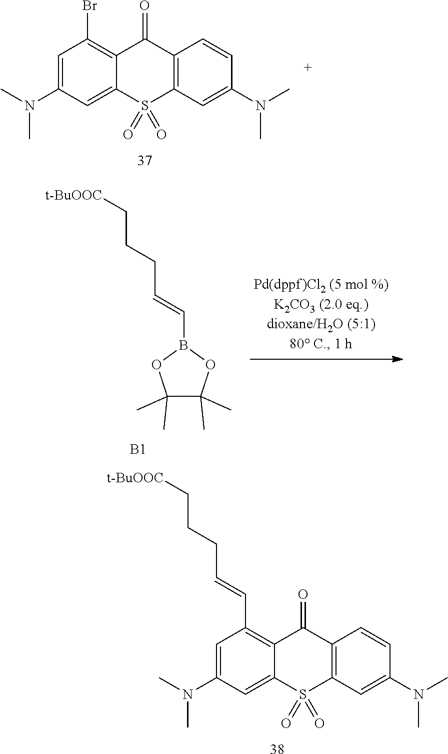

Compound 38. In a 10 mL tube, compound 37 (41 mg, 0.10 mmol), compound B1 (44 mg, 0.15 mmol, 1.5 equiv), K₂CO₃ (28 mg, 0.20 mmol, 2 equiv) and Pd(dppf) Cl₂·CH₂Cl₂ (4.1 mg, 5.0 μmol, 5 mol %) were loaded. Dioxane (1.0 mL) and water (0.2 mL) were added. The mixture was sparged with argon for 30 min, and the tube was sealed and the mixture was stirred at 80° C. for 1 h. Upon cooling, sat. aq. NH₄Cl (10 mL) was added and extracted with ethyl acetate (3×10 ml). The combined organics were washed with brine (50 mL), dried over Na₂SO₄, filtered, and evaporated. The product was isolated by flash chromatography on Biotage Isolera system (12 g Interchim SiHP 30 μm cartridge, gradient 20% to 80% ethyl acetate/hexane) and freeze-dried from dioxane to yield 44 mg (88%) of 38 as yellow solid.

¹H NMR (400 MHZ, CDCl₃): δ 8.11 (d, J=9.0 Hz, 1H), 7.42 (dt, J=15.5, 1.5 Hz, 1H), 7.26 (d, J=3.0 Hz, 1H), 7.19 (d, J=2.7 Hz, 1H), 6.86 (dd, J=9.0, 2.7 Hz, 1H), 6.78 (d, J=2.8 Hz, 1H), 5.98 (dt, J=15.5, 6.9 Hz, 1H), 3.17 (s, 6H), 3.14 (s, 6H), 2.38-2.28 (m, 4H), 1.84 (p, J=7.7 Hz, 2H), 1.45 (s, 9H).

¹³C NMR (101 MHz, CDCl₃): δ 177.8, 173.1, 152.6, 151.9, 144.6, 143.7, 140.6, 132.42, 132.40, 131.1, 120.8, 116.6, 114.9, 114.4, 104.6, 103.6, 80.1, 40.2, 40.1, 35.1, 32.5, 28.2, 24.7.

HRMS (ESI) m/z: [M+H]⁺ Calcd for C₂₇H₃₄N₂O₅S: 499.2261, found: 499.2262.

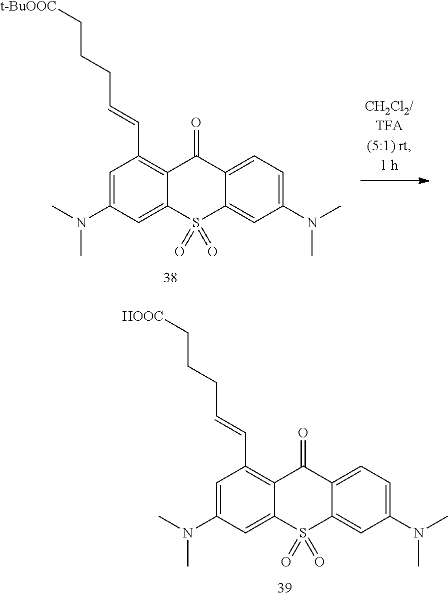

Compound 39. To a solution of compound 38 (38 mg, 0.062 mmol) in CH₂Cl₂ (1 mL) trifluoroacetic acid (200 μL) was added dropwise. The resulting reaction mixture was stirred for 60 minutes at rt, protected from light. The volatiles were removed in vacuo by coevaporation with toluene (3×10 ml). The product was isolated by flash chromatography on Biotage Isolera system (12 g Interchim SiHP 30 μm cartridge, gradient 0% to 10% methanol/dichloromethane) and freeze-dried from 1,4-dioxane to yield 24 mg (67%) of 39 as yellow solid.

¹H NMR (400 MHZ, DMSO-d₆): δ 12.03 (br s, 1H), 7.95 (d, J=8.2 Hz, 1H), 7.31 (d, J=15.6 Hz, 1H), 7.12 (d, J=2.6 Hz, 1H), 7.04 (dd, J=8.9, 1.7 Hz, 2H), 6.89 (s, 1H), 6.16 (dt, J=14.0, 6.7 Hz, 1H), 3.15 (s, 6H), 3.12 (s, 6H), 2.32 (t, J=7.4 Hz, 2H), 2.24 (q, J=6.9 Hz, 2H), 1.72 (p, J=7.4 Hz, 2H).

¹³C NMR (101 MHZ, DMSO-d₆): δ 177.3, 174.5, 152.4, 151.6, 143.30, 143.26, 140.1, 133.1, 130.7, 130.6, 119.8, 115.3, 115.2, 113.6, 103.8, 102.7, 39.6, 39.5, 33.2, 32.0, 23.9.

HRMS (ESI) m/z: [M+H]⁺ Calcd for C₂₃H₂₆N₂OSS: 443.1635, found: 443.1634.

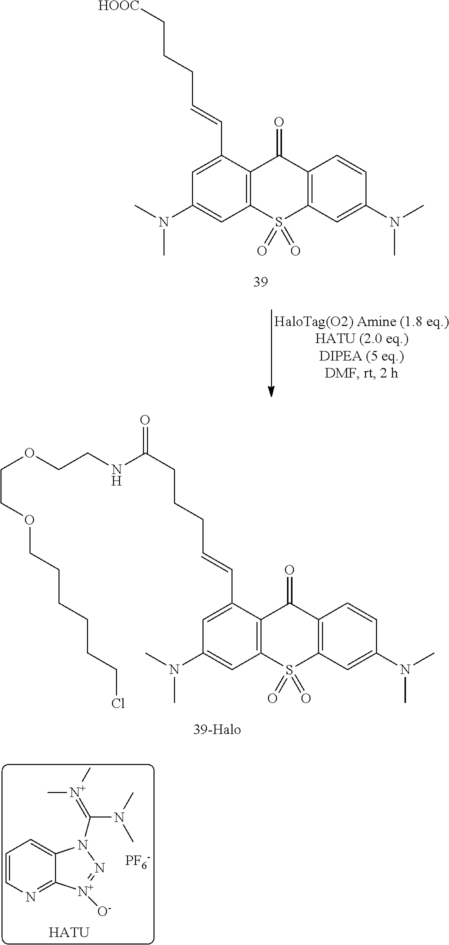

Compound 39-Halo. In an amber vial, compound 39 (10 mg, 0.023 mmol), HaloTag (02) Amine (9.3 mg, 0.042 mmol), and HATU (18 mg, 0.047 mmol) were dissolved in DMF (200 μL) and DIPEA (32 mg, 0.25 mmol) was added and stirred for 2 h at rt. The volatiles were removed in vacuo. The product was isolated by flash chromatography on a Biotage Isolera system (12 g Interchim SiHP 30 μm cartridge, eluting with ethyl acetate) and freeze-dried from dioxane to yield 11 mg (74%) 39-Halo as a yellow solid.

HRMS (ESI) m/z: [M+H]⁺ Calcd for C₃₃H₄₆ClN₃O₆S: 648.2869, found: 648.2864.

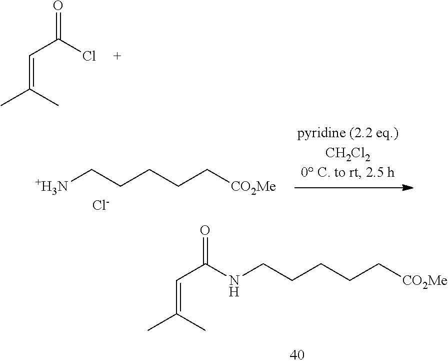

Compound 40. In a 100 mL round-bottom flask, 3,3-dimethylacryloyl chloride (0.5 mL, 4.4 mmol, 1.1 equiv) was added to a suspension of methyl 6-aminohexanoate hydrochloride (728 mg, 4 mmol) in dry dichloromethane (40 mL), cooled in ice-water bath. Pyridine (0.75 mL, 8.8 mmol, 2.2 equiv) was then added dropwise, the reaction mixture was warmed up to rt and stirred for 2.5 h. The resulting yellowish solution was then poured into sat. aq. NaHCO₃ (50 mL), extracted with dichloromethane (3×30 mL); the combined extracts were washed with 0.1 N HCl, water and brine (50 mL each) and dried over Na₂SO₄. The product was isolated by flash chromatography on Biotage Isolera system (40g RediSep Rf cartridge, gradient 30% to 100% ethyl acetate/hexane) and dried in vacuo to give 40 as colorless oil, yield 852 mg (94%).

¹H NMR (400 MHZ, CDCl₃): δ 5.54 (hept, J=1.3 Hz, 1H), 5.42 (br.s, 1H), 3.67 (s, 3H), 3.28 (td, J=7.1, 5.9 Hz, 2H), 2.32 (t, J=7.4 Hz, 2H), 2.15 (d, J=1.3 Hz, 3H), 1.83 (d, J=1.3 Hz, 3H), 1.71-1.60 (m, 2H), 1.58-1.48 (m, 2H), 1.43-1.31 (m, 2H).

¹³C NMR (101 MHZ, CDCl₃): δ 174.2, 167.1, 150.7, 118.7, 51.6, 39.0, 34.0, 29.5, 27.2, 26.6, 24.7, 19.9.

HRMS (ESI) m/z: [M+H]⁺ Calcd for C₁₂H₂₁NO₃ 228.1594; Found 228.1591.

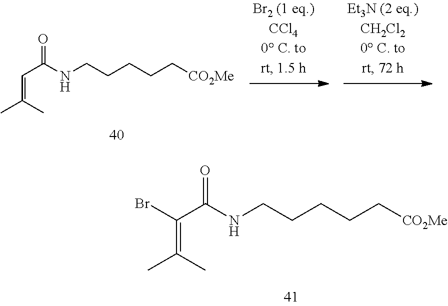 [SMILES: COC(=O)CCCCCNC(=O)C=C(C)C ; COC(=O)CCCCCNC(=O)C(Br)=C(C)C]

Compound 41. A solution of bromine (0.19 mL, 3.7 mmol, 1 equiv) in CCl₄ (2 mL) was added dropwise to a solution of 40 (843 mg, 3.7 mmol) in CCl₄ (2 mL), cooled in ice-water bath. The reaction mixture was warmed up to rt, stirred for 1.5 h, the solvents were evaporated and the residue was chased with dichloromethane (20 mL). The resulting light orange oil of the crude dibromide was dissolved in dichloromethane (5 mL), cooled in ice-water bath, and triethylamine (1 mL, 7.4 mmol, 2 equiv) in dichloromethane (1.5 mL) was added dropwise. The cold bath was removed, and the reaction mixture was left stirring at rt for 3 days. It was then poured into water (100 mL), extracted with dichloromethane (3×30 mL), the combined extracts were washed with brine (50 mL each) and dried over Na₂SO₄. The product was isolated by flash chromatography on Biotage Isolera system (40g RediSep Rf cartridge, gradient 20% to 80% ethyl acetate/hexane) and dried in vacuo to give 41 as yellowish oil, yield 817 mg (72%).

¹H NMR (400 MHZ, CDCl₃): δ 6.18 (br.s, 1H), 3.67 (s, 3H), 3.31 (td, J=7.1, 5.9 Hz, 2H),), 2.33 (t, J=7.4 Hz, 2H), 2.11 (s, 3H), 1.98 (s, 3H), 1.71-1.62 (m, 2H), 1.62-1.52 (m, 2H), 1.43-1.33 (m, 2H).

¹³C NMR (101 MHZ, CDCl₃): δ 174.1, 164.8, 143.5, 111.1, 51.7, 39.9, 34.0, 29.2, 26.51, 26.48, 24.6, 22.9.

HRMS (ESI) m/z: [M+H]⁺ Calcd for C₁₂H₂₀BrNO₃ 306.0699; Found 306.0697.

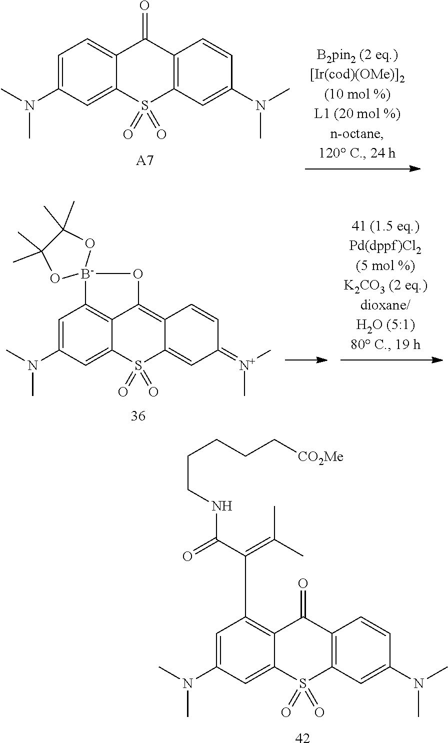

Compound 42. In a dried 10 mL crimp-top tube (a 2-5 mL Biotage microwave vial was used), the mixture of compound A7 (15 mg, 45.5 μmol), bis(pinacolato) diboron (23 mg, 91 μmol, 2 equiv), [Ir (cod)(OMe)]₂ (3 mg, 4.55 μmol, 10 mol %) and ligand L1 (8-(diisopropylsilyl) quinoline; 2.2 mg, 9.1 μmol, 20 mol %) in dry n-octane (0.6 mL) was degassed on a Schlenk line and stirred at 120° C. for 24 h. On cooling, the reaction mixture was diluted with dichloromethane, filtered through a short plug of Celite, washed with dichloromethane; the filtrate was concentrated, transferred into another 10 mL crimp-top tube and dried. Compound 41 (21 mg, 68 μmol, 1.5 equiv), Pd(dppf) Cl₂·CH₂Cl₂ (1.8 mg, 2.25 μmol, 5 mol %) and K₂CO₃ (12 mg, 90 μmol, 2 equiv) were added followed by dioxane (500 μL) and water (100 μL), the mixture was degassed and stirred at 80° C. for 19 h. The product was isolated by preparative HPLC (Interchim Uptisphere Strategy PhC4 250×21.2 mm 5 μm, solvent flow rate 18 mL/min, gradient 35% to 75% A/B, A: acetonitrile+0.1% (v/v) formic acid, B: water+0.1% (v/v) formic acid) and freeze-dried from dioxane to give 14 mg (56%) of 42 as yellow solid.

¹H NMR (400 MHZ, CDCl₃): δ 8.02 (d, J=9.0 Hz, 1H), 7.60 (br.t, J=5.9 Hz, 1H), 7.26 (d, J=2.8 Hz, 1H), 7.22 (d, J=2.7 Hz, 1H), 6.85 (dd, J=9.0, 2.7 Hz, 1H), 6.76 (d, J=2.8 Hz, 1H), 3.64 (s, 3H), 3.43-3.31 (m, 1H), 3.20-3.10 (m, 1H), 3.16 (s, 6H), 3.15 (s, 6H), 2.26 (t, J=7.6 Hz, 2H), 1.90 (s, 3H), 1.67-1.48 (m, 4H), 1.38-1.30 (m, 2H), 1.30 (s, 3H).

¹³C NMR (101 MHz, CDCl₃): δ 178.2, 174.2, 170.3, 152.9, 152.2, 143.6, 142.6, 141.3, 133.6, 131.8, 130.9, 120.6, 118.0, 117.2, 114.8, 104.8, 104.2, 51.6, 40.4, 40.3, 39.3, 34.1, 29.4, 26.6, 24.7, 21.4, 20.6.

HRMS (ESI) m/z: [M+H]⁺ Calcd for C₂₉H₃₇N₃O₆S 556.2476; Found 556.2472.

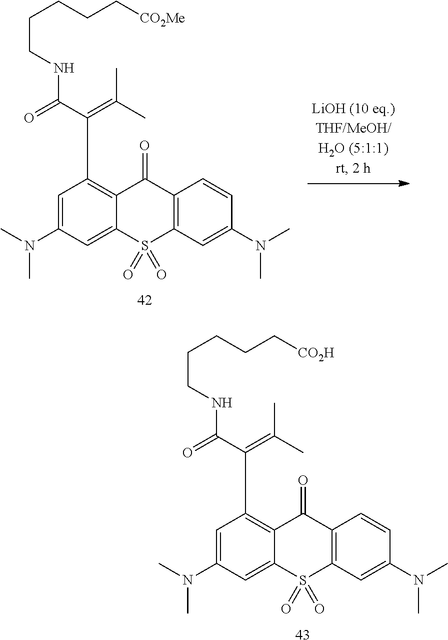

Compound 43. A solution of LiOH·H₂O (9 mg, 216 μmol, 10 equiv) in water (100 μL) was added to the solution of compound 42 (12 mg, 21.6 μmol) in tetrahydrofuran (500 μL) and methanol (100 μL), the mixture was sonicated briefly and stirred vigorously at rt for 2 h. Acetic acid (50 μL) was then added, and the mixture was evaporated to dryness. The product was isolated from the residue by preparative HPLC (Interchim Uptisphere Strategy PhC4 250×21.2 mm 5 μm, solvent flow rate 18 mL/min, gradient 30% to 70% A/B, A: acetonitrile+0.1% (v/v) formic acid, B: water +0.1% (v/v) formic acid) and freeze-dried from dioxane to give 11 mg (94%) of 43 as yellow solid.

¹H NMR (400 MHZ, CDCl₃): δ 8.02 (d, J=9.0 Hz, 1H), 7.70 (t, J=5.9 Hz, 1H), 7.26 (d, J=2.8 Hz, 1H), 7.22 (d, J=2.7 Hz, 1H), 6.85 (dd, J=9.0, 2.7 Hz, 1H), 6.77 (d, J=2.8 Hz, 1H), 3.40-3.28 (m, 1H), 3.27-3.17 (m, 1H), 3.16 (s, 6H), 3.15 (s, 6H), 2.27 (t, J=7.4 Hz, 2H), 1.90 (s, 3H), 1.66-1.47 (m, 4H), 1.38-1.27 (m, 2H), 1.29 (s, 3H).

¹³C NMR (101 MHz, CDCl₃): δ 178.2, 177.6, 170.5, 152.9, 152.2, 143.4, 142.6, 141.1, 133.3, 132.2, 131.0, 120.5, 117.8, 117.2, 114.9, 104.9, 104.2, 40.4, 40.3, 39.2, 33.8, 29.2, 26.4, 24.4, 21.5, 20.6.

HRMS (ESI) m/z: [M+H]⁺ Calcd for C₂₈H₃₅N₃O₆S 542.2319; Found 542.2315.

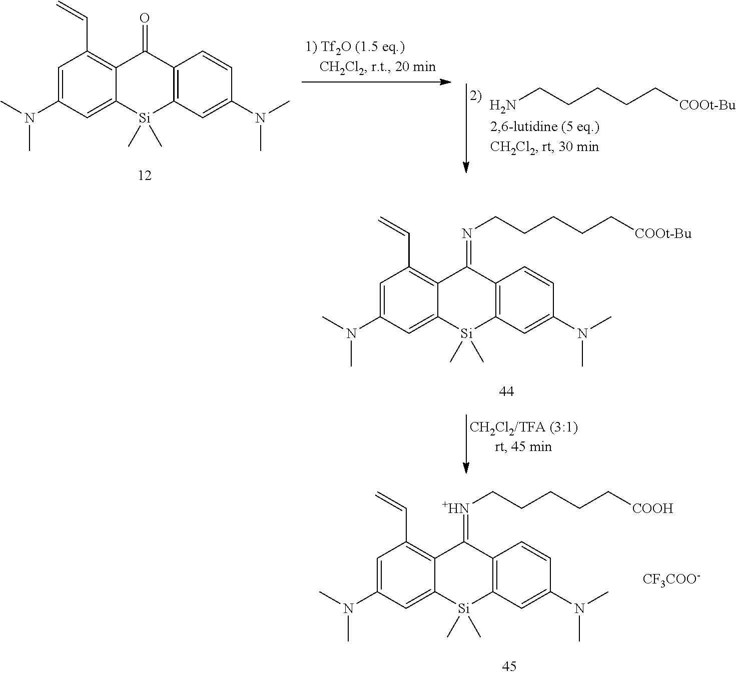

Compound 45. Compound 12 (20 mg, 0.057 mmol) was dissolved in dichloromethane (500 μL). Trifluoromethanesulfonic anhydride solution (86 μl of 1 M in dichloromethane, 0.086 mmol, 1.5 equiv) was added dropwise, and the solution stirred for 20 min at rt. The resulting blue solution was transferred dropwise to a stirring solution of tert-butyl 6-aminohexanoate (21.3 mg, 0.114 mmol, 2 equiv) and 2,6-lutidine (30.5 mg, 0.285 mmol) in dichloromethane (500 μL), cooled in an ice-water bath. An additional rinse of dichloromethane (500 μL) was used to ensure complete transfer. The solution was stirred for 30 min, then sat. aq. NaHCO₃ (10 mL) was added and the reaction mixture was extracted with ethyl acetate (3×10 mL). The combined extracts were washed with brine (50 mL), dried over Na₂SO₄, filtered, evaporated, the product was isolated by flash chromatography on a Biotage Isolera system (12 g Interchim SiHP 30 μm cartridge, 20 to 100% ethyl acetate/hexane) and freeze-dried from 1,4-dioxane to yield 20 mg (66%) of 44 as a brown oil which was used directly in the next step.

To a solution of compound 44 (20 mg, 0.038 mmol) in dichloromethane (300 μL), trifluoroacetic acid (100 μL) was added dropwise. The reaction mixture was stirred for 45 minutes at rt, protected from light. The volatiles were removed in vacuo by coevaporation with toluene (3×10 ml) and freeze-dried from dioxane to yield 22 mg (˜100%, or 66% over 2 steps) of 45 as an orange solid (mixture of (E)- and (Z)-isomers of the imine in 2:1 ratio).

¹H NMR, major (E)-isomer (400 MHz, DMSO-d₆): δ 11.99 (br.s, 1H), 11.54 (d, J=8.1 Hz, 1H), 7.76 (d, J=8.8 Hz, 1H), 7.06 (d, J=2.6 Hz, 1H), 7.04 (d, J=2.7 Hz, 1H), 6.96 (d, J=2.6 Hz, 1H), 6.91 (dd, J=8.8, 2.7 Hz, 1H), 6.74 (dd, J=17.3, 10.9 Hz, 1H), 5.88 (dd, J=17.3, 0.9 Hz, 1H), 5.45 (dd, J=10.9, 0.9 Hz, 1H), 3.40-3.20 (m, 2H), 3.10 (s, 6H), 3.07 (s, 6H), 2.05 (t, J=7.3 Hz, 2H), 1.65-1.52 (m, 2H), 1.36-1.18 (m, 4H), 0.68 (s, 3H), 0.32 (s, 3H).

¹³C NMR, major (E)-isomer (101 MHz, DMSO-d₆): δ 175.5, 173.8, 151.3, 150.9, 139.4, 137.7, 137.6, 134.5, 128.6, 125.6, 124.6, 118.3, 115.8, 115.3, 111.9, 109.0, 49.0, 39.4, 39.3, 32.9, 26.9, 25.2, 23.4,-0.5,-5.2.

HRMS (ESI) m/z: [M+H]⁺ Calcd for C₂₇H₃₇N₃O₂Si: 464.2728, found: 464.2725.

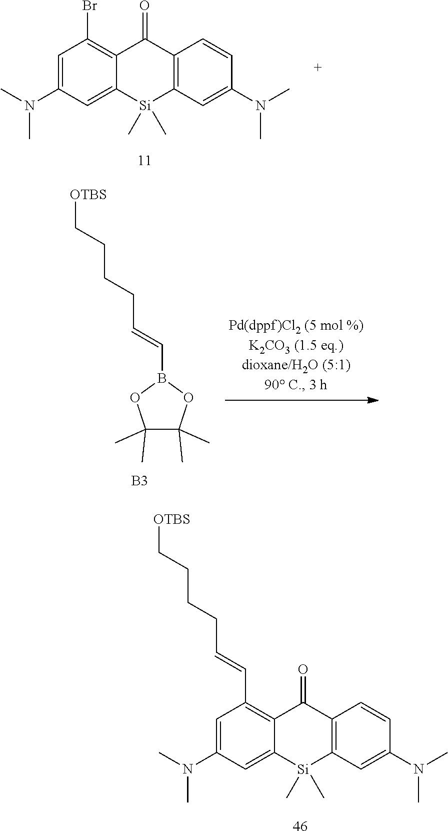

Compound 46. In a 25 mL flask, compound 11 (200 mg, 0.50 mmol), compound B3 (255 mg, 0.75 mmol, 1.5 equiv), K₂CO₃ (104 mg, 0.75 mmol, 1.5 equiv) and Pd(dppf) Cl₂·CH₂Cl₂ (21 mg, 25 μmol, 5 mol %) were loaded. Dioxane (5.0 mL) and water (1.0 mL) were added. The mixture was sparged with argon for 30 min, and the reaction was stirred at 80° C. for 3 h. Upon cooling, the reaction was diluted with ethyl acetate (20 mL) and washed with sat. aq. NH₄Cl (20 mL), brine (20 mL). The organics were dried over Na₂SO₄, filtered, and evaporated. The product was isolated by flash chromatography on Biotage Isolera system (40 g RediSep Rf cartridge, gradient 0% to 30% ethyl acetate/hexane) and freeze-dried from dioxane to yield 193 mg (72%) of 46 as yellow solid.

¹H NMR (400 MHZ, CDCl₃): δ 8.23 (d, J=8.9 Hz, 1H), 7.26 (s, 1H), 6.82 (dd, J=8.9, 2.8 Hz, 1H), 6.78-6.75 (m, 2H), 6.74 (d, J=2.8 Hz, 1H), 5.93 (dt, J=15.4, 6.8 Hz, 1H), 3.66 (t, J=6.4 Hz, 2H), 3.09 (s, 6H), 3.07 (s, 6H), 2.31 (td, J=7.0, 5.5 Hz, 2H), 1.68-1.53 (m, 4H), 0.90 (s, 9H), 0.45 (s, 6H), 0.06 (s, 6H).

¹³C NMR (101 MHz, CDCl₃): δ 188.0, 151.1, 150.7, 143.7, 141.1, 138.9, 134.1, 132.3, 131.2, 129.9, 128.3, 114.1, 113.71, 113.67, 113.2, 63.3, 40.1, 40.0, 32.9, 32.7, 26.0, 25.9, 18.4,-1.0,-5.2.

HRMS (ESI) m/z: [M+H]⁺ C₃₁H₄₈N₂O₂Si₂: 537.3327, found: 537.3327.

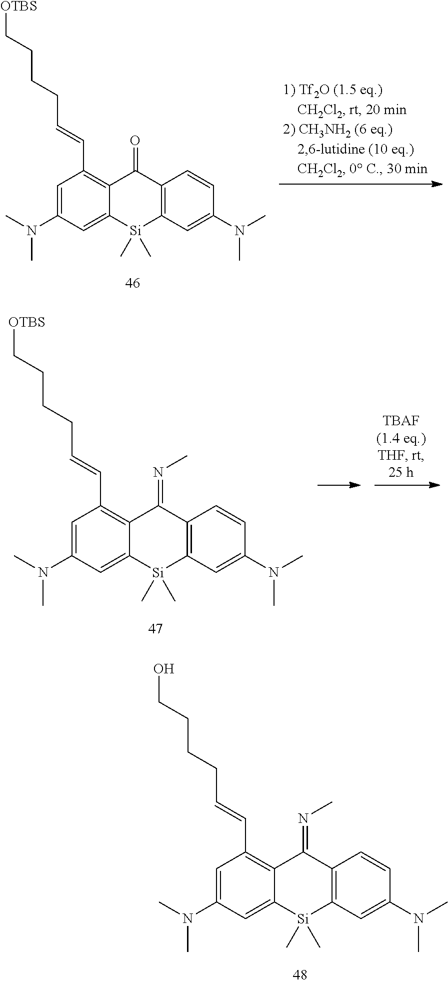 [SMILES: CN(C)c1ccc2c(c1)[Si](C)(C)c1cc(N(C)C)cc(/C=C/CCCCO[Si](C)(C)C(C)(C)C)c1C2=O ; C/N=C1\c2ccc(N(C)C)cc2[Si](C)(C)c2cc(N(C)C)cc(/C=C/CCCCO[Si](C)(C)C(C)(C)C)c21 ; C/N=C1\c2ccc(N(C)C)cc2[Si](C)(C)c2cc(N(C)C)cc(/C=C/CCCCO)c21]

Compound 48. Compound 46 (17 mg, 0.031 mmol) was dissolved in dichloromethane (500 μL). Trifluoromethanesulfonic anhydride solution (47 μL of 1 M in dichloromethane, 0.047 mmol, 1.5 equiv) was added dropwise, and the solution stirred for 20 min at rt. The resulting blue solution was transferred dropwise to a stirring solution of methylamine (93 μL of 2 M in THF, 0.186 mmol, 6 equiv) and 2,6-lutidine (33 mg, 0.31 mmol, 10 equiv) in dichloromethane (500 μL) cooled in an ice-water bath. An additional rinse of dichloromethane (500 μL) was used to ensure complete transfer. The solution was stirred for 30 min, then sat. aq. NaHCO₃ (10 mL) was added and the reaction mixture was extracted with ethyl acetate (3×10 mL). The combined extracts were washed with brine (50 mL), dried over Na₂SO₄, filtered, evaporated and the crude 47 was used directly in the next step without purification.

The crude 47 was dissolved in THF (500 μl) and tetrabutylammonium fluoride trihydrate (TBAF; 14 mg, 0.044 mmol, 1.4 equiv) was added and stirred at rt for 4 hours. The reaction was quenched with addition sat. aq. NH₄Cl (10 mL), and extracted with ethyl acetate (3×10 mL). The combined organics were washed with brine, dried over Na₂SO₄, filtered, and evaporated. The product was isolated by preparative HPLC (Interchim Uptisphere Strategy PhC4 250×21.2 mm 5 μm, solvent flow rate 18 mL/min, gradient 25% to 65% A/B, A: acetonitrile+0.1% (v/v) formic acid, B: water +0.1% (v/v) formic acid) to give 6.6 mg (49%) of 48 as an orange solid (mixture of (E)- and (Z)-isomers of the imine in 4:1 ratio).

¹H NMR, major (E)-isomer (400 MHZ, DMSO-d₆): δ 7.41 (d, J=8.5 Hz, 1H), 6.88 (d, J=2.6 Hz, 1H), 6.83 (d, J=2.8 Hz, 1H), 6.81 (d, J=2.5 Hz, 1H), 6.74 (dd, J=8.5, 2.8 Hz, 1H), 6.25 (dt, J=15.9, 6.7 Hz, 1H), 6.10 (d, J=15.9 Hz, 1H), 4.38 (br.s, 1H), 3.47-3.39 (m, 2H), 3.04 (s, 3H), 2.96 (s, 6H), 2.92 (s, 6H), 2.21-2.10 (m, 2H), 1.50-1.43 (m, 4H), 0.59 (s, 3H), 0.17 (s, 3H).

¹³C NMR, major (E)-isomer (101 MHz, DMSO-d₆): δ 167.3, 149.3, 138.1, 138.0, 135.4, 135.2, 131.7, 128.9, 128.1, 126.3, 115.5, 114.6, 113.0, 108.5, 60.5, 41.3, 39.9, 39.7, 32.6, 32.2, 25.4,-0.4,-5.3.

HRMS (ESI) m/z: [M+H]⁺ Calcd for C₂₆H₃₇N₃OSi: 436.2779, found: 436.2777.

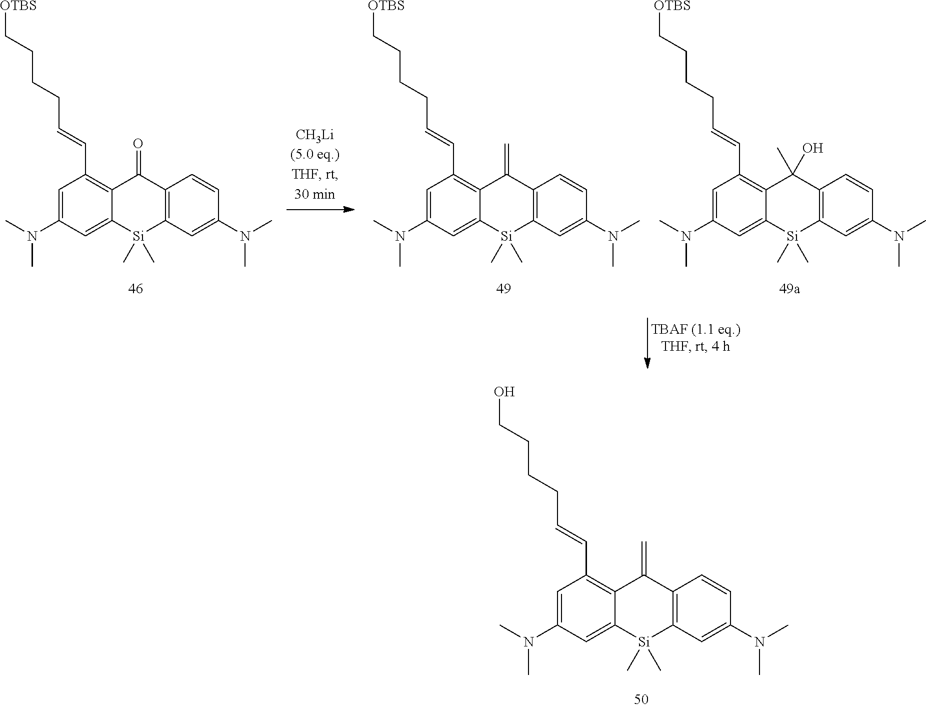

Compound 50. To a solution of compound 46 (34.6 mg, 0.064 mmol) in anhydrous THF (2.0 mL) under argon, a solution of methyllithium (0.20 ml of 1.6 M in hexane, 0.32 mmol, 5 equiv) was added dropwise and stirred at rt for 30 minutes. The reaction was quenched by slow addition of sat. aq. NH₄Cl (10 mL) and extracted with ethyl acetate (3×10 mL). The combined organics were washed with brine (50 mL), dried over Na₂SO₄, filtered, and evaporated. The product was isolated by flash chromatography on Biotage Isolera system (12 g Interchim SiHP 30 μm cartridge, gradient 0% to 40% ethyl acetate/hexane) and freeze-dried from 1,4-dioxane to yield a mixture of 49 and 49a as a colourless oil.

This mixture was dissolved in THF (1 ml), and tetrabutylammonium fluoride trihydrate (TBAF; 23 mg, 0.073 mmol, 1.1 equiv) was added and stirred at rt for 4 h. The reaction was quenched with addition sat. aq. NH₄Cl (10 mL), and extracted with ethyl acetate (3×10 mL). The combined organics were washed with brine, dried over Na₂SO₄, filtered, and evaporated. The product was isolated by flash chromatography on Biotage Isolera system (12 g Interchim SiHP 30 μm cartridge, gradient 20% to 80% ethyl acetate/hexane) and freeze-dried from dioxane to yield 14.1 mg of 46 (52%) as a red oil.

¹H NMR (400 MHZ, DMSO-d₆): δ 7.41 (d, J=8.6 Hz, 1H), 6.87 (dd, J=7.6, 2.8 Hz, 2H), 6.81 (d, J=2.7 Hz, 1H), 6.78-6.69 (m, 2H), 6.10 (dt, J=15.7, 6.9 Hz, 1H), 5.50 (d, J=2.1 Hz, 1H), 5.01 (d, J=2.2 Hz, 1H), 4.37 (t, J=5.2 Hz, 1H), 3.48-3.39 (m, 2H), 2.94 (s, 6H), 2.91 (s, 6H), 2.23-2.14 (m, 2H), 1.53-1.45 (m, 4H), 0.36 (s, 6H).

¹³C NMR (101 MHz, DMSO-d₆): δ 148.9, 148.4, 145.0, 136.3, 136.0, 135.0, 134.3, 132.6, 131.1, 129.6, 126.6, 116.6, 115.6, 115.2, 113.6, 111.3, 60.6, 40.20, 40.16, 32.5, 32.1, 25.4,-2.9.

HRMS (ESI) m/z: [M+H]⁺ Calcd for C₂₆H₃₆N₂OSi 421.2670; Found 421.2668.

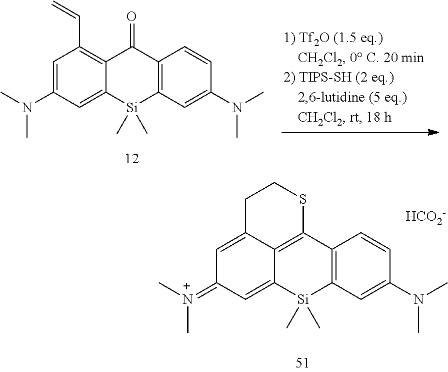

Compound 51. In a 10 mL round-bottom flask, trifluoromethanesulfonic anhydride (43 μL of 1 M solution in dichloromethane, ˜43 μmol, ˜1.5 equiv) was added to the solution of 12 (10 mg, 28.5 μmol) in dry dichloromethane (0.5 mL), cooled in ice-water bath. After stirring for 20 min at 0° C., the solution was transferred into the mixture of triisopropylsilanethiol (TIPS-SH; 12 μL, 57 μmol, 2 equiv) and 2,6-lutidine (17 μL, 143 μmol, 5 equiv) in dry dichloromethane (0.5 mL). The resulting blue solution was stirred at rt for 18 h, during which time the color of the mixture changed to violet. The solvents were evaporated and the product was isolated from the residue by preparative HPLC (Interchim Uptisphere Strategy PhC4 250×21.2 mm 5 μm, solvent flow rate 18 mL/min, gradient 40% to 80% A/B, A: acetonitrile+0.1% (v/v) formic acid, B: water+0.1% (v/v) formic acid) to give 1.4 mg (12%) of 51 as dark blue film (formate salt).

HRMS (ESI) m/z: [M]⁺ Calcd for C₂₁H₂₇N₂SSi 367.1659; Found 367.1657.

### Example 2

### Characterisation of Exemplary Compounds of the Present Invention

### General Materials and Methods

All chemical reagents (TCI, Sigma-Aldrich, Alfa Aesar) and dry solvents for synthesis (over molecular sieves, AcroSeal package, Acros Organics) were purchased from reputable suppliers and were used as received without further purification. The products were lyophilized from a suitable solvent system using Alpha 2-4 LDplus freeze-dryer (Martin Christ Gefriertrocknungsanlagen GmbH).

### Thin Layer Chromatography

Normal phase TLC was performed on silica gel 60 F₂₅₄ (Merck Millipore, Germany). For TLC on reversed phase silica gel 60 RP-18 F₂₅₄s (Merck Millipore) was used. Compounds were detected by exposing TLC plates to UV-light (254 or 366 nm) or heating with vanillin stain (6 g vanillin and 1.5 mL conc. H₂SO₄ in 100 mL ethanol), unless indicated otherwise.

### Flash Chromatography

Preparative flash chromatography was performed with an automated Isolera One system with Spektra package (Biotage AG) using commercially available cartridges of suitable size as indicated (RediSep Rf series from Teledyne ISCO, Puriflash Silica HP 30 μm series from Interchim).

### Nuclear Magnetic Resonance (NMR)

NMR spectra (¹H, ¹³C {¹H}, ¹⁹F) were recorded on a Bruker DPX 400 spectrometer. All spectra are referenced to tetramethylsilane as an internal standard (δ=0.00 ppm). Multiplicities of the signals are described as follows: s=singlet, d=doublet, t=triplet, q=quartet, m=multiplet or overlap of non-equivalent resonances; br=broad signal. Coupling constants ⁿJ_{X-Y} are given in Hz, where n is the number of bonds between the coupled nuclei X and Y (J_{H-H} are always listed as J without indices).

### Mass-Spectrometry (MS)

Low resolution mass spectra (100-1500 m/z) with electro-spray ionization (ESI) were obtained on a Shimadzu LC-MS system described below. High resolution mass spectra (HRMS) were obtained on a maxis II ETD (Bruker) with electrospray ionization (ESI) at the Mass Spectrometry Core facility of the Max-Planck Institute for Medical Research (Heidelberg, Germany).

### High-Performance Liquid Chromatography (HPLC)

Analytical liquid chromatography-mass spectrometry was performed on an LC-MS system (Shimadzu): 2× LC-20AD HPLC pumps with DGU-20A3R solvent degassing unit, SIL-20ACHT autosampler, CTO-20AC column oven, SPD-M30A diode array detector and CBM-20A communication bus module, integrated with CAMAG TLC-MS interface 2 and LCMS-2020 spectrometer with electrospray ionization (ESI, 100-1500 m/z). Analytical column: Hypersil GOLD 50×2.1 mm 1.9 μm, standard conditions: sample volume 1-2 μL, solvent flow rate 0.5 mL/min, column temperature 30° C. General method: isocratic 95:5 A: B over 2 min, then gradient 95:5-0:100 A: B over 5 min, then isocratic 0:100 A: B over 2 min; solvent A=water+0.1% v/v HCO₂H, solvent B=acetonitrile+0.1% v/v HCO₂H.

Preparative high-performance liquid chromatography was performed on a Büchi Reveleris Prep system using the suitable preparative columns and conditions as indicated for individual preparations. Method scouting was performed on a HPLC system (Shimadzu): 2× LC-20AD HPLC pumps with DGU-20A3R solvent degassing unit, CTO-20AC column oven equipped with a manual injector with a 20 μL sample loop, SPD-M20A diode array detector, RF-20A fluorescence detector and CBM-20A communication bus module; or on a Dionex Ultimate 3000 UPLC system: LPG-3400SD pump, WPS-3000SL autosampler, TCC-3000SD column compartment with 2×7-port 6-position valves and DAD-3000RS diode array detector. The test runs were performed on analytical columns with matching phases (HPLC: Interchim 250×4.6 mm 10 μm C₁₈HQ, Interchim 250×4.6 mm 5 μm PhC4, solvent flow rate 1.2 mL/min; UPLC: Interchim C₁₈HQ or PhC4 75×2.1 mm 2.2 μm, ThermoFisher Hypersil GOLD 100×2.1 mm 1.9 μm, solvent flow rate 0.5 mL/min).

### STED (Stimulated Emission Depletion) Microscopy

One- and multiphoton-activated STED and confocal counterpart images were acquired using two Abberior Expert Line (Abberior Instruments GmbH, Göttingen, Germany) fluorescence microscopes built on a motorized inverted microscope IX83 (Olympus, Tokyo, Japan), and equipped with a 100×/1.40 or a 60×/1.42 oil immersion objective lenses (Olympus). One of the microscopes is equipped with pulsed STED lasers at 595 nm and 775 nm shaped by Spatial Light Modulators (SLMs), and with 355 nm, 405 nm, 485 nm, 561 nm, and 640 nm excitation lasers. The other microscope is equipped with pulsed STED lasers at 655 nm and 775 nm, and with 520 nm, 561 nm, 640 nm, and multiphoton (Chameleon Vision II, Coherent, Santa Clara, USA) excitation lasers. The multiphoton laser is tunable in the 680 nm-1080 nm range. Spectral detection is performed in both cases with avalanche photodiodes at spectral windows adjusted for each particular fluorophore.

Imaging and image processing was done with ImSpector software, and all images are displayed as raw data.

### Superresolution Single Molecule Localization Microscopy (SMLM)

Images were acquired on a custom-built setup [K. Uno et al. Proc. Nat. Acad. Sci. 2021, 118 (14), e2100165118], equipped with a 473 nm (500 mW), a 532 nm (1 W) and a 560 nm (1 W) laser for excitation, a 405 nm (300 mW) laser for activation, a back illuminated EMCCD camera (Andor iXon 897/512×512 sensor), and a Leica HCX PL APO CS 100×/1.46 oil lens. Emission light was separated from the excitation and activation light with proper combination of a dichroic mirror and a suppression filter for each excitation laser. A movable mirror was used to switch between wide field, highly inclined and laminated optical sheet (HILO) and total internal reflection fluorescence (TIRF) illumination modes. Images were acquired with a 10-20 ms exposure time, and actual excitation laser powers in the back focal plane of ca. 50-400 mW, depending on the dye and sample properties. The 405 nm activation laser was incorporated as 100-500 us pulses, in between frames, with a power of 0.001-1 mW in the back focal plane.

All images were analyzed and processed using the ThunderSTORM plugin [M. Ovesný et al. Bioinformatics, 2014, 30 (16), 2389-2390] on ImageJ (version 1.52p).

### Example 3

### Photolysis of the Exemplary Novel Compounds of the Invention and Chemometric Analysis of the Photoactivation and Photobleaching Reaction Kinetics

Solutions in phosphate buffer (100 mM, pH=7.0)(1.66 μg mL⁻¹) were irradiated in a previously described home-built setup [K. Uno et al. Adv. Opt. Mat. 2019, 7, 1801746] with a 405 nm LED source (M405L3, Thorlabs Inc.) in combination with a 10 nm bandpass filter (FB405-10, Thorlabs Inc.). During the irradiation, samples were maintained at 20° C. and continuously stirred. The absorption and emission of irradiated solutions was monitored at desired irradiation intervals. For such purpose, excitation was performed with an LED emitting at a wavelength suitable for each compound (e.g. 470 nm or 505 nm).

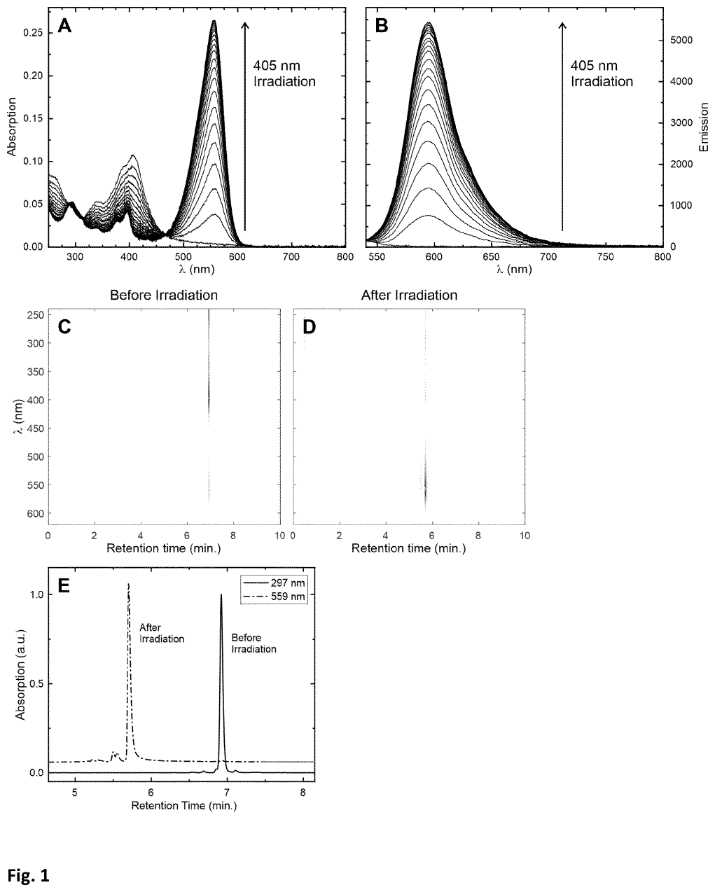

FIG. 1 shows absorption (A) and emission (B) changes during photo-induced activation of compound 13 with violet light (405 nm) in an aqueous buffered solution at pH 7 (phosphate buffer, 100 mM); HPLC 2D-maps of absorption spectra vs. retention time for samples of the solution before (C) and after (D) the photo-induced activation; and chromatograms (E) of these samples at the wavelengths corresponding to the respective absorption maxima.

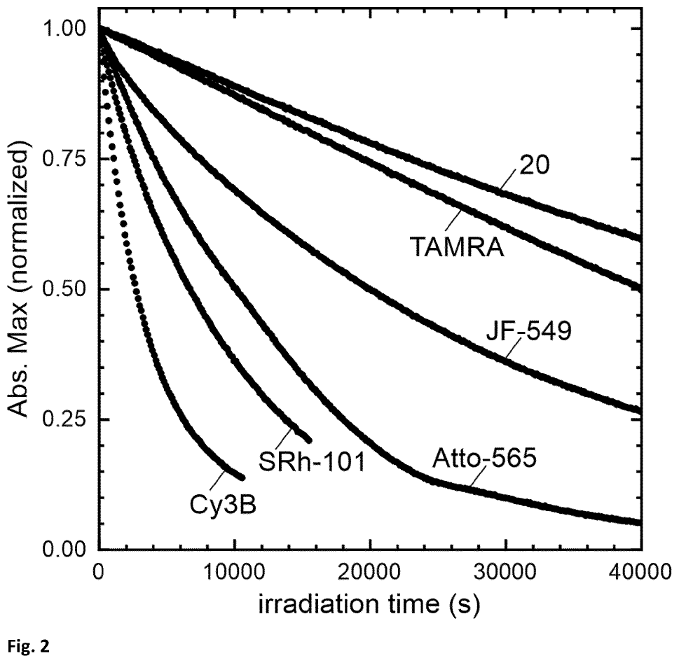

FIG. 2 shows the photo-fatigue resistance of compound 20 and established commercial fluorophores in an aqueous buffered solution at pH 7 (phosphate buffer, 100 mM). All compounds were irradiated with a green LED (Thorlabs, model M530L4, Nominal Wavelength 530 nm) under identical conditions and at similar initial concentrations. The measurement was performed as previously described in [A. N. Butkevich et al. J. Am. Chem. Soc. 2019, 141 (2), 981-989].

### Example 4

Live Cell Optical Microscopy of Cells Using Exemplary Novel Dyes of the Invention with Self-Labelling Enzymes

Stocks solutions of BG-(O⁶-benzylguanine, SNAPtag ligand) or HaloTag (O²)-derivatives (HaloTag ligand) of the dyes of the present invention (e.g. 5-Halo, 9-Halo, 20-Halo, and 20-BG) were prepared in DMSO (ca. 1 mM). U2OS cells that stably expressed Vimentin-HaloTag or Vimentin-SNAP [Ratz et al. Sci. Rep. 2015, 5, 9592; Butkevich et al. ACS Chem. Biol. 2018, 13 (2), 475-480] were grown for 12-72 h on glass coverslips. Cells were incubated for 30 min to overnight (depending on the dye and experiment) with the respective fluorescent ligands diluted from DMSO stock solutions with culture medium (without phenol red) to a final concentration of 10-500 nM. Cells were washed with cell culture medium for ca. 15-30 minutes; then the medium was changed for fresh media for imaging. If necessary, the cells were co-stained with the always-on dyes (6-SiR-CTX [J. Bucevičius et al., Chem. Sci., 2020, 11, 7313-7323] or Abberior Live 560-Tubulin). Samples were imaged by confocal or single molecule localization superresolution microscopy.

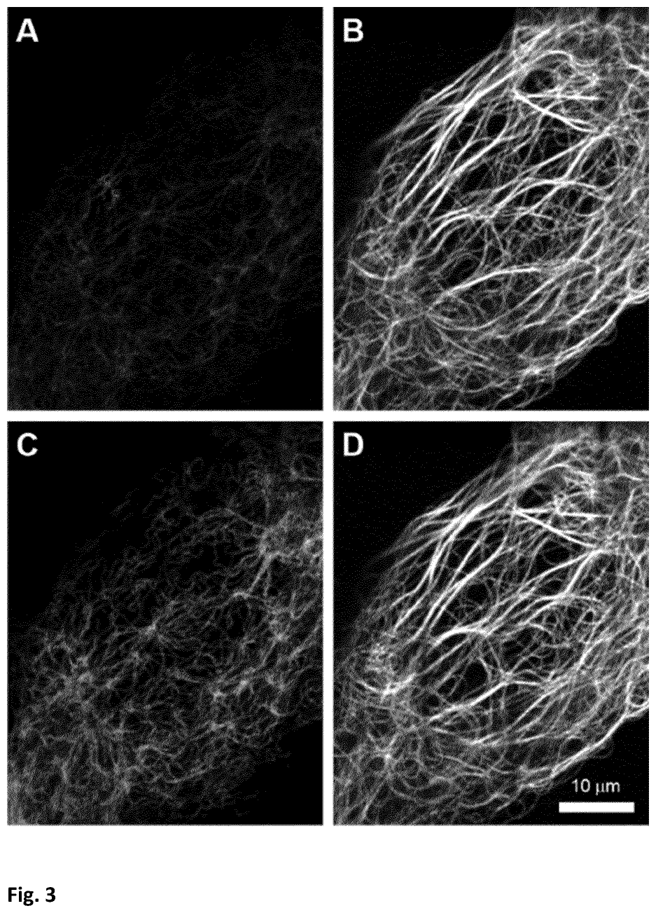

FIG. 3 shows live-cell dual-color/channel confocal imaging of vimentin filaments labelled with compound 20-Halo (A & C) and tubulin labelled with 6-SiR-CTX (B & D) in U2OS cells. Images were recorded before (A-B) and after photo-activation (C-D) with 405 nm light. Images A & C and B & D are shown on the same intensity scale, respectively. Green/orange channel (A & C): 561 nm excitation; 574-626 nm detection. Red channel (B & D): 640 nm excitation; 663-800 nm detection.

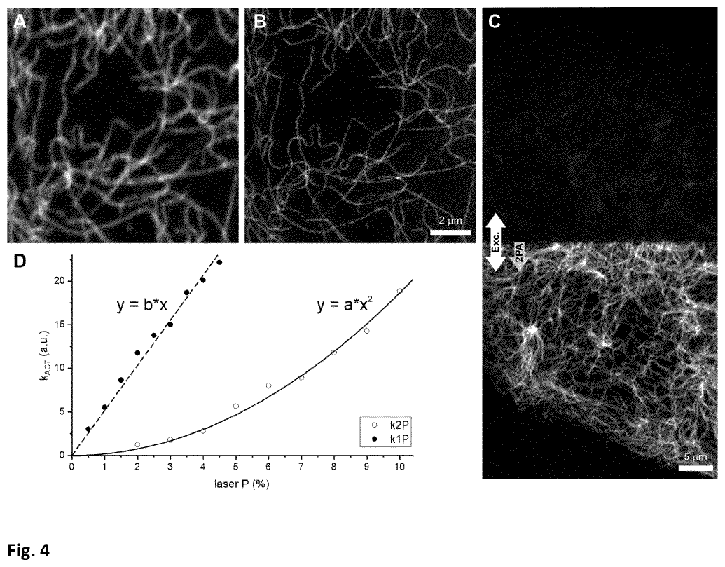

FIG. 4 shows live-cell confocal (A) and STED (B) image of vimentin filaments labelled with compound 20-Halo in U2OS cells. The compound was pre-activated by irradiation with a 405 nm laser. (C) The same samples was imaged in a confocal microscope, before pre-activation (top half of the image), and a 2-photon activation laser was switched on approximately in the middle of the scanning (bottom half of the image, as indicated by the arrows). The activation rate of selected areas on the same sample was calculated (mono-exponential fitting) by activation with variable powers of a one-photon activation laser (365 nm) or a two-photon activation laser (810 nm). The lines represent fittings to a linear or a quadratic function for one- and two-photon activation, respectively.

### Example 5

### Single Detection Channel Two-Color Multiplexing by Photoactivation

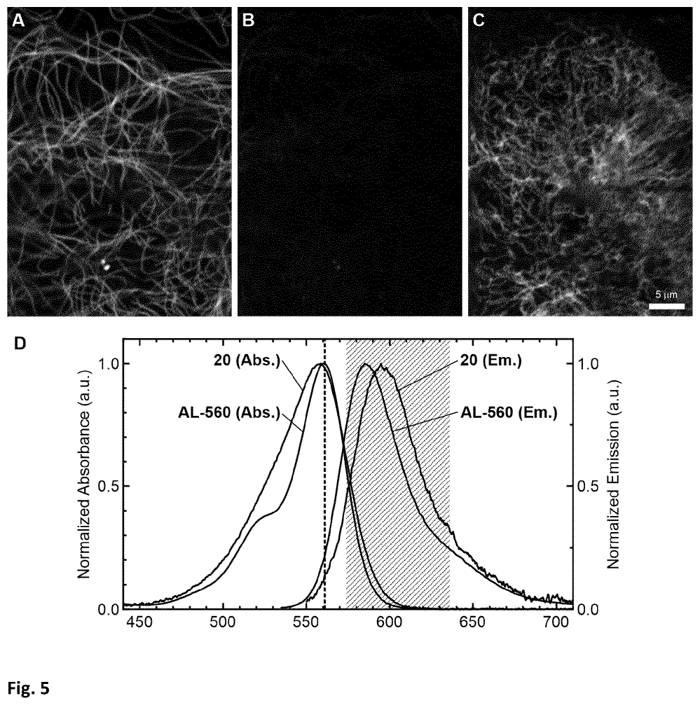

U2OS cells that stably expressed vimentin-HaloTag were labelled with 20-Halo and Abberior Live 560-Tubulin as described above. Sequential imaging was performed on a confocal setup before photoactivation (see FIG. 5, A), after photobleaching of Abberior Live 560-Tubulin with high power of 561 nm light (FIG. 5, B), and after photoactivation of 20-Halo with a 405 nm activation (FIG. 5, C). All images were acquired with the same excitation laser, 561 nm, and detection channel, 574 nm-626 nm range (FIG. 5, D). A single-channel, pseudo two-color (multiplexed) image of two different targets may be obtained from overlaying FIGS. 5A & 5C.

### Example 6

### Optical Microscopy of Cells Using Exemplary Novel Dyes of the Invention Coupled to Antibody

Amino-reactive NHS-esters of the present dyes were coupled to secondary antibodies (product #111-005-003 or 115-005-003, Jackson ImmunoResearch Europe Ltd.) using a standard coupling protocol. In brief, the reactive dye (e.g. 5-NHS, 9-NHS, or 20-NHS) was dissolved in anhydrous DMSO (ca. 2 mg/ml), and mixed with 0.5-1 mg antibody in a proportion of 5-10 equivalents (dye/protein). The pH of the solution was adjusted to ˜8.4, and stirred for 1 h in the dark. The mixture was purified using a size exclusion column (PD 10, GE Healthcare).

Cells were grown for 12-72 h on glass coverslips and then washed twice with PBS (pH 7.4), and then fixed with either methanol (MeOH) or paraformaldehyde (PFA) depending on the favored method for the chosen antibodies or the imaging structures. For MeOH fixation, the samples were treated with MeOH previously cooled to −20° C. for 5 min, and finally washed twice with PBS. PFA fixation was performed with a 4% formaldehyde solution in PBS at room temperature for 20 min, washed twice with PBS, and then treated with a quenching solution (0.1 M NH₄Cl and 0.1 M Glycine in PBS) for 5 min at room temperature. To reduce unspecific binding blocking buffer (2% BSA in PBS) was added and incubated for 30-60 min at room temperature. For samples fixed witFA, Triton 100-X was added to the blocking buffer to a concentration of 0.1%. The coverslips were overlaid with the primary antibody solution in blocking buffer and incubated in a humid chamber for 1 h at room temperature, or overnight at 4° C. Following this, the coverslips were washed with blocking buffer (3×5 min). The coverslips were then incubated with the secondary antibody in blocking buffer in a humid chamber for 45-60 min at room temperature. Lastly, coverslips were washed with blocking buffer (3×5 min), with PBS (1×5 min) and mounted with PBS or Mowiol, and sealed with nail polish or a two-component silicone resin (Picodent Twinsil, Picodent Dental-Produktions-und Vertriebs-GmbH). Samples were imaged by confocal, STED, or single molecule localization microscopy (SMLM).

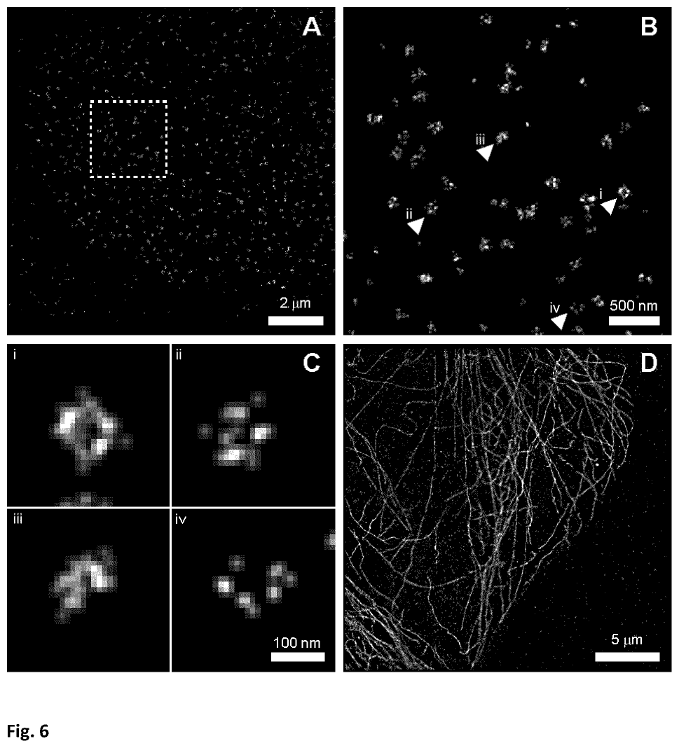

FIG. 6 shows Single Molecule Localization Microscopy superresolution images of nuclear pore complexes (A-C) and microtubules (D) in COS7 cells. Samples were fixed and immunolabelled with a primary antibody against NUP-98 from rabbit and an anti-rabbit secondary antibody labelled with 5-NHS (A-C) or a primary antibody against alpha-tubulin from mouse and an anti-mouse secondary labelled with 9-NHS (D). Figure B is a magnified area from A displayed in a dotted box, and C are selected single nuclear pore complexes indicated in B.

### Example 7

### Color Multiplexing of Two Photoactivatable Dyes by Photoactivation Kinetics

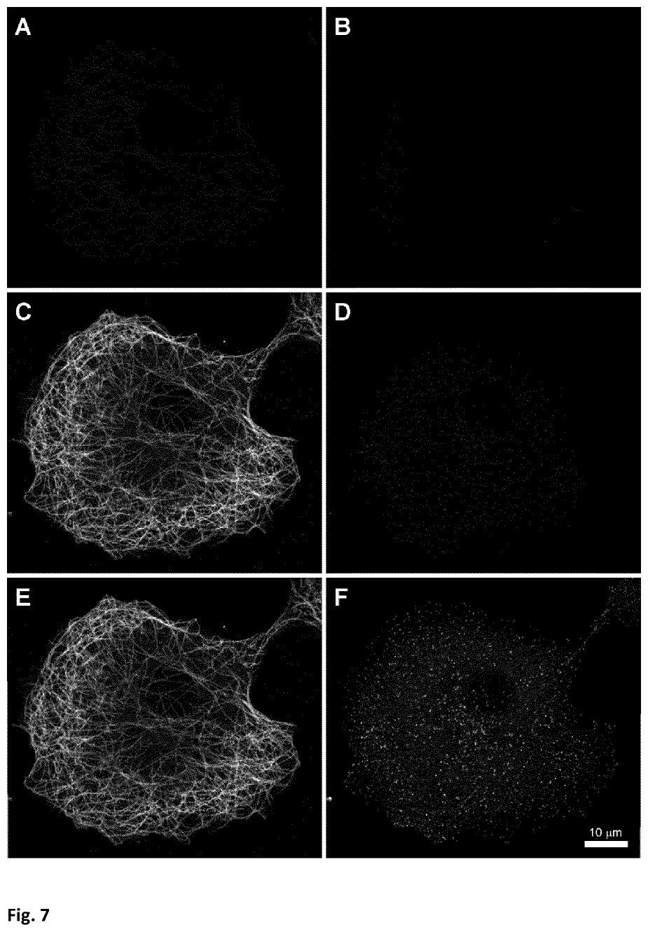

Cos7 cells were fixed with MeOH and immunolabeled as described above with a mixture of anti-clathrin primary antibody (from rabbit) and anti-alpha tubulin primary antibody (from mouse), and then with a mixture of anti-rabbit secondary antibody labelled with 5-NHS and anti-mouse secondary antibody labelled with 20-NHS. Two-color imaging was performed simultaneously (line-by-line) in two channels with excitation at 485 nm and detection in a 500-551 nm window (FIG. 7B, D, F) and excitation at 561 nm and detection in a 571-691 nm window (FIG. 7A, C, E) for compounds 5 and 20, respectively. Sequential imaging was performed on a confocal setup before photoactivation (see FIG. 7A,B), after photoactivation of 20 with low activation laser dose (FIG. 7C,D), and after photoactivation of 5 with higher activation laser dose sufficient to convert compound 5 (FIG. 7E, F) to obtain two-color image of two photoactivatable dyes multiplexed by photoactivation kinetics.

Green/orange channel (A, C & E): 561 nm excitation; 571-691 nm detection. Blue/Green channel (B, D & F): 485 nm excitation; 500-551 nm detection. Images corresponding to each channel are shown on the same intensity scale, respectively.

### Example 8

Photopatterning of a Polymer Matrix with an Exemplary Compound of the Present Invention

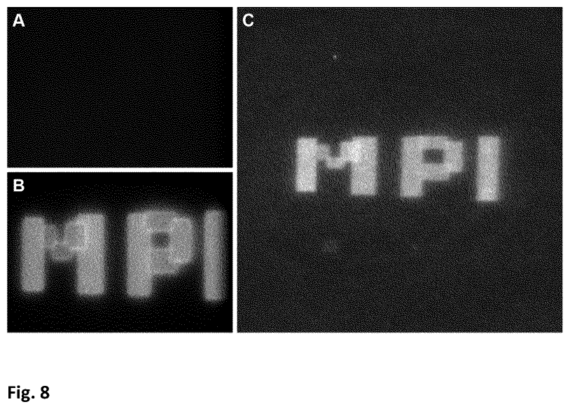

A polymer film composed of polyvinyl alcohol (PVA) and compound 5 was spin-coated on a cover slide from an aqueous mixture of PVA (2% w/v) and of compound 5 (0.05 mg/ml). The film was placed face-down towards a microscopy slide and fixed with nail polish. Photopatterning was performed on a confocal setup, where select areas were irradiated with light of 405 nm until full activation (i.e. fluorescence reached a plateau). Images of the film before and after patterning was recorded with the same fluorescence microscope (excitation 561 nm/detection 571-691 nm), see FIG. 8A-B, and in a wide-field configuration, with a commercial Cy3 filter-cube (FIG. 8C).

1. A compound, in particular a photoactivatable fluorescent dye, having the structural formula I: 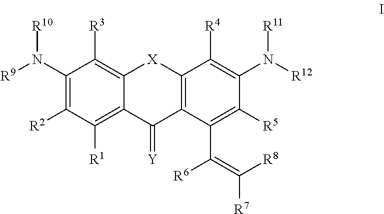
wherein:
R¹, R², R³, R⁴, R⁵, R⁶, R⁷ and R⁸, independently of each other are selected from H, halogen, SO₃H, CO₂H, CN, NO₂, CO₂R, SO₂R— with R in CO₂R or SO₂R being selected from C₁ to C₄ unsubstituted alkyl , and an unsubstituted or substituted moiety, in particular an unsubstituted or halogen-, amino-, hydroxyl-, SO₃H— and/or carboxyl-substituted moiety, which is selected from C₁-C₂₀ alkyl, C₃-C₈ cycloalkyl, C₁-C₂₀ alkoxy, C₂-C₂₀ alkylene, C₂-C₂₀ alkylyne, C₇-C₂₀ alkylaryl, phenyl and 5- or 6-membered ring heteroaryl, or a combination thereof;
and where the substituents R⁶ and R⁷, taken together with the atoms to which they are bound, may form a 5-8 membered ring structure; and/or where the substituents R⁷ and R⁸, taken together with the atoms to which they are bound, may form a 5-8 membered ring structure;
R⁹, R¹⁰, R¹¹, R¹² are:
a. independently selected from H, unsubstituted and substituted C₁-C₈ alkyl, C₃-C₈ cycloalkyl, C₁-C₈ acyl, C₁-C₈ alkoxycarbonyl, and C₇-C₁₂ alkylaryl, and unsubstituted phenyl or phenyl substituted by unsubstituted alkyl, halogen, alkoxy, NO₂, CO₂H, CO₂R and/or CONR²— with each R in CO₂R or CONR² being selected independently from C₁ to C₄ unsubstituted alkyl-; or
b. R⁹ together with R¹⁰ and a nitrogen atom to which they are bound, and/or R¹¹ together with R¹² and a nitrogen atom to which they are bound form a 3-7 membered ring structure; or
c. R⁹ and/or R¹¹ are independently selected from H and unsubstituted and substituted C₁-C₈ alkyl, C₃-C₈ cycloalkyl, and C₇-C₁₂ alkylaryl; and R¹⁰ together with R² or R³ and the atoms to which they are bound form a 5-7 membered ring structure, and/or R¹² together with R⁴ or R⁵ and the atoms to which they are bound form a 5-7 membered ring structure;
d. R⁹ together with R² and the atoms to which they are bound form a 5-7 membered ring structure, and/or R¹⁰ together with R³ and the atoms to which they are bound form a 5-7 membered ring structure, and/or R¹¹ together with R⁴ and the atoms to which they are bound form a 5-7 membered ring structure, and/or R¹² together with R⁵ and the atoms to which they are bound form a 5-7 membered ring structure;
X is independently selected from:
a. O or S atom or SO₂ group;
b. NR¹³ or P(═O)R¹³ group, where R¹³ is selected from H, unsubstituted and substituted C₁-C₁₂ alkyl, C₃-C₈ cycloalkyl, C₁-C₂₀ alkoxy, C₁-C₂₀ alkoxycarbonyl, C₂-C₂₀ acyl, C₂-C₂₀ alkylsulfonyl, C₂-C₂₀ alkylene, C₂-C₂₀ alkylyne and C₇-C₂₀ alkylaryl, phenyl and 5- or 6-membered ring heteroaryl, or a combination thereof;
c. SiR¹⁴R¹⁵ or GeR¹⁴R¹⁵ group, where R¹⁴ and R¹⁵ are each independently selected from unsubstituted and substituted C₁-C₁₂ alkyl, C₃-C₈ cycloalkyl, C₁-C₂₀ alkoxy, C₂-C₂₀ alkylene, C₂-C₂₀ alkylyne and C₇-C₂₀ alkylaryl, phenyl and 5- or 6-membered ring heteroaryl, or a combination thereof,
or where both substituents R¹⁴ and R¹⁵, taken together with the Si or Ge to which they are attached, form a 4-7 membered ring structure;
d. CR¹⁶R¹⁷ group, where R¹⁶ and R¹⁷ are each independently selected from H, F, CF₃, CN, COR¹⁸, CO₂R¹⁸, SO₂R¹⁸, CONR¹⁸R¹⁹—where R¹⁸ and R¹⁹ in COR¹⁸, CO₂R¹⁸, SO₂R¹⁸, and CONR¹⁸R¹⁹ are each independently selected from unsubstituted and substituted C₁-C₁₂ alkyl, C₃-C₈ cycloalkyl, C₁-C₂₀ alkoxy, C₂-C₂₀ alkylene, C₂-C₂₀ alkylyne and C₇-C₂₀ alkylaryl, phenyl and 5- or 6-membered ring heteroaryl, or a combination thereof, unsubstituted and substituted C₁-C₁₂ alkyl, C₃-C₈ cycloalkyl, C₁-C₂₀ alkoxy, C₂-C₂₀ alkylene, C₂-C₂₀ alkylyne and C₇-C₂₀ alkylaryl, phenyl and 5- or 6-membered ring heteroaryl, or a combination thereof,
or where both substituents R¹⁶ and R¹⁷, taken together with the C atom to which they are attached, form a 4-7 membered ring structure;
Y is independently selected from:
a. O or S atom;
b. NR²⁰ group, where R²⁰ is selected from H, unsubstituted and substituted C₁-C₁₂ alkyl, C₃-C₈ cycloalkyl, C₁-C₂₀ alkoxy, C₂-C₂₀ acyl, C₂-C₂₀ alkylsulfonyl, C₂-C₂₀ alkylene, C₂-C₂₀ alkylyne and C₇-C₂₀ alkylaryl, phenyl and 5- or 6-membered ring heteroaryl, or a combination thereof;
c. CR²¹R²² group, where R²¹ and R²² are each independently selected from H, F, CF₃, CN, COR²³, CO₂R²³, SO₂R²³, CONR²³R²⁴—where R²³ and R²⁴ in COR²³, CO₂R²³, SO₂R²³, CONR²³R²⁴ are each independently selected from unsubstituted and substituted C₁-C₁₂ alkyl, C₃-C₈ cycloalkyl, C₁-C₂₀ alkoxy, C₂-C₂₀ alkylene, C₂-C₂₀ alkylyne and C₇-C₂₀ alkylaryl, phenyl and 5- or 6-membered ring heteroaryl, or a combination thereof, unsubstituted and substituted C₁-C₁₂ alkyl, C₃-C₈ cycloalkyl, C₁-C₂₀ alkoxy, C₂-C₂₀ alkylene, C₂-C₂₀ alkylyne and C₇-C₂₀ alkylaryl, unsubstituted and substituted phenyl, unsubstituted and substituted 5- or 6-membered ring heteroaryl, or a combination thereof,
or where both substituents R²¹ and R²², taken together, form a 4-7 membered ring structure.

2. A compound, in particular a fluorescent dye, which has the structural formula II and is obtainable by irradiation with UV, visible or infrared light through a one-photon absorption process or a multiphoton absorption process from any of the compounds of general formula I of claim 1: 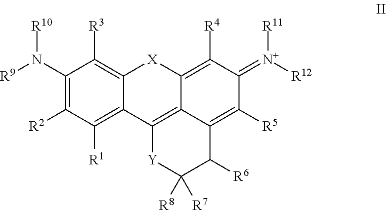
where R¹, R², R³, R⁴, R⁵, R⁶, R⁷, R⁸, R⁹, R¹⁰, R¹¹, R¹², X and Y are defined as in claim 1.

3. The compound according to claim 1, wherein the compound is covalently linked, particularly through any one of substituents R¹, R², R³, R⁴, R⁵, R⁶, R⁷, R⁸, R⁹, R¹⁰, R¹¹ and R¹² or through any of the groups X and Y, to a binding moiety M selected from:
a. a moiety selectively attachable by covalent bond to a protein or nucleic acid, particularly a moiety able to form an ester bond, an ether bond, an amide or thioamide bond, a sulfide or disulfide bond, a carbon-carbon bond, a carbon-nitrogen bond such as a Schiff base, or a moiety able to react in a click-chemistry reaction with a corresponding functional group of a protein or nucleic acid,
more particularly selected from —COCH═CH₂, —SO₂CH—CH₂, —COCH₂I, —COC═CH, —N═C═S, —CO—NHS or another active ester, biotin, an azide or a tetrazine moiety, a diazoalkane or diazoketone moiety, a diazirine moiety, an alkyne, a strained alkyne such as bicyclo[6.1.0]nonyne moiety or cyclooctyne moiety, a strained alkene such as trans-cyclooctene moiety or norbornene moiety, a maleimide; or from
b. a substrate of a haloalkane halotransferase, particularly a 1-chlorohexyl moiety as exemplarily shown below: 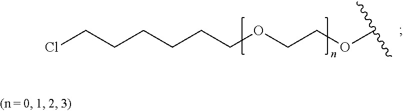
or from
c. a substrate of O⁶-alkylguanine-DNA-alkyltransferase, particularly a (substituted)(6-benzylguanine, O²-benzylcytosine or 4-benzyloxy-6-chloropyrimidine-2-amine moiety as exemplarily shown below: 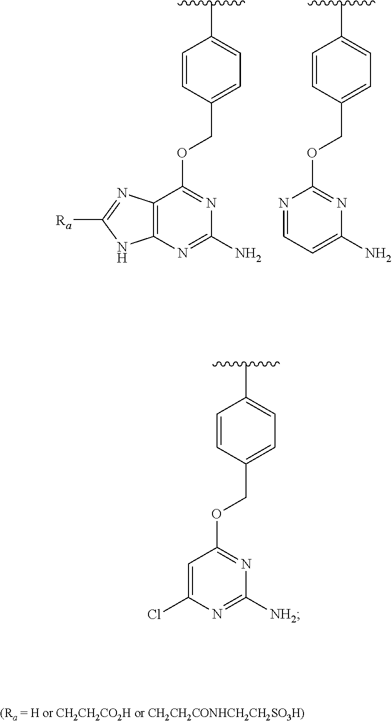
or from
d. a substrate of dihydrofolate reductase, particularly a 4-demethyltrimethoprim moiety as exemplarily shown below: 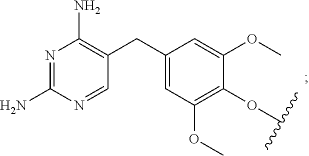 [SMILES: *Oc1c(OC)cc(Cc2cnc(N)nc2N)cc1OC]
or from
e. a moiety capable of selectively interacting non-covalently with a biomolecule, particularly a protein or nucleic acid, wherein said moiety and said biomolecule form a complex having a dissociation constant k_{D} of 10⁻⁶ mol/L or less,
more particularly, M is selected from de-N-Boc-docetaxel, de-N-Boc-cabazitaxel, de-N-Boc-larotaxel or another taxol derivative, a phalloidin derivative, a jasplakinolide derivative, a bis-benzimide DNA stain, pepstatin A or triphenylphosphonium, e.g. as shown below: 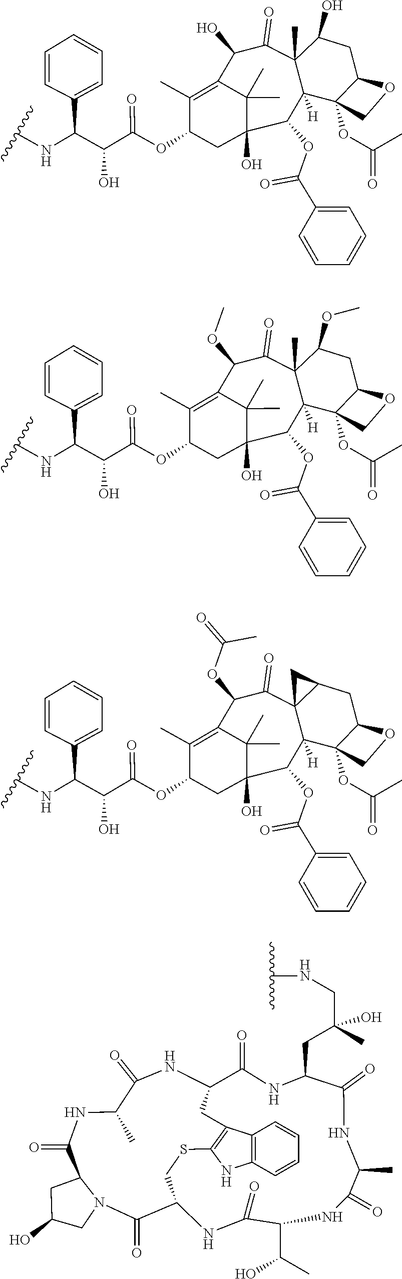 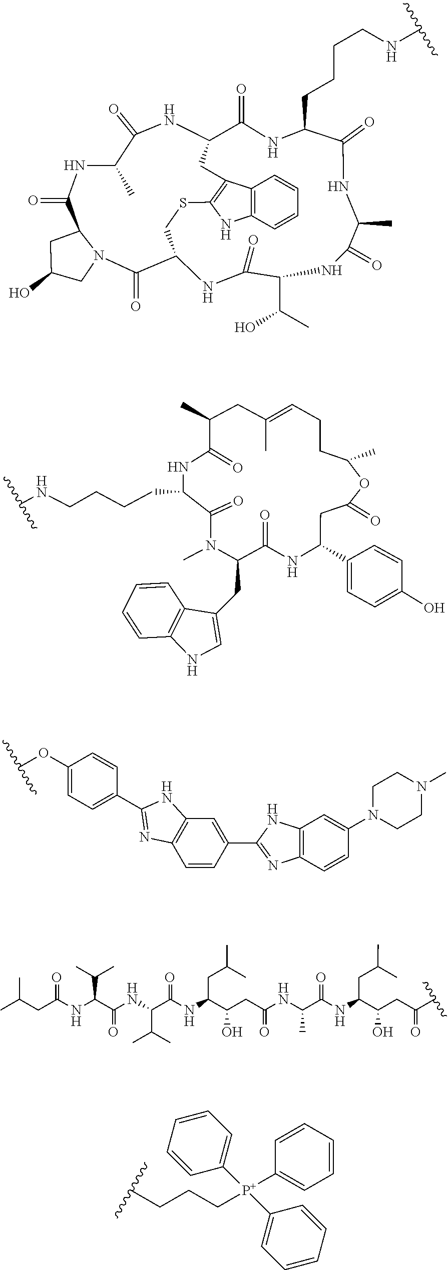
f. or wherein M is an oligonucleotide having a sequence length between 10 and 40 nucleotides;
g. or wherein M is a lipid, particularly a sphingosine derivative such as a ceramide, or a phospholipid such as dioleoylphosphatidylethanolamine (DOPE) or dipalmitoylphosphatidylethanolamine (DPPE), or a fatty acid.

4. The compound according to claim 1, having one of the structural formulas I-1-I-32 or II-1-II-32: 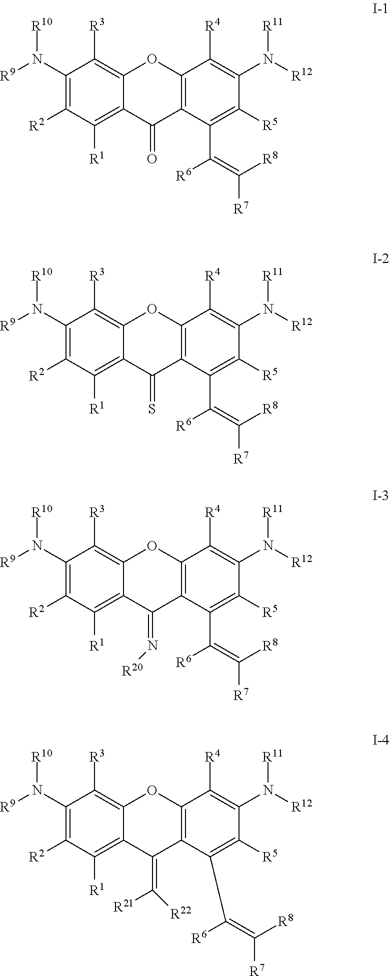 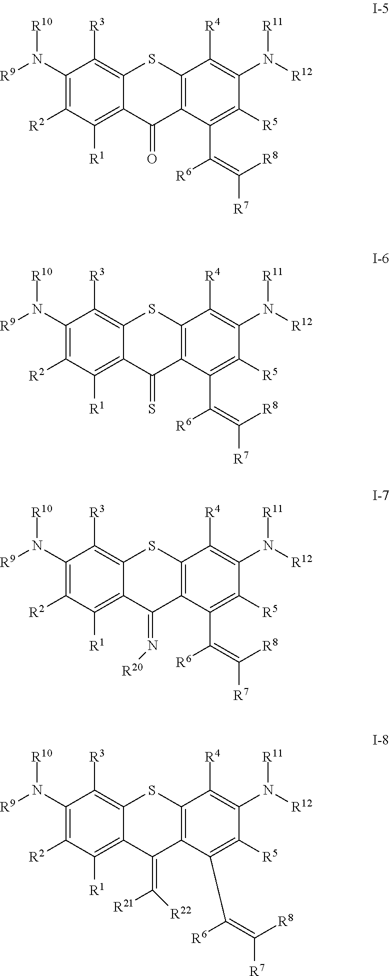 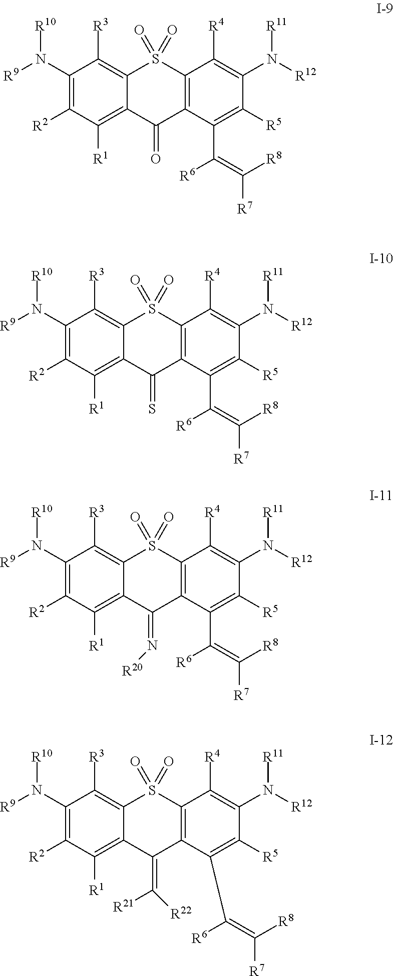 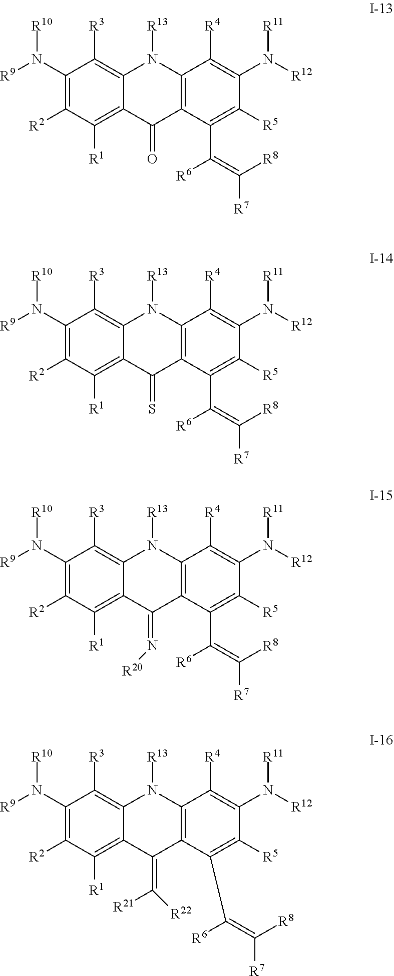 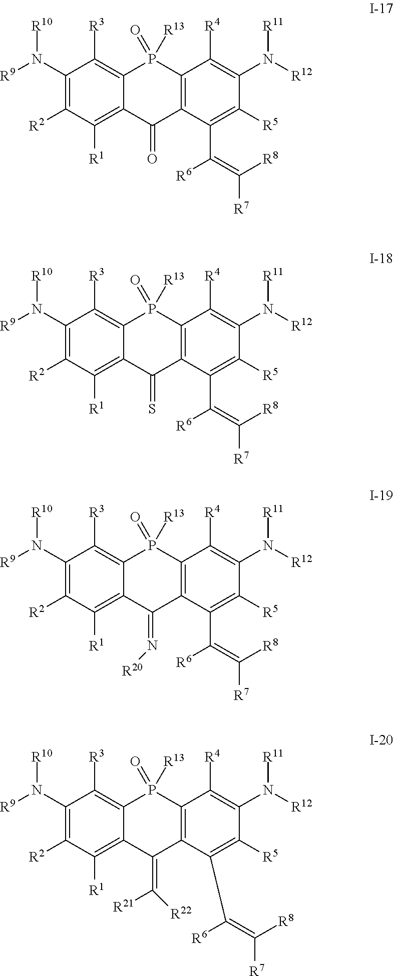 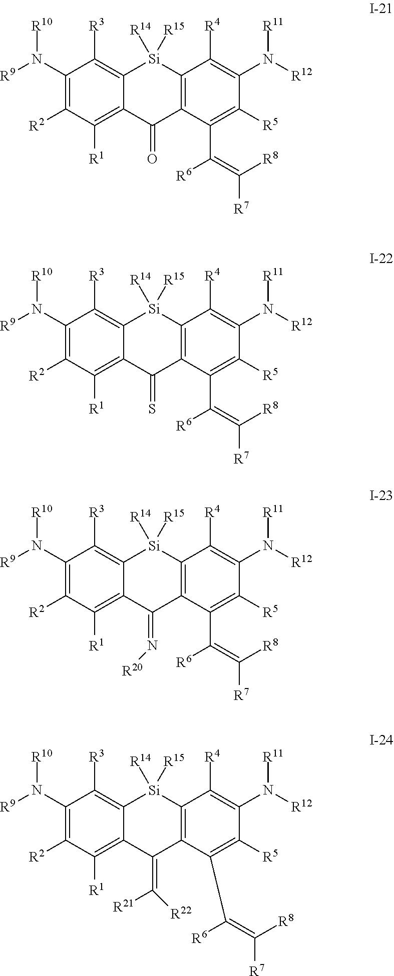 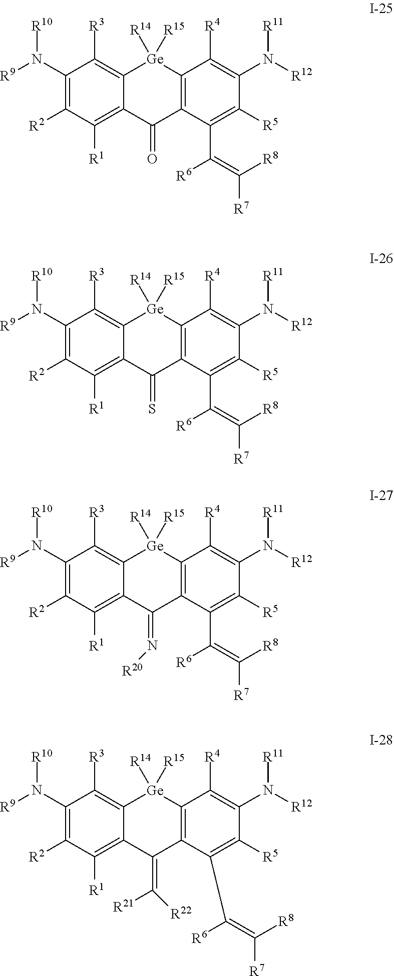 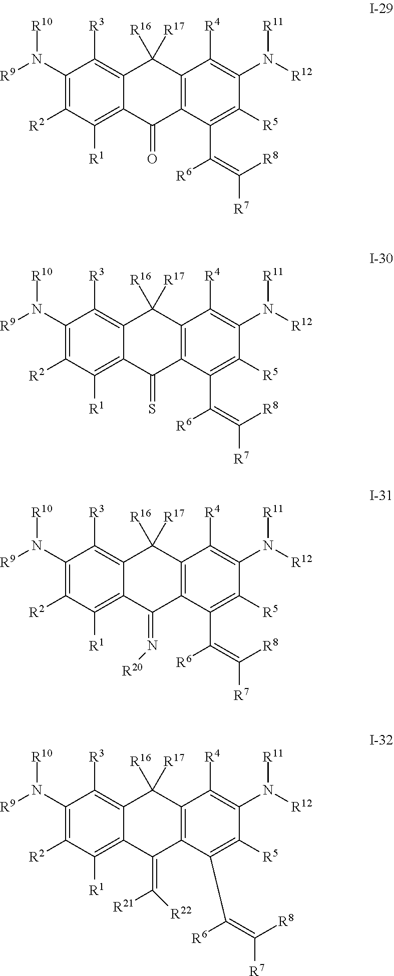 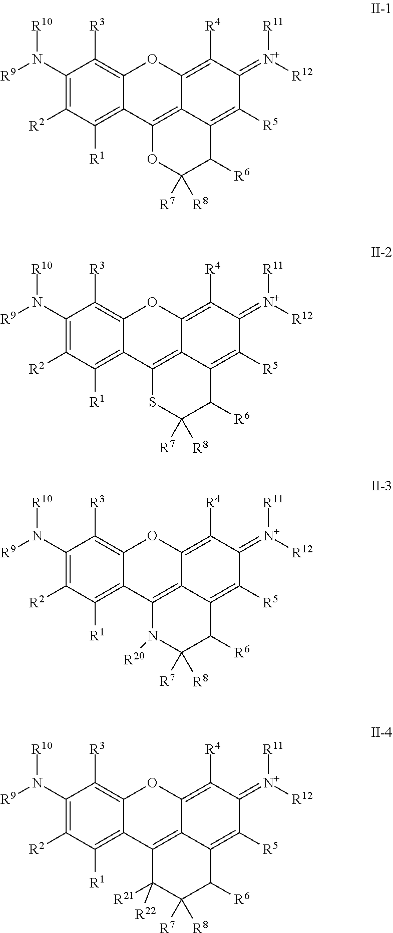 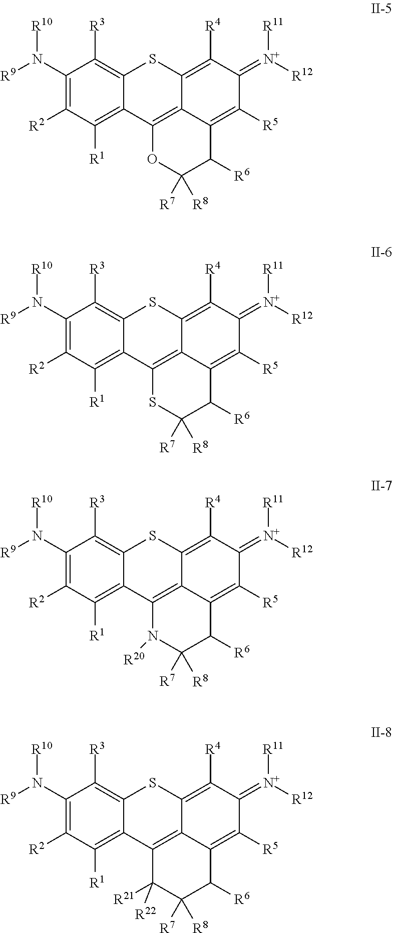 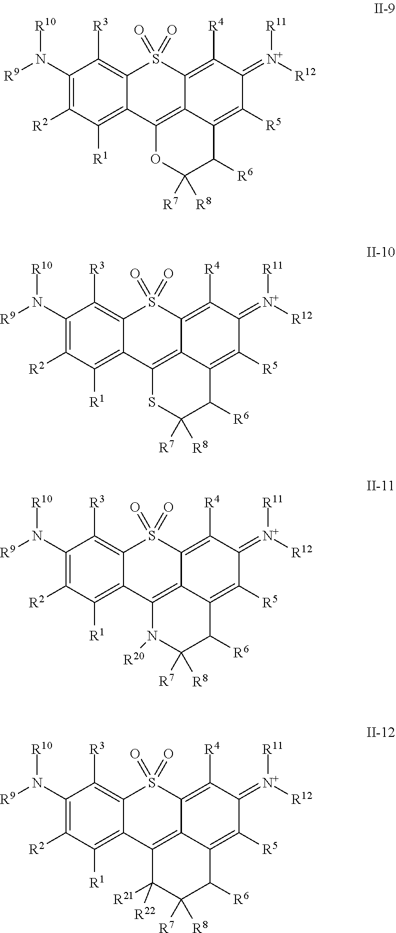 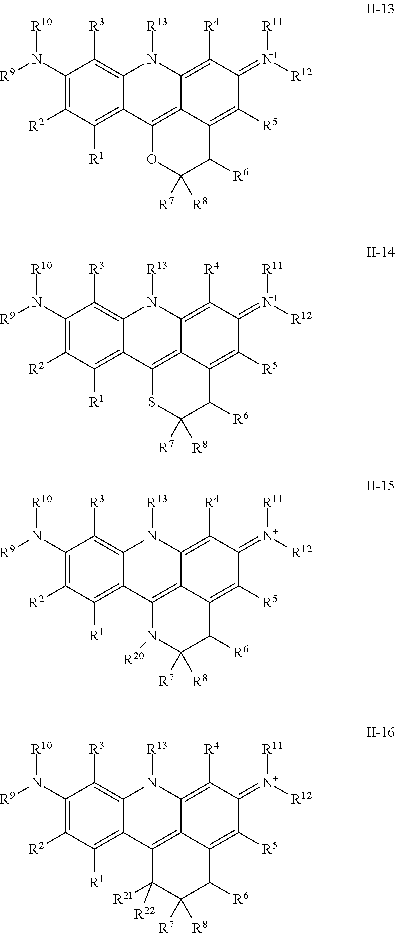 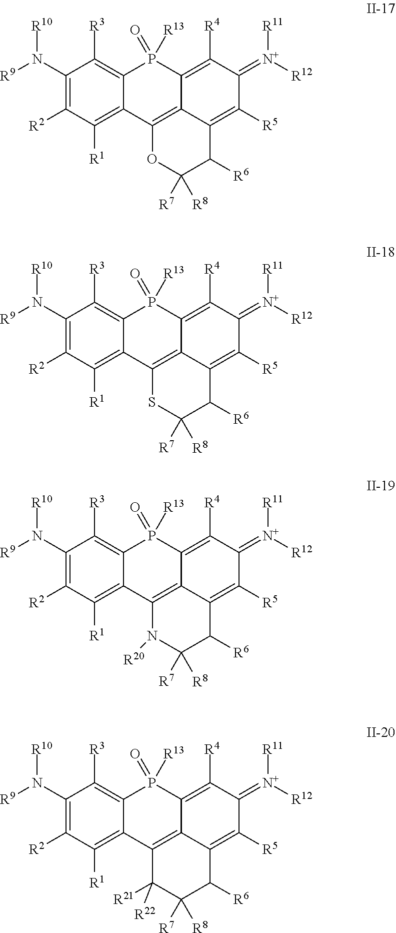 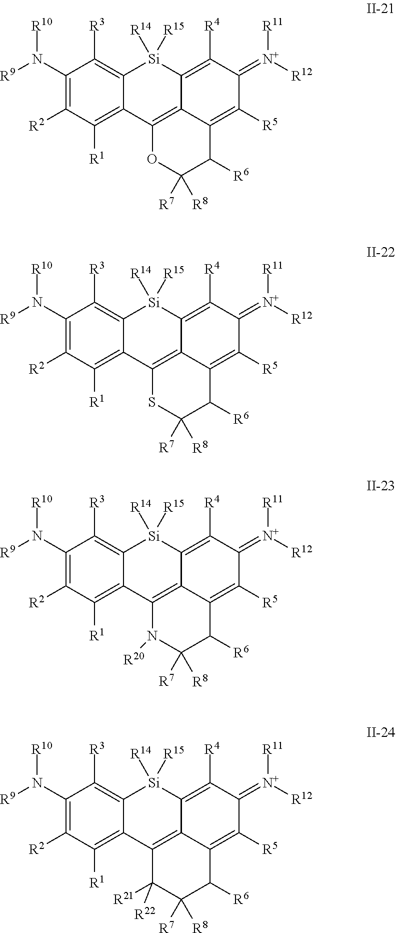 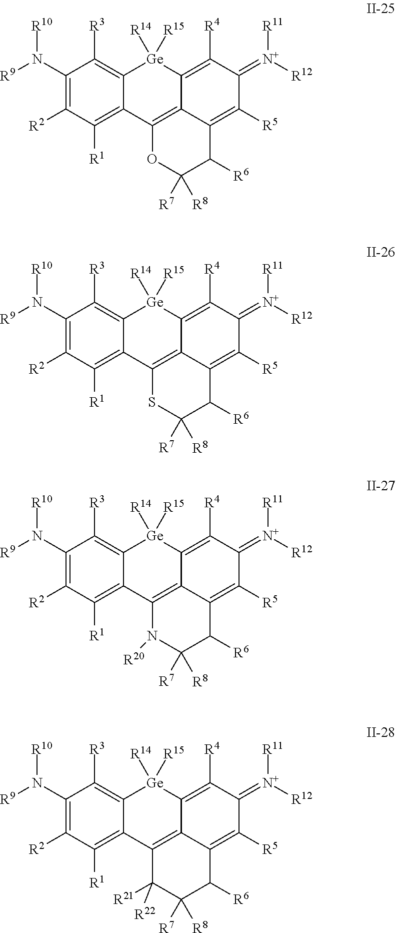 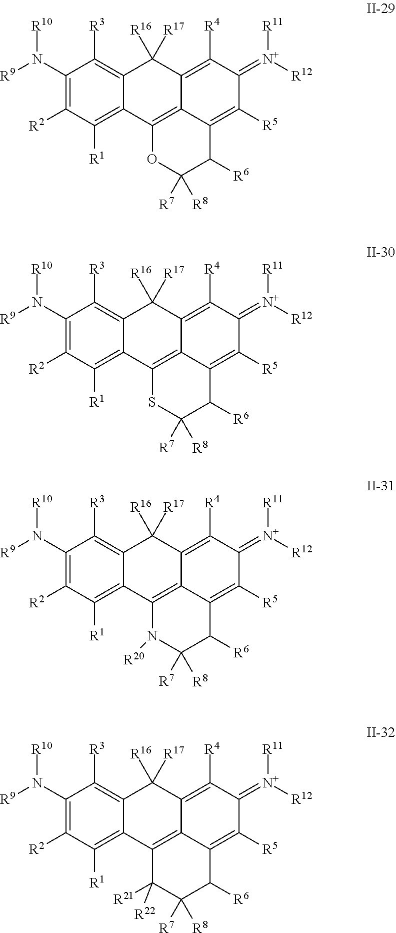
wherein any one of substituents R¹, R², R³, R⁴, R⁵, R⁶, R⁷, R⁸, R⁹, R¹⁰, R¹¹, R¹² or one of the substituents R¹³, R¹⁴, R¹⁵, R¹⁶, R¹⁷, R¹⁸, R¹⁹, R²⁰, R²¹, R²², if present, independently of any other is H or a moiety having a molecular weight between 15 and 1500 Da;
particularly wherein:
a) the substituents R⁹, R¹⁰, R¹¹, R¹² are selected from H and methyl, or any of the substituents-NR⁹R¹⁰ and —NR¹¹R¹² represents an azetidine ring, and
b) one of substituents R¹, R², R³, R⁴, R⁵, R⁶, R⁷, R⁸ or one of the R¹³, R¹⁴, R¹⁵, R¹⁶, R¹⁷, R¹⁸, R¹⁹, R²⁰, R²¹, R²², if present, is H or a moiety having a molecular weight between 15 and 1500 Da, and
c) the other substituents R¹, R², R³, R⁴, R⁵ are selected from H and F, and
d) the other substituents R⁶, R⁷, R⁸ are selected from H and methyl, and
e) the other substituents R¹³, R¹⁴, R¹⁵, if present, are selected from methyl, ethyl, isopropyl or phenyl,
f) the other substituents R¹⁶, R¹⁷ if present, are methyl,
g) the other substituents R²⁰, R²¹, R²², if present, are selected from H and methyl.

5. The compound according to claim 4, wherein said moiety having a molecular weight between 15 and 1500 Da is characterized by a general formula-L-M, wherein L is a linker covalently connecting the compound of structure I-1-I-32 or II-1-II-32 to the binding moiety M as defined above, and L is a covalent bond or a linker consisting of 1 to 50 atoms having an atomic weight of 12 or higher(in addition to the number of hydrogen atoms required to satisfy the valence rules),
particularly wherein said moiety having a molecular weight between 15 and 1500 Da is characterized by a general formula
-Lₘ^{A1}-L_{m′}^{J1}-Lₙ^{A2}-L_{n′}^{J2},-Lₚ^{A3}-L_{p′}^{J3},-L_{q}^{A4},-L_{q′}^{J4},-Mₛ, wherein
L^{A1}, L^{A2}, L^{A3} and L^{A4} independently of each other are selected from C₁ to C₁₂ unsubstituted or amino-, hydroxyl-, carboxyl- or fluoro substituted alkyl or cycloalkyl, (CH₂—CH₂—O), with r being an integer from 1 to 20, alkylaryl, alkylaryl-alkyl, and unsubstituted or alkyl-, halogen-, amino-, alkylamino-, imido-, nitro-, hydroxyl-, oxyalkyl-, carbonyl-, carboxyl-, sulfonyl- and/or sulfoxyl substituted aryl or heteroaryl;
L^{J1}, L^{J2}, L^{J3} and L^{J4} independently of each other are selected from —NRC(═O)—, —C(═O)N(R)—, —NRC(—O)O—, —OC(═O)N(R)—, —C(R)═N—, —N═C(R)—, —C(═O)—, —OC(═O)—, —C(═O)O—, —N(R)—, —O—, —P(═O)(OR)—, —P(═O)(OR)O—, —OP(═O)(OR)—, —OP(═O)(OR)O—, —S—, —SO—, —SO₂—, —SO₂N(R)—, —N(R) SO₂N(R)—, —N(R) SO₂— with R selected from H and unsubstituted or amino-, hydroxyl-, carboxyl, sulfonate or fluoro substituted C₁ to C₆ alkyl, particularly when R is selected from H and methyl;
m, m′, n, n′, p, p′, q, q′ and s independently from each other are selected from 0 and 1, and
M has the meaning defined above.

6. The compound according to claim 4, wherein said moiety is represented by one of the following structures:

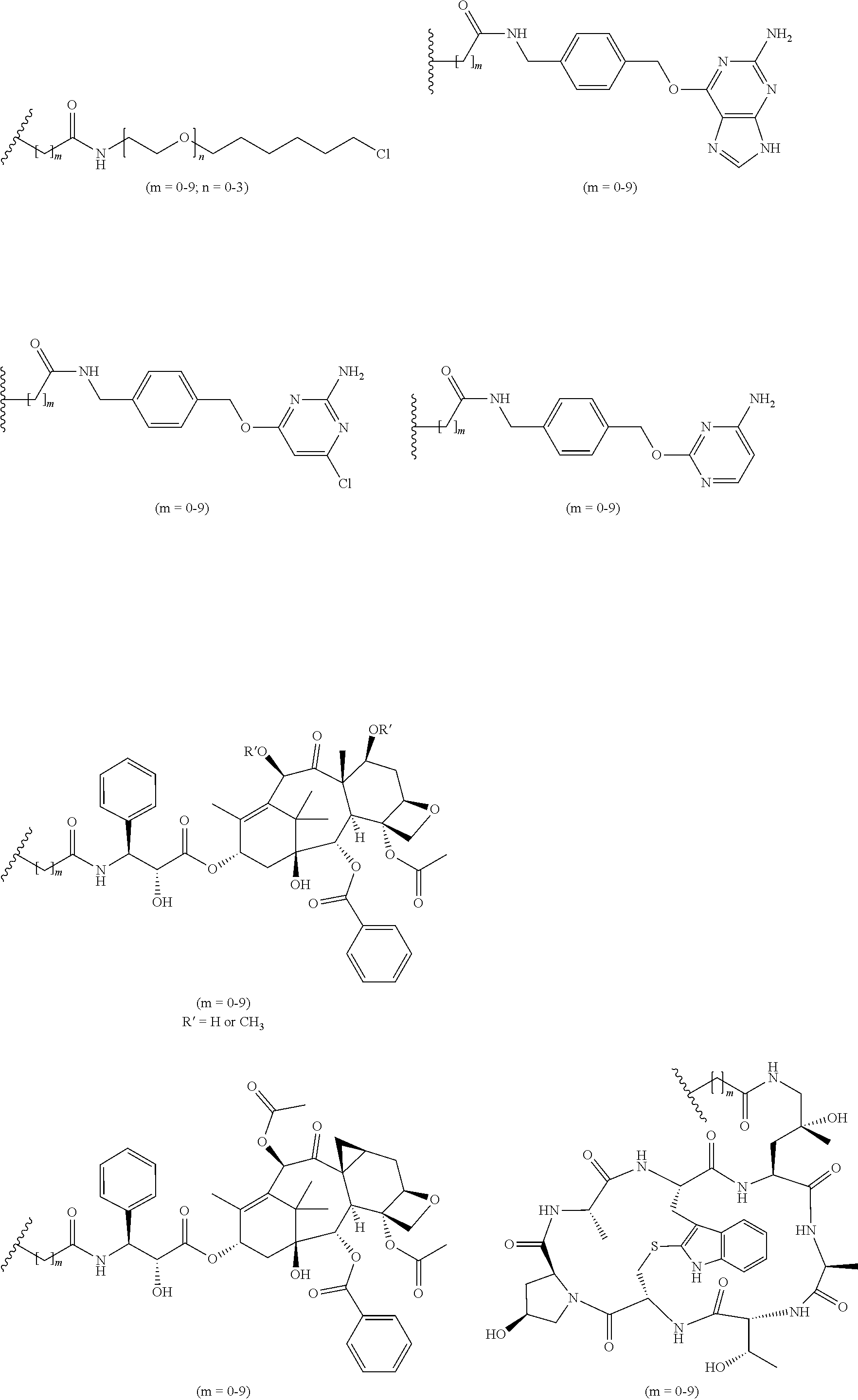

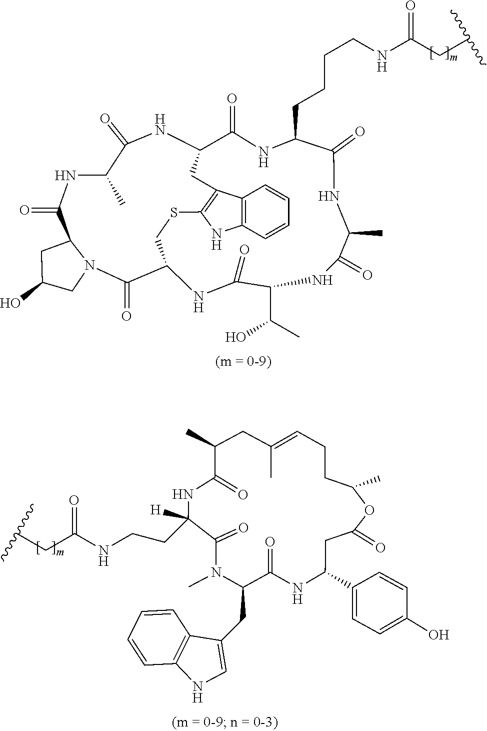

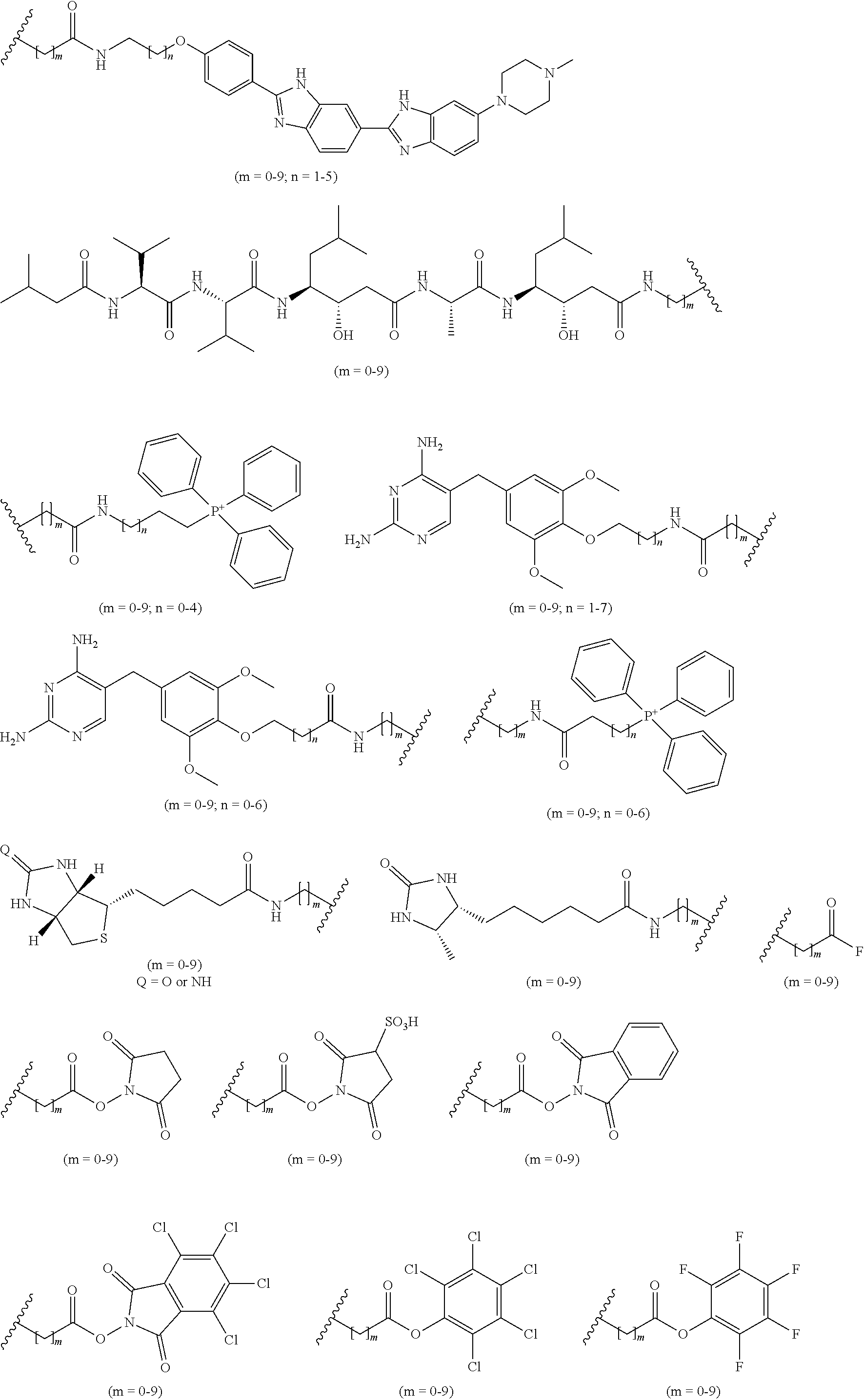

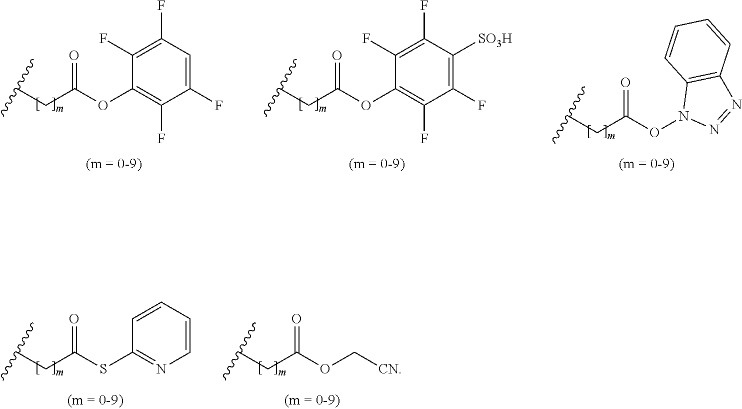

7. The compound according to claim 1, wherein
a. R⁹ and R¹⁰, and/or R¹¹ and R¹², are independently selected from H, unsubstituted and amino-, hydroxy-, carboxy-, sulfonate- and/or fluoro-substituted C₁-C₆ alkyl, C₁-C₄ acyl, C₁-C₄ alkoxycarbonyl, including tert-butyloxycarbonyl or Boc group, and C₃-C₆ cycloalkyl,
particularly R⁹ and R¹⁰, and/or R¹¹ and R¹², are independently selected from H, methyl, ethyl, n-propyl, isopropyl, n-butyl, isobutyl, tert-butyl, allyl and CH₂CF₃,
b. R⁹ together with R¹⁰, and/or R⁹ together with R¹⁰, are independently forming an unsubstituted or alkyl-, amino-, hydroxy-, carboxy-, sulfonate- and/or fluoro-substituted C₃-C₆ alkyl, particularly-(CH₂)₃—, —(CH₂)₄—, —(CH₂)₅—, —(CH₂)₂O (CH₂)₂—, —(CH₂)₂SO₂ (CH₂)₂— or —(CH₂)₂NR²³ (CH₂)₂— with R²³ being selected from H and unsubstituted C₁ to C₄ alkyl, particularly methyl;
c. R⁹ and/or R¹¹ are independently selected from H, unsubstituted and alkyl-substituted, particularly methyl-substituted, amino-, hydroxy-, carboxy-, sulfonate- and/or fluoro-substituted C₁-C₆ alkyl, C₁-C₄ acyl, C₁-C₄ alkoxycarbonyl, including tert-butyloxycarbonyl or Boc group, and C₃-C₆ cycloalkyl, and R¹⁰ together with R² or R³, and/or R¹² together with R⁴ or R⁵, is an alkyl or heteroalkyl bridge selected from —(CH₂)₂—, —(CH₂)₃—, —CH₂CH═CH— or —(CH₂)₄- or —CH₂—O—, —CH₂—NR—, —CH₂—S—, —CH₂—SO₂—, —(CH₂)₂O—, —(CH₂)₂NR—, —(CH₂)₂S—, —(CH₂)₂SO₂—, —CH₂—O—CH₂—, —CH₂NR—, —CH₂S—CH₂—, —CH₂—SO₂—CH₂—, with R selected from H and unsubstituted or amino-, hydroxyl-, carboxyl, sulfonate- or fluoro-substituted C₁ to C₆ alkyl, particularly when R is selected from H and methyl-, and a mono- or dimethyl-substituted derivative of any one of the foregoing alkyl or heteroalkyl bridge moieties;
d. R¹⁰ and/or R¹¹ are independently selected from H, unsubstituted and alkyl-, substituted, particularly methyl-substituted-, amino-, hydroxy-, carboxy-, sulfonate- and/or fluoro-substituted C₁-C₆ alkyl, C₁-C₄ acyl, C₁-C₄ alkoxycarbonyl, including tert-butyloxycarbonyl or Boc group, and C₃-C₆ cycloalkyl, and R⁹ together with R², and/or R¹² together with R⁵, form a fused annular structure according to any one of the following substructures: 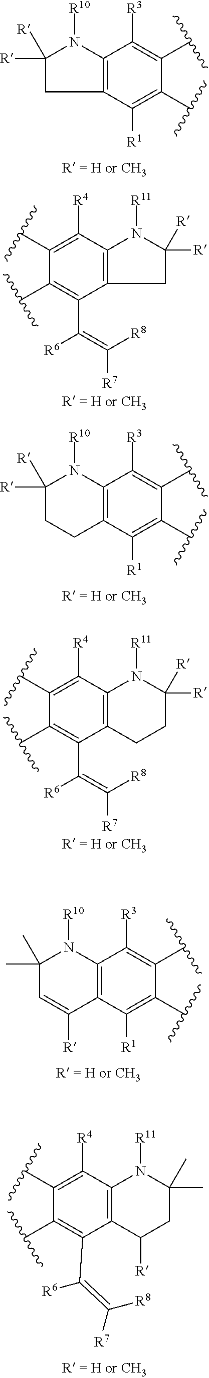
e. R⁹ and/or R¹² are independently selected from H, unsubstituted and alkyl-substituted, particularly methyl-substituted, amino-, hydroxy-, carboxy-, sulfonate- and/or fluoro-substituted C₁-C₆ alkyl, C₁-C₄ acyl, C₁-C₄ alkoxycarbonyl, including tert-butyloxycarbonyl or Boc group, and C₃-C₆ cycloalkyl, and R¹⁰ together with R³, and/or R¹¹ together with R⁴, form a fused annular structure according to any one of the following substructures: 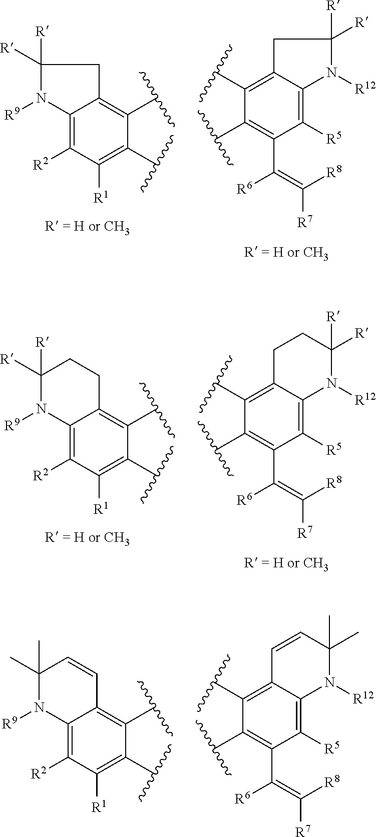
f. R⁹ together with R², and R¹⁰ together with R³, and/or R¹² together with R⁵, and R¹¹ together with R⁴, form a fused biannular structure according to any one of the following substructures:

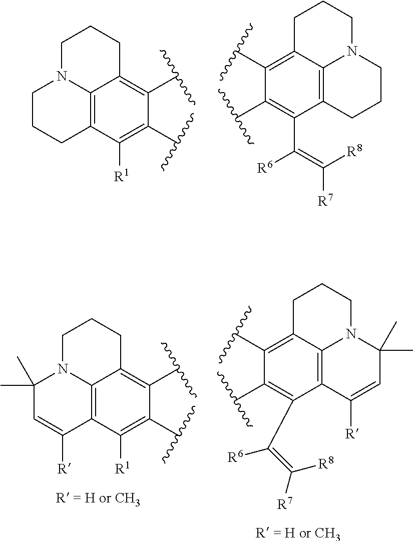

8. The compound according to claim 1, wherein R¹ is structurally identical to the substituent-CR⁶═CR⁷R⁸, in particular when the substituents R² and R⁵ are structurally identical, and/or the substituents-NR⁹R¹⁰ and —NR¹¹R¹² are structurally identical, and/or the substituents R³ and R⁴ are structurally identical;

9. The compound according to claim 1, wherein:
R¹ is H, and/or
R², R³, R⁴ and R⁵ are independently selected from H, halogen, CN, and/or
R⁹, R¹⁰, R¹¹ and R¹² are individually unsubstituted or amino-, hydroxyl- or halogen-substituted C₁ to C₄ alkyl, or C₃ to C₆ cycloalkyl, or Ro together with R¹⁰ together with the N atom to which they are bound, and R¹¹ together with R¹² together with the N atom to which they are bound form an unsubstituted or methyl-, hydroxy-, methoxy-, or halogen-substituted aziridine, azetidine, pyrrolidine, piperidine, piperazine, morpholine, thiomorpholine-S,S-dioxide, and/or
R¹³, R¹⁴, R¹⁵, if present, are selected from methyl, ethyl, isopropyl or phenyl,
R¹⁶, R¹⁷, if present, are methyl,
one of the substituents R⁶, R⁷, R⁸ and R²⁰, R²¹, R²², (if present, is selected from a) unsubstituted or amino-, hydroxyl-, carboxyl- and/or halogen-substituted C₂ to C₁₂ alkyl or C₃ to C₇ cycloalkyl; or b)-Lₘ^{A1}-L_{m′}^{J1}-Lₙ^{A2}, -L_{n′}^{J2}-Lₚ^{A3}-L_{p′}^{J3}-L_{q}^{A4}-L_{q′}^{J4}-Mₛ, wherein L^{A1}, L^{A2}, L^{A3}, L^{A4}, L^{J1}, L^{J2}, L^{J3}, L^{J4} m, m′, n, n′, p, p′, q, q′, s and M have the definitions recited above, and
the other substituents R⁶, R⁷, R⁸ and R²⁰, R²¹, R²², if present, are selected from H or methyl.

10. The compound according to claim 1, wherein the substituents —NR⁹R¹⁰ and/or —NR¹¹R¹² are represented by one of the following structures, particularly when the substituents —NR⁹R¹⁰ and —NR¹¹R¹² are structurally identical:

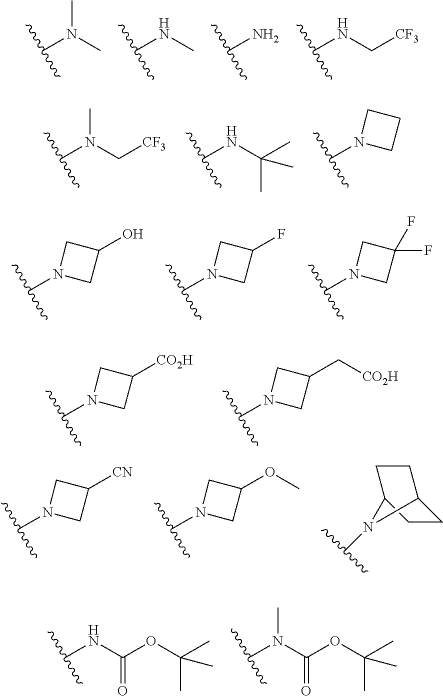

11. The compound according to claim 1, wherein the fragment
—CR⁶═CR⁷R⁸ is represented by one of the following structures:

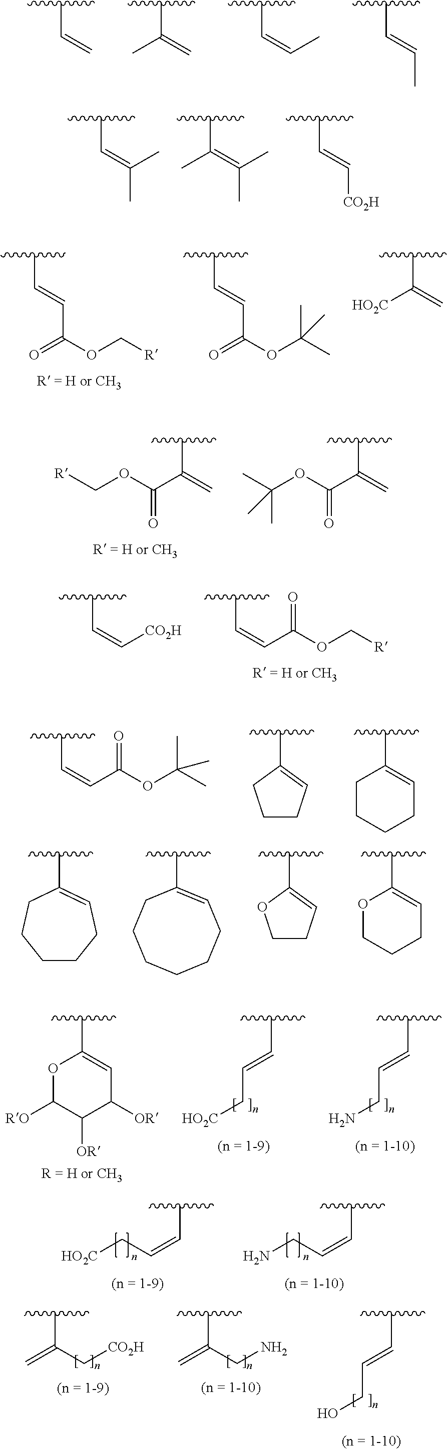

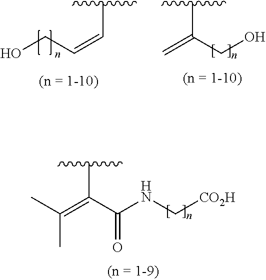

12. The compound according to claim 1, wherein the substituent
═Y is represented by one of the following structures:

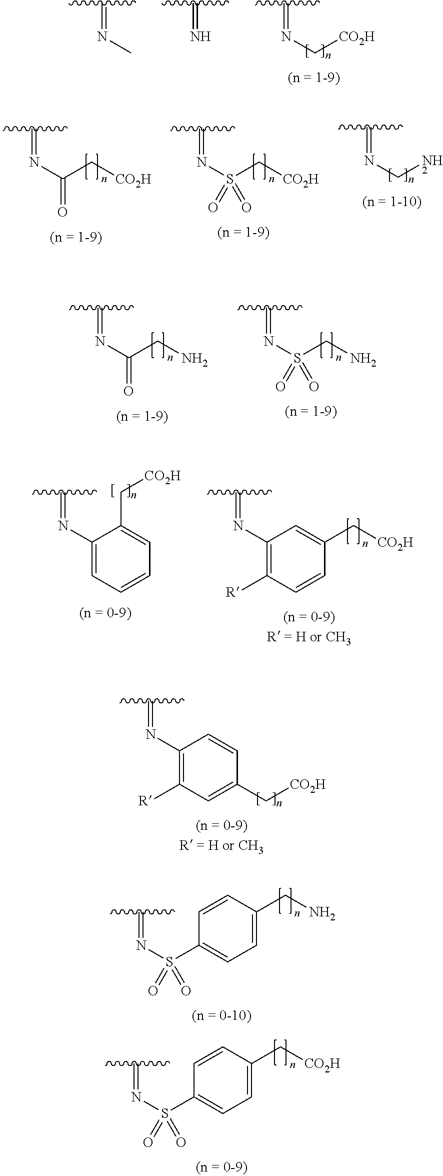

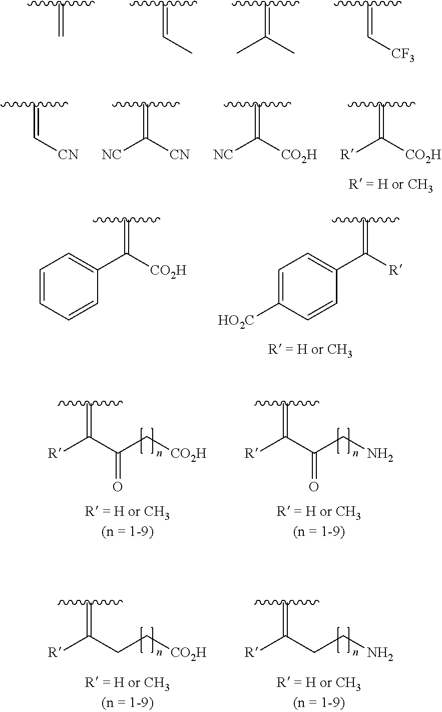

13. The compound according to any one of the preceding claim 1, wherein the group
—X— is represented by one of the following structures:

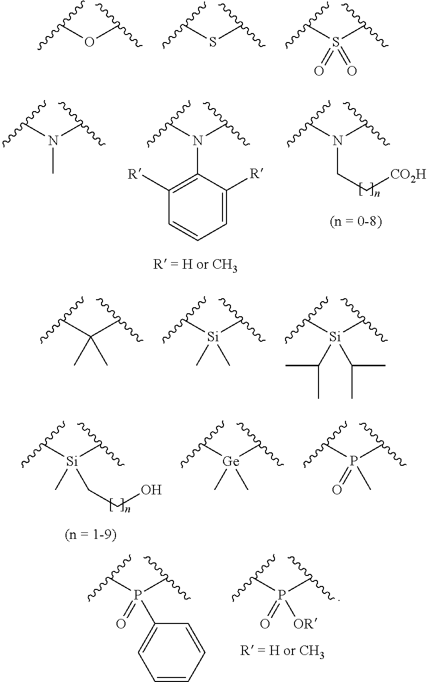

14. The compound according to claim 1 which is selected from the group of compounds below:

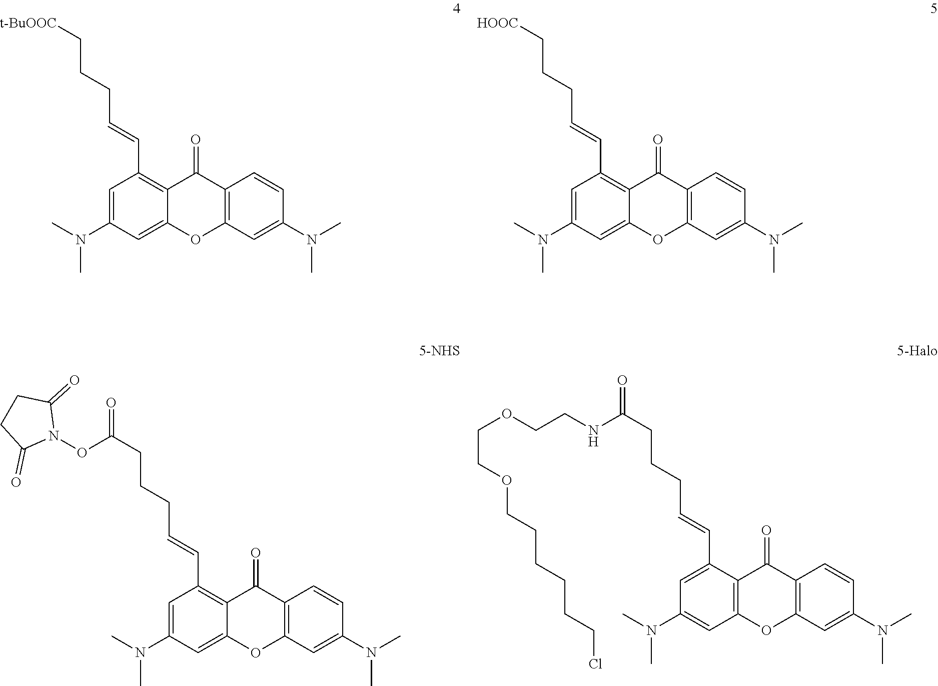

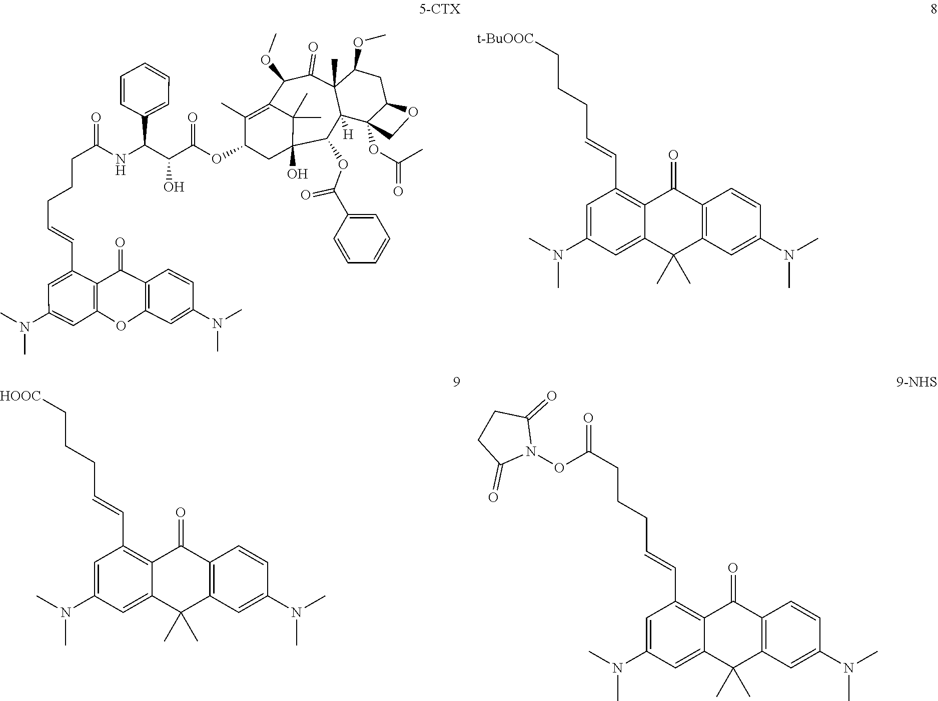

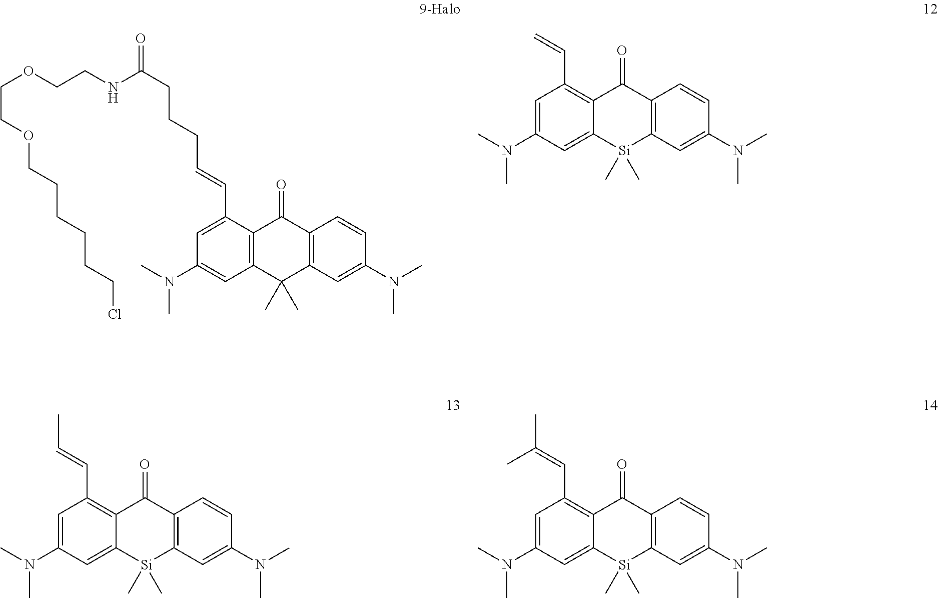

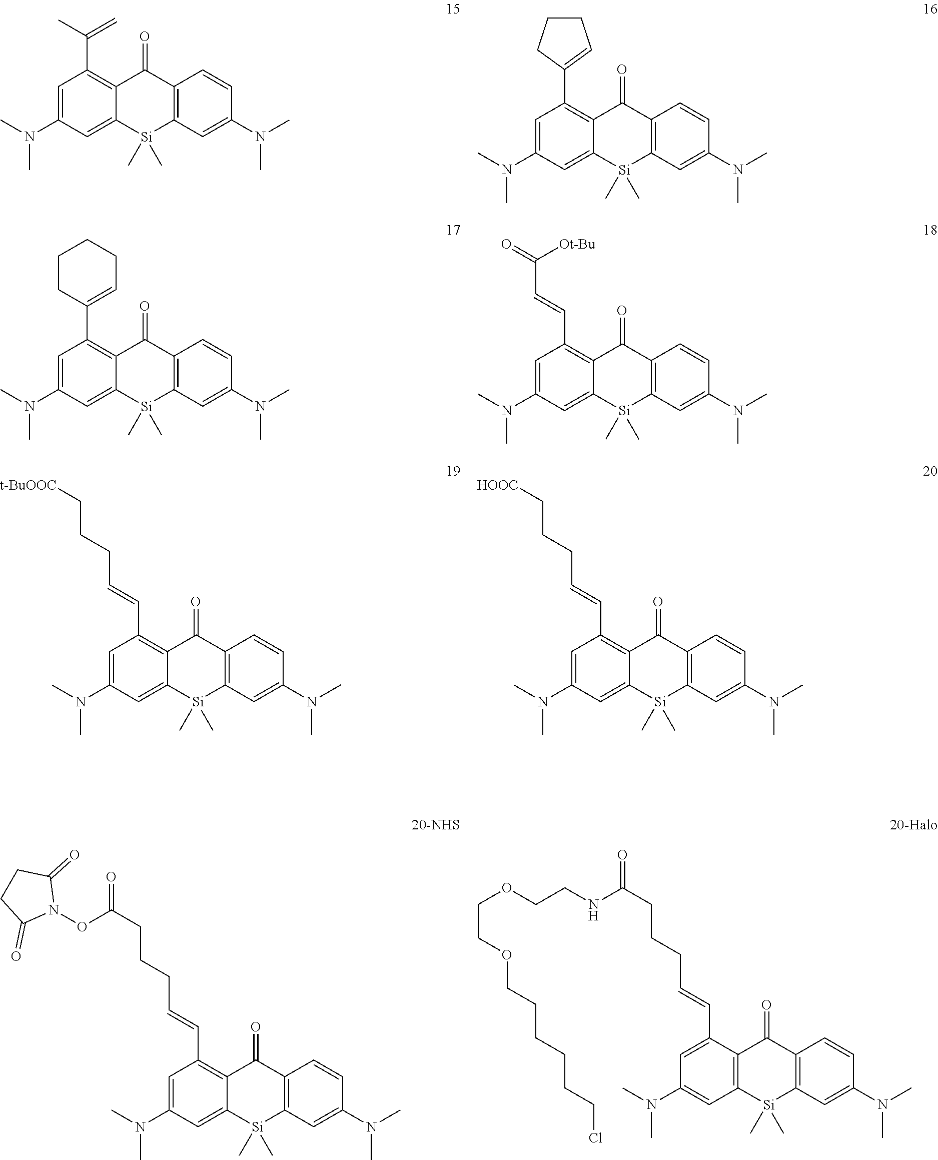

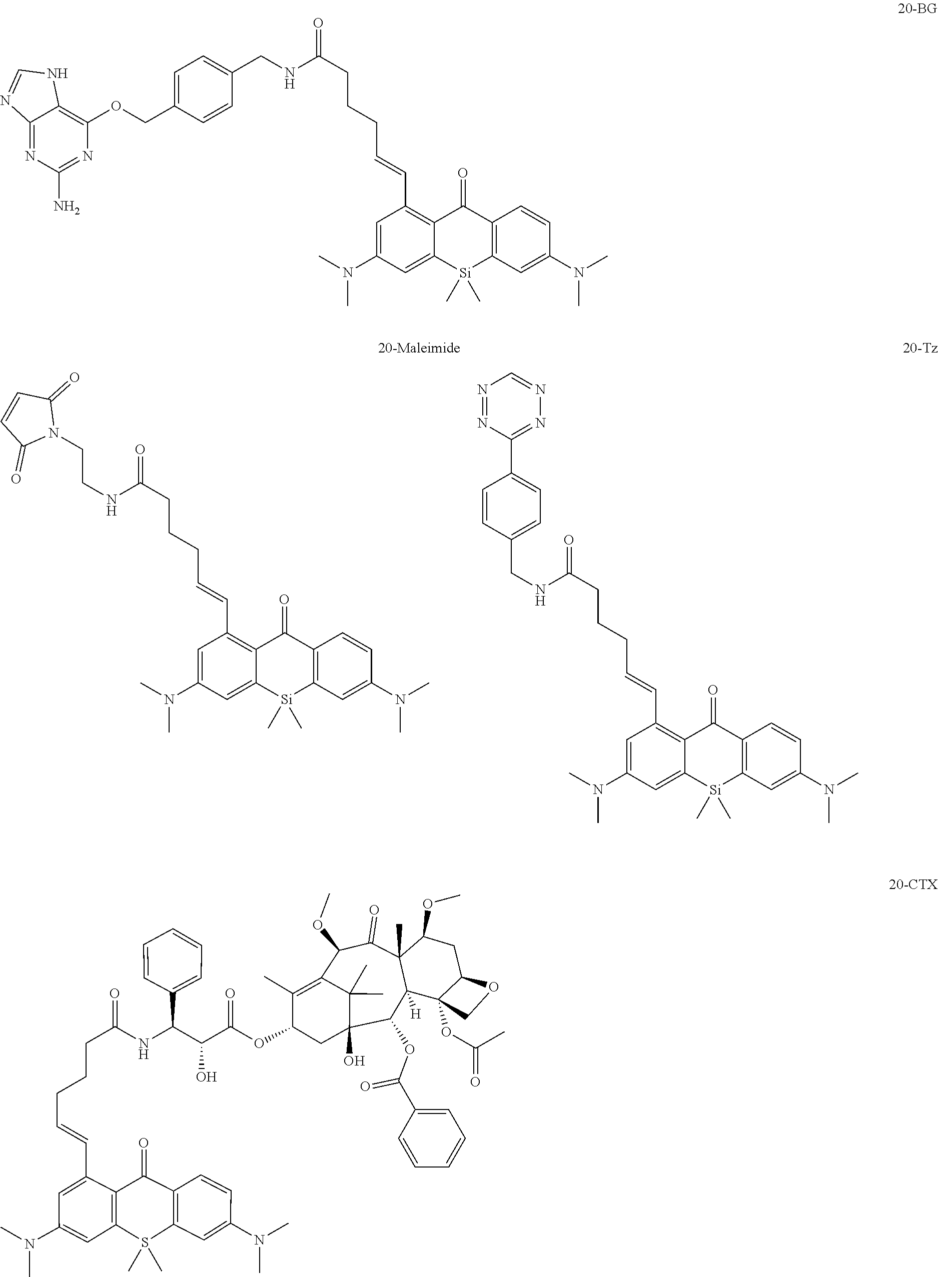

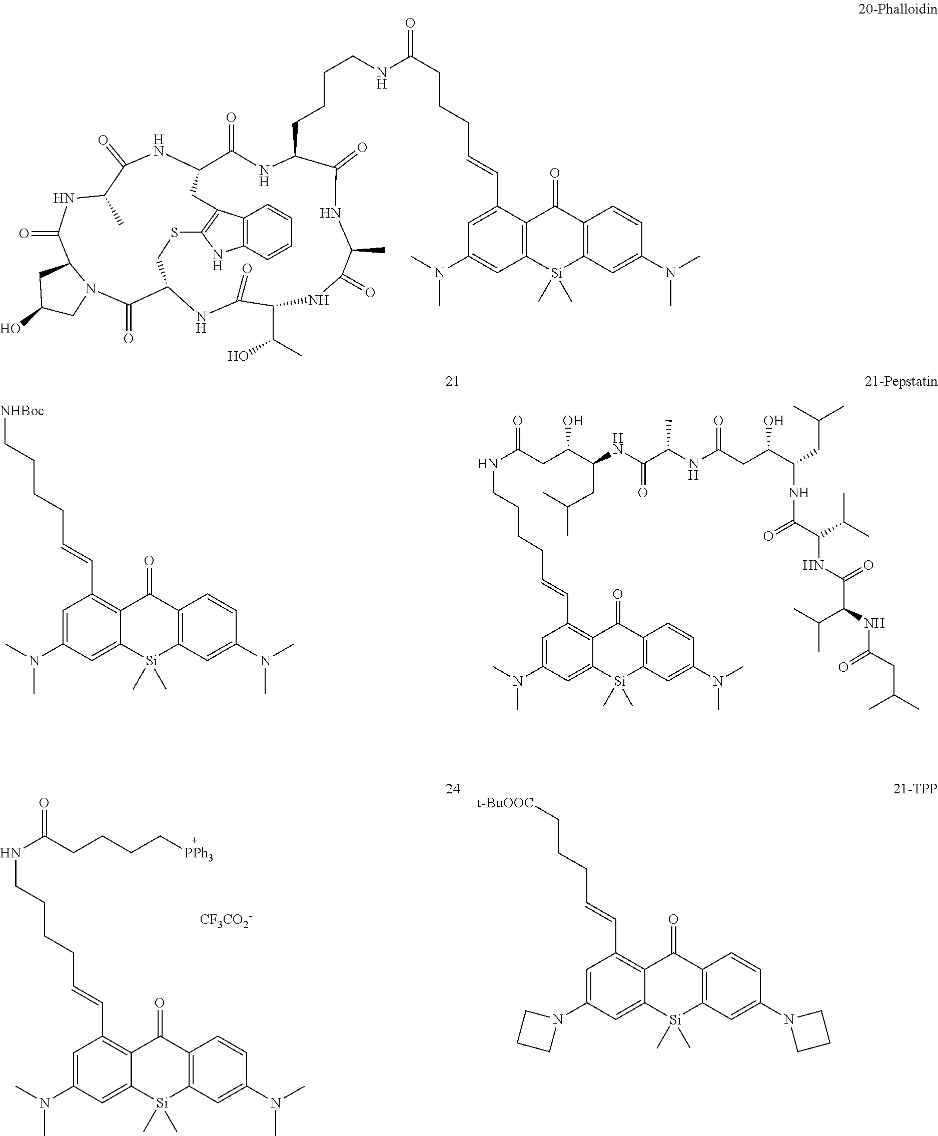

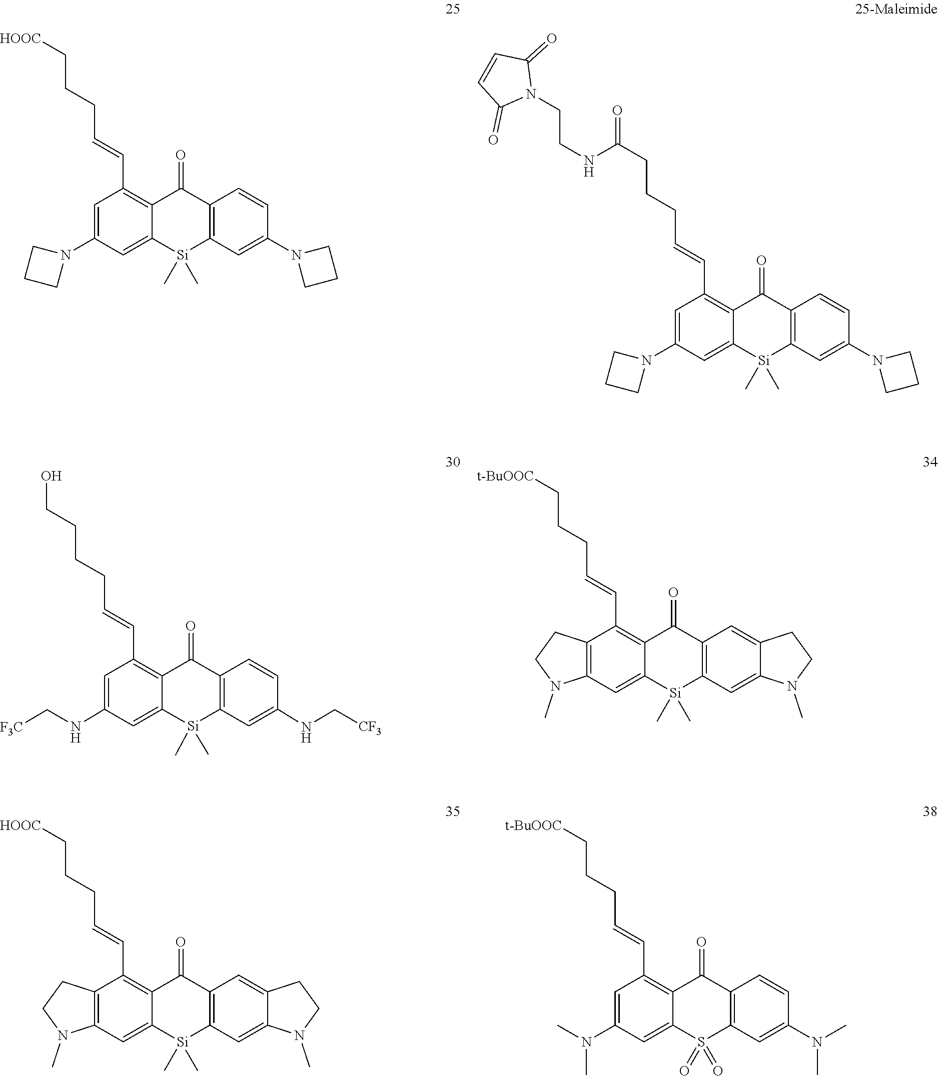

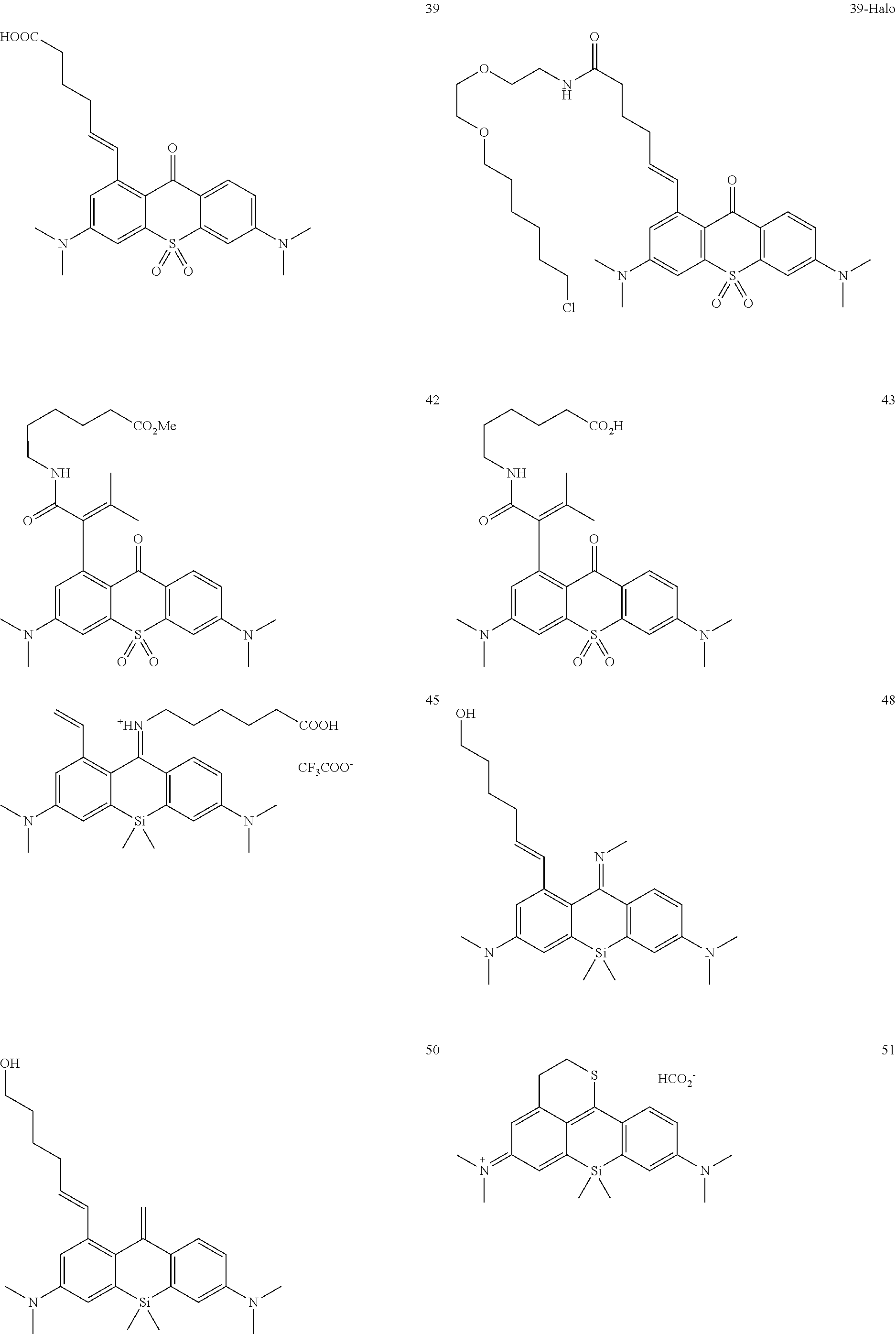

15. The compound of claim 1 in the form of a salt with organic or inorganic counterion(s), its cocrystal with another organic or inorganic compound(s), or a composition containing any of the dyes of claim 1.

16. A conjugate or bioconjugate comprising a compound according to claim 1 coupled via at least one covalent chemical bond or at least one molecular complex to a chemical entity or substance, such as amine, thiol, carboxylic acid, aldehyde, alcohol, aromatic compound, heterocycle, e.g. tetrazine, alkyne, alkene including strained and bicyclic alkenes, e. g. trans-cyclooctene, cyclopropene and norbornene derivatives, organic azide, dye, amino acid, amino acid residue coupled to any chemical entity, peptide, protein, in particular enzymes and immunoglobulins, antibody, single-domain antibody, carbohydrate including a carbohydrate residue attached to a protein, nucleic acid, toxin, lipid, virus, virus-like particle, biotin and its derivatives, a chemical tag, a recognition unit, etc.

17. A method of using the compounds or compositions according to claim 1 or of their conjugates as photoactivatable fluorescent dyes.

18. A method of using the compounds or compositions according to claim 1 in a method of staining a biological sample, in particular whole organisms, mammalian and non-mammalian cells including insect, plant, fungi, bacteria cells and viral particles.

19. A method of using the compounds or compositions according to claim 1 or of their conjugates as such or after photoactivation for tracking and monitoring dynamic processes in a sample or an object, or tracking and monitoring the behavior of single molecules within a sample or an object; in particular wherein changes in the shape, dimensions and/or the intensity of the fluorescence signal obtained after photoactivation of the compounds or compositions or of their conjugates correspond to changes of the sample or object or of its environment.

20. A method of using the compounds or compositions according to claim 1 as components in inorganic, bio-inorganic, organic or macromolecular composites as materials for optical memories, data storage, photo-lithography, photo-activatable paints and inks.

21. A method of using the compounds or compositions according to claim 1 or of their conjugates as such or after photoactivation as fluorescent tags, analytical reagents and labels in optical microscopy, imaging techniques, protein tracking, nucleic acid labeling, glycan analysis, capillary electrophoresis, flow cytometry or as a component of biosensors, or as analytical tools or reporters in microfluidic devices or nanofluidic circuitry.

22. The method according to claim 21, wherein the compounds, compositions or conjugates as such or after photoactivation are used as energy donors or acceptors (reporters) in applications based on fluorescence energy transfer (FRET) process or as energy acceptors (reporters) in applications based on bioluminescence resonance energy transfer (BRET) process.

23. The method according to claim 21, wherein the optical microscopy and imaging methods comprise single molecule switching techniques (SMS: diffraction unlimited optical resolution achieved by recording the fluorescence signals of single molecules, reversibly or irreversibly switched between emitting and non-emitting states, such as single molecule localization microscopy [SMLM], photoactivation localization microscopy [PALM, PALMIRA, fPALM], stochastic optical reconstruction microscopy [STORM], minimal photon fluxes [MINFLUX] or their parallelized implementations, fluorescence correlation spectroscopy [FCS], fluorescence recovery after photobleaching [FRAP], fluorescence lifetime imaging [FLIM], stimulated emission depletion microscopy [STED], including FastRESCue STED.

24. The method according to claim 21, wherein additional color multiplexing is achieved by using the compounds, compositions or conjugates as such or after photoactivation together with any other fluorescent dyes in a single sample or object under study, or
wherein the controlled photoactivation of spatiotemporal subpopulations of molecules of the compounds, compositions or conjugates allows imaging with the photoactivated fluorophore molecules while protecting the remaining photoactivatable fluorophores from photobleaching.
